(19) United States
(12) Patent Application Publication
(10) Pub. No.: US 2024/0042027 A1
(43) Pub. Date: Feb. 8, 2024

(54) CHIMERIC RECEPTORS AND METHODS OF USE THEREOF

(71) Applicant: Senti Biosciences, Inc., South San Francisco, CA (US)

(72) Inventors: Brian Scott GARRISON, San Jose, CA (US); Alba GONZALEZ-JUNCA, San Francisco, CA (US); Gary LEE, Castro Valley, CA (US); Assen Boyanov ROGUEV, San Francisco, CA (US); Frances Deen LIU, San Mateo, CA (US); Nicholas FRANKEL, San Francisco, CA (US)

(21) Appl. No.: 18/201,596

(22) Filed: May 24, 2023

Related U.S. Application Data
(63) Continuation of application No. PCT/US2021/060765, filed on Nov. 24, 2021.
(60) Provisional application No. 63/231,626, filed on Aug. 10, 2021.
(60) Provisional application No. 63/117,861, filed on Nov. 24, 2020.

(51) Int. Cl. A61K 39/00; A61P 35/00

(52) U.S. Cl. CPC A61K 39/4631; A61K 39/4611; A61K 39/4613; A61K 39/464482; A61P 35/00

(57) ABSTRACT

Provided herein are solid tumor antigen targets for chimeric receptors and chimeric inhibitory receptors, and methods of using the same, such as for the treatment of cancer.

### CROSS REFERENCE TO RELATED APPLICATIONS

This application is a continuation of International Application No. PCT/US2021/060765, filed Nov. 24, 2021, which claims the benefit of and priority to U.S. Provisional Application No. 63/231,626, filed Aug. 10, 2021, and to U.S. Provisional Application No. 63/117,861, filed Nov. 24, 2020, each of which are hereby incorporated by reference in their entireties for all purposes.

### SEQUENCE LISTING

The instant application contains a Sequence Listing which has been submitted in XML format and is hereby incorporated by reference in its entirety. Said Sequence Listing XML, created on Jun. 19, 2023, is named STB-023WOC1.xml, and is 248,980 bytes in size.

### FIELD OF THE INVENTION

The present application relates to solid tumor antigen targets for chimeric receptors and chimeric inhibitory receptors, and methods of using the same, such as for the treatment of cancer.

### BACKGROUND

Immunotherapies, such as chimeric antigen receptor (CAR) based adoptive cell therapies used to redirect the specificity and function of immunoresponsive cells (e.g., T cells and Natural Killer (NK) cells) have shown efficacy in patients with malignancies, with many prior studies focused on hematological malignancies (Pule et al., Nat. Med. (14):1264-1270 (2008); Maude et al., N Engl J Med. (371):1507-17 (2014); Brentjens et al., Sci Transl Med. (5):177ra38 (2013)). For example, CAR T cells have been shown to induce complete remission in patients with CD19-expressing malignancies for whom chemotherapies have led to drug resistance and tumor progression.

Unlike T cells, natural killer (NK) cells are able to eliminate abnormal cells, such as cancer cells, without priming. NK cell activity is determined by a balance of external signals from inhibitory and activating NK cell receptors. Inhibitory receptors, such as killer immunoglobulin receptors (KIRs), interact with the major histocompatibility complex (MHC) class I antigens and protect normal cells from NK cell activity (see US20180057795A1).

One challenge to developing CAR therapy for solid tumors is a lack of suitable targets. The ability to identify appropriate CAR targets is important to effectively targeting and treating the tumor without damaging normal cells that express the same target antigen. Thus, there remains a need for CAR-NK cell-based tumor therapies that target tumor cells without targeting normal cells or tissues.

### SUMMARY

In one aspect, provided herein is an isolated cell comprising: (a) an inhibitory chimeric receptor comprising an extracellular antigen-binding domain that binds to an antigen, wherein the antigen is selected from the group consisting of: VSIG2, CPM, ITM2C, SLC26A2, SLC4A4, GPA33, PLA2G2A, ABCA8, ATP1A2, CHP2, and SLC26A3; and (b) a chimeric receptor comprising one or more extracellular antigen-binding domains, wherein the one or more extracellular antigen-binding domains bind one or more additional antigens selected from the group consisting of: CEA, CEACAM1, CEACAM5, and CEACAM6.

In some aspects, the antigen-binding domain of the inhibitory chimeric receptor comprises one or more single chain variable fragments (scFvs), optionally wherein each of the one or more scFvs comprises a heavy chain variable domain (VH) and a light chain variable domain (VL), optionally wherein the VH and VL are separated by a peptide linker, optionally the peptide linker comprises the amino acid sequence of SEQ ID NO: 39 or SEQ ID NO: 77, and/or optionally wherein each of the one or more scFvs comprises the structure VH-L-VL or VL-L-VH, wherein VH is the heavy chain variable domain, L is the peptide linker, and VL is the light chain variable domain.

In some aspects, the inhibitory chimeric receptor comprises a transmembrane domain, and the transmembrane domain is selected from the group consisting of: a CD8 transmembrane domain, a CD28 transmembrane domain, a CD25 transmembrane domain, a CD7 transmembrane domain, a CD3zeta-chain transmembrane domain, a CD4 transmembrane domain, a 4-1BB transmembrane domain, an OX40 transmembrane domain, an ICOS transmembrane domain, a CTLA-4 transmembrane domain, a LAX transmembrane domain, a LAT transmembrane domain, a PD-1 transmembrane domain, a LAG-3 transmembrane domain, a TIM3 transmembrane domain, a KIR3DS1 transmembrane domain, a KIR3DL1 transmembrane domain, an NKG2D transmembrane domain, an NKG2A transmembrane domain, a TIGIT transmembrane domain, a 2B4 transmembrane domain, and a BTLA transmembrane domain.

In some aspects, the inhibitory chimeric receptor comprises a spacer region between the antigen-binding domain and the transmembrane domain, optionally wherein the spacer region has an amino acid sequence selected from the group consisting of SEQ ID NOs: 49-58; and/or

In some aspects, the inhibitory chimeric receptor comprises one or more intracellular inhibitory domains selected from the group consisting of: PD-1, CTLA4, TIGIT, LAIR1, GRB-2, Dok-1, Dok-2, SLAP, LAG3, HAVR, BTLA, LIR1, NKG2A, KIR3DL1, GITR, PD-L1, CSK, SHP-1, PTEN, CD45, CD148, PTP-MEG1, PTP-PEST, c-CBL, CBL-b, PTPN22, LAR, PTPH1, SHIP-1, RasGAP, CD94, and CD161.

In some aspects, the chimeric receptor is a CAR. In some aspects, the CAR comprises one or more intracellular signaling domains, and the one or more intracellular signaling domains are selected from the group consisting of: a CD3zeta-chain intracellular signaling domain, a CD3epsilon-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, an NKp46 intracellular signaling domain, an NKp30 intracellular signaling domain, an NKp44 intracellular signaling domain, an NKG2D intracellular signaling domain, a CD226 intracellular signaling domain, and a CD160 intracellular signaling domain. In some aspects, the CAR comprises a transmembrane domain, and the transmembrane domain is selected from the group consisting of: a CD8 transmembrane domain, a CD28 transmembrane domain, a CD25 transmembrane domain, a CD7 transmembrane domain, a CD3zeta-chain transmembrane domain, a CD4 transmembrane domain, a 4-1BB transmembrane domain, an OX40 transmembrane domain, an ICOS transmembrane domain, a CTLA-4 transmembrane domain, a LAX transmembrane domain, a LAT transmembrane domain, a PD-1 transmembrane domain, a LAG-3 transmembrane domain, a TIM3 transmembrane domain, a KIR3DS1 transmembrane domain, a KIR3DL1 transmembrane domain, an NKG2D transmembrane domain, an NKG2A transmembrane domain, a TIGIT transmembrane domain, a 2B4 transmembrane domain, and a BTLA transmembrane domain.

In some aspects, the CAR comprises a spacer region between the antigen-binding domain and the transmembrane domain, and the spacer region has an amino acid sequence selected from the group consisting of SEQ ID NOs: 49-58.

In some aspects, the antigen-binding domain of the inhibitory chimeric receptor and/or the chimeric receptor comprises one or more single chain variable fragments (scFvs), optionally wherein each of the one or more scFvs comprises a heavy chain variable domain (VH) and a light chain variable domain (VL), optionally wherein the VH and VL are separated by a peptide linker, optionally wherein the peptide linker comprises the amino acid sequence of SEQ ID NO: 39 or SEQ ID NO: 77.

In some aspects, each of the one or more scFvs comprises the structure VH-L-VL or VL-L-VH, wherein VH is the heavy chain variable domain, L is the peptide linker, and VL is the light chain variable domain.

In some aspects, the one or more additional antigen is CECAM1, optionally wherein the antigen-binding domain that binds to CEACAM1 comprises a heavy chain variable domain (VH) comprising the amino acid sequence of SEQ ID NO: 1 and a light chain variable domain (VL) comprising the amino acid sequence of SEQ ID NO: 2.

In some aspects, the one or more additional antigen is CECAM5, optionally wherein the antigen-binding domain that binds to CEACAM5 comprises a heavy chain variable domain (VH) and a light chain variable domain (VL) selected from the group consisting of:
(i) a VH comprising the amino acid sequence of SEQ ID NO: 3 and a VL comprising the amino acid sequence of SEQ. ID NO: 4;
(ii) a VH comprising the amino acid sequence of SEQ ID NO: 9 and a VL comprising the amino acid sequence of SEQ. ID NO: 10;
(iii) a VH comprising the amino acid sequence of SEQ ID NO: 11 and a VL comprising the amino acid sequence of SEQ. ID NO: 12;
(iv) a VH comprising the amino acid sequence of SEQ ID NO: 13 and a VL comprising the amino acid sequence of SEQ. ID NO: 14;
(v) a VH comprising the amino acid sequence of SEQ ID NO: 78 and a VL comprising the amino acid sequence of SEQ. ID NO: 79; and
(vi) a VH comprising the amino acid sequence of SEQ ID NO: 15 and a VL comprising the amino acid sequence of SEQ. ID NO: 16.

In some aspects, the antigen-binding domain that binds to CEACAM5 comprises a heavy chain (HC) and a light chain (LC) selected from the group consisting of:
(i) an HC comprising the amino acid sequence of SEQ ID NO: 5 and an LC comprising the amino acid sequence of SEQ. ID NO: 6; and
(ii) an HC comprising the amino acid sequence of SEQ ID NO: 7 and an LC comprising the amino acid sequence of SEQ. ID NO: 8; or
(c) the one or more additional antigen is CECAM6, optionally wherein the antigen-binding domain that binds to CEACAM6 comprises a heavy chain variable domain (VH) comprising the amino acid sequence of SEQ ID NO: 17 and a light chain variable domain (VL) comprising the amino acid sequence of SEQ ID NO 18.

In some aspects, the cell is an immunoresponsive cell. In some aspects, binding of the inhibitory chimeric receptor to the antigen is capable of inhibiting the immunoresponsive cell and/or wherein binding of the chimeric receptor to the one or more additional antigens is capable of activating the immunoresponsive cell.

In some aspects, the chimeric receptor binds to the one or more additional antigens with a low binding affinity.

In some aspects, the chimeric receptor binds to the one or more additional antigens with a binding affinity that is lower than the binding affinity with which the inhibitory chimeric receptor binds to the antigen.

In some aspects, the inhibitory chimeric receptor binds to the antigen with a low binding avidity.

In some aspects, the chimeric receptor is recombinantly expressed, optionally wherein the chimeric receptor is expressed from a vector or a selected locus from the genome of the cell.

In some aspects, the cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell. In some aspects, the cell is autologous or the cell is allogeneic.

In some aspects, provided herein is an isolated nucleic acid encoding an inhibitory chimeric receptor as provided herein.

In some aspects, provided herein is a vector comprising a nucleic acid as provided herein.

In some aspects, provided herein is a genetically modified cell comprising a nucleic acid as provided herein or vector as provided herein.

In some aspects, provided herein is a pharmaceutical composition comprising an effective amount of an isolated cell as provided herein, an isolated nucleic acid as provided herein, or a vector as provided herein, and a pharmaceutically acceptable carrier, a pharmaceutically acceptable excipient, or a combination thereof.

In some aspects, provided herein is a method of treating a subject in need thereof, the method comprising administering to the subject an effective amount of isolated cells as provided herein, an isolated nucleic acid as provided herein, a vector as provided herein, or the pharmaceutical composition as provided herein.

In some aspects, provided herein is a method of stimulating a cell-mediated immune response to a tumor cell in a subject, the method comprising administering to a subject having a tumor an effective amount of isolated cells as provided herein, an isolated nucleic acid as provided herein, a vector as provided herein, or a pharmaceutical composition as provided herein.

In some aspects, provided herein is a method of providing an anti-tumor immunity in a subject, the method comprising administering to a subject in need thereof an effective amount of isolated cells as provided herein, an isolated nucleic acid as provided herein, a vector as provided herein, or a pharmaceutical composition as provided herein.

In some aspects, provided herein is a method of reducing tumor burden in a subject, comprising administering to the subject an effective amount of isolated cells as provided herein, an isolated nucleic acid as provided herein, a vector as provided herein, or a pharmaceutical composition as provided herein. In some aspects, the method reduces the number of tumor cells, the method reduces tumor size, the method reduces tumor volume, and/or the method eradicates the tumor in the subject.

In some aspects, provided herein is a method of treating a subject having a tumor, the method comprising administering an effective amount of isolated cells as provided herein, an isolated nucleic acid as provided herein, a vector as provided herein, or a pharmaceutical composition as provided herein.

In some aspects, provided herein is a method of treating or preventing a cancer in a subject, wherein the cancer is selected from the group consisting of: colorectal carcinoma, pancreatic cancer, a lung adenocarcinoma, and gastric cancer, the method comprising administering to the subject an effective amount of the isolated cells as provided herein, an isolated nucleic acid as provided herein, a vector as provided herein, or a pharmaceutical composition as provided herein.

In some aspects, the method increases progression free survival in the subject; and or the method increases survival in the subject.

In some aspects, provided herein is a kit for treating and/or preventing a colorectal carcinoma, a pancreatic cancer, a lung adenocarcinoma, and/or a gastric cancer, comprising the isolated cells as provided herein, an isolated nucleic acid as provided herein, a vector as provided herein, or a pharmaceutical composition as provided herein. In some aspects, the kit further comprises written instructions for using the isolated cell or pharmaceutical composition for treating and/or preventing a colorectal carcinoma, a pancreatic cancer, a lung adenocarcinoma, and/or a gastric cancer in a subject, or the kit further comprises instructions for using the nucleic acid or vector for producing one or more antigen-specific cells for treating and/or preventing a colorectal carcinoma, a pancreatic cancer, a lung adenocarcinoma, and/or a gastric cancer in a subject.

In some aspects, provided herein is an inhibitory chimeric receptor comprising an extracellular antigen-binding domain that binds to an antigen selected from the group consisting of: VSIG2, CPM, ITM2C, SLC26A2, SLC4A4, GPA33, PLA2G2A, ABCA8, ATP1A2, CHP2, and SLC26A3.

In some embodiments, the antigen is VSIG2. In some embodiments, the antigen is CPM. In some embodiments, the antigen is ITM2C. In some embodiments, the antigen is SLC26A2. In some embodiments, the antigen is SLC4A4. In some embodiments, the antigen is GPA33. In some embodiments, the antigen is PLA2G2A. In some embodiments, the antigen is ABCA8. In some embodiments, the antigen is ATP1A2. In some embodiments, the antigen is CHP2. In some embodiments, the antigen is SLC26A3.

In some embodiments, when expressed on a cell, the inhibitory chimeric receptor inhibits one or more activities of the cell.

In some embodiments, the antigen is not expressed on a tumor cell, or the antigen is expressed on a tumor cell at a level that is lower than expression on a non-tumor cell.

In some embodiments, the antigen is expressed on a non-tumor cell, or the antigen is expressed on a non-tumor cell at a level that is higher than expression on a corresponding tumor cell.

In some embodiments, the antigen is expressed on a non-tumor cell derived from a tissue selected from the group consisting of: lung, pancreas, gastrointestinal tract, colon, brain, neuronal tissue, endocrine, bone, bone marrow, immune system, muscle, liver, gallbladder, pancreas, kidney, urinary bladder, male reproductive organs, female reproductive organs, adipose, soft tissue, and skin.

In some embodiments, the inhibitory chimeric receptor comprises one or more intracellular inhibitory domains selected from the group consisting of: PD-1, CTLA4, TIGIT, LAIR1, GRB-2, Dok-1, Dok-2, SLAP, LAG3, HAVR, BTLA, LIR1, NKG2A, KIR3DL1, GITR, PD-L1, CSK, SHP-1, PTEN, CD45, CD148, PTP-MEG1, PTP-PEST, c-CBL, CBL-b, PTPN22, LAR, PTPH1, SHIP-1, RasGAP, CD94, and CD161.

In some embodiments, the antigen-binding domain comprises one or more antibodies, antigen-binding fragments of an antibody, F(ab) fragments, F(ab′) fragments, single chain variable fragments (scFvs), or single-domain antibodies (sdAbs).

In some embodiments, the antigen-binding domain comprises one or more single chain variable fragments (scFvs). In some embodiments, each of the one or more scFvs comprises a heavy chain variable domain (VH) and a light chain variable domain (VL). In some embodiments, the VH and VL are separated by a peptide linker. In some embodiments, the peptide linker comprises the amino acid sequence of SEQ ID NO: 39 or SEQ ID NO: 77. In some embodiments, each of the one or more scFvs comprises the structure VH-L-VL or VL-L-VH, wherein VH is the heavy chain variable domain, L is the peptide linker, and VL is the light chain variable domain.

In another aspect, provided herein is an isolated cell comprising the inhibitory chimeric receptor of any one of the above embodiments. In some embodiments, the inhibitory chimeric receptor is recombinantly expressed. In some embodiments, the inhibitory chimeric receptor is expressed from a vector or a selected locus from the genome of the cell. In some embodiments, the cell further comprises a chimeric receptor comprising one or more extracellular antigen-binding domains, wherein the one or more extracellular antigen-binding domains bind one or more antigens selected from the group consisting of: CEA, CEACAM1, CEACAM5, and CEACAM6.

In another aspect, provided herein is an isolated cell comprising:
(a) an inhibitory chimeric receptor comprising an extracellular antigen-binding domain that binds to an antigen, wherein the antigen is selected from the group consisting of: VSIG2, CPM, ITM2C, SLC26A2, SLC4A4, GPA33, PLA2G2A, ABCA8, ATP1A2, CHP2, and SLC26A3; and
(b) a chimeric receptor comprising one or more extracellular antigen-binding domains, wherein the one or more extracellular antigen-binding domains bind one or more additional antigens selected from the group consisting of: CEA, CEACAM1, CEACAM5, and CEACAM6.

In some embodiments, the chimeric receptor is a chimeric T cell receptor or a chimeric antigen receptor (CAR). In some embodiments, the chimeric receptor is a CAR. In some embodiments, the CAR comprises one or more intracellular signaling domains, and the one or more intracellular signaling domains are selected from the group consisting of a CD3zeta-chain intracellular signaling domain, a CD3epsilon-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, an NKp46 intracellular signaling domain, an NKp30 intracellular signaling domain, an NKp44 intracellular signaling domain, an NKG2D intracellular signaling domain, a CD226 intracellular signaling domain, and a CD160 intracellular signaling domain.

In some embodiments, the CAR comprises a transmembrane domain, and the transmembrane domain is selected from the group consisting of a CD8 transmembrane domain, a CD28 transmembrane domain, a CD25 transmembrane domain, a CD7 transmembrane domain, a CD3 zeta-chain transmembrane domain, a CD4 transmembrane domain, a 4-1BB transmembrane domain, an OX40 transmembrane domain, an ICOS transmembrane domain, a CTLA-4 transmembrane domain, a LAX transmembrane domain, a LAT transmembrane domain, a PD-1 transmembrane domain, a LAG-3 transmembrane domain, a TIM3 transmembrane domain, a KIR3DS1 transmembrane domain, a KIR3DL1 transmembrane domain, an NKG2D transmembrane domain, an NKG2A transmembrane domain, a TIGIT transmembrane domain, a 2B4 transmembrane domain, and a BTLA transmembrane domain.

In some embodiments, the CAR comprises a spacer region between the antigen-binding domain and the transmembrane domain, and the spacer region has an amino acid sequence selected from the group consisting of SEQ ID NOs: 49-58.

In some embodiments, the antigen-binding domain of the inhibitory chimeric receptor and/or the chimeric receptor comprises one or more antibodies, antigen-binding fragments of an antibody, F(ab) fragments, F(ab′) fragments, single chain variable fragments (scFvs), or single-domain antibodies (sdAbs).

In some embodiments, the antigen-binding domain of the inhibitory chimeric receptor and/or the chimeric receptor comprises one or more single chain variable fragments (scFvs). In some embodiments, each of the one or more scFvs comprises a heavy chain variable domain (VH) and a light chain variable domain (VL). In some embodiments, the VH and VL are separated by a peptide linker. In some embodiments, the peptide linker comprises the amino acid sequence of SEQ ID NO: 39 or SEQ ID NO: 77. In some embodiments, each of the one or more scFvs comprises the structure VH-L-VL or VL-L-VH, wherein VH is the heavy chain variable domain, L is the peptide linker, and VL is the light chain variable domain.

In some embodiments, the cell is an immunoresponsive cell. In some embodiments, binding of the inhibitory chimeric receptor to the antigen is capable of inhibiting the immunoresponsive cell. In some embodiments, binding of the chimeric receptor to the one or more additional antigens is capable of activating the immunoresponsive cell.

In some embodiments, the chimeric receptor binds to the one or more additional antigens with a low binding affinity.

In some embodiments, the chimeric receptor binds to the one or more additional antigens with a binding affinity that is lower than the binding affinity with which the inhibitory chimeric receptor binds to the antigen. In some embodiments, the inhibitory chimeric receptor binds to the antigen with a low binding avidity.

In some embodiments, the chimeric receptor is recombinantly expressed. In some embodiments, the chimeric receptor is expressed from a vector or a selected locus from the genome of the cell.

In some embodiments, the cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell.

In some embodiments, the cell is autologous. In some embodiments, the cell is allogeneic.

In another aspect, provided herein is an isolated nucleic acid encoding the inhibitory chimeric receptor of any one of the above inhibitory chimeric receptors.

In another aspect, provided herein is a vector comprising the nucleic acid of the above aspect.

In another aspect, provided herein is a genetically modified cell comprising the nucleic acid or the vector of the above aspects.

In another aspect, provided herein is a method of treating a subject in need thereof, the method comprising administering the isolated cells of any one of the above embodiments.

In another aspect, provided herein is a method of stimulating a cell-mediated immune response to a tumor cell in a subject, the method comprising administering to a subject having a tumor the isolated cells of any one of the above embodiments.

In another aspect, provided herein is a method of providing an anti-tumor immunity in a subject, the method comprising administering to a subject in need thereof the isolated cells of any one of the above embodiments.

In another aspect, provided herein is a method of reducing tumor burden in a subject, comprising administering to the subject the isolated cells of any one of the above embodiments. In some embodiments, the method reduces the number of tumor cells. In some embodiments, the method reduces tumor size. In some embodiments, the method reduces tumor volume. In some embodiments, the method eradicates the tumor in the subject.

In another aspect, provided herein is a method of treating a subject having a tumor, the method comprising administering the isolated cells of any one of the above embodiments.

In another aspect, provided herein is a method of treating or preventing a colorectal carcinoma in a subject, comprising administering to the subject the isolated cells of any one of the above embodiments.

In another aspect, provided herein is a method of treating or preventing pancreatic cancer in a subject, comprising administering to the subject the isolated cells of any one of the above embodiments.

In another aspect, provided herein is a method of treating or preventing a lung adenocarcinoma in a subject, comprising administering to the subject the isolated cells of any one of the above embodiments.

In another aspect, provided herein is a method of treating or preventing gastric cancer in a subject, comprising administering to the subject the isolated cells of any one of the above embodiments.

In some embodiments of the above aspects, the isolated cells are administered at an effective amount. In some embodiments of the above aspects, the method increases progression free survival in the subject. In some embodiments of the above aspects, the method increases survival in the subject.

In another aspect, provided herein is a pharmaceutical composition comprising an effective amount of the isolated cell of any one of the above embodiments, and a pharmaceutically acceptable carrier, a pharmaceutically acceptable excipient, or a combination thereof. In some embodiments, the pharmaceutical composition is for treating and/or preventing a colorectal carcinoma, a pancreatic cancer, a lung adenocarcinoma, and/or a gastric cancer.

In another aspect, provided herein is a kit for treating and/or preventing a colorectal carcinoma, a pancreatic cancer, a lung adenocarcinoma, and/or a gastric cancer, comprising the isolated cell of any one of the above embodiments, or the pharmaceutical composition of the above aspect. In some embodiments, the kit further comprises written instructions for using the isolated cell for treating and/or preventing a colorectal carcinoma, a pancreatic cancer, a lung adenocarcinoma, and/or a gastric cancer in a subject.

In another aspect, provided herein is a kit for treating and/or preventing a colorectal carcinoma, a pancreatic cancer, a lung adenocarcinoma, and/or a gastric cancer, comprising the isolated nucleic acid or the vector of the above aspects. In some embodiments, the kit further comprises written instructions for using the nucleic acid for producing one or more antigen-specific cells for treating and/or preventing a colorectal carcinoma, a pancreatic cancer, a lung adenocarcinoma, and/or a gastric cancer in a subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present disclosure will become better understood with regard to the following description, and accompanying drawings.

FIG. 1 depicts a schematic representation of CEA-family members, their structure, and functions.

FIG. 2 depicts gene expression of CEACAM5 and CEACAM6 in colorectal carcinoma (CRC) and normal tissues.

FIG. 3A depicts gene expression of CEACAM5 in colorectal carcinoma (CRC) and normal tissues. FIG. 3B depicts gene expression of CEACAM6 in colorectal carcinoma (CRC) and normal tissues.

FIG. 4 depicts protein expression of CEACAM1, CEACAM5, and CEACAM6 in patient samples in a tissue microarray including both cancer and normal tissue samples.

FIG. 5 depicts the flow cytometry based analysis of CEACAM1, CEACAM5, and CEACAM6 expression in a panel of human colon carcinoma cell lines obtained from ATCC.

FIG. 6A depicts analysis of NOT targets using bulk RNAseq gene expression data for ABCA8. FIG. 6B depicts analysis of NOT targets using bulk RNAseq gene expression data for ATP1A2.

FIG. 7A depicts analysis of the single cell RNAseq dataset GSE144735 after dimensionality reduction and clustering, with different cell types annotated and colored. FIG. 7B depicts expression of ABCA8 in normal tissue (FIG. 7A), with highlighted expression in stromal cells.

FIG. 8 depicts gene expression of VSIG2 in colorectal carcinoma (CRC) and normal tissues.

FIG. 9A depicts gene expression analysis of VSIG2, showing expression in the epithelial cells in normal and tumor tissue in single cell RNAseq dataset GSE144735. FIG. 9B depicts gene expression analysis of VSIG2, showing overall gene expression in normal and tumor tissue in single cell RNAseq dataset GSE132465.

FIG. 10A depicts comparative gene expression analysis of VSIG2 and CEACAM5. FIG. 10B depicts comparative gene expression analysis of VSIG2 and CEACAM5, presented as the relative fraction for each population. FIG. 10C depicts comparative gene expression analysis of VSIG2 and CEACAM5 in normal gastrointestinal tissue (ileum, colon, and rectum). FIG. 10D depicts comparative gene expression analysis of VSIG2 and CEACAM5 in normal lung epithelial tissue. FIG. 10E depict comparative gene expression analysis of VSIG2 and CEACAM5 in normal kidney and liver tissue.

FIG. 11 depicts expression analysis of potential NOT target VSIG2 expression across a range of colon normal and cancer subtypes (datasets: GSE81861, GSE144735, GSE132465).

FIG. 12A depicts VSIG2 expression in healthy lung epithelial cells (EGAS00001004344). FIG. 12B compares expression of VSIG2 and CEACAM5 and CEACAM6 in healthy lung epithelial cells.

FIG. 13A depicts tissue microarrays (TMA) of gastrointestinal tumor and healthy gastrointestinal tissue stained for VSIG2. FIG. 13B is a representative healthy tissue sample of the TMA stained for VSIG2. FIG. 13C is a representative healthy tissue sample of the TMA stained for VSIG2.

FIG. 14A depicts multiplexed IHC analysis of healthy tissue stained for VSIG2 (pink) and CEACAM5 (white). Nuclei are stained with DAPI (blue). FIG. 14B depicts multiplexed IHC analysis of healthy tissue stained for VSIG2 (pink) and CEACAM5 (white). Nuclei are stained with DAPI (blue).

FIG. 15 depicts localization analysis of VSIG2 (pink) and CEACAM5 (white) in grade 3 stage IIB colon carcinoma. Nuclei are stained with DAPI (blue).

FIG. 16A depicts gene expression analysis of VSIG2 and CEACAM5 in stromal and immune cells in colon carcinoma samples from two cohorts. FIG. 16B depicts gene expression analysis of VSIG2 and CEACAM5 in stromal and immune cells in colon carcinoma samples from two cohorts. FIG. 16C depicts gene expression analysis of VSIG2 and CEACAM5 in stromal and immune cells in colon carcinoma samples from two cohorts.

FIG. 17A depicts gene expression analysis of CPM in colon cancer and normal tissue (datasets: GSE81861, GSE144735, GSE132465). FIG. 17B depicts comparative expression of CPM and CEACAM5 and CEACAM6 in lung normal tissue (dataset EGAS00001004344). FIG. 17C depicts comparative expression of CPM and CEACAM5 and CEACAM6 in healthy lung cells (dataset EGAS00001004344).

FIG. 18A FIG. 18B and depict expression analysis of potential NOT target GPA33. FIG. 18A depicts GPA33 expression across a range of normal and CRC cellular subtypes (datasets: GSE81861, GSE144735, GSE132465). FIG. 18B depicts GPA33 gene expression analysis in lung normal tissue (dataset EGAS00001004344).

FIG. 19A and FIG. 19B depict expression analysis of potential NOT target PLA2G2A. FIG. 19A depicts PLA2G2A expression across a range of normal and CRC cellular subtypes (datasets: GSE81861, GSE144735, GSE132465). FIG. 19B depicts PLA2G2A gene expression analysis in lung normal tissue (dataset EGAS00001004344).

FIG. 20A and FIG. 20B depict expression analysis of potential NOT target ITM2C. FIG. 20A depicts ITM2C expression across a range of normal and CRC cellular subtypes (datasets: GSE81861, GSE144735, GSE132465). FIG. 20B depicts ITM2C gene expression analysis in lung normal tissue.

FIG. 21A and FIG. 21B depict expression analysis of potential NOT target CHP2. FIG. 21A depicts CHP2 expression across a range of normal and CRC cellular subtypes (datasets: GSE81861, GSE144735, GSE132465). FIG. 21B depicts CHP2 gene expression analysis in lung normal tissue.

FIG. 22A and FIG. 22B depict expression analysis of potential NOT target SLC26A2. FIG. 22A depicts SLC26A2 expression across a range of normal and CRC cellular subtypes (datasets: GSE81861, GSE144735, GSE132465). FIG. 22B depicts SLC26A2 gene expression analysis in lung normal tissue.

FIG. 23A and FIG. 23B depict expression analysis of potential NOT target SLC4A4. FIG. 23A depicts SLC4A4 expression across a range of normal and CRC cellular subtypes (datasets: GSE81861, GSE144735, GSE132465). FIG. 23B depicts SLC4A4 gene expression analysis in lung normal tissue.

FIG. 24A and FIG. 24B depict expression analysis of potential NOT target SLC26A3. FIG. 24A depicts SLC26A3 expression across a range of normal and CRC cellular subtypes (datasets: GSE81861, GSE144735, GSE132465). FIG. 24B depicts SLC26A3 gene expression analysis in lung normal tissue.

FIGS. 25A-25B depict LDH activity for parental, un-transduced LS174t cells (FIG. 25A) and LDH activity of mKATE-expressing target cells (FIG. 25B), each following co-culture with CEA aCAR transduced T cells from a first donor (“T cell donor 1”).

FIGS. 26A-26B depict LDH activity for parental, un-transduced LS174t cells (FIG. 26A) and LDH activity of mKATE-expressing target cells (FIG. 26B), each following co-culture with CEA aCAR transduced T cells from a second donor (“T cell donor 2”).

FIGS. 27A-27F depict cytokine activity (IL-2, FIG. 27A; IFN-gamma, FIG. 27B; TNF-alpha, FIG. 27C; caspase 3, FIG. 27D; perforin, FIG. 27E; and granzyme B, FIG. 27F) for CEA aCAR-transduced T cells following co-culture with target cells.

FIG. 28 depicts killing activity of CEA aCAR-transduced NK cells transduced with a first set of CEA aCARs, as determined by a flow-based assay.

FIG. 29 depicts killing activity of CEA aCAR-transduced NK cells transduced with a second set of CEA aCARs, as determined by a flow-based assay.

FIG. 30 depicts killing activity at various effector cell to target cell (E:T) ratios, for NK cells transduced with two different CEA aCARs, as determined by a flow-based assay.

FIGS. 31A-31C depict surface marker activation (NKp46, FIG. 31A; CD16, FIG. 31B; and CD107a, FIG. 31C) of CEA aCAR-transduced NK cells in culture with target cells.

FIG. 32 depicts killing activity of transduced CEA aCAR NK cells by a real-time fluorescence-based assay.

FIGS. 33A-33B depict fold change of tumor bioluminescence (FIG. 33A) and a representative tumor image from day 16 post-treatment (FIG. 33B) following treatment of mice with NK cells expressing a CEA aCAR.

FIG. 34 depicts killing activity of NK cells transduced with various CEA aCAR constructs, as measured by a real-time fluorescence-based assay.

FIG. 35 depicts killing activity of NK cells transduced with various CEA aCAR constructs, as measured by a real-time fluorescence-based assay.

FIG. 36 depicts representative images taken on day 2 post NK cell treatment, for mice intraperitoneally inoculated with target cells of a colorectal cancer cell line and treated with NK cells transduced with various CEA aCAR construct.

FIG. 37 depicts representative images taken on day 13 post NK cell treatment, for mice intraperitoneally inoculated with target cells of a colorectal cancer cell line and treated with NK cells transduced with a CEA aCAR construct.

FIGS. 38A-38D provide expression levels of various CEA-CAR lentiviral and retroviral constructs on transduced NK cells (3 days after transduction). FIGS. 38A, 38B, and 38C provide flow cytometry histograms depicting expression levels (38A, for lentiviral constructs at 3 days post-transduction; 38B, for retroviral constructs 3 days post-transduction; and 38C, for retroviral constructs 11 days post transduction). Dotted line represents the negative threshold (based on the expression of un-transduced “no virus” NK cells). FIG. 38D shows timecourses of expression from the retroviral constructs, with the left panel providing a timecourse of percent expression, and the right panel providing a time course of mean fluorescent intensity (MFI).

FIG. 39A-39C show CEA CAR expression from various retroviral transduction systems peudotyped with baboon endogenous virus envelope (BaEv). FIG. 39A shows expression from a first retroviral backbone (“backbone 1. FIG. 39B shows expression from the first retroviral vector backbone (“backbone 1”) and having an alternative nucleotide sequence (“new codon optimization”). FIG. 39C shows expression from a second retroviral back bone (“backbone 2”).

FIGS. 40A-40B provide transduction efficiency (FIG. 40A) and CAR expression (FIG. 40B) for CEA CARs having various CAR architectures.

FIGS. 41A-41B show killing of CEA CAR NK cells (derived from two different donors, donor 7, FIG. 41A and donor 13, FIG. 41B) at effector cell to target cell ratios of 1:1.

FIGS. 42A-42B show killing of CEA CAR NK cells (derived from two different donors, donor 7, FIG. 42A and donor 13, FIG. 42B) at effector cell to target cell ratios of 1:2.

FIG. 43 shows CEA CAR expression by NK cells, for various CAR architectures.

FIG. 44 shows NK cell activation as measured by granzyme B and interferon gamma for CEA CAR NK cells co-cultured with taret cells (colorectal cancer cell line Ls174t).

FIGS. 45A-45B show the fold change in tumor burden as calculated at 5 days post NK cell treatment (FIG. 45A) and representative images (FIG. 45B), as measured by bioluminescent imaging for tumor-bearing mice treated with CEA CAR NK cells.

FIG. 46 shows the tumor response rates for the mice in the study.

FIGS. 47A-47B show NK cell CAR expression levels for NK cells expressing a CEA aCAR and an off-target iCAR (FIG. 47A) and NK cells expressing a CEA aCAR and an inhibitory CAR specific to model safety antigen HER2 (FIG. 47B).

FIG. 48 shows NK cell expression levels of an inhibitory CAR (specific to safety antigen VSIG2) and an activating anti-CEA CAR.

FIG. 49 shows killing of HER2+Ls174t cells co-cultured with NK cells expressing an off-target iCAR and a CEA aCAR, or a HER2 iCAR and a CEA aCAR.

FIGS. 50A-50B show phosphorylation levels of NK cell activation markers for NK cells expressing aCAR/iCAR combinations, following co-culture with target cells expressing a safety antigen.

FIG. 51 shows percent target cell reduction as measured via flow cytometry after an overnight co-culture with NK cells expressing an aCAR/iCAR combination or an aCAR alone.

### DETAILED DESCRIPTION

The practice of the present disclosure will employ, unless otherwise indicated, conventional methods of molecular biology, chemistry, biochemistry, virology, and immunology, within the skill of the art. Such techniques are explained fully in the literature. See, e.g., Hepatitis C Viruses: Genomes and Molecular Biology (S. L. Tan ed., Taylor & Francis, 2006); Fundamental Virology, 3^{rd} Edition, vol. I & II (B. N. Fields and D. M. Knipe, eds.); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

As used herein, the term “activating an immunoresponsive cell” refers to the induction of signal transduction or changes in protein expression in the cell that results in the initiation of an immune response. For example, when CD3 chains cluster in response to ligand binding and immunoreceptor tyrosine-based inhibition motifs (ITAMs) a signal transduction cascade is produced. In certain embodiments, when an endogenous TCR or an exogenous CAR binds antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

As used herein, the term “stimulates” or “stimulating an immune response” refers to generating a signal that results in a immune response by one or more cell types or cell populations. Immunostimulatory activity may include pro-inflammatory activity. In various embodiments, the immune response occurs after immune cell (e.g., T-cell or NK cell) activation or concomitantly mediated through receptors including, but not limited to, CD28, CD137 (4-1BB), OX40, CD40 and ICOS, and their corresponding ligands, including B7-1, B7-2, OX-40L, and 4-1BBL. Such polypeptides may be present in the tumor microenvironment and can activate immune responses to neoplastic cells. In various embodiments, promoting, stimulating, or otherwise agonizing pro-inflammatory polypeptides and/or their ligands may enhance the immune response of an immunoresponsive cell. Without being bound to a particular theory, receiving multiple stimulatory signals (e.g., co-stimulation) is important to mount a robust and long-term cell mediated immune response, such as a T cell mediated immune response where T cells can become inhibited and unresponsive to antigen (also referred to as “T cell anergy”) in the absence of co-stimulatory signals. Without receiving these stimulatory signals, T cells quickly become inhibited and unresponsive to antigen. While the effects of the variety of co-stimulatory signals, particularly in combination with one another, can vary and remain only partially understood, they co-stimulation generally results in increasing gene expression in order to generate long-lived, proliferative, and anti-apoptotic resistant cells, such as T cells or NK cells, that robustly respond to antigen, for example in meditating complete and/or sustained eradication of targets cells expressing a cognate antigen.

As used herein, the term “chimeric antigen receptor” or alternatively a “CAR” refers to a recombinant polypeptide construct comprising at least an extracellular antigen-binding domain, a transmembrane domain and a cytoplasmic signaling domain (also referred to herein as “an intracellular signaling domain”) comprising a functional signaling domain.

As used herein, the term “activating CAR” or “aCAR” refers to CAR constructs/architectures capable of inducing signal transduction or changes in protein expression in the activating CAR-expressing cell that initiate, activate, stimulate, or increase an immune response upon binding to a cognate aCAR ligand.

As used herein, the term “inhibitory CAR” or “iCAR” refers to CAR constructs/architectures capable of inducing signal transduction or changes in protein expression in the inhibitory CAR-expressing cell that prevent, attenuate, inhibit, reduce, decrease, inhibit, or suppress an immune response upon binding to a cognate iCAR ligand, such as reduced activation of immunoresponsive cells receiving or having received one or more stimulatory signals, including co-stimulatory signals.

As used herein, the term “enzymatic inhibitory domain” refers to a protein domain that inhibits an intracellular signal transduction cascade, for example a native T cell activation cascade. In some embodiments, the enzymatic inhibitory domain of a chimeric inhibitory receptor of the present disclosure comprises at least a portion of an extracellular domain, a transmembrane domain, and/or an intracellular domain. In some embodiments, the enzymatic inhibitory domain comprises at least a portion of an enzyme. In some embodiments, the enzyme is selected from CSK, SHP-1, PTEN, CD45, CD148, PTP-MEG1, PTP-PEST, c-CBL, CBL-b, PTPN22, LAR, PTPH1, SHIP-1, and RasGAP (see e.g., Stanford et al., Regulation of TCR signaling by tyrosine phosphatases: from immune homeostasis to autoimmunity, Immunology, 2012 September; 137(1): 1-19). In some embodiments, the portion of the enzyme comprises an enzyme domain(s), an enzyme fragment(s), or a mutant(s) thereof. In some embodiments, the portion of the enzyme is a catalytic domain of the enzyme. In some embodiments, the enzyme domain(s), enzyme fragment(s), or mutants(s) thereof are selected to maximize efficacy and minimize basal inhibition.

As used herein, the term “intracellular signaling domain” refers to the functional portion of a protein which acts by transmitting information within the cell to regulate cellular activity via defined signaling pathways by generating second messengers or functioning as effectors by responding to such messengers.

As used herein, the term “extracellular antigen-binding domain” or “antigen-binding domain” (ABD) refers to a polypeptide sequence or polypeptide complex that specifically recognizes or binds to a given antigen or epitope, such as the polypeptide sequence or polypeptide complex portion of the chimeric proteins described herein that provide, for example, the VSIG2-specific binding. An ABD (or antibody, antigen-binding fragment, and/or the chimeric protein including the same) is said to “recognize” the epitope (or more generally, the antigen) to which the ABD specifically binds, and the epitope is said to be the “recognition specificity” or “binding specificity” of the ABD. The ABD is said to bind to its specific antigen or epitope with a particular affinity. As described herein, “affinity” refers to the strength of interaction of non-covalent intermolecular forces between one molecule and another. The affinity, i.e., the strength of the interaction, can be expressed as a dissociation equilibrium constant (K_{D}), wherein a lower K_{D} value refers to a stronger interaction between molecules. K_{D} values of antibody constructs are measured by methods well known in the art including, but not limited to, bio-layer interferometry (e.g. Octet/FORTEBIO®), surface plasmon resonance (SPR) technology (e.g. Biacore®), and cell binding assays (e.g., Flow-cytometry). Specific binding, as assessed by affinity, can refer to a binding molecule with an affinity between an ABD and its cognate antigen or epitope in which the K_{D} value is below 10⁻⁶M, 10⁻⁷M, 10⁻⁸M, 10⁻⁹M, or 10⁻¹⁰M. Specific binding can also include recognition and binding of a biological molecule of interest (e.g., a polypeptide) while not specifically recognizing and binding other molecules in a sample, for example, a biological sample, which naturally includes a polypeptide of the present disclosure. In certain embodiments, specific binding refers to binding between an ABD, antibody, or antigen-binding fragment to an epitope or antigen or antigenic determinant in such a manner that binding can be displaced or competed with a second preparation of identical or similar epitope, antigen or antigenic determinant.

An ABD can be an antibody. The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

An ABD can be an antigen-binding fragment of an antibody. As used herein, the term “antigen-binding fragment” refers to at least one portion of an intact antibody, or recombinant variants thereof, that is sufficient to confer recognition and specific binding of the antigen-binding fragment to a target, such as an antigen or epitope. Examples of antigen-binding fragments include, but are not limited to, Fab, Fab′, F(ab′)2, Fv, scFv, linear antibodies, single domain antibodies such as sdAb (either VL or VH), camelid V_{H}H domains, and multi-specific antibodies formed from antigen-binding fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1 136, 2005). Antigen binding fragments can also be grafted into scaffolds based on polypeptides such as a fibronectin type III (Fn3)(see U.S. Pat. No. 6,703,199, which describes fibronectin polypeptide minibodies).

The number of ABDs in a binding molecule, such as the chimeric proteins described herein, defines the “valency” of the binding molecule. A binding molecule having a single ABD is “monovalent”. A binding molecule having a plurality of ABDs is said to be “multivalent”. A multivalent binding molecule having two ABDs is “bivalent.” A multivalent binding molecule having three ABDs is “trivalent.” A multivalent binding molecule having four ABDs is “tetravalent.” In various multivalent embodiments, all of the plurality of ABDs have the same recognition specificity and can be referred to as a “monospecific multivalent” binding molecule. In other multivalent embodiments, at least two of the plurality of ABDs have different recognition specificities. Such binding molecules are multivalent and “multispecific.” In multivalent embodiments in which the ABDs collectively have two recognition specificities, the binding molecule is “bispecific.” In multivalent embodiments in which the ABDs collectively have three recognition specificities, the binding molecule is “trispecific.” In multivalent embodiments in which the ABDs collectively have a plurality of recognition specificities for different epitopes present on the same antigen, the binding molecule is “multiparatopic.” Multivalent embodiments in which the ABDs collectively recognize two epitopes on the same antigen are “biparatopic.”

In various multivalent embodiments, multivalency of the binding molecule improves the avidity of the binding molecule for a specific target. As described herein, “avidity” refers to the overall strength of interaction between two or more molecules, e.g. a multivalent binding molecule for a specific target, wherein the avidity is the cumulative strength of interaction provided by the affinities of multiple ABDs. Avidity can be measured by the same methods as those used to determine affinity, as described above. In certain embodiments, the avidity of a binding molecule for a specific target is such that the interaction is a specific binding interaction, wherein the avidity between two molecules has a K_{D} value below 10⁻⁶ M, 10⁻⁷ M, 10⁻⁸ M, 10⁻⁹ M, or 10⁻¹⁰ M. In certain embodiments, the avidity of a binding molecule for a specific target has a K_{D} value such that the interaction is a specific binding interaction, wherein the one or more affinities of individual ABDs do not have has a K_{D} value that qualifies as specifically binding their respective antigens or epitopes on their own. In certain embodiments, the avidity is the cumulative strength of interaction provided by the affinities of multiple ABDs for separate antigens on a shared specific target or complex, such as separate antigens found on an individual cell. In certain embodiments, the avidity is the cumulative strength of interaction provided by the affinities of multiple ABDs for separate epitopes on a shared individual antigen.

As used herein, the term “single-chain variable fragment” or “scFv” refers to a fusion protein comprising at least one antigen-binding fragment comprising a variable region of a light chain and at least one antigen-binding fragment comprising a variable region of a heavy chain, wherein the light and heavy chain variable regions are contiguously linked via a short flexible polypeptide linker, capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, “variable region” refers to a variable sequence that arises from a recombination event, for example, following V, J, and/or D segment recombination in an immunoglobulin gene in a B cell or T cell receptor (TCR) gene in a T cell. In immunoglobulin genes, variable regions are typically defined from the antibody chain from which they are derived, e.g., VH refers to the variable region of an antibody heavy chain and VL refers to the variable region of an antibody light chain. A select VH and select VL can associate together to form an antigen-binding domain that confers antigen specificity and binding affinity.

The term “complementarity determining region” or “CDR,” as used herein, refers to the sequences within antibody variable regions VH and VL which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al, (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL. In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences.

The term “framework region” or “FR,” as used herein, refers to the generally conserved sequences within antibody variable regions VH and VL that act as a scaffold for interspersed CDRs, typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In specific embodiments, the FRs are human sequences. In various embodiments, the FRs are naturally occurring sequences. In various embodiments, the FRs are synthesized sequences including, but not limited, rationally designed sequences.

As used herein, the term “antibody heavy chain” refers to the larger of the two types of polypeptide chains present in antibody molecules in their naturally occurring conformations, and which normally determines the class to which the antibody belongs.

As used herein, the term “antibody light chain” refers to the smaller of the two types of polypeptide chains present in antibody molecules in their naturally occurring conformations. Kappa (κ) and lambda (λ) light chains refer to the two major antibody light chain isotypes.

As used herein, the term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

As used herein, the term “antigen” or “Ag” refers to a molecule that provokes an immune response. This immune response may involve either antibody production, or the activation of specific immunologically competent cells, or both. The skilled artisan will understand that any macromolecule, including virtually all proteins or peptides, can serve as an antigen.

As used herein, the term “anti-tumor effect” or “anti-tumor activity” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place, such as in a prophylactic therapy or treatment.

As used herein, the term “autologous” refers to any material derived from the same subject to whom it is later to be re-introduced into the subject.

As used herein, the term “allogeneic” refers to any material derived from a different animal of the same species as the subject to whom the material is introduced. Two or more subjects are said to be allogeneic to one another when the genes at one or more loci are not identical. In some embodiments, allogeneic material from individuals of the same species may be sufficiently genetically distinct, e.g., at particular genes such as MHC alleles, to interact antigenically. In some embodiments, allogeneic material from individuals of the same species may be sufficiently genetically similar, e.g., at particular genes such as MHC alleles, to not interact antigenically.

Isolated nucleic acid molecules of the present disclosure include any nucleic acid molecule that encodes a polypeptide of the present disclosure, or fragment thereof. Such nucleic acid molecules need not be 100% homologous or identical with an endogenous nucleic acid sequence, but will typically exhibit substantial identity. Nucleic acids having “substantial identity” or “substantial homology” to an endogenous sequence are typically capable of hybridizing with at least one strand of a double-stranded nucleic acid molecule. As used herein, “hybridize” refers to pairing to form a double-stranded molecule between complementary polynucleotide sequences (e.g., a gene described herein), or portions thereof, under various conditions of stringency. For example, stringent salt concentration may be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency may be accomplished by combining these various conditions as needed.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). Preferably, such a sequence is at least about 60%, about 80%, about 85%, about 90%, about 95%, about 99%, or about 100% homologous or identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

As used herein, the term “ligand” refers to a molecule that binds to a receptor. In particular, the ligand binds a receptor on another cell, allowing for cell-to-cell recognition and/or interaction.

The terms “effective amount” and “therapeutically effective amount” are used interchangeably herein, and refer to an amount of a compound, formulation, material, or composition, as described herein effective to achieve a particular biological result. In some embodiments, an “effective amount” or a “therapeutically effective amount” is an amount sufficient to arrest, ameliorate, or inhibit the continued proliferation, growth, or metastasis of a disease or disorder of interest, e.g., a myeloid disorder.

As used herein, the term “immunoresponsive cell” refers to a cell that functions in an immune response (e.g., an immune effector response) or a progenitor, or progeny thereof. Examples of immune effector cells include, without limitation, alpha/beta T cells, gamma/delta T cells, B cells, natural killer (NK) cells, natural killer T (NKT) cells, mast cells, and myeloid-derived phagocytes.

As used herein, the term “immune effector response” or “immune effector function” refers to a function or response, e.g., of an immunoresponsive cell, that enhances or promotes an immune attack of a target cell. For example, an immune effector function or response may refer to a property of a T cell or NK cell that promotes killing or the inhibition of growth or proliferation, of a target cell. In the case of a T cell, primary stimulation and co-stimulation are examples of immune effector function or response.

As used herein, the term “flexible polypeptide linker” or “linker” refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Gly-Ser)ₙ (SEQ ID NO: 139) or (Gly-Gly-Gly-Ser)ₙ (SEQ ID NO: 140), where n is a positive integer equal to or greater than 1. For example, n=1, n=2, n=3, n=4, n=5, n=6, n=7, n=8, n=9, or n=10. In some embodiments, the flexible polypeptide linkers include, but are not limited to, Gly₄Ser (SEQ ID NO: 37) or (Gly₄Ser)₃ (SEQ ID NO: 39). In other embodiments, the linkers include multiple repeats of (Gly₂Ser) (SEQ ID NO: 27), (GlySer) or (Gly₃Ser) (SEQ ID NO: 32). In some embodiments, the flexible polypeptide linkers include a Whitlow linker (e.g., GSTSGSGKPGSGEGSTKG [SEQ ID NO:42]). Also included within the scope of the present disclosure are linkers described, for example, in WO2012/138475.

As used herein, the terms “treat”, “treatment” and “treating” refer to the reduction or amelioration of the progression, severity and/or duration of a proliferative disorder (e.g., cancer), or the amelioration of one or more symptoms (preferably, one or more discernible symptoms) of a proliferative disorder resulting from the administration of one or more therapies (e.g., one or more therapeutic agents such as a CAR of the present disclosure). In some embodiments, reduction or amelioration refers to the amelioration of at least one measurable physical parameter of a proliferative disorder, such as growth of a tumor, not necessarily discernible by the patient. In other embodiments, the terms “treat”, “treatment”, and “treating” refer to the inhibition of the progression of a proliferative disorder, either physically by, e.g., stabilization of a discernible symptom, physiologically by, e.g., stabilization of a physical parameter, or both. In some embodiments, reduction or amelioration include reduction or stabilization of tumor size or cancerous cell count.

As used herein, the term “subject” is intended to include living organisms in which an immune response can be elicited (e.g., mammals, human).

### Other Interpretational Conventions

Ranges recited herein are understood to be shorthand for all of the values within the range, inclusive of the recited endpoints. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### Solid Tumor Antigens

Certain aspects of the present disclosure relate to chimeric receptors and cells, such as immunoresponsive cells, that have been genetically modified to express one or more of such chimeric receptors that bind to an antigen of interest, and to methods of using such receptors and cells to treat and/or prevent solid malignancies, such as lung cancer, pancreatic cancer, gastrointestinal cancer, colon cancer, brain cancer, cancer of the neuronal tissue, endocrine tumors, bone cancer, cancer of the bone marrow, cancer of the immune system, muscle cancer, live cancer, gallbladder cancer, kidney cancer, urinary bladder cancer, cancer of the male reproductive organs, cancer of the female reproductive organs, adipose cancer, soft tissue cancer, and skin cancer, and other pathologies where an antigen-specific immune response is desired. Malignant cells have developed a series of mechanisms to protect themselves from immune recognition and elimination. The present disclosure provides immunogenicity within the tumor microenvironment for treating such malignant cells.

Certain aspects of the present disclosure related to chimeric receptors that specifically bind one or more antigens expressed on a myeloid cell useful for treating solid tumor malignancies, and to immunoresponsive cells genetically modified to express such chimeric receptors. Solid cancers are clonal diseases caused by genetic and epigenetic alterations that disrupt key processes such as cell proliferation and differentiation. Solid tumor malignancies can be chronic or acute.

In certain embodiments, the present disclosure relates to solid tumor antigens and combinations of solid tumor antigens that are suitable for use in chimeric receptors (e.g., chimeric TCRs or CARs) to increase efficacy and/or reduce off-tumor toxicity in the treatment of the solid tumor. In certain embodiments, the solid tumor antigen is a CEA-family member. In certain embodiments, the solid tumor antigen is a CEA-family member selected from the group consisting of CEA, CEACAM1, CEACAM5, and CEACAM6. As used herein, “CEA” refers to a family of highly related proteins (CD66 proteins), including, without limitation CEACAM1 (CD66a), CEACAM5 (CD66e), and CEACAM6 (CD66c). In certain embodiments, an antibody or antigen-binding fragment that binds CEA binds more than one CD66 protein.

Table 1 provides CEA-family antigens suitable for use in chimeric receptors described in the methods and compositions presented herein.

TABLE 1
Solid Tumor Antigens

| | UniProt | | |
|---|---|---|---|
| Antigen | Accession No. | Name | Short Description |
| CEA | — | Carcinoembryonic | Highly related cell surface glycoproteins |
| | | antigen-related | that function in cell adhesion |
| | | cell adhesion | |
| | | molecule | |
| CEACAM1 | P13688 | Carcinoembryonic | Cell surface glycoprotein that plays a role |
| | | antigen-related | as a coinhibitory receptor in immune |
| | | cell adhesion | response, insulin action and functions as |
| | | molecule 1 | an activator during angiogenesis |
| CEACAM5 | P06731 | Carcinoembryonic | Cell surface glycoprotein that plays a role |
| | | antigen-related | in cell adhesion, intracellular signaling |
| | | cell adhesion | and tumor progression |
| | | molecule 5 | |
| CEACAM6 | P40199 | Carcinoembryonic | Cell surface glycoprotein that plays a role |
| | | antigen-related | in cell adhesion and tumor progression |
| | | cell adhesion | |
| | | molecule 6 | |

In some embodiments, the solid tumor antigen is a CEACAM1 antigen. CEACAM1 is also known in the art as BGP, BGP1, BGP1, or CD66a. In some embodiments, the solid tumor antigen is a CEACAM5 antigen. CEACAM5, was previously known in the art as CEA. At present CEACAM5 is also known as Meconium Antigen 100, Carcinoembryonic Antigen, or CD66e. In some embodiments, the solid tumor antigen is a CEACAM6 antigen. CEACAM6 is also known in the art as CEAL, NCA, Normal Cross-Reacting Antigen, Non-Specific Crossreacting Antigen, or CD66c.

### Chimeric Receptors

Certain aspects of the present disclosure relate to chimeric receptors and nucleic acids that encode such chimeric receptors that bind to an antigen of interest.

### Antibodies and Antigen-Binding Fragments

In some embodiments, chimeric receptors comprise one or more antigen binding domains capable of binding a solid tumor antigen, e.g., a CEA-family member antigen (such as listed in Table 1). Antigen binding domains of the chimeric receptors can comprise antibody sequences, or antigen-binding fragments thereof, of the representative anti-CEA antibodies provided in Table 2.

TABLE 2
Exemplary solid tumor antigen binders

| Solid Tumor | Antibody | |
|---|---|---|
| Antigen | Clone | Clone Sequences |
| CEACAM1 | MRG1 | VH: |
| | | QVQLQQSGAELVRPGTSVKVSCKASGYAFTNNLIEWVKQRPGQG |
| | | LEWIGVINPGSGDTNYNEKFKGKATLTADKSSNTAYMQLSSLTSD |
| | | DSAVYFCARGDYYGGFAVDYWGQGTSVTVSS (SEQ ID NO: 1) |
| | | VL: |
| | | DIQMTQTTSSLSASLGDRVTISCRTSQDIGNYLNWYQQKPDGTVK |
| | | LLIYYTSRLHSGVPSRFSGSGSGTDYSLTISNLEQEDIATYFCQQGK |
| | | SLPRTFGGGTKLEI (SEQ ID NO: 2) |
| CEACAM5 | Labetuzumab | VH: |
| | (i.e., hMN14) | EVOLVESGGGVVQPGRSLRLSCSASGFDFTTYWMSWVRQAPGK |
| | | GLEWIGEIHPDSSTINYAPSLKDRFTISRDNAKNTLFLQMDSLRPE |
| | | DTGVYFCASLYFGFPWFAYWGQGTPVTVSS (SEQ ID NO: 3) |
| | | VL: |
| | | DIQLTQSPSSLSASVGDRVTITCKASQDVGTSVAWYQQKPGKAPK |
| | | LLIYWTSTRHTGVPSRFSGSGSGTDFTFTISSLQPEDIATYYCQQYS |
| | | LYRSFGQGTKVEIK (SEQ ID NO: 4) |
| CEACAM5 | Cibisatamab | HC: |
| | | QVQLVQSGAEVKKPGASVKVSCKASGYTFTEFGMNWVRQAPGQ |
| | | GLEWMGWINTKTGEATYVEEFKGRVTFTTDTSTSTAYMELRSLR |
| | | SDDTAVYYCARWDFAYYVEAMDYWGQGTTVTVSSASTKGPSVF |
| | | PLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKTT |
| | | PPVLDSDGSFFLVSKLTVDKSRWQQGNVFSCSVMHEALHNHYTQ |
| | | KSLSLS (SEQ ID NO: 5) |
| | | LC: |
| | | DIQMTQSPSSLSASVGDRVTITCKASAAVGTYVAWYQQKPGKAP |
| | | KLLIYSASYRKRGVPSRFSGSGSGTDFTLTISSLQPEDFATYYCHQ |
| | | YYTYPLFTFGQGTKLEIKRTVAAPSVFIFPPSDEQLKSGTASVVCL |
| | | LNNFYPREAKVQWKVDNALQSGNSQESVTEQDSKDSTYSLSSTL |
| | | TLSKADYEKHKVYAEVTHQGLSSPVTKSFNRGEC (SEQ ID NO: 6) |
| CEACAM5 | Tusamitamab | HC: |
| | | EVQLQESGPGLVKPGGSLSLSCAASGFVFSSYDMSWVRQTPERGL |
| | | EWVAYISSGGGITYAPSTVKGRFTVSRDNAKNTLYLQMNSLTSED |
| | | TAVYYCAAHYFGSSGPFAYWGQGTLVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQ |
| | | DWLNGKEYKCKVSNKALPAPIEKTISKAKGQPREPQVYTLPPSRD |
| | | ELTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | (SEQ ID NO: 7) |
| | | LC: |
| | | DIQMTQSPASLSASVGDRVTITCRASENIFSYLAWYQQKPGKSPKL |
| | | LVYNTRTLAEGVPSRFSGSGSGTDFSLTISSLQPEDFATYYCQHHY |
| | | GTPFTFGSGTKLEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC (SEQ ID NO: 8) |
| CEACAM5 | BW431/26 | VH: |
| | | QLQESGPGLVRPSQTLSLTCTVSGFTISSGYSWHWVRQPPGRGLE |
| | | WIGYIQYSGITNYNPSLKSRVTMLVDTSKNQFSLRLSSVTAADTA |
| | | VYYCAREDYDYHWYFDVWGQGSLVTVSS (SEQ ID NO: 9) |
| | | VL: |
| | | GVHSDIQMTQSPSSLSASVGDRVTITCSTSSSVSYMHWYQQKPGK |
| | | APKLLIYSTSNLASGVPSRFSGSGSGTDFTFTISSLQPEDIATYYCH |
| | | QWSSYPTFGQGTKVEIKR (SEQ ID NO: 10) |
| CEACAM5 | A5B7 | VH: |
| | | MDFQVQIFSFLLISASVIMSRGQTVLSQSPAILSASPGEKVTMTCRA |
| | | SSSVTYIHWYQQKPGSSPKSWIYATSNLASGVPARFSGSGSGTSYS |
| | | LTISRVEAEDAATYYCQHWSSKPPTFGGGTKLEIKR (SEQ ID NO: 11) |
| | | VL: |
| | | MDFQVQIFSFLLISASVIMSRGQTVLSQSPAILSASPGEKVTMTCRA |
| | | SSSVTYIHWYQQKPGSSPARFSGSGTSYSLTISRVEAEDAATYYCQ |
| | | HWSSKPPTFGGGTKLEIKR (SEQ ID NO: 12) |
| CEACAM5 | MFE23 | VH: |
| | | ETVIKYLLPTAAAGLLLLAAQPAMAQVKLQQSGAELVRSGTSVKLSC |
| | | TASGFNIKDSYMHWLRQGPEQGLEWIGWIDPENGDTEYAPKFQGKA |
| | | TFTTDTSSNTAYLQLSSLTSEDTAVYYCNEGTPTGPYYFDYWGQGTT |
| | | VTVSS (SEQ ID NO: 13) |
| | | VL: |
| | | ENVLTQSPAIMSASPGEKVTITCSASSSVSYMHWFQQKPGTSPKLWIY |
| | | STSNLASGVPARFSGSGSGTSYSLTISRMEAEDAATYYCQQRSSYPLT |
| | | FGAGTKLELKRAA (SEQ ID NO: 14) |
| CEACAM5 | hMFE23 | VH: |
| | | QVKLEQSGAEVVKPGASVKLSCKASGFNIKDSYMHWLRQGPGQRLE |
| | | WIGWIDPENGDTEYAPKFQGKATFTTDTSANTAYLGLSSLRPEDTAV |
| | | YYCNEGTPTGPYYFDYWGQGTLVTVSS (SEQ ID NO: 78) |
| | | VL: |
| | | ENVLTQSPSSMSVSVGDRVTIACSASSSVPYMHWLQQKPGKSPKLLI |
| | | YLTSNLASGVPSRFSGSGSGTDYSLTISSVQPEDAATYYCQQRSSYPL |
| | | TFGGGTKLEIK (SEQ ID NO: 79) |
| Glycosylated | FM4 (also | VH: |
| | referred to as | EVKLVESGGGLVQPGGSLRLSCSISGFTFTDYYMNWVRQSPGKALE |
| CEACAM5 | “MG7”) | WLGFIRNKVNGDTTEYSASVKGRFTISRDISQSILYLQMNTLRTEDSA |
| | | TYYCARDKGIAYYFDYWGQGTTLTVSS (SEQ ID NO: 15) |
| | | VL: |
| | | QIVLSQSPAILFASPGEKVTMTCRASSSVSYIHWYQQKPGSSPKPWIH |
| | | GTSNLASGVPARFSGSGSGTSYSLTISRMEAEDAATYYCQQWSSNLS |
| | | TFGGGTKLEIK (SEQ ID NO: 16) |
| CEACAM6 | Tinurilimab | HC: |
| | | QVTLRESGPALVKPTQTLTLTCTFSGFSLSTYGIGVGWIRQPPGKA |
| | | LEWLAHIWWNDNKYYSTSLKTRLTISKDTSKNQVVLTMTNMDP |
| | | VDTATYYCARISLPYFDYWGQGTTLTVSSASTKGPSVFPLAPCSRS |
| | | TSESTAALGCLVKDYFPEPVTVSWNSGALTSGVHTFPAVLQSSGL |
| | | YSLSSVVTVPSSNFGTQTYTCNVDHKPSNTKVDKTVERKCCVECP |
| | | PCPAPPVAGPSVFLFPPKPKDTLMISRTPEVTCVVVDVSHEDPEVQ |
| | | FNWYVDGVEVHNAKTKPREEQFNSTFRVVSVLTVVHQDWLNGK |
| | | EYKCKVSNKGLPAPIEKTISKTKGQPREPQVYTLPPSREEMTKNQ |
| | | VSLTCLVKGFYPSDIAVEWESNGQPENNYKTTPPMLDSDGSFFLY |
| | | SKLTVDKSRWQQGNVFSCSVMHEALHNHYTQKSLSLSPG (SEQ |
| | | ID NO: 17) |
| | | LC: |
| | | DIQLTQSPSFLSASVGDRVTITCKASQNVGTAVAWYQQKPGKAPK |
| | | LLIYSASNRYTGVPSRFSGSGSGTEFTLTISSLQPEDFATYYCQQYS |
| | | SYPLTFGGGTKVEIKRTVAAPSVFIFPPSDEQLKSGTASVVCLLNN |
| | | FYPREAKVQWKVDNALQSGNSQESVTEQDSKDSTYSLSSTLTLSK |
| | | ADYEKHKVYACEVTHQGLSSPVTKSFNRGEC (SEQ ID NO: 18) |

Certain aspects of the present disclosure relate to chimeric receptors (e.g., CAR or chimeric TCR) comprising an extracellular antigen-binding domain that binds to one or more antigens of the present disclosure. In some embodiments, the antigen-binding domains are derived from an antibody, or antigen-binding fragment thereof. In some embodiments, the antigen-binding domains comprise the CDR sequences of an antibody or antigen-binding fragment thereof of Table 2. CDR sequences and known systems for defining them, e.g., Kabat, are discussed in detail above.

Suitable antibodies of the present disclosure include any antibody, whether natural or synthetic, full length or a fragment thereof, monoclonal or polyclonal, that binds sufficiently strongly and specifically to a solid tumor antigen, e.g., CEA, CEACAM1, CEACAM5, or CEACAM6. In some embodiments, the antibody may have a K_{D} of at most about at most 10⁻⁶ M, at most about 10⁻⁷ M, at most about 10⁻⁸ M, at most about 10⁻⁹ M, at most about 10⁻¹⁰ M, at most about 10⁻¹¹ M, or at most about 10⁻¹² M.

In some embodiments, antibodies and derivatives thereof that may be used include, without limitation, polyclonal antibodies, monoclonal antibodies, chimeric antibodies, human antibodies, humanized, antibodies primatized (CDR-grafted) antibodies, veneered antibodies, single-chain antibodies, phage-produced antibodies (e.g., from phage display libraries), and functional binding fragments of antibodies. For example, antibody fragments capable of binding to a solid tumor antigen, or portions thereof, include, without limitation, Fv, Fab, Fab′ and F(ab′)₂ fragments. Such fragments can be produced by enzymatic cleavage or by recombinant techniques. For example, and not by way of limitation, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH, domain and hinge region of the heavy chain.

Methods of raising an antibody targeting a specific antigen are generally known in the art. Synthetic and engineered antibodies are described in, e.g., U.S. Pat. No. 4,816,567, EP0125023B1, U.S. Pat. No. 4,816,397, EP0120694B1, WO 86/01533, EP0194276B1, U.S. Pat. No. 5,225,539, EP0239400B1, EP0451216B1, EP0519596A1 and U.S. Pat. No. 4,946,778.

In some embodiments, commercially available antibodies may be used for binding to a solid tumor antigen. The CDRs of the commercially available antibodies are readily accessible by one skilled in the art using conventional sequencing technology. Further, one skilled in the art is able to construct nucleic acids encoding scFvs and chimeric receptors (e.g., CARs and TCRs) based on the CDRs of such commercially available antibodies.

In some embodiments, a chimeric receptor comprises an antigen-binding domain that specifically binds CEA.

In some embodiments, a chimeric receptor comprises an antigen-binding domain that specifically binds CEACAM1. In some embodiments, the CEACAM1-specific antigen-binding domain is derived from an anti-CEACAM1 antibody, such as the MRG1 antibody or an antigen-binding fragment thereof. In certain embodiments, the CEACAM1-specific antigen-binding domain comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:1, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:2. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the VH and VL sequences of SEQ ID NOs: 1 and 2, respectively. The antigen-binding domain may be an scFv that comprises a light chain variable domain (VL) and a heavy chain variable domain (VH). In some embodiments, the chimeric receptor may have a multispecific antigen-binding domain. For example, the chimeric receptor may be specific for CEACAM1 and one or more additional antigens. In some embodiments, the chimeric receptor may be specific for CEACAM1 and CEACAM5. In some embodiments, the chimeric receptor may be specific for CEACAM1 and CEACAM6. In some embodiments, the chimeric receptor may be specific for CEACAM5 and CEACAM6.

In some embodiments, a chimeric receptor comprises an antigen-binding domain that specifically binds CEACAM5. In some embodiments, the CEACAM5-specific antigen-binding domain is derived from an anti-CEACAM5 antibody, such as labetuzimab (i.e., hMN14) or an antigen-binding fragment thereof. In certain embodiments, the CEACAM5-specific antigen-binding domain comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:3, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:4. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the VH and VL sequences of SEQ ID NOs: 3 and 4, respectively. The antigen-binding domain may be an scFv that comprises a light chain variable domain (VL) and a heavy chain variable domain (VH). In some embodiments, the chimeric receptor may have a multispecific antigen-binding domain. For example, the chimeric receptor may be specific for CEACAM5 and one or more additional antigens.

In some embodiments, the CEACAM5-specific antigen-binding domain is derived from an anti-CEACAM5 antibody, such as cibisatamab or an antigen-binding fragment thereof. In certain embodiments, the CEACAM5-specific antigen-binding domain comprises a heavy chain (HC) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 5, and a light chain (LC) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 6. In certain embodiments, the second antigen-binding site comprises a heavy chain variable domain (VH) and a light chain variable domain (VL) of the HC and LC sequences of SEQ ID NOs: 5 and 6, respectively. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the HC and LC sequences of SEQ ID NOs: 5 and 6, respectively. The antigen-binding domain may be an scFv that comprises a light chain variable domain and a heavy chain variable domain. In some embodiments, the chimeric receptor may have a multispecific antigen-binding domain. For example, the chimeric receptor may be specific for CEACAM5 and one or more additional antigens.

In some embodiments, the CEACAM5-specific antigen-binding domain is derived from an anti-CEACAM5 antibody, such as tusamitamab or an antigen-binding fragment thereof. In certain embodiments, the CEACAM5-specific antigen-binding domain comprises a heavy chain (HC) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 7, and a light chain (LC) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 8. In certain embodiments, the second antigen-binding site comprises a heavy chain variable domain (VH) and a light chain variable domain (VL) of the HC and LC sequences of SEQ ID NOs: 7 and 8, respectively. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the HC and LC sequences of SEQ ID NOs: 7 and 8, respectively. The antigen-binding domain may be an scFv that comprises a light chain variable domain and a heavy chain variable domain. In some embodiments, the chimeric receptor may have a multispecific antigen-binding domain. For example, the chimeric receptor may be specific for CEACAM5 and one or more additional antigens.

In some embodiments, the CEACAM5-specific antigen-binding domain is derived from an anti-CEACAM5 antibody, such as BW431/26 or an antigen-binding fragment thereof. In certain embodiments, the CEACAM5-specific antigen-binding domain comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 9, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 10. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the VH and VL sequences of SEQ ID NOs: 9 and 10, respectively. The antigen-binding domain may be an scFv that comprises a light chain variable domain (VL) and a heavy chain variable domain (VH). In some embodiments, the chimeric receptor may have a multispecific antigen-binding domain. For example, the chimeric receptor may be specific for CEACAM5 and one or more additional antigens.

In some embodiments, the CEACAM5-specific antigen-binding domain is derived from an anti-CEACAM5 antibody, such as A5B7 or an antigen-binding fragment thereof. In certain embodiments, the CEACAM5-specific antigen-binding domain comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 11, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 12. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the VH and VL sequences of SEQ ID NOs: 11 and 12, respectively. The antigen-binding domain may be an scFv that comprises a light chain variable domain (VL) and a heavy chain variable domain (VH). In some embodiments, the chimeric receptor may have a multispecific antigen-binding domain. For example, the chimeric receptor may be specific for CEACAM5 and one or more additional antigens.

In some embodiments, the CEACAM5-specific antigen-binding domain is derived from an anti-CEACAM5 antibody, such as MFE23 or an antigen-binding fragment thereof. In certain embodiments, the CEACAM5-specific antigen-binding domain comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 13, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 14. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the VH and VL sequences of SEQ ID NOs: 13 and 14, respectively. The antigen-binding domain may be an scFv that comprises a light chain variable domain (VL) and a heavy chain variable domain (VH). In some embodiments, the chimeric receptor may have a multispecific antigen-binding domain. For example, the chimeric receptor may be specific for CEACAM5 and one or more additional antigens.

In some embodiments, the CEACAM5-specific antigen-binding domain is derived from an anti-CEACAM5 antibody, such as hMFE23 or an antigen-binding fragment thereof. In certain embodiments, the CEACAM5-specific antigen-binding domain comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 78, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 79. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the VH and VL sequences of SEQ ID NOs: 78 and 79, respectively. The antigen-binding domain may be an scFv that comprises a light chain variable domain (VL) and a heavy chain variable domain (VH). In some embodiments, the chimeric receptor may have a multispecific antigen-binding domain. For example, the chimeric receptor may be specific for CEACAM5 and one or more additional antigens.

In some embodiments, the CEACAM5-specific antigen-binding domain is derived from an anti-CEACAM5 antibody capable of specifically binding glycosylated CEACAM5. In some embodiments, the glycosylated CEACAM5-specific antigen-binding domain is derived from an anti-glycosylated CEACAM5 antibody, such as FM4 (also referred to herein as “MG7”) or an antigen-binding fragment thereof. In certain embodiments, the CEACAM5-specific antigen-binding domain comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 15, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 16. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the VH and VL sequences of SEQ ID NOs: 15 and 16, respectively. The antigen-binding domain may be an scFv that comprises a light chain variable domain (VL) and a heavy chain variable domain (VH). In some embodiments, the chimeric receptor may have a multispecific antigen-binding domain. For example, the chimeric receptor may be specific for CEACAM5 and one or more additional antigens.

In some embodiments, a chimeric receptor comprises an antigen-binding domain that specifically binds CEACAM6. In some embodiments, the CEACAM6-specific antigen-binding domain is derived from an anti-CEACAM6 antibody, such as tinurilimab or an antigen-binding fragment thereof. In certain embodiments, the CEACAM6-specific antigen-binding domain comprises a heavy chain (HC) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 17, and a light chain (LC) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 18. In certain embodiments, the second antigen-binding site comprises a heavy chain variable domain (VH) and a light chain variable domain (VL) of the HC and LC sequences of SEQ ID NOs: 17 and 18, respectively. In certain embodiments, the second antigen-binding site comprises the heavy chain CDR1, CDR2, and CDR3 and the light chain CDR1, CDR2, and CDR3, determined under Kabat, Chothia, MacCallum, or any other CDR determination method known in the art, of the HC and LC sequences of SEQ ID NOs: 17 and 18, respectively. The antigen-binding domain may be an scFv that comprises a light chain variable domain and a heavy chain variable domain. In some embodiments, the chimeric receptor may have a multispecific antigen-binding domain. For example, the chimeric receptor may be specific for CEACAM6 and one or more additional antigens.

### T Cell Receptor (TCR)

Certain aspects of the present disclosure relate to chimeric receptors that specifically bind to an antigen expressed on a solid tumor cell. In some embodiments, the chimeric receptor is a chimeric T cell receptor (TCR). TCRs of the present disclosure are disulfide-linked heterodimeric proteins containing two variable chains expressed as part of a complex with the invariant CD3 chain molecules. TCRs are found on the surface of T cells, and are responsible for recognizing antigens as peptides bound to major histocompatibility complex (MHC) molecules. In certain embodiments, a TCR of the present disclosure comprises an alpha chain encoded by TRA and a beta chain encoded by TRB. In certain embodiments, a TCR comprises a gamma chain and a delta chain (encoded by TRG and TRD, respectively).

Each chain of a TCR is composed of two extracellular domains: a variable (V) region and a constant (C) region. The constant region is proximal to the cell membrane, followed by a transmembrane region and a short cytoplasmic tail. The variable region binds to the peptide/MHC complex. Each of the variable regions has three complementarity determining regions (CDRs).

In certain embodiments, a TCR can form a receptor complex with three dimeric signaling modules CD3δ/ε, CD3γ/ε, and CD247ζ/ζ or CD247ζ/η. When a TCR complex engages with its antigen and MHC (peptide/MHC), the T cell expressing the TCR complex is activated.

In some embodiments, a TCR of the present disclosure is a recombinant TCR. In certain embodiments, the TCR is a non-naturally occurring TCR. In certain embodiments, the TCR differs from a naturally occurring TCR by at least one amino acid residue. In some embodiments, the TCR differs from a naturally occurring TCR by at least 2 amino acid residues, at least 3 amino acid residues, at least 4 amino acid residues, at least 5 amino acid residues, at least 6 amino acid residues, at least 7 amino acid residues, at least 8 amino acid residues, at least 9 amino acid residues, at least 10 amino acid residues, at least 11 amino acid residues, at least 12 amino acid residues, at least 13 amino acid residues, at least 14 amino acid residues, at least 15 amino acid residues, at least 20 amino acid residues, at least 25 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, or more amino acid residues. In certain embodiments, the TCR is modified from a naturally occurring TCR by at least one amino acid residue. In some embodiments, the TCR is modified from a naturally occurring TCR by at least 2 amino acid residues, at least 3 amino acid residues, at least 4 amino acid residues, at least 5 amino acid residues, at least 6 amino acid residues, at least 7 amino acid residues, at least 8 amino acid residues, at least 9 amino acid residues, at least 10 amino acid residues, at least 11 amino acid residues, at least 12 amino acid residues, at least 13 amino acid residues, at least 14 amino acid residues, at least 15 amino acid residues, at least 20 amino acid residues, at least 25 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, or more amino acid residues.

### Chimeric TCRs

In some embodiments, a TCR of the present disclosure comprises one or more antigen-binding domains that may be grafted to one or more constant domain of a TCR chain, for example a TCR alpha chain or TCR beta chain, to create a chimeric TCR that binds specifically to a target antigen of the present disclosure (e.g., a solid tumor antigen). Without wishing to be bound by theory, it is believed that chimeric TCRs may signal through the TCR complex upon antigen binding. For example, an antibody or antibody fragment (e.g., scFv) can be grafted to the constant domain, e.g., at least a portion of the extracellular constant domain, the transmembrane domain and the cytoplasmic domain, of a TCR chain, such as the TCR alpha chain and/or the TCR beta chain. As another example, the CDRs of an antibody or antibody fragment may be grafted into a TCR alpha chain and/or beta chain to create a chimeric TCR that binds specifically to an antigen of the present disclosure (e.g., a solid tumor antigen). Such chimeric TCRs may be produced by methods known in the art (e.g., Willemsen R A et al., Gene Therapy 2000; 7:1369-1377; Zhang T et al., Cancer Gene Ther 2004 11: 487-496; and Aggen et al., Gene Ther. 2012 April; 19(4): 365-74).

### Chimeric Antigen Receptors (CARs)

Certain aspects of the present disclosure relate to chimeric receptors that specifically bind to an antigen expressed on a solid tumor, e.g., a tumor of the lung, pancreas, gastrointestinal tract, colon, brain, neuronal tissue, endocrine, bone, bone marrow, immune system, muscle, liver, gallbladder, kidney, urinary bladder, male reproductive organs, female reproductive organs, adipose, soft tissue, or skin. In some embodiments, the chimeric receptor is a chimeric antigen receptor (CAR). In some embodiments, a CAR (or immunoresponsive cells genetically engineered to comprise one or more CARs, see below) specifically binds a CEA-family member tumor antigen, such as any of the CEA antigens of Table 1. In some embodiments, a CAR (or immunoresponsive cells genetically engineered to comprise one or more CARs) can comprise antibody sequences, or antigen-binding fragments thereof, of the representative anti-CEA antibodies provided in Table 2. In some embodiments, a CAR (or immunoresponsive cells genetically engineered to comprise one or more CARs) can comprise an scFv derived from antibodies capable of binding to a solid tumor antigen, such as the representative anti-CEA scFvs provided in Table 3.

In some embodiments, CARs are engineered receptors that graft or confer a specificity of interest onto an immune effector cell. In certain embodiments, CARs can be used to graft the specificity of an antibody onto an immunoresponsive cell, such as a T cell. In some embodiments, CARs of the present disclosure comprise an extracellular antigen-binding domain (e.g., an scFv) fused to a transmembrane domain, fused to one or more intracellular signaling domains.

In some embodiments, binding of the chimeric antigen receptor to its cognate ligand is sufficient to induce activation of the immunoresponsive cell. In some embodiments, binding of the chimeric antigen receptor to its cognate ligand is sufficient to induce stimulation of the immunoresponsive cell. In some embodiments, activation of an immunoresponsive cell results in killing of target cells. In some embodiments, activation of an immunoresponsive cell results in cytokine or chemokine expression and/or secretion by the immunoresponsive cell. In some embodiments, stimulation of an immunoresponsive cell results in cytokine or chemokine expression and/or secretion by the immunoresponsive cell. In some embodiments, stimulation of an immunoresponsive cell induces differentiation of the immunoresponsive cell. In some embodiments, stimulation of an immunoresponsive cell induces proliferation of the immunoresponsive cell.

A CAR of the present disclosure may be a first, second, or third generation CAR. “First generation” CARs comprise a single intracellular signaling domain, generally derived from a T cell receptor chain. “First generation” CARs generally have the intracellular signaling domain from the CD3-zeta (CD3ζ) chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add a second intracellular signaling domain from one of various co-stimulatory molecules (e.g., CD28, 4-1BB, ICOS, OX40) to the cytoplasmic tail of the CAR to provide additional signals to the T cell. “Second generation” CARs provide both co-stimulation (e.g., CD28 or 4-1BB) and activation (CD3ζ). Preclinical studies have indicated that “Second Generation” CARs can improve the anti-tumor activity of immunoresponsive cell, such as a T cell. “Third generation” CARs have multiple intracellular co-stimulation signaling domains (e.g., CD28 and 4-1BB) and an intracellular activation signaling domain (CD3ζ).

In some embodiments, the extracellular antigen-binding domain of a CAR of the present disclosure binds to one or more antigens expressed on a cell, such as a solid tumor cell, with a dissociation constant (K_{D}) of about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ M or less, about 2×10⁻⁹ M or less, or about 1×10⁻⁹ M or less. In some embodiments, the K_{D} ranges from about is about 2×10⁻⁷ M to about 1×10⁻⁹ M.

Binding of the extracellular antigen-binding domain of a CAR of the present disclosure can be determined by, for example, an enzyme-linked immunosorbent assay (ELISA), a radioimmunoassay (RIA), FACS analysis, a bioassay (e.g., growth inhibition), or a Western Blot assay. Each of these assays generally detect the presence of protein-antibody complexes of particular interest by employing a labeled reagent (e.g., an antibody or scFv) specific for the complex of interest. For example, the scFv can be radioactively labeled and used in an RIA assay. The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet).

In some embodiments, CARs of the present disclosure comprise an extracellular antigen-binding domain that binds to one or more antigens expressed on a solid tumor (e.g., a tumor of the lung, pancreas, gastrointestinal tract, colon, brain, neuronal tissue, endocrine, bone, bone marrow, immune system, muscle, liver, gallbladder, kidney, urinary bladder, male reproductive organs, female reproductive organs, adipose, soft tissue, or skin) cell, a transmembrane domain, and one or more intracellular signaling domains. In some embodiments, the extracellular antigen-binding domain comprises an scFv. In some embodiments, the extracellular antigen-binding domain comprises a Fab fragment, which may be crosslinked. In certain embodiments, the extracellular binding domain is a F(ab)₂ fragment.

### Extracellular Antigen-Binding Domain

In some embodiments, the extracellular antigen-binding domain of a CAR of the present disclosure specifically binds to one or more antigens expressed on a solid tumor cell, such as a tumor of the lung, pancreas, gastrointestinal tract, colon, brain, neuronal tissue, endocrine, bone, bone marrow, immune system, muscle, liver, gallbladder, kidney, urinary bladder, male reproductive organs, female reproductive organs, adipose, soft tissue, or skin cell. In certain embodiments, the extracellular antigen-binding domain binds to one or more antigens expressed on a solid tumor cell (solid tumor antigens). In some embodiments, the one or more solid tumor antigens are human polypeptides.

Antigen-binding domains of the present disclosure can include any domain that binds to the antigen including, without limitation, a monoclonal antibody, a polyclonal antibody, a recombinant antibody, a bispecific antibody, a conjugated antibody, a human antibody, a humanized antibody, and a functional fragment thereof, including but not limited to a single-domain antibody (sdAb) such as a heavy chain variable domain (VH), a light chain variable domain (VL) and a variable domain (V_{H}H) of camelid derived nanobody, and to an alternative scaffold known in the art to function as antigen-binding domain, such as a recombinant fibronectin domain, a T cell receptor (TCR), a recombinant TCR with enhanced affinity, or a fragment thereof, e.g., single chain TCR, and the like. In some instances, it is beneficial for the antigen-binding domain to be derived from the same species in which the CAR will ultimately be used in. For example, for use in humans, it may be beneficial for the antigen-binding domain of the CAR to comprise human or humanized residues for the antigen-binding domain of an antibody or antibody fragment.

In some embodiments, the extracellular antigen-binding domain comprises an antibody. In certain embodiments, the antibody is a human antibody. In certain embodiments, the antibody is a humanized antibody. In certain embodiments, the antibody is a chimeric antibody. In some embodiments, the extracellular antigen-binding domain comprises an antigen-binding fragment of an antibody.

In some embodiments, the extracellular antigen-binding domain comprises a F(ab) fragment. In certain embodiments, the extracellular antigen-binding domain comprises a F(ab′) fragment.

In some embodiments, the extracellular antigen-binding domain comprises an scFv.

Various scFvs derived from antibodies capable of binding to a solid tumor antigen are provided in Table 3. In some embodiments, the extracellular antigen-binding domain comprises an scFv as provided in Table 3.

TABLE 3

| scFv | Amino Acid Sequence | SEQ ID NO |
|---|---|---|
| hMN14 scFv | DIQLTQSPSSLSASVGDRVTITCKASQDVGTSVAWYQQKPGKAPKLLIYWTS | 19 |
| (VL-VH) | TRHTGVPSRFSGSGSGTDFTFTISSLQPEDIATYYCQQYSLYRSFGQGTKVEIK | |
| | GGSGSGGSGSGGSGSEVQLVESGGGVVQPGRSLRLSCSASGFDFTTYWMSW | |
| | VRQAPGKGLEWIGEIHPDSSTINYAPSLKDRFTISRDNAKNTLFLQMDSLRPE | |
| | DTGVYFCASLYFGFPWFAYWGQGTPVTVSS | |
| hMFE23 scFv | QVKLEQSGAEVVKPGASVKLSCKASGFNIKDSYMHWLRQGPGQRLEWI | 20 |
| (VH-VL) | GWIDPENGDTEYAPKFQGKATFTTDTSANTAYLGLSSLRPEDTAVYYCN | |
| | EGTPTGPYYFDYWGQGTLVTVSSGGGGSGGGGSGGGGSENVLTQSPSSM | |
| | SVSVGDRVTIACSASSSVPYMHWLQQKPGKSPKLLIYLTSNLASGVPSRF | |
| | SGSGSGTDYSLTISSVQPEDAATYYCQQRSSYPLTFGGGTKLEIK | |
| MG7 (“FM4”) | EVKLVESGGGLVQPGGSLRLSCSISGFTFTDYYMNWVRQSPGKALEWLGFI | 21 |
| scFv (VL-VH) | RNKVNGDTTEYSASVKGRFTISRDISQSILYLQMNTLRTEDSATYYCARDKG | |
| | IAYYFDYWGQGTTLTVSSGGGGSGGGGSGGGGSQIVLSQSPAILFASPGEKV | |
| | TMTCRASSSVSYIHWYQQKPGSSPKPWIHGTSNLASGVPARFSGSGSGTSYS | |
| | LTISRMEAEDAATYYCQQWSSNLSTFGGGTKLEIK | |
| MRG1 scFv | QVQLQQSGAELVRPGTSVKVSCKASGYAFTNNLIEWVKQRPGQGLEWIGVI | 22 |
| (VH-VL) | NPGSGDTNYNEKFKGKATLTADKSSNTAYMQLSSLTSDDSAVYFCARGDY | |
| | YGGFAVDYWGQGTSVTVSSGGGGSGGGGSGGGGSDIQMTQTTSSLSASLG | |
| | DRVTISCRTSQDIGNYLNWYQQKPDGTVKLLIYYTSRLHSGVPSRFSGSGSG | |
| | TDYSLTISNLEQEDIATYFCQQGKSLPRTFGGGTKLEI | |
| Tusamitamab | DIQMTQSPASLSASVGDRVTITCRASENIFSYLAWYQQKPGKSPKLLVYNTR | 23 |
| scFv (VH-VL) | TLAEGVPSRFSGSGSGTDFSLTISSLQPEDFATYYCQHHYGTPFTFGSGTKLEI | |
| | KRGGGGSGGGGSGGGGSEVQLQESGPGLVKPGGSLSLSCAASGFVFSSYDM | |
| | SWVRQTPERGLEWVAYISSGGGITYAPSTVKGRFTVSRDNAKNTLYLQMNS | |
| | LTSEDTAVYYCAAHYFGSSGPFAYWGQGTLVTVSSA | |
| Tusamitamab | EVQLQESGPGLVKPGGSLSLSCAASGFVFSSYDMSWVRQTPERGLEWVAYI | 24 |
| scFv (VL-VH) | SSGGGITYAPSTVKGRFTVSRDNAKNTLYLQMNSLTSEDTAVYYCAAHYFG | |
| | SSGPFAYWGQGTLVTVSSAGGGGSGGGGSGGGGSDIQMTQSPASLSASVGD | |
| | RVTITCRASENIFSYLAWYQQKPGKSPKLLVYNTRTLAEGVPSRFSGSGSGT | |
| | DFSLTISSLQPEDFATYYCQHHYGTPFTFGSGTKLEIKR | |
| A5B7 scFv | EVQLLESGGGLVQPGGSLRLSCATSGFTFTDYYMNWVRQAPGKGLEWLGFI | 25 |
| (VH-VL) | GNKANGYTTEYSASVKGRFTISRDKSKSTLYLQMNTLQAEDSAIYYCTRDR | |
| | GLRFYFDYWGQGTLVTVSSASTKGPSVFPLAPCSRSTSESTAALGCLVKDYF | |
| | PEPVTVLYLQMNTLQAEDSAIYYCTRDRGLRFYFDYWGQGTLVTVSSGGG | |
| | GSGGGGSGGGGSQTVLTQSPSSLSVSVGDRVTITCRASSSVTYIHWYQQKPG | |
| | LAPKSLIYATSNLASGVPSRFSGSGSGTDYTFTISSLQPEDIATYYCQHWSSKP | |
| | PTFGQGTKVEVKRTV | |
| A5B7 scFv | QTVLTQSPSSLSVSVGDRVTITCRASSSVTYIHWYQQKPGLAPKSLIYATSNL | 26 |
| (VL-VH) | ASGVPSRFSGSGSGTDYTFTISSLQPEDIATYYCQHWSSKPPTFGQGTKVEVK | |
| | RTVGGGGSGGGGSGGGGSEVQLLESGGGLVQPGGSLRLSCATSGFTFTDYY | |
| | MNWVRQAPGKGLEWLGFIGNKANGYTTEYSASVKGRFTISRDKSKSTLYLQ | |
| | MNTLQAEDSAIYYCTRDRGLRFYFDYWGQGTLVTVSSASTKGPSVFPLAPC | |
| | SRSTSESTAALGCLVKDYFPEPVTVLYLQMNTLQAEDSAIYYCTRDRGLRFY | |
| | FDYWGQGTLVTVSS | |

In some embodiments, the extracellular antigen-binding domain comprises two single chain variable fragments (scFvs). In some embodiments, each of the two scFvs binds to a distinct epitope on the same antigen. In some embodiments, the extracellular antigen-binding domain comprises a first scFv and a second scFv. In some embodiments, the first scFv and the second scFv bind distinct epitopes on the same antigen. In certain embodiments, the scFv is a human scFv. In certain embodiments, the scFv is a humanized scFv. In certain embodiments, the scFv is a chimeric scFv. In certain embodiments, the scFv comprises a heavy chain variable domain (VH) and a light chain variable domain (VL). In certain embodiments, the VH and VL are separated by a peptide linker. In some embodiments, the peptide linker comprises an amino acid sequence as shown in Table 4.

TABLE 4
Suitable peptide linkers for use

| | | SEQ |
|---|---|---|
| Linker | Amino Acid Sequence | ID NO: |
| (G₂S)₁ scFv linker | GGS | 27 |
| (G₂S)₂ scFv linker | GGSGGS | 28 |
| (G₂S)₃ scFv linker | GGSGGSGGS | 29 |
| (G₂S)₄ scFv linker | GGSGGSGGSGGS | 30 |
| (G₂S)₅ scFv linker | GGSGGSGGSGGSGGS | 31 |
| (G₃S)₁ scFv linker | GGGS | 32 |
| (G₃S)₂ scFv linker | GGGSGGGS | 33 |
| (G₃S)₃ scFv linker | GGGSGGGSGGGS | 34 |
| (G₃S)₄ scFv linker | GGGSGGGSGGGSGGGS | 35 |
| (G₃S)₅ scFv linker | GGGSGGGSGGGSGGGSGGGS | 36 |
| (G₄S)₁ linker | GGGGS | 37 |
| (G₄S)₂ linker | GGGGSGGGGS | 38 |
| (G₄S)₃ linker | GGGGSGGGGSGGGGS | 39 |
| (G₄S)₄ linker | GGGGSGGGGSGGGGSGGGGS | 40 |
| (G₄S)₅ linker | GGGGSGGGGSGGGGSGGGGSGGGGS | 41 |
| Whitlow linker | GSTSGSGKPGSGEGSTKG | 42 |
| scFv linker 2 | EAAAKEAAAKEAAAKEAAAK | 43 |
| (GGSGS)₃ scFv | GGSGSGGSGSGGSGS | 77 |
| linker | | |

In certain embodiments, the peptide linker is encoded by a nucleic acid comprising the sequence of GGCGGAGGCGGATCAGGTGGCGGAGGAAGTGGCGGCGGAGGATCT (SEQ ID NO: 44).

In certain embodiments, the scFv comprises the structure VH-L-VL or VL-L-VH, wherein VH is the heavy chain variable domain, L is the peptide linker, and VL is the light chain variable domain.

In some embodiments, each of the one or more scFvs comprises the structure VH-L-VL or VL-L-VH, wherein VH is the heavy chain variable domain, L is the peptide linker, and VL is the light chain variable domain. When there are two or more scFv linked together, each scFv can be linked to the next scFv with a peptide linker. In some embodiments, each of the one or more scFvs is separated by a peptide linker. In some embodiments, the peptide linker separating each of the scFvs comprises an amino acid sequence as shown in Table 4.

In some embodiments, the peptide linker comprises an amino acid sequence of SEQ ID NO: 27. In some embodiments, the peptide linker comprises an amino acid sequence of SEQ ID NO: 28. In some embodiments, the peptide linker comprises an amino acid sequence of SEQ ID NO: 29. In some embodiments, the peptide linker comprises an amino acid sequence of SEQ ID NO: 30. In some embodiments, the peptide linker comprises an amino acid sequence of SEQ ID NO: 31. In some embodiments, the peptide linker comprises an amino acid sequence of SEQ ID NO: 32. In some embodiments, the peptide linker comprises an amino acid sequence of SEQ ID NO: 33. In some embodiments, the peptide linker comprises an amino acid sequence of SEQ ID NO: 34. In some embodiments, the peptide linker comprises an amino acid sequence of SEQ ID NO: 35. In some embodiments, the peptide linker comprises an amino acid sequence of SEQ ID NO: 36. In some embodiments, the peptide linker comprises an amino acid sequence of SEQ ID NO: 37. In some embodiments, the peptide linker comprises an amino acid sequence of SEQ ID NO: 38. In some embodiments, the peptide linker comprises an amino acid sequence of SEQ ID NO: 39. In some embodiments, the peptide linker comprises an amino acid sequence of SEQ ID NO: 40. In some embodiments, the peptide linker comprises an amino acid sequence of SEQ ID NO: 41. In some embodiments, the peptide linker comprises an amino acid sequence of SEQ ID NO: 42. In some embodiments, the peptide linker comprises an amino acid sequence of SEQ ID NO:43.

In some embodiments, the cell comprises a first chimeric receptor and a second chimeric receptor. The antigen binding domain of the first chimeric receptor and the antigen binding domain of the second chimeric receptor can be an appropriate antigen biding domain described herein or known in the art. For example, the first or second antigen binding domain can be one or more antibodies, antigen-binding fragments of an antibody, F(ab) fragments, F(ab′) fragments, single chain variable fragments (scFvs), or single-domain antibodies (sdAbs). In some embodiments, the antigen-binding domain of the first chimeric receptor and/or the second chimeric receptor comprises two single chain variable fragments (scFvs). In some embodiments, each of the two scFvs binds to a distinct epitope on the same antigen.

In some embodiments, the extracellular antigen-binding domain comprises a single-domain antibody (sdAb). In certain embodiments, the sdAb is a humanized sdAb. In certain embodiments, the sdAb is a chimeric sdAb.

In some embodiments, a CAR of the present disclosure may comprise two or more antigen-binding domains, three or more antigen-binding domains, four or more antigen-binding domains, five or more antigen-binding domains, six or more antigen-binding domains, seven or more antigen-binding domains, eight or more antigen-binding domains, nine or more antigen-binding domains, or ten or more antigen-binding domains. In some embodiments, each of the two or more antigen-binding domains binds the same antigen. In some embodiments, each of the two or more antigen-binding domains binds a different epitope of the same antigen. In some embodiments, each of the two or more antigen-binding domains binds a different antigen. In some embodiments, the two or more antigen-binding domains provide the CAR with logic gating, such as OR logic gating.

In some embodiments, the CAR comprises two antigen-binding domains. In some embodiments, the two antigen-binding domains are attached to one another via a flexible linker. In some embodiments, each of the two-antigen-binding domains may be independently selected from an antibody, an antigen-binding fragment of an antibody, an scFv, a sdAb, a recombinant fibronectin domain, a T cell receptor (TCR), a recombinant TCR with enhanced affinity, and a single chain TCR. In some embodiments, the CAR comprising two antigen-binding domains is a bispecific CAR or a tandem CAR (tanCAR).

In certain embodiments, the bispecific CAR or tanCAR comprises an antigen-binding domain comprising a bispecific antibody or antibody fragment (e.g., scFv). In some embodiments, within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁—VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁ VH₁—VH₂—VL₂. In some embodiments, a linker is disposed between the two antibodies or antibody fragments (e.g., scFvs), for example, between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser)n linker (SEQ ID NO: 138), wherein n is 1, 2, 3, 4, 5, or 6. In general, the linker between the two scFvs should be long enough to avoid mispairing between the domains of the two scFvs. In some embodiments, a linker is disposed between the VL and VH of the first scFv. In some embodiments, a linker is disposed between the VL and VH of the second scFv. In constructs that have multiple linkers, any two or more of the linkers may be the same or different. Accordingly, in some embodiments, a bispecific CAR or tanCAR comprises VLs, VHs, and may further comprise one or more linkers in an arrangement as described herein.

In some embodiments, the bivalent receptor comprises a CEA CAR and a CEACAM1 CAR. In some embodiments, the bivalent receptor comprises a CEA CAR and a CEACAM5 CAR. In some embodiments, the bivalent receptor comprises a CEA CAR and a CEACAM6 CAR. In some embodiments, the bivalent receptor comprises a CEACAM1 CAR and a CEACAM5 CAR. In some embodiments, the bivalent receptor comprises a CEACAM1 CAR and a CEACAM6 CAR. In some embodiments, the bivalent receptor comprises a CEACAM5 CAR and a CEACAM6 CAR.

In some embodiments, the bivalent receptor comprises a CEA CAR and a VSIG2 CAR. In some embodiments, the bivalent receptor comprises a CEA CAR and a CPM CAR. In some embodiments, the bivalent receptor comprises a CEA CAR and a ITM2C CAR. In some embodiments, the bivalent receptor comprises a CEA CAR and a SLC26A2 CAR. In some embodiments, the bivalent receptor comprises a CEA CAR and a SLC4A4 CAR. In some embodiments, the bivalent receptor comprises a CEA CAR and a GPA33 CAR. In some embodiments, the bivalent receptor comprises a CEA CAR and a PLA2G2A CAR. In some embodiments, the bivalent receptor comprises a CEA CAR and an ABCA8 CAR. In some embodiments, the bivalent receptor comprises a CEA CAR and an ATP1A2 CAR. In some embodiments, the bivalent receptor comprises a CEA CAR and a CHP2 CAR. In some embodiments, the bivalent receptor comprises a CEA CAR and an SLC26A3 CAR.

In some embodiments, the bivalent receptor comprises a CEACAM1 CAR and a VSIG2 CAR. In some embodiments, the bivalent receptor comprises a CEACAM1 CAR and a CPM CAR. In some embodiments, the bivalent receptor comprises a CEACAM1 CAR and a ITM2C CAR. In some embodiments, the bivalent receptor comprises a CEACAM1 CAR and a SLC26A2 CAR. In some embodiments, the bivalent receptor comprises a CEACAM1 CAR and a SLC4A4 CAR. In some embodiments, the bivalent receptor comprises a CEACAM1 CAR and a GPA33 CAR. In some embodiments, the bivalent receptor comprises a CEACAM1 CAR and a PLA2G2A CAR. In some embodiments, the bivalent receptor comprises a CEACAM1 CAR and a ABCA8 CAR. In some embodiments, the bivalent receptor comprises a CEACAM1 CAR and an ATP1A2 CAR. In some embodiments, the bivalent receptor comprises a CEACAM1 CAR and a CHP2 CAR. In some embodiments, the bivalent receptor comprises a CEACAM1 CAR and an SLC26A3 CAR.

In some embodiments, the bivalent receptor comprises a CEACAM5 CAR and a VSIG2 CAR. In some embodiments, the bivalent receptor comprises a CEACAM5 CAR and a CPM CAR. In some embodiments, the bivalent receptor comprises a CEACAM5 CAR and an ITM2C CAR. In some embodiments, the bivalent chimeric antigen receptor comprises a CEACAM5 CAR and a SLC26A2 CAR. In some embodiments, the bivalent receptor comprises a CEACAM5 CAR and a SLC4A4 CAR. In some embodiments, the bivalent receptor comprises a CEACAM5 CAR and a GPA33 CAR. In some embodiments, the bivalent receptor comprises a CEACAM5 CAR and a PLA2G2A CAR. In some embodiments, the bivalent receptor comprises a CEACAM5 CAR and an ABCA8 CAR. In some embodiments, the bivalent receptor comprises a CEACAM5 CAR and an ATP1A2 CAR. In some embodiments, the bivalent receptor comprises a CEACAM5 CAR and a CHP2 CAR. In some embodiments, the bivalent receptor comprises a CEACAM5 CAR and an SLC26A3 CAR.

In some embodiments, the bivalent receptor comprises a CEACAM6 CAR and a VSIG2 CAR. In some embodiments, the bivalent receptor comprises a CEACAM6 CAR and a CPM CAR. In some embodiments, the bivalent receptor comprises a CEACAM6 CAR and an ITM2C CAR. In some embodiments, the bivalent receptor comprises a CEACAM6 CAR and a SLC26A2 CAR. In some embodiments, the bivalent receptor comprises a CEACAM6 CAR and a SLC4A4 CAR. In some embodiments, the bivalent receptor comprises a CEACAM6 CAR and a GPA33 CAR. In some embodiments, the bivalent receptor comprises a CEACAM6 CAR and a PLA2G2A CAR. In some embodiments, the bivalent receptor comprises a CEACAM6 CAR and an ABCA8 CAR. In some embodiments, the bivalent receptor comprises a CEACAM6 CAR and an ATP1A2 CAR. In some embodiments, the bivalent receptor comprises a CEACAM6 CAR and a CHP2 CAR. In some embodiments, the bivalent receptor comprises a CEACAM6 CAR and a CHP2 CAR. In some embodiments, the bivalent receptor comprises a CEACAM6 CAR and an SLC26A3 CAR.

In some embodiments, the bivalent chimeric receptor comprises a CAR with an antigen binding domain targeting any antigen provided in Table 1. In some embodiments, the bivalent chimeric receptor comprises a CAR with an antigen binding domain derived from an antibody as provided in Table 2. In some embodiments, the bivalent chimeric receptor comprises a CAR with an antigen binding domain including an scFv as provided in Table 2. In some embodiments, the bivalent chimeric receptor comprises a CAR with two or more antigen binding domains targeting any antigen pair provided in Table 5. In some embodiments, the bivalent chimeric antigen receptor comprises a CAR with any combination of two or more antigen binding domains as described herein.

In some embodiments, chimeric receptors comprise a bicistronic chimeric antigen receptor system, e.g., a chimeric antigen receptor system that comprises an activating CAR and an inhibitory CAR. In some embodiments, the bicistronic chimeric antigen receptor system comprises a CAR with an antigen binding domain targeting any antigen provided in Table 1 and an inhibitory CAR, e.g., with an antigen binding domain targeting any antigen provided in Table 8. In some embodiments, the bicistronic chimeric antigen receptor system comprises a CAR with an antigen binding domain derived from an antibody provided in Table 2 and an inhibitory CAR, e.g., with an antigen binding domain targeting any antigen provided in Table 7. In some embodiments, the bicistronic chimeric antigen receptor system comprises a CAR with two or more antigen binding domains targeting any antigen pair provided in Table 5, wherein at least one antigen binding domain is an activating CAR, and at least one antigen binding domain is an inhibitory CAR. In some embodiments, the bicistronic chimeric antigen receptor system comprises a CAR with any combination of two or more antigen binding domains as described herein, wherein at least one antigen binding domain is an activating CAR, and at least one antigen binding domain is an inhibitory CAR.

### Transmembrane Domain

In some embodiments, the transmembrane domain of a CAR of the present disclosure comprises a hydrophobic alpha helix that spans at least a portion of a cell membrane. It has been shown that different transmembrane domains can result in different receptor stability. After antigen recognition, receptors cluster and a signal is transmitted to the cell. In some embodiments, the transmembrane domain of a CAR of the present disclosure can comprise the transmembrane domain of a CD8 polypeptide, a CD28 polypeptide, a CD25 polypeptide, a CD7 polypeptide, a CD3-zeta polypeptide, a CD4 polypeptide, a 4-1BB polypeptide, an OX40 polypeptide, an ICOS polypeptide, a CTLA-4 polypeptide, a LAX polypeptide, a LAT polypeptide, a PD-1 polypeptide, a LAG-3 polypeptide, a TIM3 polypeptide, a KIR3DS1 polypeptide, a KIR3DL1 polypeptide, an NKG2D polypeptide, an NKG2A polypeptide, a TIGIT polypeptide, a 2B4 polypeptide, a BTLA polypeptide, a LIR-1 (LILRB1) polypeptide, or can be a synthetic peptide, or any combination thereof.

In some embodiments, the transmembrane domain is derived from a CD8 polypeptide. Any suitable CD8 polypeptide may be used. Exemplary CD8 polypeptides include, without limitation, NCBI Reference Nos. NP_001139345 and AAA92533.1. In some embodiments, the transmembrane domain is derived from a CD28 polypeptide. Any suitable CD28 polypeptide may be used. Exemplary CD28 polypeptides include, without limitation, NCBI Reference Nos. NP_006130.1 and NP_031668.3. In some embodiments, the transmembrane domain is derived from a CD3-zeta polypeptide. Any suitable CD3-zeta polypeptide may be used. Exemplary CD3-zeta polypeptides include, without limitation, NCBI Reference Nos. NP_932170.1 and NP_001106862.1. In some embodiments, the transmembrane domain is derived from a CD4 polypeptide. Any suitable CD4 polypeptide may be used. Exemplary CD4 polypeptides include, without limitation, NCBI Reference Nos. NP_000607.1 and NP_038516.1. In some embodiments, the transmembrane domain is derived from a 4-1BB polypeptide. Any suitable 4-1BB polypeptide may be used. Exemplary 4-1BB polypeptides include, without limitation, NCBI Reference Nos. NP_001552.2 and NP_001070977.1. In some embodiments, the transmembrane domain is derived from an OX40 polypeptide. Any suitable OX40 polypeptide may be used. Exemplary OX40 polypeptides include, without limitation, NCBI Reference Nos. NP_003318.1 and NP_035789.1. In some embodiments, the transmembrane domain is derived from an ICOS polypeptide. Any suitable ICOS polypeptide may be used. Exemplary ICOS polypeptides include, without limitation, NCBI Reference Nos. NP_036224 and NP_059508. In some embodiments, the transmembrane domain is derived from a CTLA-4 polypeptide. Any suitable CTLA-4 polypeptide may be used. Exemplary CTLA-4 polypeptides include, without limitation, NCBI Reference Nos. NP_005205.2 and NP_033973.2. In some embodiments, the transmembrane domain is derived from a PD-1 polypeptide. Any suitable PD-1 polypeptide may be used. Exemplary PD-1 polypeptides include, without limitation, NCBI Reference Nos. NP_005009 and NP_032824. In some embodiments, the transmembrane domain is derived from a LAG-3 polypeptide. Any suitable LAG-3 polypeptide may be used. Exemplary LAG-3 polypeptides include, without limitation, NCBI Reference Nos. NP_002277.4 and NP_032505.1. In some embodiments, the transmembrane domain is derived from a 2B4 polypeptide. Any suitable 2B4 polypeptide may be used. Exemplary 2B4 polypeptides include, without limitation, NCBI Reference Nos. NP_057466.1 and NP_061199.2. In some embodiments, the transmembrane domain is derived from a BTLA polypeptide. Any suitable BTLA polypeptide may be used. Exemplary BTLA polypeptides include, without limitation, NCBI Reference Nos. NP_861445.4 and NP_001032808.2. Any suitable LIR-1 (LILRB1) polypeptide may be used. Exemplary LIR-1 (LILRB1) polypeptides include, without limitation, NCBI Reference Nos. NP_001075106.2 and NP_001075107.2.

In some embodiments, the transmembrane domain comprises a polypeptide comprising an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_001139345, AAA92533.1, NP_006130.1, NP 031668.3, NP_932170.1, NP 001106862.1, NP_000607.1, NP 038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2, or fragments thereof. In some embodiments, the homology may be determined using standard software such as BLAST or FASTA. In some embodiments, the polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2 that is at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or at least 240 amino acids in length.

Further examples of suitable polypeptides from which a transmembrane domain may be derived include, without limitation, the transmembrane region(s) of the alpha, beta or zeta chain of the T-cell receptor, CD27, CD3 epsilon, CD45, CD5, CD9, CD16, CD22, CD33, CD37, CD64, CD80, CD86, CD134, CD137, CD154, KIRDS2, CD2, CD27, LFA-1 (CD11a, CD18), GITR, CD40, BAFFR, HVEM (LIGHTR), SLAMF7, NKp80 (KLRF1), NKp44, NKp30, NKp46, CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMFI, CD150, IPO-3), BLAME (SLAMF8), SELPLG (CD162), LTBR, PAG/Cbp, NKG2D, and NG2C. In some embodiments, a transmembrane domain may comprise any of the amino acid sequences listed in Table 5, or an amino acid sequence that is at least 90%, at least 91%, at least 92%, at least 93%, at least 94%, at least 95%, at least 96%, at least 97%, at least 98%, or at least 99% identical to any of the amino acid sequences listed in Table 5.

TABLE 5

| Amino Acid | SEQ ID | |
|---|---|---|
| Sequence | NO: | Description |
| IYIWAPLAGTC | 45 | CD8 Transmembrane |
| GVLLLSLVIT | | Domain |
| FWVLVVVGGVL | 46 | CD28 Transmembrane |
| ACYSLLVTVAF | | Domain |
| IIFWV | | |
| VAAILGLGLVL | 47 | OX40 Transmembrane |
| GLLGPLAILL | | Domain |
| FLVIIVILSAL | 48 | 2B4 Transmembrane |
| FLGTLACFCV | | Domain |

### Spacer Region

In some embodiments, a CAR of the present disclosure can also comprise a spacer region that links the extracellular antigen-binding domain to the transmembrane domain. The spacer region may be flexible enough to allow the antigen-binding domain to orient in different directions to facilitate antigen recognition. In some embodiments, the spacer region may be a hinge from a human protein. For example, the spacer (also referred to herein as “hinge”) may be a human Ig (immunoglobulin) hinge, including without limitation an IgG4 hinge, an IgG2 hinge, a CD8a hinge, or an IgD hinge. In some embodiments, the spacer region may comprise an IgG4 hinge, an IgG2 hinge, an IgD hinge, a CD28 hinge, a KIR2DS2 hinge, an LNGFR hinge, or a PDGFR-beta extracellular linker. In some embodiments, the spacer region is localized between the antigen-binding domain and the transmembrane domain. In some embodiments, a spacer region may comprise any of the amino acid sequences listed in Table 6, or an amino acid sequence that is at least 90%, at least 91%, at least 92%, at least 93%, at least 94%, at least 95%, at least 96%, at least 97%, at least 98%, or at least 99% identical to any of the amino acid sequences listed in Table 6. In some embodiments, nucleic acids encoding any of the spacer regions of the present disclosure may comprise any of the nucleic acid sequences listed in Table 7, or a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 7.

TABLE 6

| | SEQ | |
|---|---|---|
| | ID | |
| Amino Acid Sequence | NO: | Description |
| AAAIEVMYPPPYLDNEKSNG | 49 | CD28 hinge |
| TIIHVKGKHLCPSPLFPGPS | | |
| KP | | |
| TTTPAPRPPTPAPTIALQPL | 50 | CD8 hinge |
| SLRPEACRPAAGGAVHTRGL | | |
| DFACD | | |
| ESKYGPPCPSCP | 51 | IgG4 minimal hinge |
| ESKYGPPAPSAP | 52 | IgG4 minimal |
| | | hinge, no |
| | | disulfides |
| ESKYGPPCPPCP | 53 | IgG4 S228P |
| | | minimal hinge, |
| | | enhanced disulfide |
| | | formation |
| EPKSCDKTHTCP | 54 | IgG1 minimal hinge |
| AAAFVPVFLPAKPTTTPAPR | 55 | Extended CD8a hinge |
| PPTPAPTIASQPLSLRPEAC | | |
| RPAAGGAVHTRGLDFACDIY | | |
| IWAPLAGTCGVLLLSLVITL | | |
| YCNHRN | | |
| ACPTGLYTHSGECCKACNLG | 56 | LNGFR hinge |
| EGVAQPCGANQTVCEPCLDS | | |
| VTFSDVVSATEPCKPCTECV | | |
| GLQSMSAPCVEADDAVCRCA | | |
| YGYYQDETTGRCEACRVCEA | | |
| GSGLVESCQDKQNTVCEECP | | |
| DGTYSDEADAEC | | |
| ACPTGLYTHSGECCKACNLG | 57 | Truncated LNGFR |
| | | hinge |
| EGVAQPCGANQTVC | | (TNFR-Cys1) |
| AVGQDTQEVIVVPHSLPFKV | 58 | PDGFR-beta |
| | | extracellular |
| | | linker |

TABLE 7

| | SEQ ID | Description |
|---|---|---|
| Nucleic Acid Sequence | NO: | |
| GCAGCAGCTATCGAGGTGAT | 59 | CD28 hinge |
| GTATCCTCCGCCCTACCTGG | | |
| ATAATGAAAAGAGTAATGGG | | |
| ACTATCATTCATGTAAAAGG | | |
| GAAGCATCTTTGTCCTTCTC | | |
| CCCTTTTCCCCGGTCCGTCT | | |
| AAACCT | | |
| ACCACAACACCAGCTCCTAG | 60 | CD8 hinge |
| ACCTCCAACTCCTGCTCCTA | | |
| CAATCGCCCTGCAGCCACTG | | |
| TCTCTGAGGCCTGAAGCTTG | | |
| TAGACCTGCTGCTGGCGGAG | | |
| CCGTGCATACCAGAGGACTG | | |
| GATTTCGCCTGCGAC | | |
| GAAAGCAAGTACGGTCCACC | 61 | IgG4 minimal |
| TTGCCCTAGCTGTCCG | | hinge |
| GAATCCAAGTACGGCCCCCC | 62 | IgG4 minimal |
| AGCGCCTAGTGCCCCA | | hinge, no |
| | | disulfides |
| GAATCTAAATATGGCCCGCC | 63 | IgG4 S228P |
| ATGCCCGCCTTGCCCA | | minimal hinge, |
| | | enhanced |
| | | disulfide |
| | | formation |
| GAACCGAAGTCTTGTGATAA | 64 | IgG1 minimal |
| AACTCATACGTGCCCG | | hinge |
| GCTGCTGCTTTCGTACCCGT | 65 | Extended CD8a |
| GTTCCTCCCTGCTAAGCCTA | | hinge |
| CGACTACCCCCGCACCGAGA | | |
| CCACCCACGCCAGCACCCAC | | |
| GATTGCTAGCCAGCCCCTTA | | |
| GTTTGCGACCAGAAGCTTGT | | |
| CGGCCTGCTGCTGGTGGCGC | | |
| GGTACATACCCGCGGCCTTG | | |
| ATTTTGCTTGCGATATATAT | | |
| ATCTGGGCGCCTCTGGCCGG | | |
| AACATGCGGGGTCCTCCTCC | | |
| TTTCTCTGGTTATTACTCTC | | |
| TACTGTAATCACAGGAAT | | |
| GCCTGCCCGACCGGGCTCTA | 66 | LNGFR hinge |
| CACTCATAGCGGGGAATGTT | | |
| GTAAGGCATGTAACTTGGGT | | |
| GAGGGCGTCGCACAGCCCTG | | |
| CGGAGCTAACCAAACAGTGT | | |
| GCGAACCCTGCCTCGATAGT | | |
| GTGACGTTCTCTGATGTTGT | | |
| ATCAGCTACAGAGCCTTGCA | | |
| AACCATGTACTGAGTGCGTT | | |
| GGACTTCAGTCAATGAGCGC | | |
| TCCATGTGTGGAGGCAGATG | | |
| ATGCGGTCTGTCGATGTGCT | | |
| TACGGATACTACCAAGACGA | | |
| GACAACAGGGCGGTGCGAGG | | |
| CCTGTAGAGTTTGTGAGGCG | | |
| GGCTCCGGGCTGGTGTTTTC | | |
| ATGTCAAGACAAGCAAAATA | | |
| CGGTCTGTGAAGAGTGCCCT | | |
| GATGGCACCTACTCAGACGA | | |
| AGCAGATGCAGAATGC | | |
| GCCTGCCCTACAGGACTCTA | 67 | Truncated |
| CACGCATAGCGGTGAGTGTT | | LNGFR hinge |
| GTAAAGCATGCAACCTCGGG | | (TNFR-Cys1) |
| GAAGGTGTAGCCCAGCCATG | | |
| CGGGGCTAACCAAACCGTTT | | |
| GC | | |
| GCTGTGGGCCAGGACACGCA | 68 | PDGFR-beta |
| GGAGGTCATCGTGGTGCCAC | | extracellular |
| ACTCCTTGCCCTTTAAGGTG | | linker |

In some embodiments, the spacer region comprises the sequence shown in SEQ ID NO: 49. In some embodiments, the spacer region comprises the sequence shown in SEQ ID NO: 50. In some embodiments, the spacer region comprises the sequence shown in SEQ ID NO: 51. In some embodiments, the spacer region comprises the sequence shown in SEQ ID NO: 52. In some embodiments, the spacer region comprises the sequence shown in SEQ ID NO: 53. In some embodiments, the spacer region comprises the sequence shown in SEQ ID NO: 54. In some embodiments, the spacer region comprises the sequence shown in SEQ ID NO: 55. In some embodiments, the spacer region comprises the sequence shown in SEQ ID NO: 56. In some embodiments, the spacer region comprises the sequence shown in SEQ ID NO: 57. In some embodiments, the spacer region comprises the sequence shown in SEQ ID NO: 58.

In some embodiments, a CAR of the present disclosure may further include a short oligopeptide or polypeptide linker that is between 2 amino acid residues and 10 amino acid residues in length, and that may form the linkage between the transmembrane domain and the cytoplasmic region of the CAR. A non-limiting example of a suitable linker is a glycine-serine doublet. In some embodiments, the linker comprises the amino acid sequence of GGCKJSGGCKJS (SEQ ID NO: 69).

### Intracellular Signaling Domains

In some embodiments, a CAR of the present disclosure comprises one or more cytoplasmic domains or regions. The cytoplasmic domain or region of the CAR may include an intracellular signaling domain. An intracellular signaling domain is typically responsible for activation of one or more effector functions of an immune cell (e.g., a T cell or an NK cell) that has been engineered to express a CAR of the present disclosure. For example, an effector function of a T cell may be cytolytic activity or helper activity, such as the secretion of cytokines. Accordingly, in some embodiments the term “intracellular signaling domain” refers to the portion of a protein which transduces an effector function signal and directs the cell to perform a specialized function. While the entire intracellular signaling domain may be employed, in many instances it is not necessary to use the entire chain. In embodiments where a truncated portion of the intracellular signaling domain is used, such a truncated portion may be used in place of the corresponding intact chain as long as the truncated portion transduces the effector function signal.

Examples of suitable intracellular signaling domains that may be used in CARs of the present disclosure include, without limitation, cytoplasmic sequences of the T cell receptor (TCR) and co-receptors that act in concert to initiate signal transduction following antigen receptor engagement, as well as any derivative or variant of these sequences and any recombinant sequence that has the same functional capability.

Without wishing to be bound by theory, it is believed that signals generated through the TCR alone are insufficient for full activation of the T cell and that a secondary and/or costimulatory signal is thus also required for full activation. Accordingly, T cell activation may be mediated by two distinct classes of cytoplasmic signaling sequences, those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

In some embodiments, a primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs). Examples of suitable ITAM-containing primary intracellular signaling domains that that may be used in the CARs of the present disclosure include, without limitation, those of CD3-zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d.

In some embodiments, a CAR of the present disclosure comprises an intracellular signaling domain, e.g., a primary signaling domain of CD3-zeta polypeptide. A CD3-zeta polypeptide of the present disclosure may have an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_932170 or NP_001106864.2, or fragments thereof. In some embodiments, the CD3-zeta polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_932170 or NP_001106864.2 that is at least 20, at least 30, at least 40, at least 50, at least 60, at least 70, at least 80, at least 90, at least 100, at least 110, at least 120, at least 130, at least 140, at least 150, or at least 160, at least 170, or at least 180 amino acids in length.

In other embodiments, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In one embodiment, a primary signaling domain comprises one, two, three, four or more ITAM motifs.

In some embodiments, the intracellular signaling domain of a CAR of the present disclosure can comprise the CD3-zeta signaling domain by itself or it can be combined with any other desired intracellular signaling domain(s) useful in the context of a CAR of the present disclosure. For example, the intracellular signaling domain of the CAR can comprise a CD3-zeta chain portion and a costimulatory signaling domain. The costimulatory signaling domain may refer to a portion of the CAR comprising the intracellular domain of a costimulatory molecule. A costimulatory molecule of the present disclosure is a cell surface molecule other than an antigen receptor or its ligands that may be required for an efficient response of lymphocytes to an antigen. Examples of suitable costimulatory molecules include, without limitation, CD97, CD2, ICOS, CD27, CD154, CD8, OX40, 4-1BB, CD28, ZAP40, CD30, GITR, HVEM, DAP10, DAP12, MyD88, 2B4, CD40, PD-1, lymphocyte function-associated antigen-1 (LFA-1), CD7, LIGHT, NKG2C, B7-H3, and a ligand that specifically binds with CD83, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, CDS, ICAM-1, (CDiia/CD18), BAFFR, KIRD3S1, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, and the like.

Non-limiting examples of intracellular signalling domains (ICDs) are provided in Table 8.

TABLE 8

| | SEQ | |
|---|---|---|
| | ID | |
| Amino Acid Sequence | NO: | Description |
| KRGRKKLLYIFKQPFMRPV | 70 | 4-1BB ICD |
| QTTQEEDGCSCRFPEEEE | | |
| GGCEL | | |
| RSKRSRLLHSDYMNMTPRR | 71 | CD28 ICD |
| PGPTRKHYQPYAPPRDFA | | |
| AYRS | | |
| ALYLLRRDQRLPPDAHKPP | 72 | OX40 ICD |
| GGGSFRTPIQEEQADAHS | | |
| TLAKI | | |
| WRRKRKEKQSETSPKEFLT | 73 | 2B4 ICD |
| IYEDVKDLKTRRNHEQEQ | | |
| TFPGGGSTIYSMIQSQSS | | |
| APTSQEPAYTLYSLIQPS | | |
| RKSGSRKRNHSPSFNSTI | | |
| YEVIGKSQPKAQNPARLS | | |
| RKELENFDVYS | | |
| RVKFSRSADAPAYKQGQN | 74 | CD3z ICD |
| QLYNELNLGRREEYDVLD | | |
| KRRGRDPEMGGKPRRKNP | | |
| QEGLYNELQKDKMAEAYS | | |
| EIGMKGERRRGKGHDGLY | | |
| QGLSTATKDTYDALHMQ | | |
| ALPPR | | |

In some embodiments, the intracellular signaling sequences within the cytoplasmic portion of a CAR of the present disclosure may be linked to each other in a random or specified order. In some embodiments, a short oligopeptide or polypeptide linker, for example, between 2 amino acids and 10 amino acids (e.g., 2 amino acids, 3 amino acids, 4 amino acids, 5 amino acids, 6 amino acids, 7 amino acids, 8 amino acids, 9 amino acids, or 10 amino acids) in length may form the linkage between intracellular signaling sequences. In one embodiment, a glycine-serine doublet can be used as a suitable linker. In one embodiment, a single amino acid, e.g., an alanine or a glycine, can be used as a suitable linker.

In some embodiments, the intracellular signaling domain comprises two or more costimulatory signaling domains, e.g., two costimulatory signaling domains, three costimulatory signaling domains, four costimulatory signaling domains, five costimulatory signaling domains, six costimulatory signaling domains, seven costimulatory signaling domains, eight costimulatory signaling domains, nine costimulatory signaling domains, 10 costimulatory signaling domains, or more costimulatory signaling domains. In one embodiment, the intracellular signaling domain comprises two costimulatory signaling domains. In some embodiments, the two or more costimulatory signaling domains are separated by a linker of the present disclosure. In one embodiment, the linker is a glycine residue. In another embodiment, the linker is an alanine residue.

In some embodiments, a CAR of the present disclosure further includes an epitope tag. An epitope tag is a polypeptide sequence included within a polypeptide as a label that can be detected, for example, by a monoclonal antibody. Examples of epitope tags include a FLAG tag, a strep tag, an HA tag, a V5 tag, and a myc tag. An exemplary epitope tag is a myc tag of amino acid sequence EQKLISEEDL (SEQ ID NO: 75).

In some embodiments, a cell of the present disclosure expresses a CAR that includes an antigen-binding domain that binds a target antigen of the present disclosure, a transmembrane domain of the present disclosure, a primary signaling domain, and one or more costimulatory signaling domains.

### Natural Killer CARs (NK CARs)

In some embodiments, a CAR of the present disclosure comprises one or more components of a natural killer (NK) cell, thereby forming an NK CAR. The NK component may be a transmembrane domain, a hinge domain, or a cytoplasmic domain from any suitable natural killer cell receptor, including without limitation, a killer cell immunoglobulin-like receptor (KIR), such as KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1, KIR3DS1, KIR3DL2, KIR3DL3, KIR2DP1, and KIRS DPI; a natural cytotoxicity receptor (NCR), such as NKp30, NKp44, NKp46; a signaling lymphocyte activation molecule (SLAM) family of immune cell receptor, such as CD48, CD229, 2B4, CD84, NTB-A, CRACC, BLAME, and CD2F-10; an Fc receptor (FcR), such as CD16, and CD64; and an Ly49 receptor, such as LY49A and LY49C. In some embodiments, the NK-CAR may interact with an adaptor molecule or intracellular signaling domain, such as DAP12. The structural components as described above of a CAR are also applicable to the structure of an NK CAR.

Exemplary configurations and sequences of CARs comprising NK receptor components are described in International Patent Publication WO2014/145252, published Sep. 18, 2014.

One advantage of CAR NK cell therapy relative to CAR T therapy is the significantly decreased risk of inducing graft versus host disease (GvHD). Accordingly, as no severe toxicities are observed or expected to occur with CAR NK cells, treatment can be administered without requiring hospitalization, significantly reducing the huge indirect costs associated with CAR-T cell-based therapy due to hospitalization post-treatment (Oberschmidt et al. (2017), Front Immunol, 8:654).

### Chimeric Inhibitory Receptors

Certain aspects of the present disclosure relate to chimeric inhibitory receptors. Chimeric inhibitory receptors are useful, for example, as NOT logic gates for controlling cell activity, such as immune cell activity. In some embodiments, chimeric inhibitory receptors of the present disclosure specifically bind to one or more antigens that are expressed on normal cells but not on tumor cells.

In some embodiments, the chimeric inhibitory receptor comprises an antigen-binding domain, a transmembrane domain of the present disclosure (e.g., any suitable transmembrane domain used in conjunction with a chimeric receptor of the present disclosure), and an intracellular domain. In some embodiments, the chimeric inhibitory receptor may inhibit one or more activities of a cell, such as an immunoresponsive cell.

In some embodiments, the chimeric inhibitory receptor may comprise an enzymatic inhibitory domain. When the chimeric inhibitory receptor is located proximal to a receptor, such as an immune receptor in a cell membrane, binding of a cognate antigen to the antigen-binding domain activates the enzymatic inhibitory domain to inhibit activation of the receptor. As used herein, the term “enzymatic inhibitory domain” refers to a protein domain that inhibits an intracellular signal transduction cascade, for example a native T cell activation cascade. The disclosed chimeric inhibitory receptors thus can be engineered to contain appropriate antigen-binding domains that will reduce, for example, immune responses in the presence of the cognate antigen. Uses of chimeric inhibitory receptors of the present disclosure include, but are not limited to, reducing immune responses, controlling T cell activation, and controlling CAR-NK or CAR-T responses.

In some embodiments, the enzymatic inhibitory domain of a chimeric inhibitory receptor of the present disclosure comprises at least a portion of an extracellular domain, a transmembrane domain, and/or an intracellular domain. In some embodiments, the enzymatic inhibitory domain comprises at least a portion of an enzyme. In some embodiments, the enzyme is selected from CSK, SHP-1, PTEN, CD45, CD148, PTP-MEG1, PTP-PEST, c-CBL, CBL-b, PTPN22, LAR, PTPH1, SHIP-1, and RasGAP (see e.g., Stanford et al., Regulation of TCR signaling by tyrosine phosphatases: from immune homeostasis to autoimmunity, Immunology, 2012 September; 137(1): 1-19). In some embodiments, the portion of the enzyme comprises an enzyme domain(s), an enzyme fragment(s), or a mutant(s) thereof. In some embodiments, the portion of the enzyme is a catalytic domain of the enzyme. In some embodiments, the enzyme domain(s), enzyme fragment(s), or mutants(s) thereof are selected to maximize efficacy and minimize basal inhibition.

In some embodiments, the enzymatic inhibitory domain comprises one or more modifications that modulate basal inhibition. Examples of modifications include, but are not limited to, truncation mutation(s), amino acid substitution(s), introduction of locations for post-translational modification (examples of which are known to those having skill in the art), and addition of new functional groups. In some embodiments, the enzyme domain(s), enzyme fragment(s), or mutants(s) thereof are selected to maximize efficacy and minimize basal inhibition. In some embodiments, the one or more modifications reduce basal inhibition. In other embodiments, the one or more modifications increase basal inhibition.

In some embodiments, the enzymatic inhibitory domain inhibits, for example, immune receptor activation upon recruitment of a chimeric inhibitory receptor of the present disclosure proximal to an immune receptor. In some embodiments, the immune receptor is a naturally occurring immune receptor. In some embodiments, the immune receptor is a naturally occurring antigen receptor. In some embodiments, the immune receptor is selected from a T cell receptor, a pattern recognition receptor (PRR), a NOD-like receptor (NLR), a Toll-like receptor (TLR), a killer activated receptor (KAR), a killer inhibitor receptor (KIR), a complement receptor, an Fc receptor, a B cell receptor, and a cytokine receptor. In some embodiments, the immune receptor is a T cell receptor. In some embodiments, the immune receptor is a chimeric immune receptor. In some embodiments, the chimeric immune receptor is a chimeric TCR or a CAR.

In some embodiments, a chimeric inhibitory receptor of the present disclosure may also comprise one or more intracellular inhibitory co-signaling domains. In some embodiments, the intracellular inhibitory co-signaling domains comprise an inhibitory domain. In some embodiments, the one or more intracellular inhibitory co-signaling domains comprise one or more ITIM-containing protein, or fragment(s) thereof. ITIMs are conserved amino acid sequences found in cytoplasmic tails of many inhibitory immune receptors. In some embodiments, the one or more ITIM-containing proteins, or fragments thereof, are selected from PD-1, CTLA4, TIGIT, and LAIR1. In some embodiments, the one or more intracellular inhibitory co-signaling domains comprise one or more non-ITIM scaffold proteins, or a fragment(s) thereof. In some embodiments, the one or more non-ITIM scaffold proteins, or fragments thereof, are selected from GRB-2, Dok-1, Dok-2, SLAP, LAG3, HAVR, BTLA, GITR, and PD-L1. Further examples suitable intracellular inhibitory co-signaling domains include, without limitation, PD-L1, TIM3, LIR1, NKG2A, VISTA, CD160, 2B4, CD80, CD86, B7-H3 (CD276), B7-H4 (VTCN1), HVEM (TNFRSF14 or CD270), KIR, KIR3DL1, A2aR, MHC class I, MHC class II, GAL9, adenosine, and TGF beta.

In some embodiments, the inhibitory chimeric receptor binds an antigen that is expressed on a non-tumor cell. Exemplary antigens for use in a chimeric inhibitory receptor are described in Table 9.

TABLE 9

| | UniProt | | |
|---|---|---|---|
| Antigen | Accession No. | Name | Short Description |
| VSIG2 | Q96IQ7 | V-set and | Contains immunoglobulin domain, high |
| | | immunoglobulin | expression in glandular cells of the |
| | | domain-containing | gastrointestinal tract |
| | | protein 2 | |
| CPM | P14384 | Carboxypeptidase M | Removes C-terminal basic residues (Arg or |
| | | | Lys) from peptides and proteins; believed to |
| | | | function in the control of peptide hormone and |
| | | | growth factor activity at the cell surface |
| ITM2C | Q9NQX7 | Integral membrane | Negative regulator of amyloid-beta peptide |
| | | protein 2C | production |
| SLC26A2 | P50443 | Sulfate transporter | Sulfate transporter that may function in |
| | | | endochondral bone formation |
| SLC4A4 | Q9Y6R1 | Electrogenic sodium | Electrogenic sodium/bicarbonate cotransporter |
| | | bicarbonate | that may regulate bicarbonate influx/efflux at |
| | | cotransporter 1 | the basolateral membrane of cells and regulate |
| | | | intracellular pH |
| GPA33 | Q99795 | Cell surface A33 | May function in cell-cell recognition and |
| | | antigen | signaling |
| PLA2G2A | P14555 | Phospholipase A2, | Secretory calcium-dependent phospholipase |
| | | membrane associated | A2 that primarily targets extracellular |
| | | | phospholipids |
| ABCA8 | O94911 | ATP-binding cassette | ATP-dependent lipophilic drug transporter |
| | | sub-family A | |
| | | member 8 | |
| ATP1A2 | P50993 | Sodium/potassium- | Catalytic component of the active enzyme, |
| | | transporting ATPase | which catalyzes the hydrolysis of ATP coupled |
| | | subunit alpha-2 | with the exchange of sodium and potassium |
| | | | ions across the plasma membrane |
| CHP2 | O43745 | Calcineurin B | Cofactor in cell pH regulation by controlling |
| | | homologous protein 2 | plasma membrane-type Na+/H+ exchange |
| | | | activity. |
| SLC26A3 | P40879 | Chloride anion | Chloride/bicarbonate exchanger |
| | | exchanger | |

In some embodiments, the chimeric inhibitory receptor binds a VSIG2 antigen. In some embodiments, the chimeric inhibitory receptor binds a CPM antigen. In some embodiments, the chimeric inhibitory receptor binds an ITM2C antigen. In some embodiments, the chimeric inhibitory receptor binds a SLC26A2 antigen. In some embodiments, the chimeric inhibitory receptor binds a SLC4A4 antigen. In some embodiments, the chimeric inhibitory receptor binds a GPA33 antigen. In some embodiments, the chimeric inhibitory receptor binds a PLA2G2A antigen. In some embodiments, the chimeric inhibitory receptor binds an ABCA8 antigen. In some embodiments, the chimeric inhibitory receptor binds an ATP1A2 antigen. In some embodiments, the chimeric inhibitory receptor binds a CHP2 antigen. In some embodiments, the chimeric inhibitory receptor binds an SLC26A3.

In some embodiments, the chimeric inhibitory receptor is a multispecific receptor comprising two or more antigen-binding domains, such that the chimeric inhibitory receptor can bind two or more antigens. Alternatively, a cell can be edited to express two or more chimeric inhibitory receptors that bind to different antigens. Exemplary antigen pairs of chimeric inhibitory receptors are found in Table 10. In some embodiments, the bicistronic chimeric receptor system comprises a CAR with two or more antigen binding domains targeting any antigen pair provided in Table 10, wherein the antigen pair comprises a CEA-family member and an inhibitory antigen.

### Immunoresponsive Cells

Certain aspects of the present disclosure relate to a cell, such as an immunoresponsive cell, that has been genetically engineered to comprise one or more chimeric receptors of the present disclosure or one or more nucleic acids encoding such chimeric receptors, and to methods of using such cells for treating solid tumors.

In some embodiments, an immunoresponsive cell is genetically engineered to comprise one or more CARs (or nucleic acids encoding the same) that specifically binds a CEA-family member tumor antigen, such as any of the CEA antigens of Table 1. In some embodiments, an immunoresponsive cell is genetically engineered to comprise one or more CARs (or nucleic acids encoding the same) that comprise antibody sequences, or antigen-binding fragments thereof, of the representative anti-CEA antibodies provided in Table 2. In some embodiments, an immunoresponsive cell is genetically engineered to comprise one or more CARs (or nucleic acids encoding the same) that comprise an scFv derived from antibodies capable of binding to a solid tumor antigen, such as the representative anti-CEA scFvs provided in Table 3. In some embodiments, CARs that specifically binds a CEA-family member tumor antigen can be considered an activating CAR (“aCAR”), as described herein.

In some embodiments, the cell is a mammalian cell. In some embodiments, the mammalian cell is a primary cell. In some embodiments, the mammalian cell is a cell line. In some embodiments, the mammalian cell is a bone marrow cell, a blood cell, a skin cell, bone cell, a muscle cell, a lung cell, a cell of the gastrointestinal tract, a brain cell, a neuronal cell, a fat cell, a liver cell, or a heart cell. In some embodiments, the cell is a stem cell. Exemplary stem cells include, without limitation embryonic stem cells (ESCs), induced pluripotent stem cells (iPSCs), adult stem cells, and tissue-specific stem cells, such as hematopoietic stem cells (blood stem cells), mesenchymal stem cells (MSC), neural stem cells, epithelial stem cells, or skin stem cells. In some embodiments, the cell is a cell that is derived or differentiated from a stem cell of the present disclosure. In some embodiments, the cell is an immune cell. Immune cells of the present disclosure may be isolated or differentiated from a stem cell of the present disclosure (e.g., from an ESC or iPSC). Exemplary immune cells include, without limitation, T cells (e.g., helper T cells, cytotoxic T cells, memory T cells, regulatory T cells, natural killer T cells, alpha beta T cells, and gamma delta T cells), B cells, natural killer (NK) cells, dendritic cells, myeloid cells, macrophages, and monocytes. In some embodiments, the cell is a neuronal cell. Neuronal cells of the present disclosure may be isolated or differentiated from a stem cell of the present disclosure (e.g., from an ESC or iPSC). Exemplary neuronal cells include, without limitation, neural progenitor cells, neurons (e.g., sensory neurons, motor neurons, cholinergic neurons, GABAergic neurons, glutamatergic neurons, dopaminergic neurons, or serotonergic neurons), astrocytes, oligodendrocytes, and microglia.

In some embodiments, the cell is an immunoresponsive cell. Immunoresponsive cells of the present disclosure may be isolated or differentiated from a stem cell of the present disclosure (e.g., from an ESC or iPSC). Exemplary immunoresponsive cells of the present disclosure include, without limitation, cells of the lymphoid lineage. The lymphoid lineage, comprising B cells, T cells, and natural killer (NK) cells, provides for the production of antibodies, regulation of the cellular immune system, detection of foreign agents in the blood, detection of cells foreign to the host, and the like. Examples of immunoresponsive cells of the lymphoid lineage include, without limitation, T cells, Natural Killer (NK) cells, embryonic stem cells, pluripotent stem cells, and induced pluripotent stem cells (e.g., those from which lymphoid cells may be derived or differentiated). T cells can be lymphocytes that mature in the thymus and are chiefly responsible for cell-mediated immunity. T cells are involved in the adaptive immune system. In some embodiments, T cells of the present disclosure can be any type of T cells, including, without limitation, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells), natural killer T cells, mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of one or more chimeric receptors, such as a chimeric TCRs or CARs.

Natural killer (NK) cells can be lymphocytes that are part of cell-mediated immunity and act during the innate immune response. NK cells do not require prior activation in order to perform their cytotoxic effect on target cells.

In some embodiments, an immunoresponsive cell of the present disclosure is a T cell. T cells of the present disclosure may be autologous, allogeneic, or derived in vitro from engineered progenitor or stem cells.

In some embodiments, an immunoresponsive cell of the present disclosure is a universal T cell with deficient TCR-αβ. Methods of developing universal T cells are described in the art, for example, in Valton et al., Molecular Therapy (2015); 23 9, 1507-1518, and Torikai et al., Blood 2012 119:5697-5705.

In some embodiments, an immunoresponsive cell of the present disclosure is an isolated immunoresponsive cell comprising one or more chimeric receptors of the present disclosure. In some embodiments, the immunoresponsive cell comprises one or more, two or more, three or more, four or more, five or more, six or more, seven or more, eight or more, nine or more, or ten or more chimeric receptors of the present disclosure.

In some embodiments, an immunoresponsive cell is a T cell. In some embodiments, an immunoresponsive cell is a Natural Killer (NK) cell.

### Cells Expressing Multiple Chimeric Receptors

In some embodiments, a cell of the present disclosure (e.g., an immunoresponsive cell) comprises two or more chimeric receptors of the present disclosure. In some embodiments, the cell comprises two or more chimeric receptors, wherein one of the two or more chimeric receptors is a chimeric inhibitory receptor. In some embodiments, the cell comprises three or more chimeric receptors, wherein at least one of the three or more chimeric receptors is a chimeric inhibitory receptor. In some embodiments, the cell comprises four or more chimeric receptors, wherein at least one of the four or more chimeric receptors is a chimeric inhibitory receptor. In some embodiments, the cell comprises five or more chimeric receptors, wherein at least one of the five or more chimeric receptors is a chimeric inhibitory receptor.

In some embodiments, each of the two or more chimeric receptors comprise a different antigen-binding domain, e.g., that binds to the same antigen or to a different antigen. In some embodiments each antigen bound by the two or more chimeric receptors are expressed on the same cell type (e.g., same solid tumor cell type). In one embodiment, the cell comprises a first chimeric receptor that targets a first antigen and includes an intracellular signaling domain having a costimulatory signaling domain but not a primary signaling domain, and a second chimeric receptor that targets a second, different, antigen and includes an intracellular signaling domain having a primary signaling domain but not a costimulatory signaling domain. Without wishing to be bound by theory, it is believed that placement of a costimulatory signaling domain (e.g., 4-1BB, CD28, or OX-40) onto the first chimeric receptor, and placement of a primary signaling domain (e.g., CD3-zeta chain) on the second chimeric receptor may limit chimeric receptor activity to cells where both targets are expressed. Accordingly, in some embodiments, a cell of the present disclosure (e.g., an immunoresponsive cell) includes: (a) a first chimeric receptor comprising an antigen-binding domain that binds a first antigen, a transmembrane domain, and a costimulatory signaling domain; and (b) a second chimeric receptor comprising an antigen-binding domain that binds a second antigen, a transmembrane domain, and a primary signaling domain. In some embodiments, a cell of the present disclosure (e.g., an immunoresponsive cell) includes: (a) a first chimeric receptor comprising an antigen-binding domain that binds a first antigen, a transmembrane domain, and a primary signaling domain; and (b) a second chimeric receptor comprising an antigen-binding domain that binds a second antigen, a transmembrane domain, and a costimulatory signaling domain. In some embodiments, a cell of the present disclosure (e.g., an immunoresponsive cell) includes: (a) a first chimeric receptor comprising an antigen-binding domain that binds a first antigen, a transmembrane domain, a primary signaling domain and a costimulatory domain; and (b) a second chimeric receptor comprising an antigen-binding domain that binds a second antigen, a transmembrane domain, a primary signaling domain and a costimulatory domain. In embodiments where both the first chimeric receptor and the second chimeric receptor each comprise a costimulatory signaling domain, the costimulatory signaling domain of the first chimeric receptor and the costimulatory signaling domain of the second chimeric receptor may be derived from the same protein, such as from 4-1BB, CD28, or OX40. Alternatively, the costimulatory signaling domain of the first chimeric receptor may be derived from a different protein than that of the costimulatory signaling domain of the second chimeric receptor.

In embodiments where a cell of the present disclosure (e.g., an immunoresponsive cell) expresses two or more distinct chimeric receptors, the antigen-binding domain of each of the different chimeric receptors may be designed such that the antigen-binding domains do not interact with one another. For example, a cell of the present disclosure (e.g., an immunoresponsive cell) expressing a first chimeric receptor and a second chimeric receptor may comprise a first chimeric receptor that comprises an antigen-binding domain that does not form an association with the antigen-binding domain of the second chimeric receptor. For example, the antigen-binding domain of the first chimeric receptor may comprise an antibody fragment, such as an scFv, while the antigen-binding domain of the second chimeric receptor may comprise a V_{H}H.

Without wishing to be bound by theory, it is believed that in cells having a plurality of chimeric membrane embedded receptors that each comprise an antigen-binding domain, interactions between the antigen-binding domains of each of the receptors can be undesirable, because such interactions may inhibit the ability of one or more of the antigen-binding domains to bind their cognate antigens. Accordingly, in embodiments where cells of the present disclosure (e.g., immunoresponsive cells) express two or more chimeric receptors, the chimeric receptors comprise antigen-binding domains that minimize such inhibitory interactions. In one embodiment, the antigen-binding domain of one chimeric receptor comprises an scFv and the antigen-binding domain of the second chimeric receptor comprises a single VH domain, e.g., a camelid, shark, or lamprey single VH domain, or a single VH domain derived from a human or mouse sequence.

In some embodiments, when present on the surface of a cell, binding of the antigen-binding domain of the first chimeric receptor to its cognate antigen is not substantially reduced by the presence of the second chimeric receptor. In some embodiments, binding of the antigen-binding domain of the first chimeric receptor to its cognate antigen in the presence of the second chimeric receptor is 85%, 90%, 95%, 96%, 97%, 98%, or 99% of binding of the antigen-binding domain of the first chimeric receptor to its cognate antigen in the absence of the second chimeric receptor. In some embodiments, when present on the surface of a cell, the antigen-binding domains of the first chimeric receptor and the second chimeric receptor associate with one another less than if both were scFv antigen-binding domains. In some embodiments, the antigen-binding domains of the first chimeric receptor and the second chimeric receptor associate with one another 85%, 90%, 95%, 96%, 97%, 98%, or 99% less than if both were scFv antigen-binding domains.

In embodiments where a cell of the present disclosure (e.g., an immunoresponsive cell) comprises two or more distinct chimeric receptors of the present disclosure that bind to different antigens, the two or more chimeric receptor provide the cell with logic gating, such as OR logic gating, AND logic gating, NOT logic gating, or any combination of such logic gating. Accordingly, in certain embodiments, a cell of the present disclosure (e.g., an immunoresponsive cell) comprises two or more chimeric receptors, and binding of the first chimeric receptor to the first antigen is capable of activating the cell. In some embodiments, a cell of the present disclosure (e.g., an immunoresponsive cell) comprises two or more chimeric receptors, and binding of the second chimeric receptor to the second antigen is capable of stimulating the cell. In some embodiments, a cell of the present disclosure (e.g., an immunoresponsive cell) comprises two or more chimeric receptors, and binding of the first chimeric receptor to the first antigen and binding of the second chimeric receptor to the second antigen are required for activating the cell. In some embodiments, a cell of the present disclosure (e.g., an immunoresponsive cell) comprises two or more chimeric receptors, and binding of the first chimeric receptor to the first antigen and binding of the second chimeric receptor to the second antigen are required for simulating the cell. In some embodiments, a cell of the present disclosure (e.g., an immunoresponsive cell) comprises two or more chimeric receptors, and the cell exhibits a greater degree of cytolytic activity against cells that are positive for both the first antigen and the second antigen as compared to the cytolytic activity against cells that are positive for only the first antigen or only the second antigen. In some embodiments, a cell of the present disclosure (e.g., an immunoresponsive cell) comprises two or more chimeric receptors, and binding of the first chimeric receptor to the first antigen or binding of the second chimeric receptor to the second antigen is capable of activating the immunoresponsive cell.

In some embodiments, a cell of the present disclosure (e.g., an immunoresponsive cell) comprises a split chimeric receptor system, such as a split CAR system. Exemplary split chimeric receptor systems are described in WO2014/055442 and WO2014/055657. In some embodiments, a split chimeric receptor system comprises a cell expressing a first chimeric receptor having a first antigen-binding domain and a costimulatory domain (e.g., 4-1BB), as well as a second chimeric receptor having a second antigen-binding domain and an intracellular signaling domain (e.g., CD3-zeta). In such embodiments, when the cell encounters the first antigen, the costimulatory domain is activated, and the cell proliferates. Additionally, when the cell encounters the second antigen, the intracellular signaling domain is activated and cell-killing activity is induced. Accordingly, in some embodiments, a cell of the present disclosure (e.g., an immunoresponsive cell) is only fully activated in the presence of both antigens.

In certain embodiments, a cell of the present disclosure (e.g., an immunoresponsive cell) exhibits a greater degree of cytolytic activity against cells that are positive for both the first antigen and the second antigen as compared to against cells that are singly positive for the first antigen. In certain embodiments, the first chimeric receptor binds to a first antigen with a low binding affinity or a low binding avidity. In certain embodiments, the first chimeric receptor binds to the first antigen at an epitope of low accessibility. In certain embodiments, first chimeric receptor binds to the first antigen with a binding affinity that is lower compared to the binding affinity with which the second chimeric receptor binds to the second antigen. In some embodiments, the first chimeric receptor binds to the first antigen with a binding affinity that is at least 5-fold lower compared to the binding affinity with which the second chimeric receptor binds to the second antigen. In some embodiments, the first chimeric receptor binds to the first antigen with a binding affinity that is at least 10-fold, 20-fold, 30-fold, 40-fold, 50-fold, 60-fold, 70-fold, 80-fold, 90-fold, 100-fold, 200-fold, 5000-fold, 1000-fold, 5000-fold, or 10000-fold lower compared to the binding affinity with which the second chimeric receptor binds to the second antigen.

In some embodiments, pairing choices should favor redundant expression of the two target antigens in the tumor in order to minimize the risk of antigen escape. Accordingly, in some embodiments, a cell of the present disclosure (e.g., an immunoresponsive cell) comprises (i) a first chimeric receptor that binds to a first antigen and (ii) a second chimeric receptor that binds to a second antigen, wherein the combination of both chimeric receptors binding to their targets antigens produces a therapeutic effect. In embodiments, binding to only one target antigen does not achieve a therapeutic effect.

In some embodiments, a cell of the present disclosure (e.g., an immunoresponsive cell) comprises one or more chimeric receptors that bind a CEA-family antigen, and optionally the cell also comprises an inhibitory chimeric receptor. In some embodiments, the chimeric receptor binds a CEACAM1 antigen. In some embodiments, the chimeric receptor binds a CEACAM5 antigen. In some embodiments, the chimeric receptor binds a CEACAM6 antigen. In some embodiments, the chimeric receptor binds to a CEA-family antigen as described in Table 1. In some embodiments, the chimeric receptor that binds to a CEA-family antigen comprises an antigen binding domain derived from an antibody selected from: MRG1, Labetuzumab (i.e., hMN14), Cibisatamab, Tusamitamab, BW431/26, A5B7, MFE23, hMFE23, FM4 (also referred to as “MG7”), Tinurilimab. In some embodiments, the antigen binding domain of the chimeric receptor that binds to a CEA-family antigen includes an scFv comprising an amino acid sequence as provided in Table 3. In some embodiments, the chimeric receptor that binds to a CEA-family antigen comprises an amino acid sequence as shown in Table 28 and/or is encoded by a polynucleotide sequence as provided in Table 28. In some embodiments, the inhibitory chimeric receptor binds a VSIG2 antigen. In some embodiments, the inhibitory chimeric receptor binds a CPM antigen. In some embodiments, the inhibitory chimeric receptor binds an ITM2C antigen. In some embodiments, the inhibitory chimeric receptor binds a SLC26A2 antigen. In some embodiments, the inhibitory chimeric receptor binds a SLC4A4 antigen. In some embodiments, the inhibitory chimeric receptor binds a GPA33 antigen. In some embodiments, the inhibitory chimeric receptor binds a PLA2G2A antigen. In some embodiments, the inhibitory chimeric receptor binds an ABCA8 antigen. In some embodiments, the inhibitory chimeric receptor binds an ATP1A2 antigen. In some embodiments, the inhibitory chimeric receptor binds a CHP2 antigen. In some embodiments, the inhibitory chimeric receptor binds an SLC26A3 antigen. In some embodiments, the chimeric receptor is a multispecific receptor comprising two or more antigen-binding domains, such that the chimeric receptor can bind two or more antigens.

In some embodiments, an immunoresponsive cell may comprise one or more tumor-targeting chimeric receptors and one or more inhibitory chimeric receptors that targets an antigen that is not expressed on the tumor. Combinations of tumor-targeting chimeric receptors and inhibitory chimeric receptors in the same immunoresponsive cell may be used to reduce on-target off-tumor toxicity. For instance, if a healthy cell expresses both an antigen that is recognized by a tumor-targeting chimeric receptor and an antigen that is recognized by an inhibitory chimeric receptor, an immunoresponsive cell expressing the tumor antigen may bind to the healthy cell. In such a case, the inhibitory chimeric antigen will also bind its cognate ligand on the healthy cell and the inhibitory function of the inhibitory chimeric receptor will reduce, decrease, prevent, or inhibit the activation of the immunoresponsive cell via the tumor-targeting chimeric receptor.

In some embodiments, the inhibitory chimeric receptor binds a VSIG2 antigen. In some embodiments, the inhibitory chimeric receptor binds a CPM antigen. In some embodiments, the inhibitory chimeric receptor binds an ITM2C antigen. In some embodiments, the inhibitory chimeric receptor binds a SLC26A2 antigen. In some embodiments, the inhibitory chimeric receptor binds a SLC4A4 antigen. In some embodiments, the inhibitory chimeric receptor binds a GPA33 antigen. In some embodiments, the inhibitory chimeric receptor binds a PLA2G2A antigen. In some embodiments, the inhibitory chimeric receptor binds an ABCA8 antigen. In some embodiments, the inhibitory chimeric receptor binds an ATP1A2 antigen. In some embodiments, the inhibitory chimeric receptor binds a CHP2 antigen. In some embodiments, the inhibitory chimeric receptor binds an SLC26A3 antigen.

Alternatively, a cell can express two or more chimeric receptors that bind to different antigens. Exemplary pairs of antigens are shown in Table 10.

TABLE 10

| Antigen 1 | Antigen 2 | Antigen 1 | Antigen 2 |
|---|---|---|---|
| CEACAM1 | CEACAM5 | CEACAM6 | SLC4A4 |
| CEACAM1 | CEACAM6 | CEACAM6 | GPA33 |
| CEACAM1 | VSIG2 | CEACAM6 | PLA2G2A |
| CEACAM1 | CPM | CEACAM6 | ABCA8 |
| CEACAM1 | ITM2C | CEACAM6 | ATP1A2 |
| CEACAM1 | SLC26A2 | CEACAM5 | SLC26A2 |
| CEACAM1 | SLC4A4 | CEACAM5 | SLC4A4 |
| CEACAM1 | GPA33 | CEACAM5 | GPA33 |
| CEACAM1 | PLA2G2A | CEACAM5 | PLA2G2A |
| CEACAM1 | CHP2 | CEACAM5 | ABCA8 |
| CEACAM1 | SLC26A3 | CEACAM5 | ATP1A2 |
| CEACAM1 | ABCA8 | CEACAM5 | CHP2 |
| CEACAM1 | ATP1A2 | CEACAM5 | SLC26A3 |
| CEACAM5 | CEACAM6 | CEACAM6 | VSIG2 |
| CEACAM5 | VSIG2 | CEACAM6 | CPM |
| CEACAM5 | CPM | CEACAM6 | ITM2C |
| CEACAM5 | ITM2C | CEACAM6 | SLC26A2 |
| | | CEACAM6 | CHP2 |
| | | CEACAM6 | SLC26A3 |

In some embodiments, the immunoresponsive cell comprises a bicistronic chimeric antigen receptor system construct. In some embodiments, the bicistronic chimeric antigen receptor system construct comprises a CEA CAR and a CEACAM1 CAR. In some embodiments, the bicistronic chimeric antigen receptor system construct comprises a CEA CAR and a CEACAM5 CAR. In some embodiments, the bicistronic chimeric antigen receptor system construct comprises a CEA CAR and a CEACAM6 CAR. In some embodiments, the bicistronic chimeric antigen receptor system construct comprises a CEACAM1 CAR and a CEACAM5 CAR. In some embodiments, the bicistronic chimeric antigen receptor system construct comprises a CEACAM1 CAR and a CEACAM6 CAR. In some embodiments, the bicistronic chimeric antigen receptor system construct comprises a CEACAM5 CAR and a CEACAM6 CAR.

In some embodiments, the bicistronic chimeric antigen receptor system construct comprises a CEA CAR and a VSIG2 CAR. In some embodiments, the bicistronic chimeric antigen receptor system construct comprises a CEA CAR and a CPM CAR. In some embodiments, the bicistronic chimeric antigen receptor system construct comprises a CEA CAR and a ITM2C CAR. In some embodiments, the bicistronic chimeric antigen receptor system construct comprises a CEA CAR and a SLC26A2 CAR. In some embodiments, the bicistronic chimeric antigen receptor system construct comprises a CEA CAR and a SLC4A4 CAR. In some embodiments, the bicistronic chimeric antigen receptor system construct comprises a CEA CAR and a GPA33 CAR. In some embodiments, the bicistronic chimeric antigen receptor system construct comprises a CEA CAR and a PLA2G2A CAR. In some embodiments, the bicistronic chimeric antigen receptor system construct comprises a CEA CAR and an ABCA8 CAR. In some embodiments, the bicistronic chimeric antigen receptor system construct comprises a CEA CAR and an ATP1A2 CAR. In some embodiments, the bicistronic chimeric antigen receptor system construct comprises a CEA CAR and a CHP2 CAR. In some embodiments, the bicistronic chimeric antigen receptor system construct comprises a CEA CAR and an SLC26A3 CAR.

In some embodiments, the bicistronic chimeric antigen receptor system construct comprises a CEACAM1 CAR and a VSIG2 CAR. In some embodiments, the bicistronic chimeric antigen receptor system construct comprises a CEACAM1 CAR and a CPM CAR. In some embodiments, the bicistronic chimeric antigen receptor system construct comprises a CEACAM1 CAR and a ITM2C CAR. In some embodiments, the bicistronic chimeric antigen receptor system construct comprises a CEACAM1 CAR and a SLC26A2 CAR. In some embodiments, the bicistronic chimeric antigen receptor system construct comprises a CEACAM1 CAR and a SLC4A4 CAR. In some embodiments, the bicistronic chimeric antigen receptor system construct comprises a CEACAM1 CAR and a GPA33 CAR. In some embodiments, the bicistronic chimeric antigen receptor system construct comprises a CEACAM1 CAR and a PLA2G2A CAR. In some embodiments, the bicistronic chimeric antigen receptor system construct comprises a CEACAM1 CAR and a ABCA8 CAR. In some embodiments, the bicistronic chimeric antigen receptor system construct comprises a CEACAM1 CAR and an ATP1A2 CAR. In some embodiments, the bicistronic chimeric antigen receptor system construct comprises a CEACAM1 CAR and a CHP2 CAR. In some embodiments, the bicistronic chimeric antigen receptor system construct comprises a CEACAM1 CAR and an SLC26A3 CAR.

In some embodiments, the bicistronic chimeric antigen receptor system construct comprises a CEACAM5 CAR and a VSIG2 CAR. In some embodiments, the bicistronic chimeric antigen receptor system construct comprises a CEACAM5 CAR and a CPM CAR. In some embodiments, the bicistronic chimeric antigen receptor system construct comprises a CEACAM5 CAR and an ITM2C CAR. In some embodiments, the bicistronic chimeric antigen receptor system construct comprises a CEACAM5 CAR and a SLC26A2 CAR. In some embodiments, the bicistronic chimeric antigen receptor system construct comprises a CEACAM5 CAR and a SLC4A4 CAR. In some embodiments, the bicistronic chimeric antigen receptor system construct comprises a CEACAM5 CAR and a GPA33 CAR. In some embodiments, the bicistronic chimeric antigen receptor system construct comprises a CEACAM5 CAR and a PLA2G2A CAR. In some embodiments, the bicistronic chimeric antigen receptor system construct comprises a CEACAM5 CAR and an ABCA8 CAR. In some embodiments, the bicistronic chimeric antigen receptor system construct comprises a CEACAM5 CAR and an ATP1A2 CAR. In some embodiments, the bicistronic chimeric antigen receptor system construct comprises a CEACAM5 CAR and a CHP2 CAR. In some embodiments, the bicistronic chimeric antigen receptor system construct comprises a CEACAM5 CAR and an SLC26A3 CAR.

In some embodiments, the bicistronic chimeric antigen receptor system construct comprises a CEACAM6 CAR and a VSIG2 CAR. In some embodiments, the bicistronic chimeric antigen receptor system construct comprises a CEACAM6 CAR and a CPM CAR. In some embodiments, the bicistronic chimeric antigen receptor system construct comprises a CEACAM6 CAR and an ITM2C CAR. In some embodiments, the bicistronic chimeric antigen receptor system construct comprises a CEACAM6 CAR and a SLC26A2 CAR. In some embodiments, the bicistronic chimeric antigen receptor system construct comprises a CEACAM6 CAR and a SLC4A4 CAR. In some embodiments, the bicistronic chimeric antigen receptor system construct comprises a CEACAM6 CAR and a GPA33 CAR. In some embodiments, the bicistronic chimeric antigen receptor system construct comprises a CEACAM6 CAR and a PLA2G2A CAR. In some embodiments, the bicistronic chimeric antigen receptor system construct comprises a CEACAM6 CAR and an ABCA8 CAR. In some embodiments, the bicistronic chimeric antigen receptor system construct comprises a CEACAM6 CAR and an ATP1A2 CAR. In some embodiments, the bicistronic chimeric antigen receptor system construct comprises a CEACAM6 CAR and a CHP2 CAR. In some embodiments, the bicistronic chimeric antigen receptor system construct comprises a CEACAM6 CAR and a CHP2 CAR. In some embodiments, the bicistronic chimeric antigen receptor system construct comprises a CEACAM6 CAR and an SLC26A3 CAR.

In some embodiments, the bicistronic chimeric antigen receptor system construct comprises any pair of antigens provided in Table 10.

### Cells Expressing Chimeric Inhibitory Receptors

In some embodiments, a cell of the present disclosure (e.g., an immunoresponsive cell) comprises one or more chimeric inhibitory receptors of the present disclosure. In some embodiments, each of the one or more chimeric inhibitory receptors comprises an antigen-binding domain that binds an antigen expressed on normal cells but not on tumor cells, such as solid tumor cells. In some embodiments, the one or more chimeric inhibitory receptors bind antigens that are expressed on a non-tumor cell derived from a tissue selected from the group consisting of brain, neuronal tissue, endocrine, bone, bone marrow, immune system, endothelial tissue, muscle, lung, liver, gallbladder, pancreas, gastrointestinal tract, kidney, urinary bladder, male reproductive organs, female reproductive organs, adipose, soft tissue, and skin.

In some embodiments, a chimeric inhibitory receptor may be used, for example, with one or more chimeric receptors (e.g., chimeric TCRs or CARs) expressed on a cell of the present disclosure (e.g., an immunoresponsive cell) as NOT logic gates to control, modulate, or otherwise inhibit one or more activities of the one or more chimeric receptors. In some embodiments, a chimeric receptor of the present disclosure may inhibit one or more activities of a cell of the present disclosure (e.g., an immunoresponsive cell). In some embodiments, the chimeric inhibitory receptor is combined with one or more chimeric receptors of the present disclosure to combine OR logic gating with NOT logic gating and/or AND logic gating with NOT logic gating.

In some embodiments, the chimeric inhibitory receptor binds one or more antigens selected from VSIG2, CPM, ITM2C, SLC26A2, SLC4A4, GPA33, PLA2G2A, ABCA8, ATP1A2, CHP2, and SLC26A3.

In some embodiments, the chimeric receptor binds a CEA antigen and the chimeric inhibitory receptor binds a VSIG2, CPM, ITM2C, SLC26A2, SLC4A4, GPA33, PLA2G2A, ABCA8, ATP1A2, CHP2, or SLC26A3 antigen.

In some embodiments, the chimeric receptor binds a CEACAM1 antigen and the chimeric inhibitory receptor binds a VSIG2, CPM, ITM2C, SLC26A2, SLC4A4, GPA33, PLA2G2A, ABCA8, ATP1A2, CHP2, or SLC26A3 antigen.

In some embodiments, the chimeric receptor binds a CEACAM5 antigen and the chimeric inhibitory receptor binds a VSIG2, CPM, ITM2C, SLC26A2, SLC4A4, GPA33, PLA2G2A, ABCA8, ATP1A2, CHP2, or SLC26A3 antigen.

In some embodiments, the chimeric receptor binds a CEACAM6 antigen and the chimeric inhibitory receptor binds a VSIG2, CPM, ITM2C, SLC26A2, SLC4A4, GPA33, PLA2G2A, ABCA8, ATP1A2, CHP2, or SLC26A3 antigen.

In some embodiments, the chimeric inhibitory receptor binds a VSIG2 antigen and the chimeric receptor binds a CEA antigen. In some embodiments, the chimeric inhibitory receptor binds a VSIG2 antigen and the chimeric receptor binds a CEACAM1 antigen. In some embodiments, the chimeric inhibitory receptor binds a VSIG2 antigen and the chimeric receptor binds a CEACAM5 antigen. In some embodiments, the chimeric inhibitory receptor binds a VSIG2 antigen and the chimeric receptor binds a CEACAM6 antigen.

In some embodiments, the chimeric inhibitory receptor binds a CPM antigen and the chimeric receptor binds a CEA antigen. In some embodiments, the chimeric inhibitory receptor binds a CPM antigen and the chimeric receptor binds a CEACAM1 antigen. In some embodiments, the chimeric inhibitory receptor binds a CPM antigen and the chimeric receptor binds a CEACAM5 antigen. In some embodiments, the chimeric inhibitory receptor binds a CPM antigen and the chimeric receptor binds a CEACAM6 antigen.

In some embodiments, the chimeric inhibitory receptor binds an ITM2C antigen and the chimeric receptor binds a CEA antigen. In some embodiments, the chimeric inhibitory receptor binds an ITM2C antigen and the chimeric receptor binds a CEACAM1 antigen. In some embodiments, the chimeric inhibitory receptor binds an ITM2C antigen and the chimeric receptor binds a CEACAM5 antigen. In some embodiments, the chimeric inhibitory receptor binds an ITM2C antigen and the chimeric receptor binds a CEACAM6 antigen.

In some embodiments, the chimeric inhibitory receptor binds a SLC26A2 antigen and the chimeric receptor binds a CEA antigen. In some embodiments, the chimeric inhibitory receptor binds a SLC26A2 antigen and the chimeric receptor binds a CEACAM1 antigen. In some embodiments, the chimeric inhibitory receptor binds a SLC26A2 antigen and the chimeric receptor binds a CEACAM5 antigen. In some embodiments, the chimeric inhibitory receptor binds a SLC26A2 antigen and the chimeric receptor binds a CEACAM6 antigen.

In some embodiments, the chimeric inhibitory receptor binds a SLC4A4 antigen and the chimeric receptor binds a CEA antigen. In some embodiments, the chimeric inhibitory receptor binds a SLC4A4 antigen and the chimeric receptor binds a CEACAM1 antigen. In some embodiments, the chimeric inhibitory receptor binds a SLC4A4 antigen and the chimeric receptor binds a CEACAM5 antigen. In some embodiments, the chimeric inhibitory receptor binds a SLC4A4 antigen and the chimeric receptor binds a CEACAM6 antigen.

In some embodiments, the chimeric inhibitory receptor binds a GPA33 antigen and the chimeric receptor binds a CEA antigen. In some embodiments, the chimeric inhibitory receptor binds a GPA33 antigen and the chimeric receptor binds a CEACAM1 antigen. In some embodiments, the chimeric inhibitory receptor binds a GPA33 antigen and the chimeric receptor binds a CEACAM5 antigen. In some embodiments, the chimeric inhibitory receptor binds a GPA33 antigen and the chimeric receptor binds a CEACAM6 antigen.

In some embodiments, the chimeric inhibitory receptor binds a PLA2G2A antigen and the chimeric receptor binds a CEA antigen. In some embodiments, the chimeric inhibitory receptor binds a PLA2G2A antigen and the chimeric receptor binds a CEACAM1 antigen. In some embodiments, the chimeric inhibitory receptor binds a PLA2G2A antigen and the chimeric receptor binds a CEACAM5 antigen. In some embodiments, the chimeric inhibitory receptor binds a PLA2G2A antigen and the chimeric receptor binds a CEACAM6 antigen.

In some embodiments, the chimeric inhibitory receptor binds an ABCA8 antigen and the chimeric receptor binds a CEA antigen. In some embodiments, the chimeric inhibitory receptor binds an ABCA8 antigen and the chimeric receptor binds a CEACAM1 antigen. In some embodiments, the chimeric inhibitory receptor binds an ABCA8 antigen and the chimeric receptor binds a CEACAM5 antigen. In some embodiments, the chimeric inhibitory receptor binds an ABCA8 antigen and the chimeric receptor binds a CEACAM6 antigen.

In some embodiments, the chimeric inhibitory receptor binds an ATP1A2 antigen and the chimeric receptor binds a CEA antigen. In some embodiments, the chimeric inhibitory receptor binds an ATP1A2 antigen and the chimeric receptor binds a CEACAM1 antigen. In some embodiments, the chimeric inhibitory receptor binds an ATP1A2 antigen and the chimeric receptor binds a CEACAM5 antigen. In some embodiments, the chimeric inhibitory receptor binds an ATP1A2 antigen and the chimeric receptor binds a CEACAM6 antigen.

In some embodiments, the chimeric inhibitory receptor binds a CHP2 antigen and the chimeric receptor binds a CEA antigen. In some embodiments, the chimeric inhibitory receptor binds a CHP2 antigen and the chimeric receptor binds a CEACAM1 antigen. In some embodiments, the chimeric inhibitory receptor binds a CHP2 antigen and the chimeric receptor binds a CEACAM5 antigen. In some embodiments, the chimeric inhibitory receptor binds a CHP2 antigen and the chimeric receptor binds a CEACAM6 antigen.

In some embodiments, the chimeric inhibitory receptor binds an SLC26A3 antigen and the chimeric receptor binds a CEA antigen. In some embodiments, the chimeric inhibitory receptor binds an SLC26A3 antigen and the chimeric receptor binds a CEACAM1 antigen. In some embodiments, the chimeric inhibitory receptor binds an SLC26A3 antigen and the chimeric receptor binds a CEACAM5 antigen. In some embodiments, the chimeric inhibitory receptor binds an SLC26A3 antigen and the chimeric receptor binds a CEACAM6 antigen.

In some embodiments, the inhibitory chimeric receptor comprises an antigen binding domain derived from an anti-VSIG2 antibody. The VSIG2 antibody can be any suitable VSIG2 antibody known in the art.

In some embodiments, the inhibitory chimeric receptor comprises an antigen binding domain derived from an anti-CPM antibody. The CPM antibody can be any suitable CPM antibody known in the art.

In some embodiments, the inhibitory chimeric receptor comprises an antigen binding domain derived from an anti-ITM2C antibody. The ITM2C antibody can be any suitable ITM2C antibody known in the art.

In some embodiments, the inhibitory chimeric receptor comprises an antigen binding domain derived from an anti-SLC26A2 antibody. The SLC26A2 antibody can be any suitable SLC26A2 antibody known in the art.

In some embodiments, the inhibitory chimeric receptor comprises an antigen binding domain derived from an anti-SLC4A4 antibody. The SLC4A4 antibody can be any suitable SLC4A4 antibody known in the art.

In some embodiments, the inhibitory chimeric receptor comprises an antigen binding domain derived from an anti-GPA33 antibody. The GPA33 antibody can be any suitable GPA33 antibody known in the art.

In some embodiments, the inhibitory chimeric receptor comprises an antigen binding domain derived from an anti-PLA2G2A antibody. The PLA2G2A antibody can be any suitable PLA2G2A antibody known in the art.

In some embodiments, the inhibitory chimeric receptor comprises an antigen binding domain derived from an anti-ABCA8 antibody. The ABCA8 antibody can be any suitable ABCA8 antibody known in the art.

In some embodiments, the inhibitory chimeric receptor comprises an antigen binding domain derived from an anti-ATP1A2 antibody. The ATP1A2 antibody can be any suitable ATP1A2 antibody known in the art.

In some embodiments, the inhibitory chimeric receptor comprises an antigen binding domain derived from an anti-CHP2 antibody. The CHP2 antibody can be any suitable CHP2 antibody known in the art.

In some embodiments, the inhibitory chimeric receptor comprises an antigen binding domain derived from an anti-SLC26A3 antibody. The SLC26A3 antibody can be any suitable SLC26A3 antibody known in the art.

### Co-Stimulatory Ligands

In some embodiments, a cell of the present disclosure (e.g., an immunoresponsive cell, e.g., an NK cell) can further include one or more recombinant or exogenous co-stimulatory ligands. For example, the cell can be further transduced with one or more co-stimulatory ligands, such that the cell co-expresses or is induced to co-express one or more chimeric receptors of the present disclosure and one or more co-stimulatory ligands. Without wishing to be bound by theory, it is believed that the interaction between the one or more chimeric receptors and the one or more co-stimulatory ligands may provide a non-antigen-specific signal important for full activation of the cell. Examples of suitable co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Examples of suitable TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta (LTP), CD257/B cell-activating factor (B AFF)/Bly s/THANK/Tall-1, glucocorticoid-induced TNF Receptor ligand (GITRL), and TNF-related apoptosis-inducing ligand (TRAIL), LIGHT (TNFSF 14). The immunoglobulin (Ig) superfamily is a large group of cell surface and soluble proteins that are involved in the recognition, binding, or adhesion processes of cells. These proteins share structural features with immunoglobulins and possess an immunoglobulin domain (fold). Examples of suitable immunoglobulin superfamily ligands include, without limitation, CD80 and CD86, both ligands for CD28, PD-L1/(B7-H1) that ligands for PD-1. In certain embodiments, the one or more co-stimulatory ligands are selected from 4-1BBL, CD80, CD86, CD70, OX40L, CD48, TNFRSF14, PD-L1, and combinations thereof.

### Effector Molecules

In some embodiments, a cell of the present disclosure (e.g., an immunoresponsive cell, e.g., an NK cell) comprises one or more chimeric receptors and may further include one or more immunomodulatory effector molecules. Non-limiting examples of effector molecules encompassed by the present disclosure include cytokines, antibodies, chemokines, nucleotides, peptides, enzymes, and oncolytic viruses. For example, cells may be engineered to express and secrete in a regulated manner at least one, two, three or more of the following immunomodulatory effector molecules: IL-12, IL-16, IFN-β, IFN-γ, IL-2, IL-15, IL-7, IL-36γ, IL-18, IL-10, IL-21, OX40-ligand, CD40L, anti-PD-1 antibodies, anti-PD-L1 antibodies, anti-CTLA-4 antibodies, anti-TGFβ antibodies, anti-TNFR2, MIP1α (CCL3), MIP1β (CCL5), CCL21, CpG oligodeoxynucleotides, and anti-tumor peptides (e.g., anti-microbial peptides having anti-tumor activity, see, e.g., Gaspar, D. et al. Front Microbiol. 2013; 4: 294; Chu, H. et al. PLoS One. 2015; 10(5): e0126390, and website:aps.unmc.edu/AP/main.php).

In some embodiments, the effector molecule is an immunomodulatory effector molecule that: stimulates T cell signaling, activity and/or recruitment, stimulates antigen presentation and/or processing, stimulates natural killer cell-mediated cytotoxic signaling, activity and/or recruitment, stimulates dendritic cell differentiation and/or maturation, stimulates immune cell recruitment, stimulates macrophage signaling, stimulates stroma degradation, stimulates immunostimulatory metabolite production, or stimulates Type I interferon signaling; and at least one protein including an effector molecule that inhibits negative costimulatory signaling, inhibits pro-apoptotic signaling of anti-tumor immune cells, inhibits T regulatory (Treg) cell signaling, activity and/or recruitment, inhibits tumor checkpoint molecules, activates stimulator of interferon genes (STING) signaling, inhibits myeloid-derived suppressor cell signaling, activity and/or recruitment, degrades immunosuppressive factors/metabolites, inhibits vascular endothelial growth factor signaling, or directly kills tumor cells.

In some embodiments, the one or more effector molecules includes a cytokine selected from IL-15, IL-12 (e.g., an IL12p70 fusion protein), IL-18, and IL-21.

### Chemokine Receptors

In some embodiments, a cell of the present disclosure (e.g., an immunoresponsive cell, e.g., an NK cell) comprises one or more chimeric receptors and may further include one or more chemokine receptors. For example, transgenic expression of chemokine receptor CCR2b or CXCR2 in cells, such as T cells, enhances trafficking to CCL2-secreting or CXCL1-secreting solid tumors (Craddock et al, J Immunother. 2010 October; 33(8):780-8 and Kershaw et al. Hum Gene Ther. 2002 Nov. 1; 13(16): 1971-80). Without wishing to be bound by theory, it is believed that chemokine receptors expressed on chimeric receptor-expressing cells of the present disclosure may recognize chemokines secreted by tumors and improve targeting of the cell to the tumor, which may facilitate the infiltration of the cell to the tumor and enhance the antitumor efficacy of the cell. Chemokine receptors of the present disclosure may include a naturally occurring chemokine receptor, a recombinant chemokine receptor, or a chemokine-binding fragment thereof. Examples of suitable chemokine receptors that may expressed on a cell of the present disclosure include, without limitation, a CXC chemokine receptor, such as CXCR1, CXCR2, CXCR3, CXCR4, CXCR5, CXCR6, or CXCR7; a CC chemokine receptor, such as CCR1, CCR2, CCR3, CCR4, CCR5, CCR6, CCR7, CCR8, CCR9, CCR10, or CCR11; a CX3C chemokine receptor, such as CX3CR1; an XC chemokine receptor, such as XCR1; and chemokine-binding fragments thereof. In some embodiments, the chemokine receptor to be expressed on the cell is chosen based on the chemokines secreted by the tumor.

### Chimeric Receptor Regulation

Some embodiments of the present disclosure relate to regulating one or more chimeric receptor activities of chimeric receptor-expressing cells of the present disclosure. There are several ways chimeric receptor activities can be regulated. In some embodiments, a regulatable chimeric receptor, wherein one or more chimeric receptor activities can be controlled, may be desirable to optimize the safety and/or efficacy of the chimeric receptor therapy. For example, inducing apoptosis using a caspase fused to a dimerization domain (see, e.g., Di et al., N Engl. J. Med. 2011 Nov. 3; 365(18): 1673-1683) can be used as a safety switch in the chimeric receptor therapy. In some embodiments, a chimeric receptor-expressing cell of the present disclosure can also express an inducible Caspase-9 (iCaspase-9) that, upon administration of a dimerizer drug, such as rimiducid (IUPAC name: [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl)butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl)butanoyl]piperidine-2-carboxylate), induces activation of the Caspase-9 and results in apoptosis of the cells. In some embodiments, the iCaspase-9 contains a binding domain that comprises a chemical inducer of dimerization (CID) that mediates dimerization in the presence of the CID, which results in inducible and selective depletion of the chimeric receptor-expressing cells.

Alternatively, in some embodiments a chimeric receptor of the present disclosure may be regulated by utilizing a small molecule or an antibody that deactivates or otherwise inhibits chimeric receptor activity. For example, an antibody may delete the chimeric receptor-expressing cells by inducing antibody dependent cell-mediated cytotoxicity (ADCC). In some embodiments, a chimeric receptor-expressing cell of the present disclosure may further express an antigen that is recognized by a molecule that is capable of inducing cell death by ADCC or complement-induced cell death. For example, a chimeric receptor-expressing cell of the present disclosure may further express a receptor capable of being targeted by an antibody or antibody fragment. Examples of suitable receptors that may be targeted by an antibody or antibody fragment include, without limitation, EpCAM, VEGFR, integrins (e.g., αvβ, α4, αI¾β3, α4β7, α5β1, αvβ, αv), members of the TNF receptor superfamily (e.g., TRAIL-R1 and TRAIL-R2), PDGF receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof.

In some embodiments, a chimeric receptor-expressing cell of the present disclosure may also express a truncated epidermal growth factor receptor (EGFR) that lacks signaling capacity but retains an epitope that is recognized by molecules capable of inducing ADCC (e.g., WO2011/056894).

In some embodiments, a chimeric receptor-expressing cell of the present disclosure further includes a highly expressing compact marker/suicide gene that combines target epitopes from both CD32 and CD20 antigens in the chimeric receptor-expressing cell, which binds an anti-CD20 antibody (e.g., rituximab) resulting in selective depletion of the chimeric receptor-expressing cell by ADCC. Other methods for depleting chimeric receptor-expressing cells of the present disclosure my include, without limitation, administration of a monoclonal anti-CD52 antibody that selectively binds and targets the chimeric receptor-expressing cell for destruction by inducing ADCC. In some embodiments, the chimeric receptor-expressing cell can be selectively targeted using a chimeric receptor ligand, such as an anti-idiotypic antibody. In some embodiments, the anti-idiotypic antibody can cause effector cell activity, such as ADCC or ADC activity. In some embodiments, the chimeric receptor ligand can be further coupled to an agent that induces cell killing, such as a toxin. In some embodiments, a chimeric receptor-expressing cell of the present disclosure may further express a target protein recognized by a cell depleting agent of the present disclosure. In some embodiments, the target protein is CD20 and the cell depleting agent is an anti-CD20 antibody. In such embodiments, the cell depleting agent is administered once it is desirable to reduce or eliminate the chimeric receptor-expressing cell. In some embodiments, the cell depleting agent is an anti-CD52 antibody.

In some embodiments, a regulated chimeric receptor comprises a set of polypeptides, in which the components of a chimeric receptor of the present disclosure are partitioned on separate polypeptides or members. For example, the set of polypeptides may include a dimerization switch that, when in the presence of a dimerization molecule, can couple the polypeptides to one another to form a functional chimeric receptor.

### Chimeric Receptor-Encoding Nucleic Acid Constructs

Certain aspects of the present disclosure relate to nucleic acids (e.g., isolated nucleic acids) encoding one or more chimeric receptors of the present disclosure. In some embodiments, the nucleic acid is an RNA construct, such as a messenger RNA (mRNA) transcript or a modified RNA. In some embodiments, the nucleic acid is a DNA construct.

In some embodiments, a nucleic acid of the present disclosure encodes a chimeric receptor that comprises one or more antigen-binding domain, where each domain binds to a target antigen (e.g., a solid tumor antigen), a transmembrane domain, and one or more intracellular signaling domains. In some embodiments, the nucleic acid encodes a chimeric receptor that comprises an antigen-binding domain, a transmembrane domain, a primary signaling domain (e.g., CD3-zeta domain), and one or more costimulatory signaling domains. In some embodiments, the nucleic acid further comprises a nucleotide sequence encoding a spacer region. In some embodiments, the antigen-binding domain is connected to the transmembrane domain by the spacer region. In some embodiments, the spacer region comprises a nucleic acid sequence selected from any of the nucleic acid sequences listed in Table 9. In some embodiments, the nucleic acid further comprises a nucleotide sequence encoding a leader sequence.

The nucleic acids of the present disclosure may be obtained using any suitable recombinant methods known in the art, including, without limitation, by screening libraries from cells expressing the gene of interest, by deriving the gene of interest from a vector known to include the gene, or by isolating the gene of interest directly from cells and tissues containing the gene using standard techniques. Alternatively, the gene of interest may be produced synthetically.

In some embodiments, a nucleic acid of the present disclosure in comprised within a vector. In some embodiments, a nucleic acid of the present disclosure is expressed in a cell via transposons, a CRISPR/Cas9 system, a TALEN, or a zinc finger nuclease.

In some embodiments, expression of a nucleic acid encoding a chimeric receptor of the present disclosure may be achieved by operably linking the nucleic acid to a promoter and incorporating the construct into an expression vector. A suitable vector can replicate and integrate in eukaryotic cells. Typical cloning vectors contain transcription and translation terminators, initiation sequences, and promoters useful for regulating expression of the desired nucleic acid.

In some embodiments, expression constructs of the present disclosure may also be used for nucleic acid immunization and gene therapy, using standard gene delivery protocols (e.g., U.S. Pat. Nos. 5,399,346, 5,580,859, and 5,589,466). In some embodiments, a vector of the present disclosure is a gene therapy vector.

A nucleic acid of the present disclosure can be cloned into a number of types of vectors. For example, the nucleic acid can be cloned into a vector including, without limitation, a plasmid, a phagemid, a phage derivative, an animal virus, or a cosmid. In some embodiments, the vector may be an expression vector, a replication vector, a probe generation vector, or a sequencing vector.

In some embodiments, the plasmid vector comprises a transposon/transposase system to incorporate the nucleic acids of the present disclosure into the host cell genome. Methods of expressing proteins in immune cells using a transposon and transposase plasmid system are generally described in Chicaybam L, Hum Gene Ther. 2019 April; 30(4):511-522. doi: 10.1089/hum.2018.218; and Ptackova P, Cytotherapy. 2018 April; 20(4):507-520. doi: 10.1016/j.jcyt.2017.10.001, each of which are hereby incorporated by reference in their entirety. In some embodiments, the transposon system is the Sleeping Beauty transposon/transposase or the piggyBac transposon/transposase.

In some embodiments, an expression vector of the present disclosure may be provided to a cell in the form of a viral vector. Suitable viral vector systems are well known in the art. For example, viral vectors may be derived from retroviruses (e.g., gammaretroviruses and lentiviruses), adenoviruses, adeno-associated viruses, and herpes viruses. In some embodiments, a vector of the present disclosure is a retroviral vector. Types of retroviral vectors include lentiviral vectors and gammaretroviral vectors. In some embodiments, a vector of the present disclosure is a lentiviral vector. Lentiviral vectors are derived from lentiviruses, such as human immunodeficiency virus (HIV), and can be suitable for long-term gene transfer as such vectors generally allow long-term, stable integration of a transgene and its propagation in daughter cells. Lentiviral vectors can be advantageous over other retroviral vectors (e.g., murine leukemia viruses) for transducing certain cell types because lentiviral vectors can typically transduce non-proliferating cells. In some embodiments, a vector of the present disclosure is a gammaretroviral vector. Gammaretroviral vectors are derived from viruses of the genus Gammaretrovirus, which includes murine leukemia virus (MLV) and feline leukemia virus.

Viral particles produced using retroviral vectors (e.g., lentiviral and gammaretroviral vectors) are typically pseudotyped to include an envelope protein that is exogenous to the retrovirus. A common envelope protein used for pseudotyping is the Vesicular stomatitis virus (VSV) G glycoprotein. In some embodiments, the retroviral vector (e.g., lentiviral vector or gammaretrovirus vector) is pseudotyped using a baboon endogenous retrovirus (BaEV) envelope protein. The BaEV envelope protein may be a wild-type BaEV envelope, or may be a variant BaEV envelope, such as a chimeric BaEV having the cytoplasmic tail replaced with that of the MLV-A virus or a version truncated to remove the R peptide at the C-terminus (e.g., Anais Girard-Gagnepain, et al. Blood 2014; 124 (8): 1221-1231. doi: https://doi.org/10.1182/blood-2014-02-558163).

In some embodiments, a vector of the present disclosure is an adenoviral vector (A5/35). In some embodiments, a vector of the present disclosure contains an origin of replication functional in at least one organism, a promoter sequence, convenient restriction endonuclease sites, and one or more selectable markers (e.g., WO01/96584; WO01/29058; and U.S. Pat. No. 6,326,193). A number of viral based systems have been developed for gene transfer into mammalian cells. A selected gene can be inserted into a vector and packaged in retroviral particles using techniques known in the art. The recombinant virus can then be isolated and delivered to mammalian cells either in vivo or ex vivo. A number of retroviral systems are known in the art.

In some embodiments, vectors of the present disclosure include additional promoter elements, such as enhancers that regulate the frequency of transcriptional initiation. Enhancers are typically located in a region that is 30 bp to 110 bp upstream of the start site, although a number of promoters have been shown to contain functional elements downstream of the start site as well. The spacing between promoter elements may be flexible, so that promoter function is preserved when elements are inverted or moved relative to one another. For example, in the thymidine kinase (tk) promoter the spacing between promoter elements can be increased to 50 bp apart before activity begins to decline. Depending on the promoter, individual elements may function either cooperatively or independently to activate transcription. Exemplary promoters may include, without limitation, the SFFV gene promoter, the EFS gene promoter, the CMV IE gene promoter, the EF1a promoter, the ubiquitin C promoter, and the phosphoglycerokinase (PGK) promoter.

In some embodiments, a promoter that is capable of expressing a nucleic acid of the present disclosure in a mammalian cell, such as an immunoresponsive cell of the present disclosure, is the EF1a promoter. The native EF1a promoter drives expression of the alpha subunit of the elongation factor-1 complex, which is responsible for the enzymatic delivery of aminoacyl tRNAs to the ribosome. The EF1a promoter has been widely used in mammalian expression plasmids and has been shown to be effective in driving chimeric receptor expression from nucleic acids cloned into a lentiviral vector.

In some embodiments, a promoter that is capable of expressing a nucleic acid of the present disclosure in a mammalian cell, such as an immunoresponsive cell of the present disclosure, is a constitutive promoter. For example, a suitable constitutive promoter is the immediate early cytomegalovirus (CMV) promoter. The CMV promoter is a strong constitutive promoter that is capable of driving high levels of expression of any polynucleotide sequence operatively linked to the promoter. Other suitable constitutive promoters include, without limitation, a ubiquitin C (UbiC) promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, an actin promoter, a myosin promoter, an elongation factor-1a promoter, a hemoglobin promoter, and a creatine kinase promoter.

In some embodiments, a promoter that is capable of expressing a nucleic acid of the present disclosure in a mammalian cell, such as an immunoresponsive cell of the present disclosure, is an inducible promoter. Use of an inducible promoter may provide a molecular switch that is capable of inducing or repressing expression of a nucleic acid of the present disclosure when the promoter is operatively linked to the nucleic acid. Examples of inducible promoters include, without limitation, a metallothionine promoter, a glucocorticoid promoter, a progesterone promoter, and a tetracycline promoter.

In some embodiments, a vector of the present disclosure may further comprise a signal sequence to facilitate secretion, a polyadenylation signal and transcription terminator, an element allowing episomal replication, and/or elements allowing for selection. An exemplary signal sequence is provided in Table 11.

TABLE 11

| Amino Acid Sequence | SEQ ID NO: | Description |
|---|---|---|
| MALPVTALLLP | 76 | CD8 Signal |
| LALLLHAARP | | Sequence |

In some embodiments, a vector of the present disclosure can further comprise a selectable marker gene and/or reporter gene to facilitate identification and selection of chimeric receptor-expressing cells from a population of cells that have been transduced with the vector. In some embodiments, the selectable marker may be encoded by a nucleic acid that is separate from the vector and used in a co-transfection procedure. Either selectable marker or reporter gene may be flanked with appropriate regulator sequences to allow expression in host cells. Examples of selectable markers include, without limitation, antibiotic-resistance genes, such as neo and the like.

In some embodiments, reporter genes may be used for identifying transduced cells and for evaluating the functionality of regulatory sequences. As disclosed herein, a reporter gene is a gene that is not present in or expressed by the recipient organism or tissue and that encodes a polypeptide whose expression results in an easily detectable property, such as enzymatic activity. Expression of the reporter gene can be assayed at a suitable time after the nucleic acid has been introduced into the recipient cells. Examples of reporter genes include, without limitation, genes encoding for luciferase, genes encoding for beta-galactosidase, genes encoding for chloramphenicol acetyl transferase, genes encoding for secreted alkaline phosphatase, and genes encoding for green fluorescent protein. Suitable expression systems are well known in the art and may be prepared using known techniques or obtained commercially. In some embodiments, a construct with a minimal 5′ flanking region showing the highest level of expression of the reporter gene is identified as the promoter. Such promoter regions may be linked to a reporter gene and used to evaluate agents for the ability to modulate promoter-driven transcription.

In some embodiments, a vector comprising a nuclei acid sequence encoding a chimeric receptor of the present disclosure further comprises a second nucleic acid encoding a polypeptide that increases the activity of the chimeric receptor.

In embodiments where a chimeric receptor-expressing cell comprises two or more chimeric receptors, a single nucleic acid may encode the two or more chimeric receptors under a single regulatory control element (e.g., promoter) or under separate regulatory control elements for each chimeric receptor-encoding nucleotide sequence comprised in the nucleic acid. In some embodiments where a chimeric receptor-expressing cell comprises two or more chimeric receptors, each chimeric receptor may be encoded by separate nucleic acid. In some embodiments, each separate nucleic acid comprises its own control element (e.g., promoter). In some embodiments, a single nucleic acid encodes the two or more chimeric receptors and the chimeric receptor-encoding nucleotide sequences are in the same reading frame and are expressed as a single polypeptide chain. In such embodiments, the two or more chimeric receptors may be separated by one or more peptide cleavage sites, such as auto-cleavage sites or substrates for an intracellular protease. Suitable peptide cleavage sites may include, without limitation, a T2A peptide cleavage site, a P2A peptide cleavage site, an E2A peptide cleavage sire, and an F2A peptide cleavage site. In some embodiments, the two or more chimeric receptors comprise a T2A peptide cleavage site. In some embodiments, the two or more chimeric receptors comprise an E2A peptide cleavage site. In some embodiments, the two or more chimeric receptors comprise a T2A and an E2A peptide cleavage site.

Methods of introducing and expressing genes into a cell are well known in the art. For example, in some embodiments, an expression vector can be transferred into a host cell by physical, chemical, or biological means. Examples of physical means for introducing a nucleic acid into a host cell include, without limitation, calcium phosphate precipitation, lipofection, particle bombardment, microinjection, and electroporation. Examples of chemical means for introducing a nucleic acid into a host cell include, without limitation, colloidal dispersion systems, macromolecule complexes, nanocapsules, microspheres, beads, and lipid-based systems including oil-in-water emulsions, micelles, mixed micelles, and liposomes. Examples of biological means for introducing a nucleic acid into a host cell include, without limitation, the use of DNA and RNA vectors.

In some embodiments, liposomes may be used as a non-viral delivery system to introduce a nucleic acid or vector of the present disclosure into a host cell in vitro, ex vivo, or in vivo. In some embodiments, the nucleic acid may be associated with a lipid, for example by being encapsulated in the aqueous interior of a liposome, being interspersed within the lipid bilayer of a liposome, being attached to a liposome via a linking molecule that is associated with both the liposome and the nucleic acid, being entrapped in a liposome, being complexed with a liposome, being dispersed in a solution containing a lipid, being mixed with a lipid, being combined with a lipid, being contained as a suspension in a lipid, being contained or complexed with a micelle, or otherwise being associated with a lipid. As disclosed herein, lipid-associated nucleic acid or vector compositions are not limited to any particular structure in solution. In some embodiments, such compositions may be present in a bilayer structure, as micelles or with a “collapsed” structure. Such compositions may also be interspersed in a solution, forming aggregates that are not uniform in size or shape. As disclosed herein, lipids are fatty substances that may be naturally occurring or synthetic. In some embodiments, lipids can include the fatty droplets that naturally occur in the cytoplasm or the class of compounds that contain long-chain aliphatic hydrocarbons and their derivatives, such as fatty acids, alcohols, amines, amino alcohols, and aldehydes. Suitable lipids may be obtained from commercial sources and include, without limitation, dimyristyl phosphatidylcholine (“DMPC”), dicetylphosphate (“DCP”), cholesterol, and dimyristylphosphatidylglycerol (“DMPG”). Stock solutions of lipids in chloroform or chloroform/methanol can be stored at about −20° C. Chloroform is used as the solvent, as it is more readily evaporated than methanol. As used herein, a “liposome” may encompass a variety of single and multilamellar lipid vehicles formed by the generation of enclosed lipid bilayers or aggregates. In some embodiments, liposomes can be characterized as having vesicular structures with a phospholipid bilayer membrane and an inner aqueous medium. In some embodiments, multilamellar liposomes may have multiple lipid layers separated by aqueous medium. Multilamellar liposomes can form spontaneously when phospholipids are suspended in an excess of aqueous solution. In some embodiments, lipid components may undergo self-rearrangement before the formation of closed structures and can entrap water and dissolved solutes between the lipid bilayers. In some embodiments, the lipids may assume a micellar structure or merely exist as nonuniform aggregates of lipid molecules.

In some embodiments, a nucleic acid or vector of the present disclosure is introduced into a mammalian host cell, such as an immunoresponsive cell of the present disclosure. In some embodiments, the presence of a nucleic acid or vector of the present disclosure in a host cell may be confirmed by any suitable assay known in the art, including without limitation Southern blot assays, Northern blot assays, RT-PCR, PCR, ELISA assays, and Western blot assays.

In some embodiments, a nucleic acid or vector of the present disclosure is stably transduced into an immunoresponsive cell of the present disclosure. In some embodiments, cells that exhibit stable expression of the nucleic acid or vector express the encoded chimeric receptor for at least 1 week, at least 2 weeks, at least 3 weeks, at least 4 weeks, at least 5 weeks, at least 6 weeks, at least 7 weeks, at least 8 weeks, at least 3 months, at least 6 months, at least 9 months, or at least 12 months after transduction.

In embodiments where a chimeric receptor of the present disclosure is transiently expressed in a cell, a chimeric receptor-encoding nucleic acid or vector of the present disclosure is transfected into an immunoresponsive cell of the present disclosure. In some embodiments the immunoresponsive cell expresses the chimeric receptor for about 4 days, about 5 days, about 6 days, about 7 days, about 8 days, about 9 days, about 10 days, about 11 days, about 12 days, about 13 days, about 14 days, or about 15 days after transfection.

In some embodiments, the nucleic acid construct encodes a bicistronic chimeric antigen receptor, comprising a chimeric receptor and a chimeric inhibitory receptor. In some embodiments, the nucleic acid construct comprises a multicistronic chimeric antigen receptor, comprising two or more chimeric receptors and a chimeric inhibitory receptor. In some embodiments, multiple nucleic acid constructs are used, wherein one construct encodes the chimeric receptor and one construct encodes the chimeric inhibitory receptor.

In some embodiments, the chimeric receptor binds a CEACAM6 antigen and the chimeric inhibitory receptor binds a VSIG2, CPM, ITM2C, SLC26A2, SLC4A4, GPA33, PLA2G2A, ABCA8, ATP1A2, CHP2, or SLC26A3 antigen.

In some embodiments, the chimeric inhibitory receptor binds a VSIG2 antigen and the chimeric receptor binds a CEA antigen. In some embodiments, the chimeric inhibitory receptor binds a VSIG2 antigen and the chimeric receptor binds a CEACAM1 antigen. In some embodiments, the chimeric inhibitory receptor binds a VSIG2 antigen and the chimeric receptor binds a CEACAM5 antigen. In some embodiments, the chimeric inhibitory receptor binds a VSIG2 antigen and the chimeric receptor binds a CEACAM6 antigen.

In some embodiments, the chimeric inhibitory receptor binds a CPM antigen and the chimeric receptor binds a CEA antigen. In some embodiments, the chimeric inhibitory receptor binds a CPM antigen and the chimeric receptor binds a CEACAM1 antigen. In some embodiments, the chimeric inhibitory receptor binds a CPM antigen and the chimeric receptor binds a CEACAM5 antigen. In some embodiments, the chimeric inhibitory receptor binds a CPM antigen and the chimeric receptor binds a CEACAM6 antigen.

In some embodiments, the chimeric inhibitory receptor binds an ITM2C antigen and the chimeric receptor binds a CEA antigen. In some embodiments, the chimeric inhibitory receptor binds an ITM2C antigen and the chimeric receptor binds a CEACAM1 antigen. In some embodiments, the chimeric inhibitory receptor binds an ITM2C antigen and the chimeric receptor binds a CEACAM5 antigen. In some embodiments, the chimeric inhibitory receptor binds an ITM2C antigen and the chimeric receptor binds a CEACAM6 antigen.

In some embodiments, the chimeric inhibitory receptor binds a SLC26A2 antigen and the chimeric receptor binds a CEA antigen. In some embodiments, the chimeric inhibitory receptor binds a SLC26A2 antigen and the chimeric receptor binds a CEACAM1 antigen. In some embodiments, the chimeric inhibitory receptor binds a SLC26A2 antigen and the chimeric receptor binds a CEACAM5 antigen. In some embodiments, the chimeric inhibitory receptor binds a SLC26A2 antigen and the chimeric receptor binds a CEACAM6 antigen.

In some embodiments, the chimeric inhibitory receptor binds a SLC4A4 antigen and the chimeric receptor binds a CEA antigen. In some embodiments, the chimeric inhibitory receptor binds a SLC4A4 antigen and the chimeric receptor binds a CEACAM1 antigen. In some embodiments, the chimeric inhibitory receptor binds a SLC4A4 antigen and the chimeric receptor binds a CEACAM5 antigen. In some embodiments, the chimeric inhibitory receptor binds a SLC4A4 antigen and the chimeric receptor binds a CEACAM6 antigen.

In some embodiments, the chimeric inhibitory receptor binds a GPA33 antigen and the chimeric receptor binds a CEA antigen. In some embodiments, the chimeric inhibitory receptor binds a GPA33 antigen and the chimeric receptor binds a CEACAM1 antigen. In some embodiments, the chimeric inhibitory receptor binds a GPA33 antigen and the chimeric receptor binds a CEACAM5 antigen. In some embodiments, the chimeric inhibitory receptor binds a GPA33 antigen and the chimeric receptor binds a CEACAM6 antigen.

In some embodiments, the chimeric inhibitory receptor binds a PLA2G2A antigen and the chimeric receptor binds a CEA antigen. In some embodiments, the chimeric inhibitory receptor binds a PLA2G2A antigen and the chimeric receptor binds a CEACAM1 antigen. In some embodiments, the chimeric inhibitory receptor binds a PLA2G2A antigen and the chimeric receptor binds a CEACAM5 antigen. In some embodiments, the chimeric inhibitory receptor binds a PLA2G2A antigen and the chimeric receptor binds a CEACAM6 antigen.

In some embodiments, the chimeric inhibitory receptor binds an ABCA8 antigen and the chimeric receptor binds a CEA antigen. In some embodiments, the chimeric inhibitory receptor binds an ABCA8 antigen and the chimeric receptor binds a CEACAM1 antigen. In some embodiments, the chimeric inhibitory receptor binds an ABCA8 antigen and the chimeric receptor binds a CEACAM5 antigen. In some embodiments, the chimeric inhibitory receptor binds an ABCA8 antigen and the chimeric receptor binds a CEACAM6 antigen.

In some embodiments, the chimeric inhibitory receptor binds an ATP1A2 antigen and the chimeric receptor binds a CEA antigen. In some embodiments, the chimeric inhibitory receptor binds an ATP1A2 antigen and the chimeric receptor binds a CEACAM1 antigen. In some embodiments, the chimeric inhibitory receptor binds an ATP1A2 antigen and the chimeric receptor binds a CEACAM5 antigen. In some embodiments, the chimeric inhibitory receptor binds an ATP1A2 antigen and the chimeric receptor binds a CEACAM6 antigen.

In some embodiments, the chimeric inhibitory receptor binds a CHP2 antigen and the chimeric receptor binds a CEA antigen. In some embodiments, the chimeric inhibitory receptor binds a CHP2 antigen and the chimeric receptor binds a CEACAM1 antigen. In some embodiments, the chimeric inhibitory receptor binds a CHP2 antigen and the chimeric receptor binds a CEACAM5 antigen. In some embodiments, the chimeric inhibitory receptor binds a CHP2 antigen and the chimeric receptor binds a CEACAM6 antigen.

In some embodiments, the chimeric inhibitory receptor binds an SLC26A3 antigen and the chimeric receptor binds a CEA antigen. In some embodiments, the chimeric inhibitory receptor binds an SLC26A3 antigen and the chimeric receptor binds a CEACAM1 antigen. In some embodiments, the chimeric inhibitory receptor binds an SLC26A3 antigen and the chimeric receptor binds a CEACAM5 antigen. In some embodiments, the chimeric inhibitory receptor binds an SLC26A3 antigen and the chimeric receptor binds a CEACAM6 antigen.

In some embodiments, the encoded bicistronic chimeric antigen receptor system comprises a CAR with an antigen binding domain targeting any antigen provided in Table 1. In some embodiments, the encoded bicistronic chimeric antigen receptor system comprises a CAR with an antigen binding domain derived from an antibody as provided in Table 2. In some embodiments, the encoded bicistronic chimeric antigen receptor system comprises a CAR with an antigen binding domain including an scFv as provided in Table 3. In some embodiments, the encoded bicistronic chimeric antigen receptor system comprises a CAR with two or more antigen binding domains targeting any antigen pair provided in Table 10.

In some embodiments, the nucleic acid construct encodes a bivalent chimeric antigen receptor, comprising a chimeric receptor and a chimeric inhibitory receptor. In some embodiments, the chimeric receptor binds a CEACAM6 antigen and the chimeric inhibitory receptor binds a VSIG2, CPM, ITM2C, SLC26A2, SLC4A4, GPA33, PLA2G2A, ABCA8, ATP1A2, CHP2, or SLC26A3 antigen.

In some embodiments, the chimeric inhibitory receptor binds a VSIG2 antigen and the chimeric receptor binds a CEA antigen. In some embodiments, the chimeric inhibitory receptor binds a VSIG2 antigen and the chimeric receptor binds a CEACAM1 antigen. In some embodiments, the chimeric inhibitory receptor binds a VSIG2 antigen and the chimeric receptor binds a CEACAM5 antigen. In some embodiments, the chimeric inhibitory receptor binds a VSIG2 antigen and the chimeric receptor binds a CEACAM6 antigen.

In some embodiments, the chimeric inhibitory receptor binds a CPM antigen and the chimeric receptor binds a CEA antigen. In some embodiments, the chimeric inhibitory receptor binds a CPM antigen and the chimeric receptor binds a CEACAM1 antigen. In some embodiments, the chimeric inhibitory receptor binds a CPM antigen and the chimeric receptor binds a CEACAM5 antigen. In some embodiments, the chimeric inhibitory receptor binds a CPM antigen and the chimeric receptor binds a CEACAM6 antigen.

In some embodiments, the chimeric inhibitory receptor binds an ITM2C antigen and the chimeric receptor binds a CEA antigen. In some embodiments, the chimeric inhibitory receptor binds an ITM2C antigen and the chimeric receptor binds a CEACAM1 antigen. In some embodiments, the chimeric inhibitory receptor binds an ITM2C antigen and the chimeric receptor binds a CEACAM5 antigen. In some embodiments, the chimeric inhibitory receptor binds an ITM2C antigen and the chimeric receptor binds a CEACAM6 antigen.

In some embodiments, the chimeric inhibitory receptor binds a SLC26A2 antigen and the chimeric receptor binds a CEA antigen. In some embodiments, the chimeric inhibitory receptor binds a SLC26A2 antigen and the chimeric receptor binds a CEACAM1 antigen. In some embodiments, the chimeric inhibitory receptor binds a SLC26A2 antigen and the chimeric receptor binds a CEACAM5 antigen. In some embodiments, the chimeric inhibitory receptor binds a SLC26A2 antigen and the chimeric receptor binds a CEACAM6 antigen.

In some embodiments, the chimeric inhibitory receptor binds a SLC4A4 antigen and the chimeric receptor binds a CEA antigen. In some embodiments, the chimeric inhibitory receptor binds a SLC4A4 antigen and the chimeric receptor binds a CEACAM1 antigen. In some embodiments, the chimeric inhibitory receptor binds a SLC4A4 antigen and the chimeric receptor binds a CEACAM5 antigen. In some embodiments, the chimeric inhibitory receptor binds a SLC4A4 antigen and the chimeric receptor binds a CEACAM6 antigen.

In some embodiments, the chimeric inhibitory receptor binds a GPA33 antigen and the chimeric receptor binds a CEA antigen. In some embodiments, the chimeric inhibitory receptor binds a GPA33 antigen and the chimeric receptor binds a CEACAM1 antigen. In some embodiments, the chimeric inhibitory receptor binds a GPA33 antigen and the chimeric receptor binds a CEACAM5 antigen. In some embodiments, the chimeric inhibitory receptor binds a GPA33 antigen and the chimeric receptor binds a CEACAM6 antigen.

In some embodiments, the chimeric inhibitory receptor binds a PLA2G2A antigen and the chimeric receptor binds a CEA antigen. In some embodiments, the chimeric inhibitory receptor binds a PLA2G2A antigen and the chimeric receptor binds a CEACAM1 antigen. In some embodiments, the chimeric inhibitory receptor binds a PLA2G2A antigen and the chimeric receptor binds a CEACAM5 antigen. In some embodiments, the chimeric inhibitory receptor binds a PLA2G2A antigen and the chimeric receptor binds a CEACAM6 antigen.

In some embodiments, the chimeric inhibitory receptor binds an ABCA8 antigen and the chimeric receptor binds a CEA antigen. In some embodiments, the chimeric inhibitory receptor binds an ABCA8 antigen and the chimeric receptor binds a CEACAM1 antigen. In some embodiments, the chimeric inhibitory receptor binds an ABCA8 antigen and the chimeric receptor binds a CEACAM5 antigen. In some embodiments, the chimeric inhibitory receptor binds an ABCA8 antigen and the chimeric receptor binds a CEACAM6 antigen.

In some embodiments, the chimeric inhibitory receptor binds an ATP1A2 antigen and the chimeric receptor binds a CEA antigen. In some embodiments, the chimeric inhibitory receptor binds an ATP1A2 antigen and the chimeric receptor binds a CEACAM1 antigen. In some embodiments, the chimeric inhibitory receptor binds an ATP1A2 antigen and the chimeric receptor binds a CEACAM5 antigen. In some embodiments, the chimeric inhibitory receptor binds an ATP1A2 antigen and the chimeric receptor binds a CEACAM6 antigen.

In some embodiments, the chimeric inhibitory receptor binds a CHP2 antigen and the chimeric receptor binds a CEA antigen. In some embodiments, the chimeric inhibitory receptor binds a CHP2 antigen and the chimeric receptor binds a CEACAM1 antigen. In some embodiments, the chimeric inhibitory receptor binds a CHP2 antigen and the chimeric receptor binds a CEACAM5 antigen. In some embodiments, the chimeric inhibitory receptor binds a CHP2 antigen and the chimeric receptor binds a CEACAM6 antigen.

In some embodiments, the chimeric inhibitory receptor binds an SLC26A3 antigen and the chimeric receptor binds a CEA antigen. In some embodiments, the chimeric inhibitory receptor binds an SLC26A3 antigen and the chimeric receptor binds a CEACAM1 antigen. In some embodiments, the chimeric inhibitory receptor binds an SLC26A3 antigen and the chimeric receptor binds a CEACAM5 antigen. In some embodiments, the chimeric inhibitory receptor binds an SLC26A3 antigen and the chimeric receptor binds a CEACAM6 antigen.

In some embodiments, the encoded bivalent chimeric antigen receptor comprises a CAR with an antigen binding domain targeting any antigen provided in Table 1. In some embodiments, the encoded bivalent chimeric antigen receptor comprises a CAR with an antigen binding domain derived from an antibody as provided in Table 2. In some embodiments, the encoded bivalent chimeric antigen receptor comprises a CAR with an antigen binding domain including an scFv as provided in Table 3. In some embodiments, the encoded bivalent chimeric antigen receptor comprises a CAR with two or more antigen binding domains targeting any antigen pair provided in Table 10.

### Pharmaceutical Compositions and Administration

Certain aspects of the present disclosure relate to compositions (e.g., pharmaceutical compositions) comprising one or more chimeric receptors of the present disclosure or immunoresponsive cells of the present disclosure that express such one or more chimeric receptors. In some embodiments, compositions comprising chimeric receptors or genetically modified immunoresponsive cells that express such chimeric receptors can be provided systemically or directly to a subject for the treatment of a proliferative disorder, such as a solid tumor.

Compositions comprising genetically modified cells of the present disclosure may be administered in any physiologically acceptable vehicle, for example intravascularly, although they may also be introduced into bone or other convenient sites where the genetically modified cells may find an appropriate site for regeneration and differentiation (e.g., thymus). Compositions comprising genetically modified cells of the present disclosure can comprise a purified population of cells. Methods for determining the percentage of genetically modified cells in a population of cells are well known in the art and include, without limitation, fluorescence activated cell sorting (FACS). In some embodiments, the purity of genetically modified cells in a population of cells may be about 50%, about 55%, about 60%, or about 65%, about 70%, about 75%, about 80%, about 85%, about 90%, about 95%, about 97%, about 98%, about 99%, or more of the cells in the population of cells. Dosages can be readily adjusted by those skilled in the art (e.g., a decrease in purity may require an increase in dosage).

In certain embodiments, the compositions are pharmaceutical compositions comprising genetically modified cells, such as immunoresponsive cells or their progenitors and a pharmaceutically acceptable carrier. Administration can be autologous or heterologous.

### Methods of Treatment

Certain aspects of the present disclosure relate to methods of using the chimeric receptors and genetically modified cells of the present disclosure (e.g., immunoresponsive cells, e.g., NK cells) that express such chimeric receptors to treat subjects in need thereof. In some embodiments, the methods of the present disclosure are useful for treating cancer in a subject, wherein the cancer expresses CEA (is CEA+). In some embodiments, the methods of the present disclosure are useful for treating cancer in a subject, wherein the cancer expresses CEACAM1 (is CEACAM1+). In some embodiments, the methods of the present disclosure are useful for treating cancer in a subject, wherein the cancer expresses CEACAM5 (is CEACAM5+). In some embodiments, the methods of the present disclosure are useful for treating cancer in a subject, wherein the cancer expresses CEACAM6 (is CEACAM6+). In some embodiments, the methods of the present disclosure are useful for treating cancer in a subject, such as a solid tumor.

In some embodiments, the solid tumor is a cancer of the lung, pancreas, gastrointestinal tract, and colon. In some embodiments, the solid tumor is lung adenocarcinoma. In some embodiments, the solid tumor is pancreatic cancer. In some embodiments, the solid tumor is a gastrointestinal cancer (including, without limitation, esophagus, stomach, small intestine, colon or rectum cancer). In some embodiments, the solid tumor is colon cancer. In some embodiments, the solid tumor is colorectal cancer.

In some embodiments, the methods of the present disclosure may comprise administering genetically modified cells of the present disclosure in an amount effective to achieve the desired effect, including without limitation palliation of an existing condition, prevention of a condition, treatment an existing condition, management of an existing condition, or prevention of recurrence or relapse of a condition. In some embodiments, the effective amount can be provided in one or a series of administrations of the genetically modified cells of the present disclosure (e.g., immunoresponsive cells, e.g., NK cells). In some embodiments, an effective amount can be provided in a bolus or by continuous perfusion.

As disclosed herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

In some embodiments, the method reduces the number of tumor cells. In some embodiments, the method reduces tumor size. In some embodiments, the method reduces tumor volume. In some embodiments, the method increases progression free survival in the subject. In some embodiments, the method increases survival in the subject.

### Therapeutic Treatment

In some embodiments, the methods of the present disclosure increase an immune response in a subject in need thereof. In some embodiments, the methods of the present disclosure include methods for treating and/or preventing a solid tumor in a subject. In some embodiments, the subject is a human. In some embodiments, suitable human subjects for therapy may comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., based on percentage of tumor cells, by palpation, CAT scan, sonogram, mammogram or X-ray; positive biochemical or histopathologic markers on their own are insufficient to identify this population). In some embodiments, a pharmaceutical composition of the present disclosure is administered to these subjects to elicit an anti-tumor response, with the objective of palliating their condition. In some embodiments, reduction in tumor mass occurs as a result of administration of the pharmaceutical composition, but any clinical improvement will constitute a benefit. In some embodiments, clinical improvement includes decreased risk or rate of progression or reduction in pathological consequences of the tumor. In some embodiments, a second group of suitable human subjects are “adjuvant group” subjects. These subjects are individuals who have had a history of a solid tumor, but have been responsive to another mode of therapy. The prior therapy may have included, without limitation, surgical resection, radiotherapy, and/or traditional chemotherapy. As a result, these individuals have no clinically measurable tumor. However, they are suspected of being at risk for progression of the disease, either near the original tumor site, or by metastases. In some embodiments, this group can be further subdivided into high-risk and low-risk individuals. The subdivision can be made on the basis of features observed before or after the initial treatment. These features are known in the clinical arts, and are suitably defined for each different solid tumor. Features typical of high-risk subgroups are those in which the tumor has invaded neighboring tissues, or who show involvement of lymph nodes.

In any and all aspects of increasing an immune response as described herein, any increase or decrease or alteration of an aspect of characteristic(s) or function(s) is as compared to a cell not contacted with an immunoresponsive cell as described herein.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response.

In some embodiments, a further group of subjects are those having a genetic predisposition to a solid tumor disorder, but that have not yet evidenced clinical signs of the solid tumor. For example, women testing positive for a genetic mutation associated with a cancer of the female reproductive organs (e.g., breast cancer, ovarian cancer), but still of childbearing age, may benefit from receiving one or more of the cells of the present disclosure (e.g., immunoresponsive cells, e.g. NK cells) in treatment prophylactically to prevent the occurrence of said cancer of the female reproductive organs until it is suitable to perform preventive surgery. In some embodiments, the subjects can have an advanced form of disease, in which case the treatment objective can include mitigation or reversal of disease progression, and/or amelioration of side effects. In some embodiments, the subjects may have a history of the condition, for which they have already been treated, in which case the therapeutic objective may typically include a decrease or delay in the risk of recurrence.

In some embodiments, activity of a CEA CAR (e.g., a CEACAM1 CAR, a CEACAM5 CAR, or a CEACAM6 CAR) is inhibited. In some embodiments, activity of an immune cell expressing a CEA CAR (e.g., a CEACAM1 CAR, a CEACAM5 CAR, or a CEACAM6 CAR) is inhibited. In certain embodiments, the activity of the CEA CAR is inhibited using an inhibitory chimeric receptor. In certain embodiments, the activity of the immune cell expressing the CEA CAR is inhibited using an inhibitory chimeric receptor. In some embodiments, the inhibitory chimeric receptor binds a VSIG2 antigen. In some embodiments, the inhibitory chimeric receptor binds a CPM antigen. In some embodiments, the inhibitory chimeric receptor binds an ITM2C antigen. In some embodiments, the inhibitory chimeric receptor binds a SLC26A2 antigen. In some embodiments, the inhibitory chimeric receptor binds a SLC4A4 antigen. In some embodiments, the inhibitory chimeric receptor binds a GPA33 antigen. In some embodiments, the inhibitory chimeric receptor binds a PLA2G2A antigen. In some embodiments, the inhibitory chimeric receptor binds an ABCA8 antigen. In some embodiments, the inhibitory chimeric receptor binds an ATP1A2 antigen. In some embodiments, the inhibitory chimeric receptor binds a CHP2 antigen. In some embodiments, the inhibitory chimeric receptor binds an SLC26A3 antigen. In some embodiments, the chimeric receptor is a multispecific receptor comprising two or more antigen-binding domains, such that the chimeric receptor can bind two or more antigens.

### Combination Therapies

In some embodiments, genetically modified cells of the present disclosure (e.g., immunoresponsive cells, e.g., NK cells) expressing one or more chimeric receptors of the present disclosure may be used in combination with other known agents and therapies. In some embodiments, a combination therapy of the present disclosure comprises a genetically modified cell of the present disclosure that can be administered in combination with one or more additional therapeutic agents. In some embodiments, the genetically modified cell and the one or more additional therapeutic agents can be administered simultaneously, in the same or in separate compositions, or sequentially. For sequential administration, the genetically modified cell can be administered first, and the one or more additional agents can be administered second, or the order of administration can be reversed. In some embodiments, the genetically modified cells are further modified to express one or more additional therapeutic agents.

### Kits

Certain aspects of the present disclosure relate to kits for the treatment and/or prevention of a solid tumor. In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., immunoresponsive cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing a solid tumor, e.g., for treating and/or preventing a colorectal carcinoma, a pancreatic cancer, a lung adenocarcinoma, and/or a gastric cancer. In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### EMBODIMENTS

1. An inhibitory chimeric receptor comprising an extracellular antigen-binding domain that binds to an antigen selected from the group consisting of: VSIG2, CPM, ITM2C, SLC26A2, SLC4A4, GPA33, PLA2G2A, ABCA8, ATP1A2, CHP2, and SLC26A3.
2. The inhibitory chimeric receptor of embodiment 1, wherein the antigen is VSIG2.
3. The inhibitory chimeric receptor of embodiment 1, wherein the antigen is CPM.
4. The inhibitory chimeric receptor of embodiment 1, wherein the antigen is ITM2C.
5. The inhibitory chimeric receptor of embodiment 1, wherein the antigen is SLC26A2.
6. The inhibitory chimeric receptor of embodiment 1, wherein the antigen is SLC4A4.
7. The inhibitory chimeric receptor of embodiment 1, wherein the antigen is GPA33.
8. The inhibitory chimeric receptor of embodiment 1, wherein the antigen is PLA2G2A.
9. The inhibitory chimeric receptor of embodiment 1, wherein the antigen is ABCA8.
10. The inhibitory chimeric receptor of embodiment 1, wherein the antigen is ATP1A2.
11. The inhibitory chimeric receptor of embodiment 1, wherein the antigen is CHP2.
12. The inhibitory chimeric receptor of embodiment 1, wherein the antigen is SLC26A3.
13. The inhibitory chimeric receptor of any one of embodiments 1-12, wherein when expressed on a cell the inhibitory chimeric receptor inhibits one or more activities of the cell.
14. The inhibitory chimeric receptor of any one of embodiments 1-13, wherein the antigen is not expressed on a tumor cell, or the antigen is expressed on a tumor cell at a level that is lower than expression on a non-tumor cell.
15. The inhibitory chimeric receptor of any one of embodiments 1-14, wherein the antigen is expressed on a non-tumor cell, or the antigen is expressed on a non-tumor cell at a level that is higher than expression on a corresponding tumor cell.
16. The inhibitory chimeric receptor of embodiment 15, wherein the antigen is expressed on a non-tumor cell derived from a tissue selected from the group consisting of: lung, pancreas, gastrointestinal tract, colon, brain, neuronal tissue, endocrine, bone, bone marrow, immune system, muscle, liver, gallbladder, kidney, urinary bladder, male reproductive organs, female reproductive organs, adipose, soft tissue, and skin.
17. The inhibitory chimeric receptor of any one of embodiments 1-16, wherein the inhibitory chimeric receptor comprises one or more intracellular inhibitory domains selected from the group consisting of: PD-1, CTLA4, TIGIT, LAIR1, GRB-2, Dok-1, Dok-2, SLAP, LAG3, HAVR, BTLA, LIR1, NKG2A, KIR3DL1, GITR, PD-L1, CSK, SHP-1, PTEN, CD45, CD148, PTP-MEG1, PTP-PEST, c-CBL, CBL-b, PTPN22, LAR, PTPH1, SHIP-1, RasGAP, CD94, and CD161.
18. The inhibitory chimeric receptor of any one of embodiments 1-17, wherein the antigen-binding domain comprises one or more antibodies, antigen-binding fragments of an antibody, F(ab) fragments, F(ab′) fragments, single chain variable fragments (scFvs), or single-domain antibodies (sdAbs).
19. The inhibitory chimeric receptor of any of any one of embodiments 1-18, wherein the antigen-binding domain comprises one or more single chain variable fragments (scFvs).
20. The inhibitory chimeric receptor of embodiment 19, wherein each of the one or more scFvs comprises a heavy chain variable domain (VH) and a light chain variable domain (VL).
21. The inhibitory chimeric receptor of embodiment 20, wherein the VH and VL are separated by a peptide linker.
22. The inhibitory chimeric receptor of embodiment 21, wherein the peptide linker comprises the amino acid sequence of SEQ ID NO: 39 or SEQ ID NO: 77.
23. The inhibitory chimeric receptor of any one of embodiments 20-22, wherein each of the one or more scFvs comprises the structure VH-L-VL or VL-L-VH, wherein VH is the heavy chain variable domain, L is the peptide linker, and VL is the light chain variable domain.
24. An isolated cell comprising the inhibitory chimeric receptor of any one of embodiments 1-23.
25. The isolated cell of embodiment 24, wherein the inhibitory chimeric receptor is recombinantly expressed.
26. The isolated cell of embodiment 24 or embodiment 25, wherein the inhibitory chimeric receptor is expressed from a vector or a selected locus from the genome of the cell.
27. The isolated cell of any one of embodiments 24-26, wherein the cell further comprises a chimeric receptor comprising one or more extracellular antigen-binding domains, wherein the one or more extracellular antigen-binding domains bind one or more antigens selected from the group consisting of: CEA, CEACAM1, CEACAM5, and CEACAM6.
28. An isolated cell comprising:
   (a) an inhibitory chimeric receptor comprising an extracellular antigen-binding domain that binds to an antigen, wherein the antigen is selected from the group consisting of: VSIG2, CPM, ITM2C, SLC26A2, SLC4A4, GPA33, PLA2G2A, ABCA8, ATP1A2, CHP2, and SLC26A3; and
   (b) a chimeric receptor comprising one or more extracellular antigen-binding domains, wherein the one or more extracellular antigen-binding domains bind one or more additional antigens selected from the group consisting of: CEA, CEACAM1, CEACAM5, and CEACAM6.
29. The isolated cell of embodiment 27 or embodiment 28, wherein the chimeric receptor is a chimeric T cell receptor or a chimeric antigen receptor (CAR).
30. The isolated cell of embodiment 29, wherein the chimeric receptor is a CAR.
31. The isolated cell of embodiment 30, wherein the CAR comprises one or more intracellular signaling domains, and the one or more intracellular signaling domains are selected from the group consisting of a CD3zeta-chain intracellular signaling domain, a CD3epsilon-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, an NKp46 intracellular signaling domain, an NKp30 intracellular signaling domain, an NKp44 intracellular signaling domain, an NKG2D intracellular signaling domain, a CD226 intracellular signaling domain, and a CD160 intracellular signaling domain.
32. The isolated cell of embodiment 30 or embodiment 31, wherein the CAR comprises a transmembrane domain, and the transmembrane domain is selected from the group consisting of a CD8 transmembrane domain, a CD28 transmembrane domain, a CD25 transmembrane domain, a CD7 transmembrane domain, a CD3zeta-chain transmembrane domain, a CD4 transmembrane domain, a 4-1BB transmembrane domain, an OX40 transmembrane domain, an ICOS transmembrane domain, a CTLA-4 transmembrane domain, a LAX transmembrane domain, a LAT transmembrane domain, a PD-1 transmembrane domain, a LAG-3 transmembrane domain, a TIM3 transmembrane domain, a KIR3DS1 transmembrane domain, a KIR3DL1 transmembrane domain, an NKG2D transmembrane domain, an NKG2A transmembrane domain, a TIGIT transmembrane domain, a 2B4 transmembrane domain, and a BTLA transmembrane domain.
33. The isolated cell of any one of embodiments 30-32, wherein the CAR comprises a spacer region between the antigen-binding domain and the transmembrane domain, and the spacer region has an amino acid sequence selected from the group consisting of SEQ ID NOs: 49-58.
34. The isolated cell of any one of embodiments 27-33, wherein the antigen-binding domain of the inhibitory chimeric receptor and/or the chimeric receptor comprises one or more antibodies, antigen-binding fragments of an antibody, F(ab) fragments, F(ab′) fragments, single chain variable fragments (scFvs), or single-domain antibodies (sdAbs).
35. The isolated cell of any one of embodiments 27-33, wherein the antigen-binding domain of the inhibitory chimeric receptor and/or the chimeric receptor comprises one or more single chain variable fragments (scFvs).
36. The isolated cell of embodiment 35, wherein each of the one or more scFvs comprises a heavy chain variable domain (VH) and a light chain variable domain (VL).
37. The isolated cell of embodiment 36, wherein the VH and VL are separated by a peptide linker.
38. The isolated cell of embodiment 37, wherein the peptide linker comprises the amino acid sequence of SEQ ID NO: 39 or SEQ ID NO: 77.
39. The isolated cell of any one of embodiments 36-38, wherein each of the one or more scFvs comprises the structure VH-L-VL or VL-L-VH, wherein VH is the heavy chain variable domain, L is the peptide linker, and VL is the light chain variable domain.
40. The isolated cell of any one of embodiments 28-39, wherein the one or more additional antigen is CECAM1.
41. The isolated cell of embodiment 40, wherein the antigen-binding domain that binds to CEACAM1 comprises a heavy chain variable domain (VH) comprising the amino acid sequence of SEQ ID NO: 1 and a light chain variable domain (VL) comprising the amino acid sequence of SEQ ID NO: 2.
42. The isolated cell of any one of embodiments 28-39, wherein the one or more additional antigen is CECAM5.
43. The isolated cell of embodiment 42, wherein the antigen-binding domain that binds to CEACAM5 comprises a heavy chain variable domain (VH) and a light chain variable domain (VL) selected from the group consisting of:
   (a) a VH comprising the amino acid sequence of SEQ ID NO: 3 and a VL comprising the amino acid sequence of SEQ. ID NO: 4;
   (b) a VH comprising the amino acid sequence of SEQ ID NO: 9 and a VL comprising the amino acid sequence of SEQ. ID NO: 10;
   (c) a VH comprising the amino acid sequence of SEQ ID NO: 11 and a VL comprising the amino acid sequence of SEQ. ID NO: 12;
   (d) a VH comprising the amino acid sequence of SEQ ID NO: 13 and a VL comprising the amino acid sequence of SEQ. ID NO: 14;
   (e) a VH comprising the amino acid sequence of SEQ ID NO: 78 and a VL comprising the amino acid sequence of SEQ. ID NO: 79; and
   (f) a VH comprising the amino acid sequence of SEQ ID NO: 15 and a VL comprising the amino acid sequence of SEQ. ID NO: 16.
44. The isolated cell of embodiment 42, wherein the antigen-binding domain that binds to CEACAM5 comprises a heavy chain (HC) and a light chain (LC) selected from the group consisting of:
   (a) an HC comprising the amino acid sequence of SEQ ID NO: 5 and an LC comprising the amino acid sequence of SEQ. ID NO: 6; and
   (b) an HC comprising the amino acid sequence of SEQ ID NO: 7 and an LC comprising the amino acid sequence of SEQ. ID NO: 8.
45. The isolated cell of any one of embodiments 28-39, wherein the one or more additional antigen is CECAM6.
46. The isolated cell of embodiment 45, wherein the antigen-binding domain that binds to CEACAM6 comprises a heavy chain variable domain (VH) comprising the amino acid sequence of SEQ ID NO: 17 and a light chain variable domain (VL) comprising the amino acid sequence of SEQ ID NO: 18.
47. The isolated cell of any one of embodiments 28-39, wherein the cell is an immunoresponsive cell.
48. The isolated cell of any one of embodiments 28-47, wherein binding of the inhibitory chimeric receptor to the antigen is capable of inhibiting the immunoresponsive cell.
49. The isolated cell of any one of embodiments 28-48, wherein binding of the chimeric receptor to the one or more additional antigens is capable of activating the immunoresponsive cell.
50. The isolated cell of any one of embodiments 28-49, wherein the chimeric receptor binds to the one or more additional antigens with a low binding affinity.
51. The isolated cell of any one of embodiments 28-50, wherein the chimeric receptor binds to the one or more additional antigens with a binding affinity that is lower than the binding affinity with which the inhibitory chimeric receptor binds to the antigen.
52. The isolated cell of any one of embodiments 28-51, wherein the inhibitory chimeric receptor binds to the antigen with a low binding avidity.
53. The isolated cell of any one of embodiments 27-52, wherein the chimeric receptor is recombinantly expressed.
54. The isolated cell of any one of embodiments 27-53, wherein the chimeric receptor is expressed from a vector or a selected locus from the genome of the cell.
55. The isolated cell of any one of embodiments 24-54, wherein the cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell.
56. The isolated cell of any one of embodiments 24-55, wherein the cell is autologous.
57. The isolated cell of any one of embodiments 24-55, wherein the cell is allogeneic.
58. An isolated nucleic acid encoding the inhibitory chimeric receptor of any one of embodiments 1-23.
59. A vector comprising the nucleic acid of embodiment 58.
60. A genetically modified cell comprising the nucleic acid of embodiment 58 or the vector of embodiment 59.
61. A method of treating a subject in need thereof, the method comprising administering the isolated cells of any one of embodiments 24-57.
62. A method of stimulating a cell-mediated immune response to a tumor cell in a subject, the method comprising administering to a subject having a tumor the isolated cells of any one of embodiments 24-57.
63. A method of providing an anti-tumor immunity in a subject, the method comprising administering to a subject in need thereof the isolated cells of any one of embodiments 24-57.
64. A method of reducing tumor burden in a subject, comprising administering to the subject the isolated cells of any one of embodiments 24-57.
65. The method of embodiment 64, wherein the method reduces the number of tumor cells.
66. The method of embodiment 64, wherein the method reduces tumor size.
67. The method of embodiment 64, wherein the method reduces tumor volume.
68. The method of embodiment 64, wherein the method eradicates the tumor in the subject.
69. A method of treating a subject having a tumor, the method comprising administering the isolated cells of any one of embodiments 24-57.
70. A method of treating or preventing a colorectal carcinoma in a subject, comprising administering to the subject the isolated cells of any one of embodiments 24-57.
71. A method of treating or preventing pancreatic cancer in a subject, comprising administering to the subject the isolated cells of any one of embodiments 24-57.
72. A method of treating or preventing a lung adenocarcinoma in a subject, comprising administering to the subject the isolated cells of any one of embodiments 24-57.
73. A method of treating or preventing gastric cancer in a subject, comprising administering to the subject the isolated cells of any one of embodiments 24-57.
74. The method of any one of embodiments 61-73, wherein the isolated cells are administered at an effective amount.
75. The method of any one of embodiments 69-74, wherein the method increases progression free survival in the subject.
76. The method of any one of embodiments 69-75, wherein the method increases survival in the subject.
77. A pharmaceutical composition comprising an effective amount of the isolated cell of any one of embodiments 24-57, and a pharmaceutically acceptable carrier, a pharmaceutically acceptable excipient, or a combination thereof.
78. The pharmaceutical composition of embodiment 77, wherein the pharmaceutical composition is for treating and/or preventing a colorectal carcinoma, a pancreatic cancer, a lung adenocarcinoma, and/or a gastric cancer.
79. A kit for treating and/or preventing a colorectal carcinoma, a pancreatic cancer, a lung adenocarcinoma, and/or a gastric cancer, comprising the isolated cell of any one of embodiments 24-57, or the pharmaceutical composition of embodiment 77.
80. The kit of embodiment 79, wherein the kit further comprises written instructions for using the isolated cell for treating and/or preventing a colorectal carcinoma, a pancreatic cancer, a lung adenocarcinoma, and/or a gastric cancer in a subject.
81. A kit for treating and/or preventing a colorectal carcinoma, a pancreatic cancer, a lung adenocarcinoma, and/or a gastric cancer, comprising the isolated nucleic acid of embodiment 58 or the vector of embodiment 59.
82. The kit of embodiment 81, wherein the kit further comprises written instructions for using the nucleic acid for producing one or more antigen-specific cells for treating and/or preventing a colorectal carcinoma, a pancreatic cancer, a lung adenocarcinoma, and/or a gastric cancer in a subject.

### EXAMPLES

The following are examples of methods and compositions of the present disclosure. It is understood that various other embodiments may be practiced, given the general description provided herein.

Below are examples of specific embodiments for carrying out the claimed subject matter of the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

### Example 1: Bioinformatics Screening for Solid Tumor Antigens

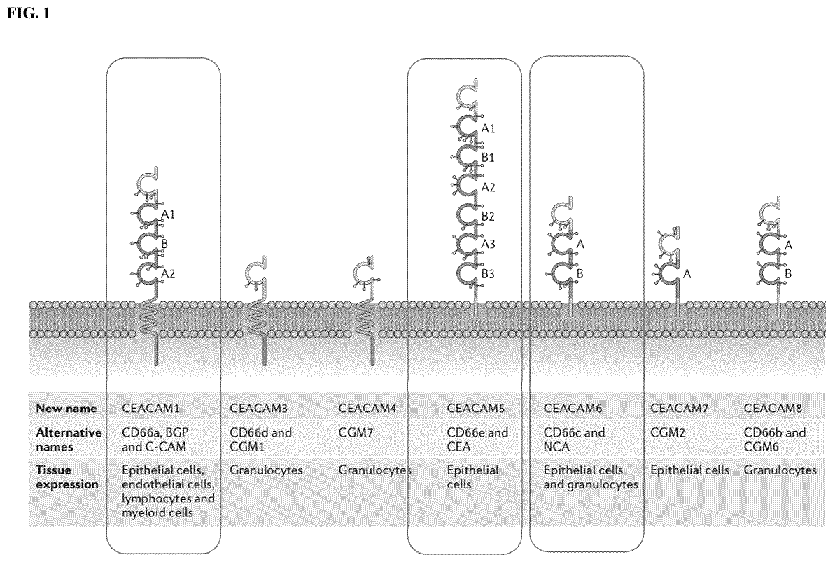

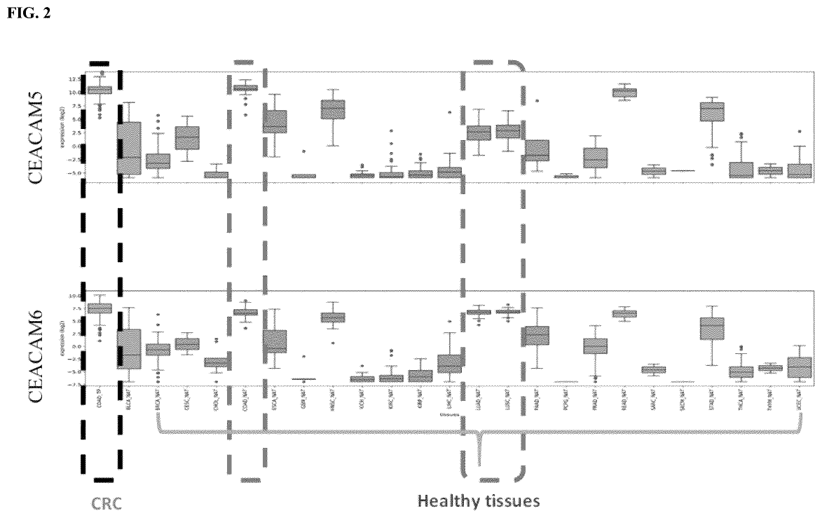

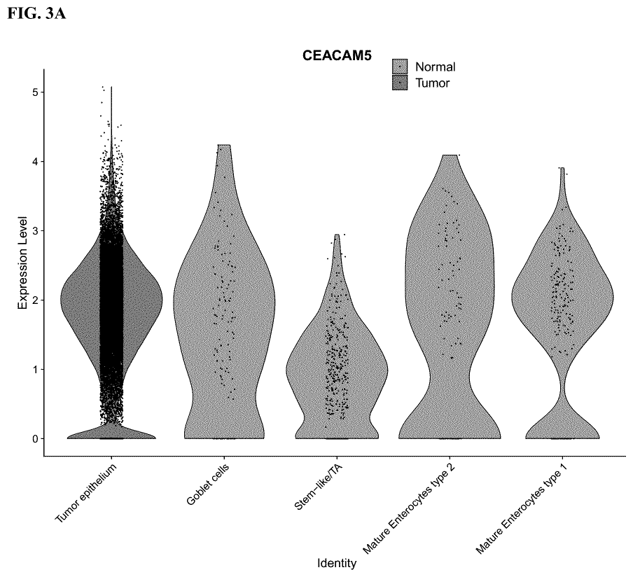

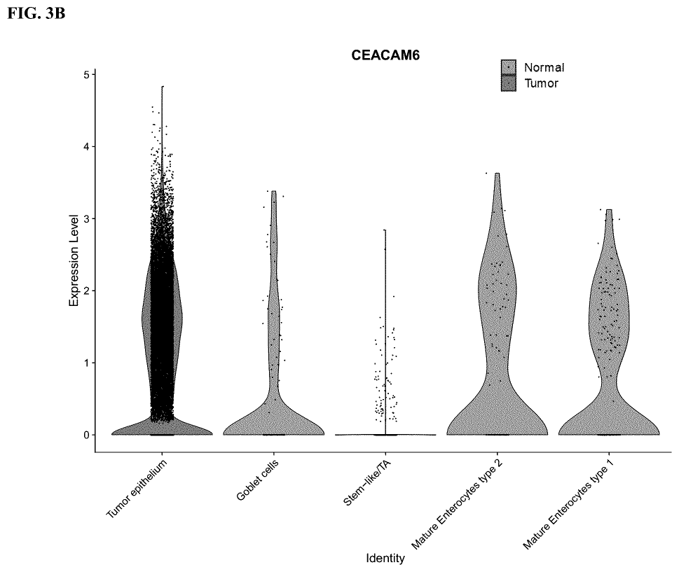

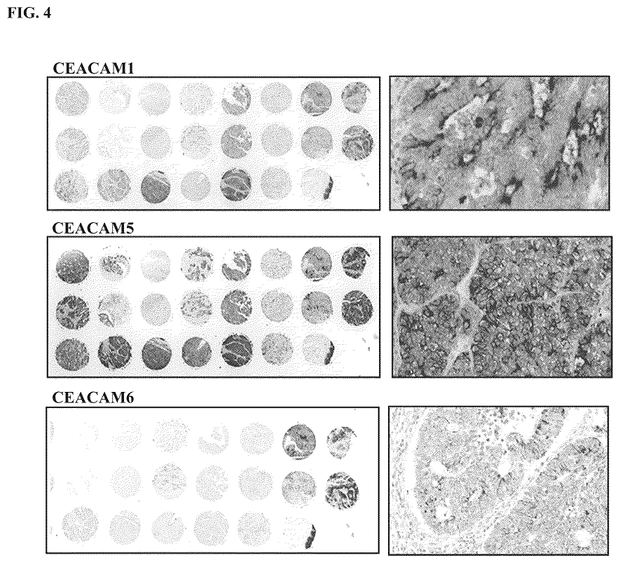

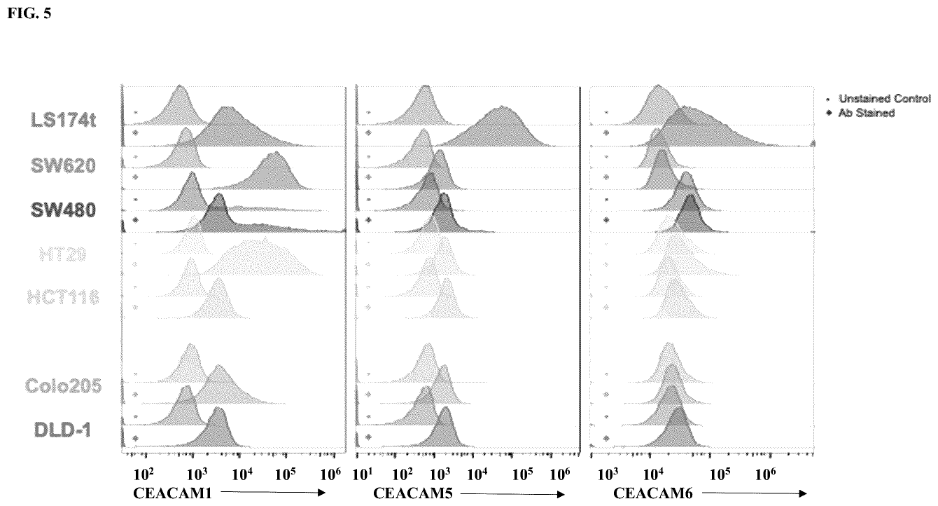

CEA was selected for further study due to its established role as a tumor antigen, coupled with its limited therapeutic efficacy due to on-target, off-tissue toxicities. Microarray data, RNA-seq data and proteomics data from selected solid tumors and normal tissue samples as well as data from off-target tissues were analyzed for expression levels of CEACAM1, CEACAM5, and CEACAM6. A comparative analysis of CEA-family members is depicted in FIG. 1. Analysis of data from the Cancer Genome Atlas (TCGA) demonstrated that CEACAM5 and CEACAM6 expression was found to be higher in both colorectal carcinoma (CRC) and normal tissues (FIG. 2). The analysis allowed for comparison across different cohorts. The y-axis in log 2 scale allowed for determination of relative differences in expression between different tumors and normal tissue. These findings were confirmed by bioinformatics analysis of patients using the RNA dataset provided of single cell RNAseq for colon cancer and healthy colon published by in Lee et al. (2020) Nat Genetics, 52(1):56-73, with expression of CEACAM5 shown in FIG. 3A and expression of CEACAM6 shown in FIG. 3B, revealing comparable expression between the cancerous and normal cells. Protein analysis in normal and cancer tissue was assessed by immunohistochemistry staining of tissue microarrays, and expression of the CEA-family proteins was confirmed at the protein level (FIG. 4). While expression was also found to be high in normal tissue, the high levels of CEACAM-family members across solid tissue types highlighted that CEACAM1, CEACAM5, and CEACAM6 were suitable targets for solid tumor therapies. For further system validation, a panel of human colon cancer cell lines were purchased from ATCC, and expression of different CEACAM isoforms (CEA family members: CEACAM1, 5 and 6) was determined using flow cytometry (FIG. 5).

### Example 2: Bioinformatics Screen for Inhibitory Antigen Targets (“NOT” Targets)

CEACAM1, CEACAM5, and CEACAM6 antigens identified in Example 1 were then paired for NOT gating. NOT targets were determined using the scRNA sequencing data using the following criteria: a NOT target had lower expression in a solid tumor tissue and a higher expression in a desired tissue. Potential NOT antigens were identified by performing differential gene expression (DEG) analysis on scRNAseq data. Targets were initially filtered by Log-fold change (LFC)>1 healthy:tumor tissue. Targets were then filtered and only putative membrane or cell surface proteins were considered. These were then manually prioritized and vetted based on literature searches etc. Similarly, suitable NOT targets were confirmed by examining expression relative to the solid tumor antigens CEACAM1, CEACAM5, or CEACAM6. Selected NOT targets had low expression in tumor cells when the solid tumor antigen expression was high, and high expression in healthy epithelial cells.

“NOT” gating targets determined by this strategy include VSIG2, CPM, ITM2C, SLC26A2, SLC4A4, GPA33, PLA2G2A, ABCA8, and ATP1A2. “NOT” targets are also described in Table 9.

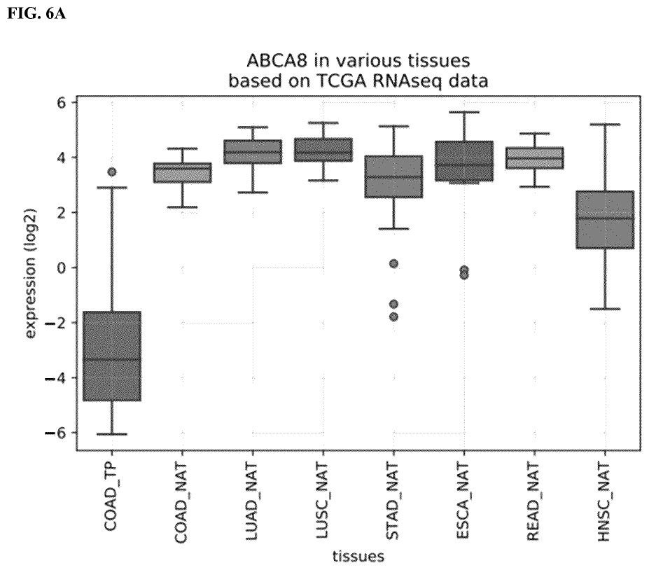

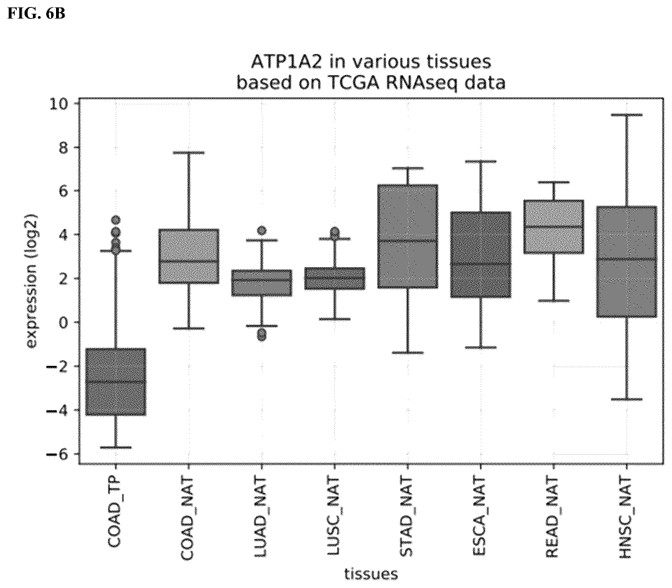

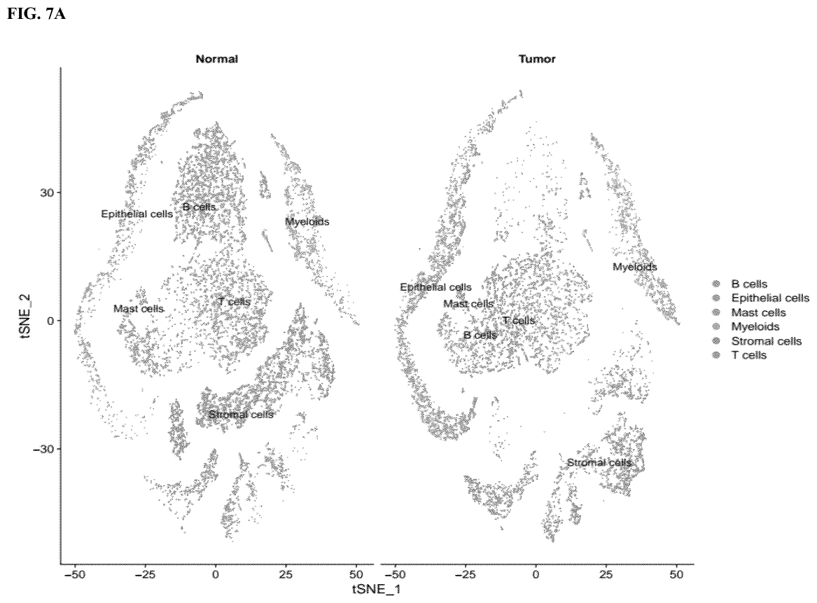

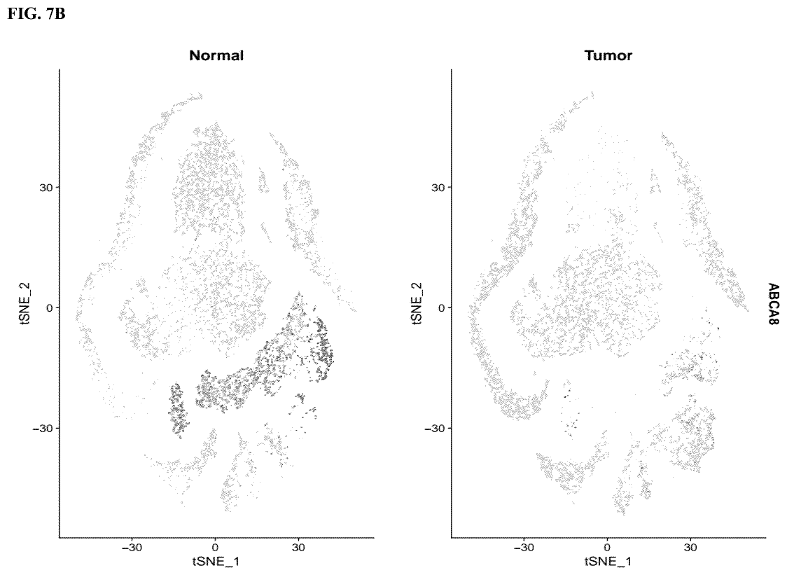

RNA analysis from the TCGA dataset is shown for ABCA8 (FIG. 6A) and ATP1A2 (FIG. 6B). Single-cell RNA sequencing (scRNAseq) results for ABCA8 are shown in FIG. 7A and FIG. 7B.

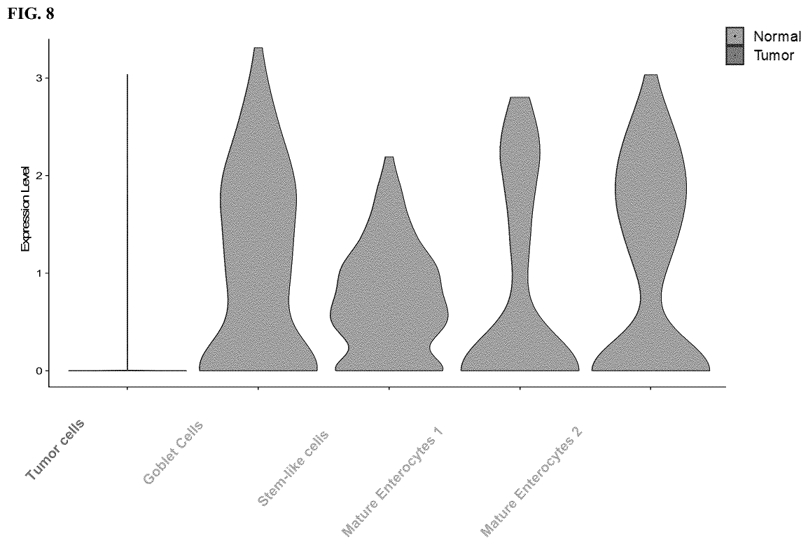

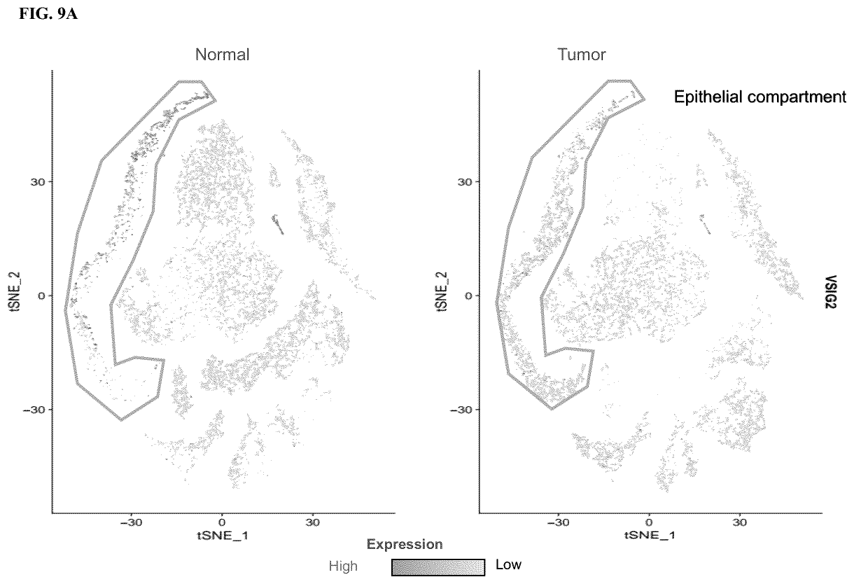

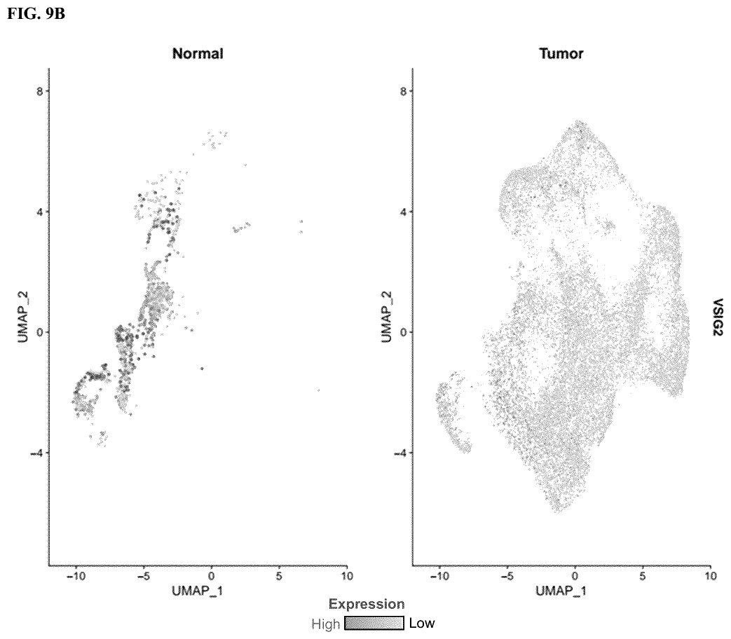

VSIG2 expression was analyzed across tumor cells and various normal using the following RNA datasets provided in Li et al. (2017) Nat. Genetics, 11(23):6861-6873 (GSE81861), Lee et al. (2020) Nat. Genetics, 52(1):56-73 (GSE132465), and GSE144735, with analysis as described in Example 1 above. As shown in FIG. 8, VSIG2 expression was not detected in tumor cells. In contrast, normal tissues showed detectable expression of VSIG2 in the four normal cell subsets examined demonstrating VSIG2 met the criteria as a NOT target. VSIG2 expression was further analyzed for specific expression in the epithelial compartment of the tissue, in an independent scRNAseq dataset. Principal component analysis (PCA) was used to identify specific subpopulations of cells scRNAseq expression of VSIG2 by overlaid. As shown in FIG. 9A-FIG. 9B, VSIG2 expression was higher in normal epithelial compartment subpopulations (FIG. 9A outlined region, left panel) and low in the epithelial compartment subpopulations of tumor cells (FIG. 9A outlined region, right panel) as well as low in tumor tissue overall, again demonstrating VSIG2 met the criteria as a NOT target.

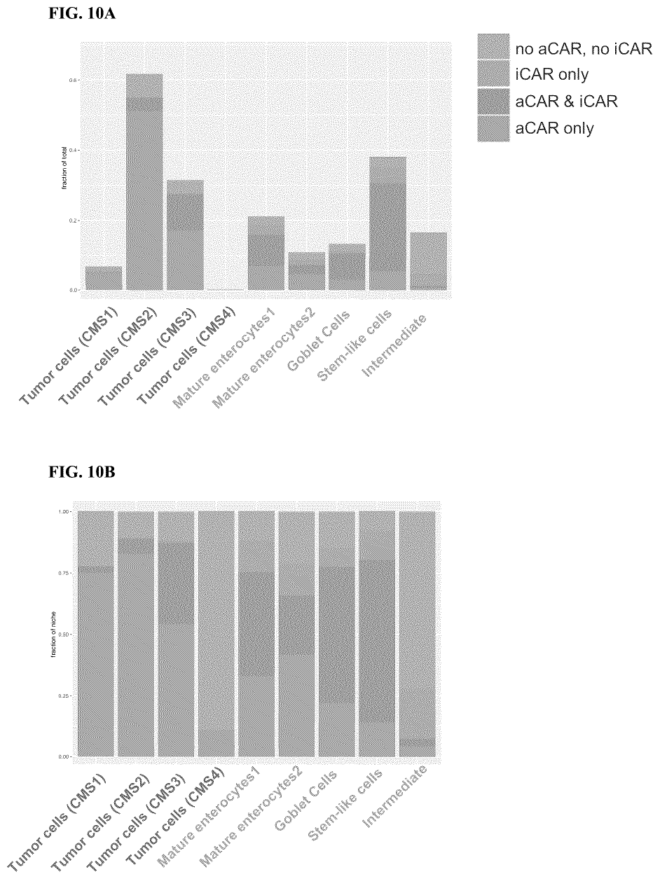

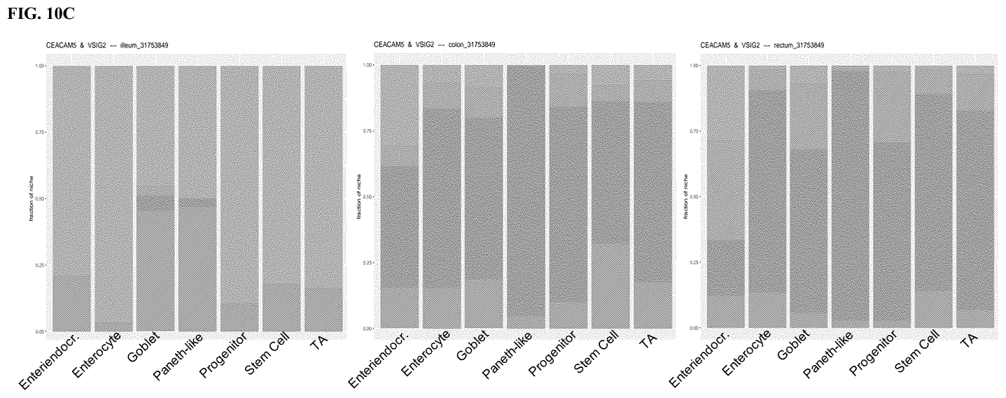

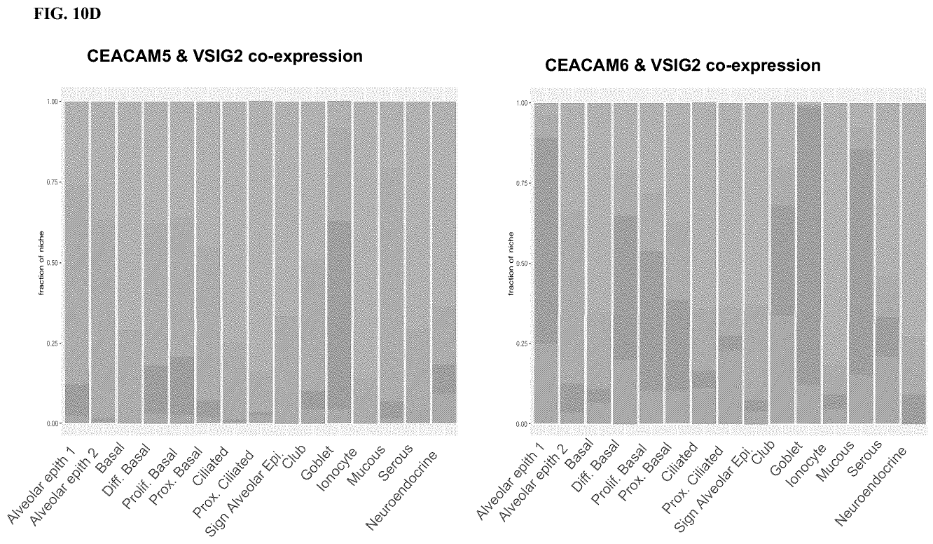

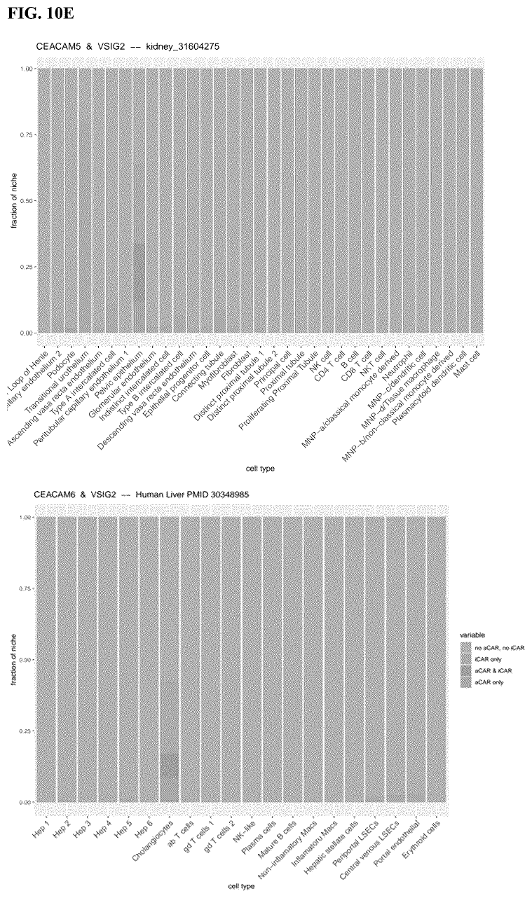

Comparative gene expression studies of VSIG2 and CEACAM5 was also examined across normal and tumor tissue. As shown in FIGS. 10A-10E, the analysis demonstrated an inverse correlation of expression across a range of tissue types. Similar expression patterns were observed for VSIG2 when compared with CEACAM6. scRNAseq analysis across a range of cell types and tissues was also performed for VSIG2, with analysis consistent with the results described above (FIG. 11). Consistent with the comparative expression analysis in FIGS. 10A-10E, VSIG2 was correlated with CEACAM5 and CEACAM6 expression in healthy lung tissue, as shown in FIG. 12A.

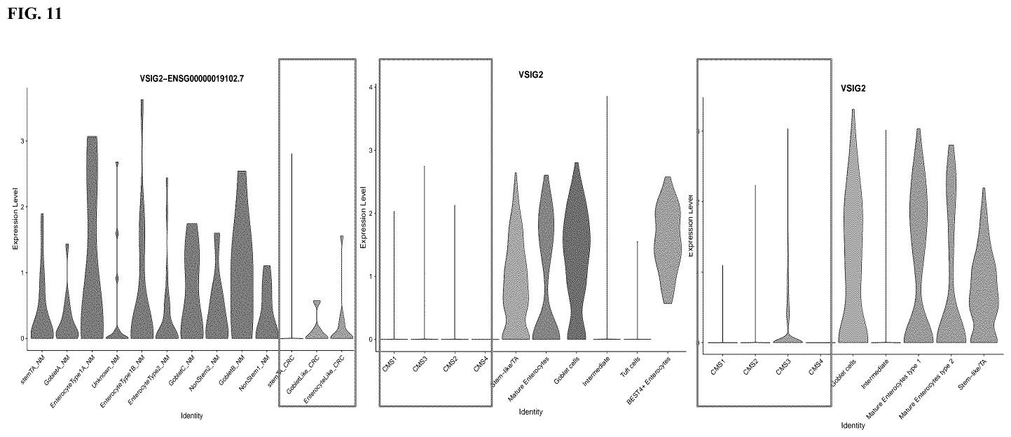

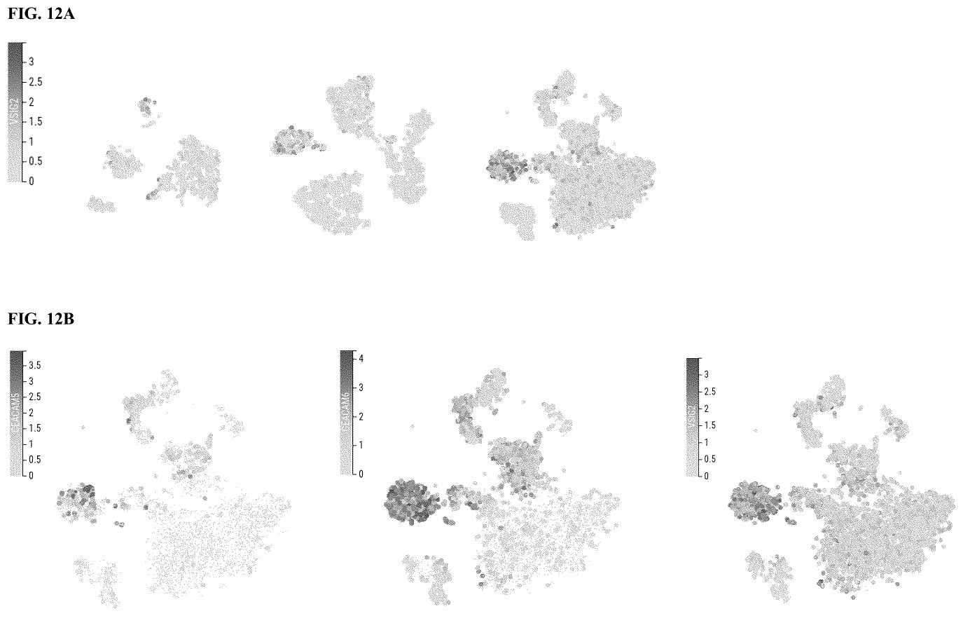

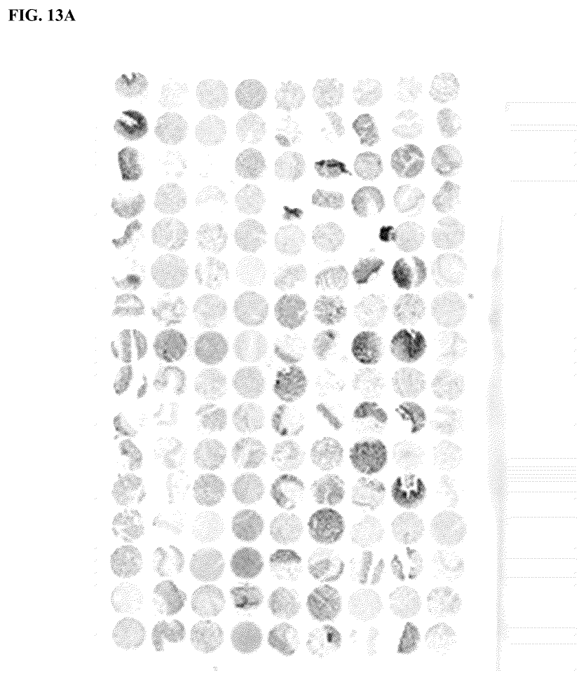

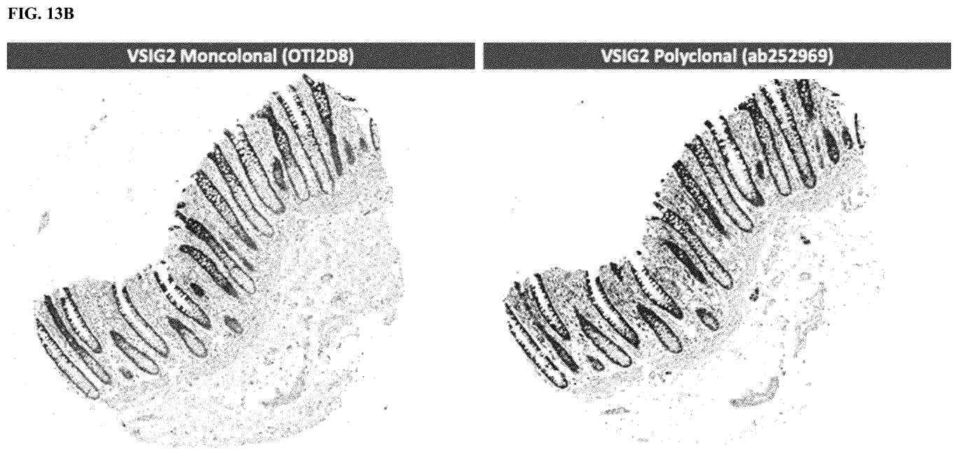

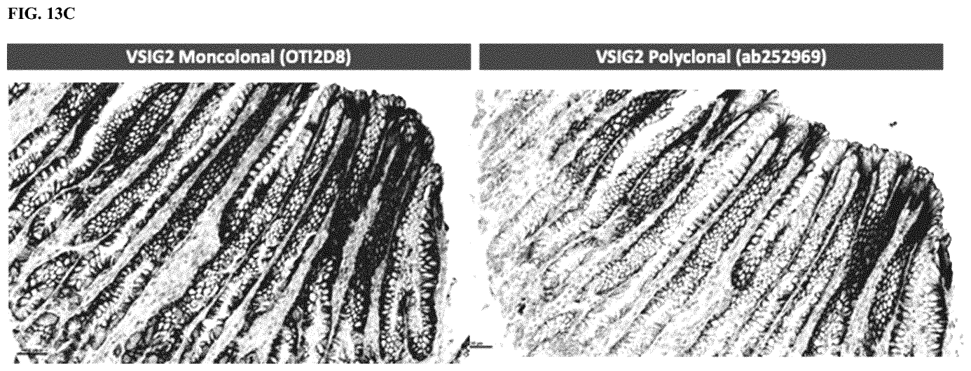

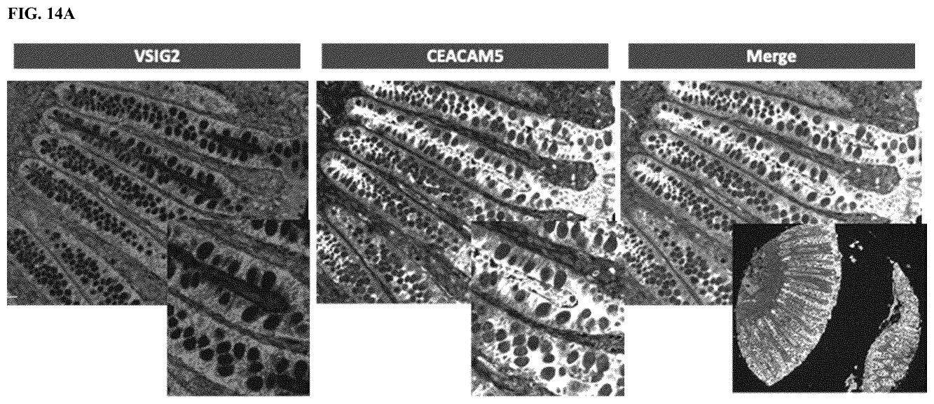

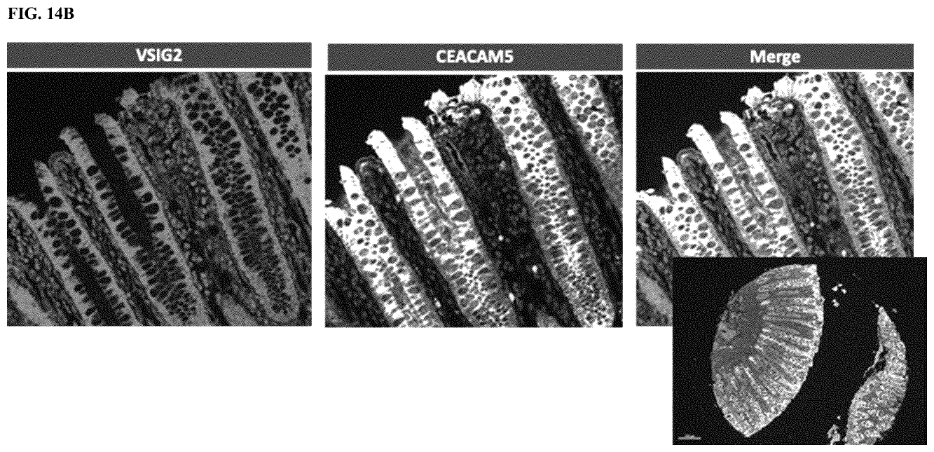

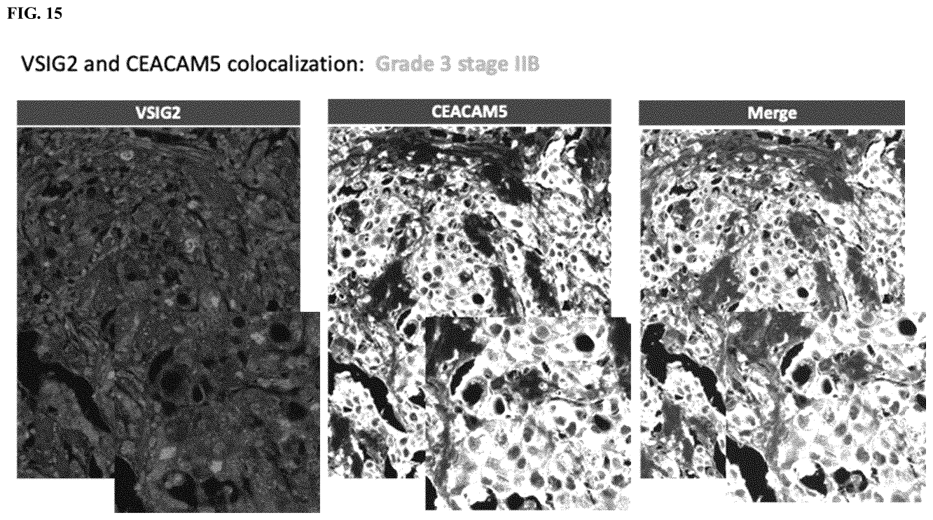

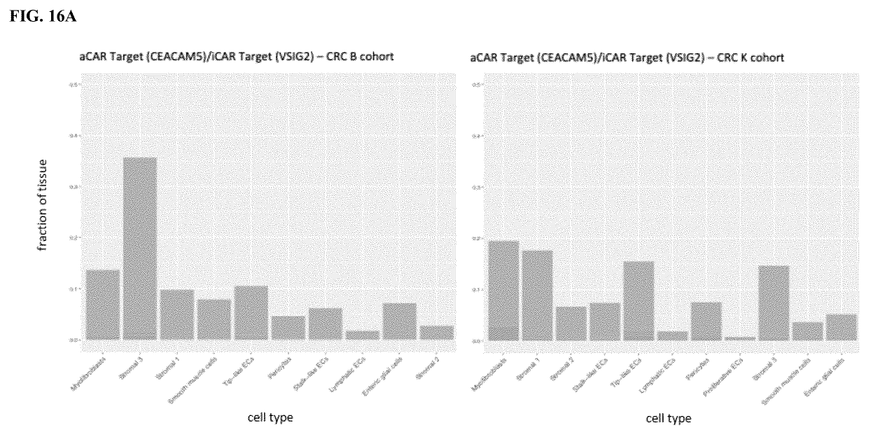

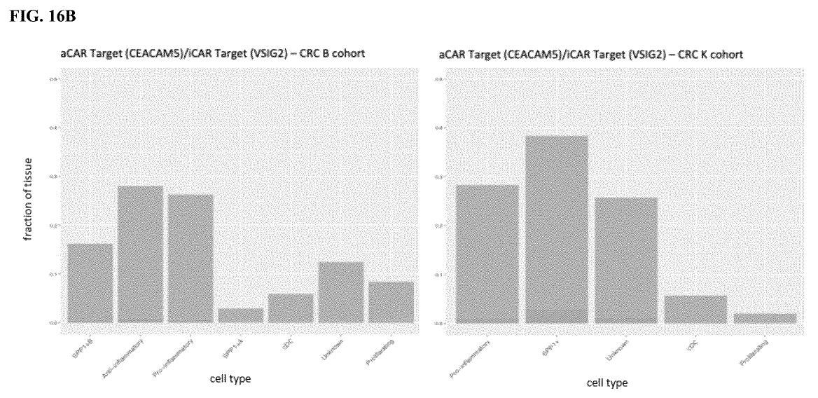

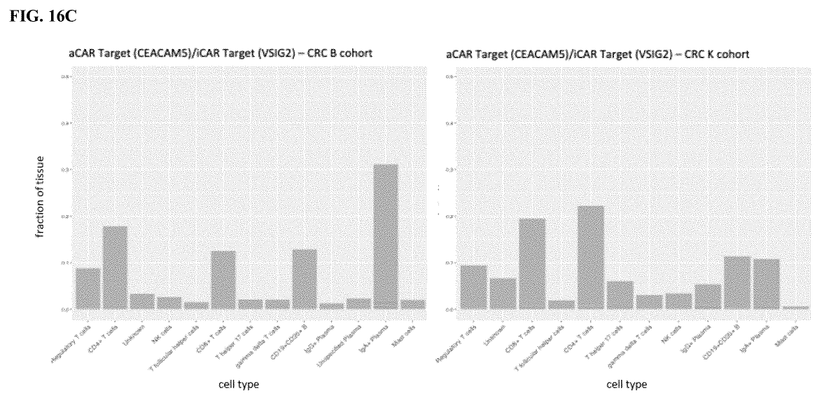

Putative VSIG2 protein expression was determined using tissue microarrays with multiple gastrointestinal tumor and healthy tissue samples (FIG. 13A). VSIG2 protein expression was determined by immunohistochemistry (IHC) using two different commercially available antibodies (monoclonal antibody OTI2D8 and polyclonal antibody ab252969). Representative images of healthy intestinal mucosa showing positive VSIG2 immunoreactivity in the epithelial cells (FIG. 13B-C). Co-localization of the aCAR Target CEACAM5 and iCAR Target VSIG2 in gastrointestinal healthy epithelial cells was observed using multiplexed IHC (FIG. 14A-14B). Critically, no co-localization of the aCAR Target CEACAM5 and iCAR Target VSIG2 in grade 3 stage IIB colon carcinoma sample was observed, with a majority of the VSIG2 signal observed to be lower or nuclear, and thus the iCAR NOT gate technology would not confer protection on the cancer tissue, but instead, would specifically protect the healthy tissue (FIG. 15). The aCAR target (CEACAM5) and iCAR target (VSIG2) co-expression in stromal compartment and immune cells in colon samples, determined by analyzing scRNAseq data from two datasets (FIG. 16A-16C).

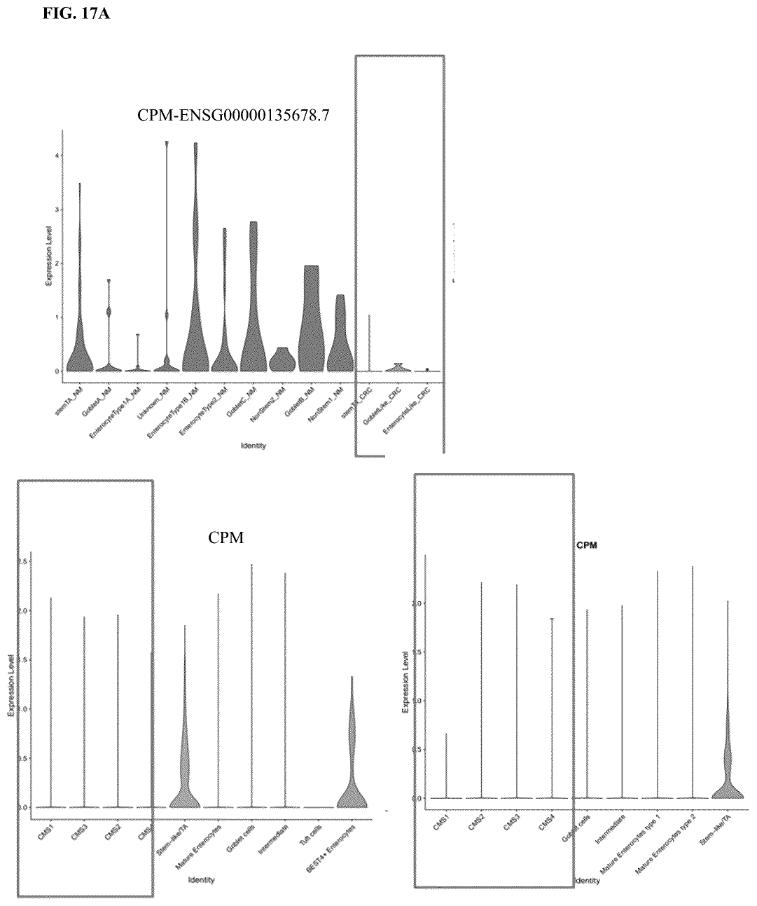

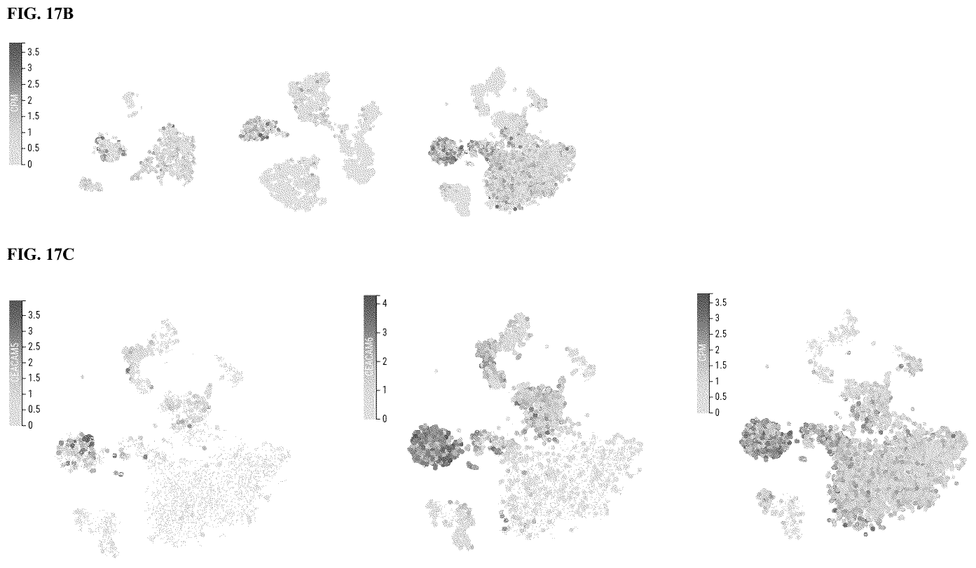

Another potential NOT target, CPM, was similarly analyzed for gene expression. Expression analysis was performed in colon cancer and normal tissue (FIG. 17A) and normal lung tissue (FIG. 17B). Comparative gene expression studies of CPM and CEACAM5 and CEACAM6 across normal and tumor tissue demonstrate an inverse correlation of expression, as shown in FIG. 17C.

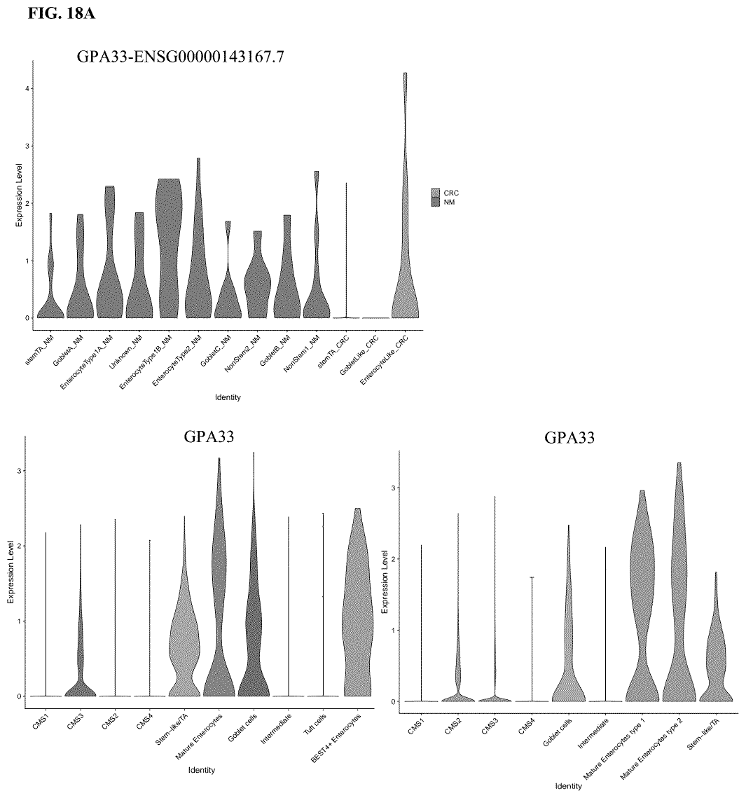

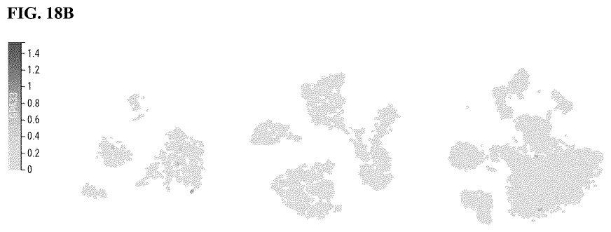

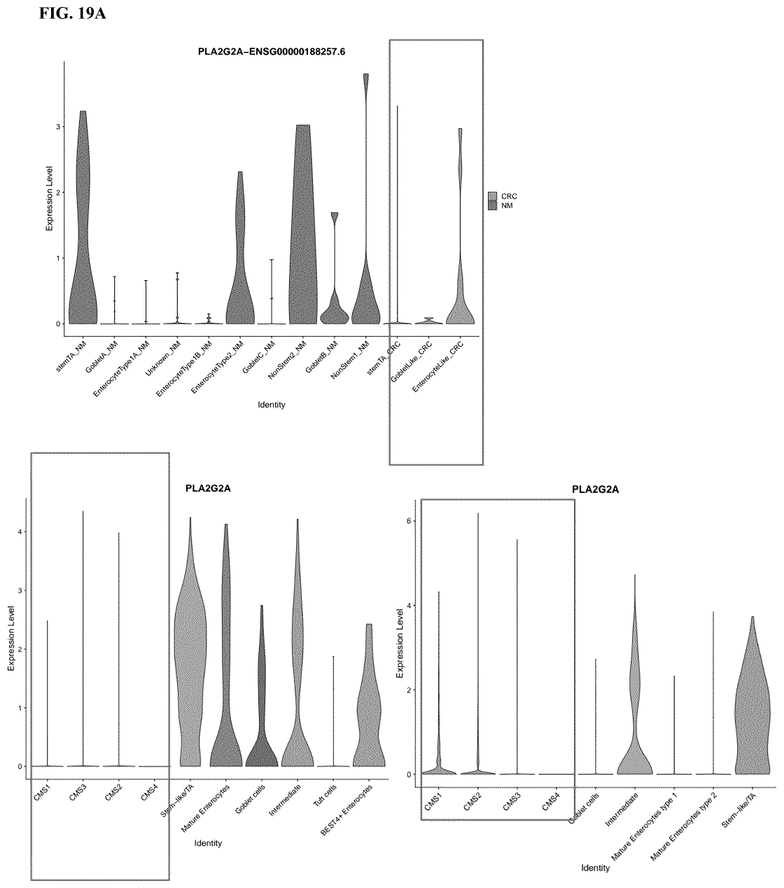

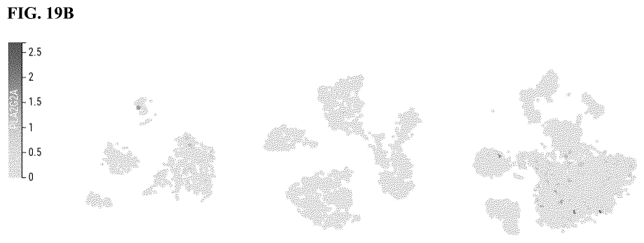

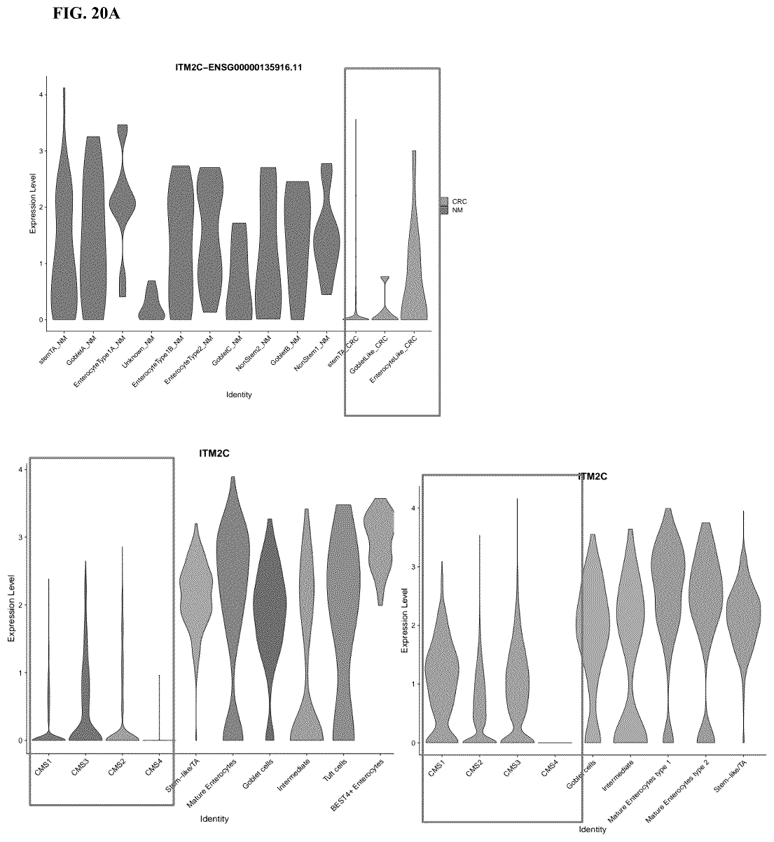

Putative gene expression analysis was also performed for NOT candidate antigens, to determine expression in normal and tumor tissues: GPA33 (FIG. 18A-FIG. 18B), PLA2G2A (FIG. 19A-FIG. 19B), ITM2C (FIG. 20A-FIG. 20B), CHP2 (FIG. 21A-FIG. 21B), SLC26A2 (FIG. 22A-FIG. 22B), SLC4A4 (FIG. 23A-FIG. 23B), and SLC26A3 (FIG. 24A-FIG. 24B).

### Example 3: CEA aCAR-Expressing T Cells Exhibit Increased Cytokine/Chemokine Induction and Target Cell Killing

T cells lentivirally transduced with constructs encoding CEA activating CARs (“aCARs”) were assessed for CAR expression, cytokine expression and target cell killing. Primary donor T cells from two distinct donors were transduced with CEA CAR constructs including a CD28 co-stimulatory domain, a CD3zeta intracellular domain and various anti-CEA scFvs (derived from hMN14, hMFE23, MG7 and MRG1) were tested. Each CAR construct encoded, in the direction from N-terminus to C-terminus, a CD8 signal sequence (SEQ ID NO:76), an scFv domain (i.e., “binder”), a Myc epitope tag (SEQ ID NO: 75), a CD8 hinge (SEQ ID NO: 50), a transmembrane domain derived from either CD28 (SEQ ID NO: 46) or OX40 (SEQ ID NO: 47), a co-stimulatory domain derived either from CD28 (SEQ ID NO: 71) or OX40 (SEQ ID NO: 72), and a CD3zeta activation domain (SEQ ID NO: 73). Descriptions of each binder, transmembrane domain, and co-stimulatory domain used in each construct are provided in Table 12. T cells were transduced with an MOI of 0.6 or 0.3 picograms (as measured by p24 assay) of lentivirus per T cell. Primary T cells were obtained from two distinct donors (referred to as “donor 1” and “donor 2”) and were used to assess construct transduction efficiency. CAR expression was assessed via flow cytometry by staining for the presence of an epitope tag (Myc). Cells were stained at 4 and 7 days post-transduction to determine stability of construct expression. Gates to determine % Myc expression were drawn using untransduced controls (No Virus) so that the <1% of Myc+ cells were in the positive gate. Median fluorescence intensity (MFI) was determined to quantify and assess levels of CAR expression. Expression levels in donor 1 and donor 2 T cells on day 4 for each CAR are shown in Table 13. Expression levels in donor 1 and donor 2 T cells on day 8 for each CAR are shown in Table 14.

TABLE 12

| | Binder (scFv), | TM & Co- |
|---|---|---|
| Construct ID | Orientation, & SEQ ID NO | Stim Domain |
| SB02463 | hMN14 | CD28 |
| | VL-VH | |
| | (SEQ ID NO: 19) | |
| SB02465 | hMFE23 | |
| | VH-VL | |
| | (SEQ ID NO: 20) | |
| SB02467 | MG7 | |
| | VL-VH | |
| | (SEQ ID NO: 21) | |
| SB02470 | MRG1 | |
| | VH-VL | |
| | (SEQ ID NO: 22) | |
| SB02779 | hMN14 | OX40 |
| | L/H | |
| | (SEQ ID NO: 19) | |
| SB02782 | hMFE23 | |
| | VH-VL | |
| | (SEQ ID NO: 20) | |
| SB02785 | MG7 | |
| | L/H | |
| | (SEQ ID NO: 21) | |
| SB02788 | MRG1 | |
| | H/L | |
| | (SEQ ID NO: 22) | |

TABLE 13

| | | Day 4 | | |
|---|---|---|---|---|
| | | | MFI of Myc+ | |
| Construct ID | MOI (pg) | % Myc+ | Cells | |
| SB02463 | 0.6 | 67.1 | 39083 | Donor 1 |
| SB02465 | 0.6 | 93.2 | 56281 | |
| SB02467 | 0.6 | 71.1 | 14712 | |
| SB02470 | 0.6 | 77.8 | 28766 | |
| SB02476 | 0.6 | 71.2 | 5370 | |
| No Virus | 0 | 0.47 | 2680 | |
| SB02463 | 0.3 | 60.1 | 36351 | |
| SB02465 | 0.3 | 91 | 47832 | |
| SB02467 | 0.3 | 67.8 | 12812 | |
| SB02470 | 0.3 | 73.2 | 26101 | |
| SB02476 | 0.3 | 64 | 4950 | |
| SB02463 | 0.6 | 58.9 | 40008 | Donor 2 |
| SB02465 | 0.6 | 89.9 | 48857 | |
| SB02467 | 0.6 | 62 | 13511 | |
| SB02470 | 0.6 | 77.9 | 26231 | |
| SB02476 | 0.6 | 70.1 | 5392 | |
| No Virus | 0 | 0.89 | 2329 | |
| SB02463 | 0.3 | 53.4 | 37398 | |
| SB02465 | 0.3 | 89.9 | 49248 | |
| SB02467 | 0.3 | 72.1 | 17351 | |
| SB02470 | 0.3 | 74.2 | 25653 | |
| SB02476 | 0.3 | 63.1 | 4910 | |

TABLE 14

Day 8

| | | | MFI of Myc+ | |
|---|---|---|---|---|
| Construct ID | MOI | % Myc+ | Cells | |
| SB02463 | 0.6 | 65 | 40931 | Donor 1 |
| SB02465 | 0.6 | 93.3 | 37665 | |
| SB02467 | 0.6 | 81.8 | 23874 | |
| SB02470 | 0.6 | 74.5 | 31633 | |
| SB02476 | 0.6 | 87.2 | 6797 | |
| No Virus | 0 | 0.21 | 2557 | |
| SB02463 | 0.3 | 58.3 | 40180 | |
| SB02465 | 0.3 | 91.1 | 33318 | |
| SB02467 | 0.3 | 78.9 | 22621 | |
| SB02470 | 0.3 | 71 | 29855 | |
| SB02476 | 0.3 | 83.1 | 6449 | |
| SB02463 | 0.6 | 61.9 | 71269 | Donor 2 |
| SB02465 | 0.6 | 94.1 | 68204 | |
| SB02467 | 0.6 | 72.1 | 41184 | |
| SB02470 | 0.6 | 81.7 | 65274 | |
| SB02476 | 0.6 | 93.7 | 12608 | |
| No Virus | 0 | 0.34 | 2389 | |
| SB02463 | 0.3 | 54.7 | 57595 | |
| SB02465 | 0.3 | 93.6 | 56880 | |
| SB02467 | 0.3 | 80.4 | 38961 | |
| SB02470 | 0.3 | 79.1 | 60171 | |
| SB02476 | 0.3 | 92.3 | 11386 | |

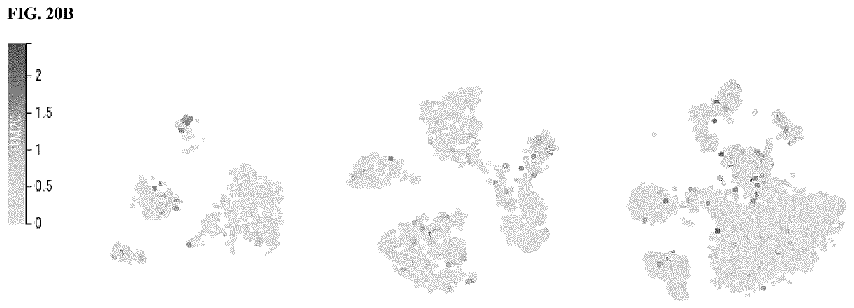

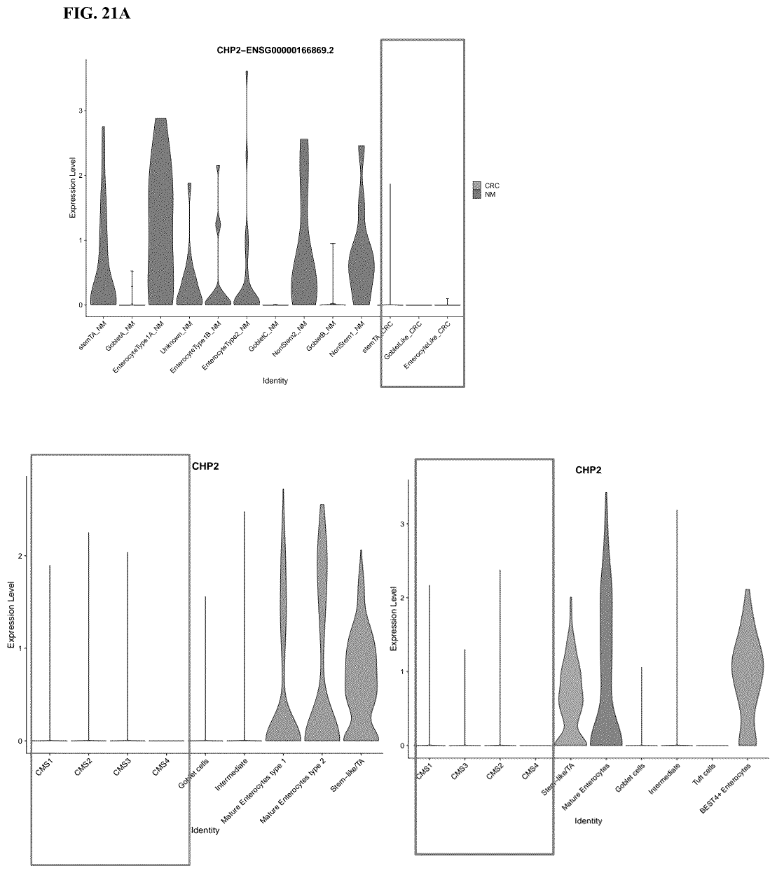

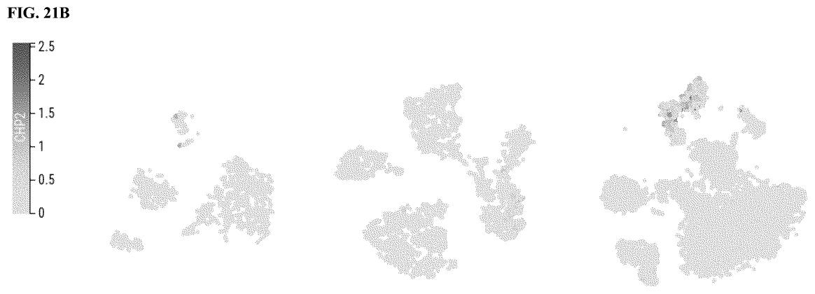

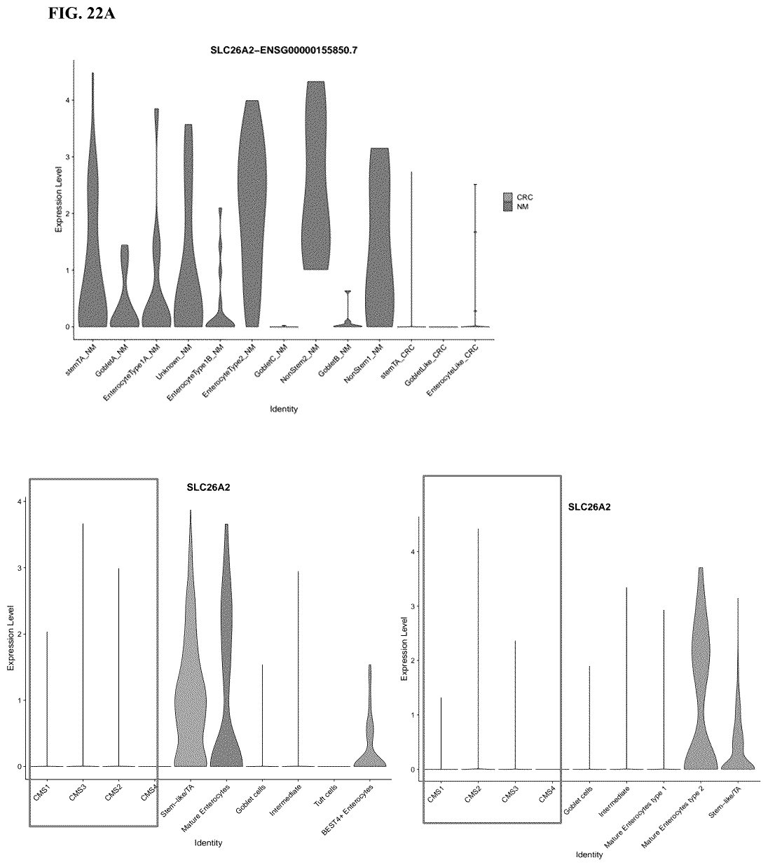

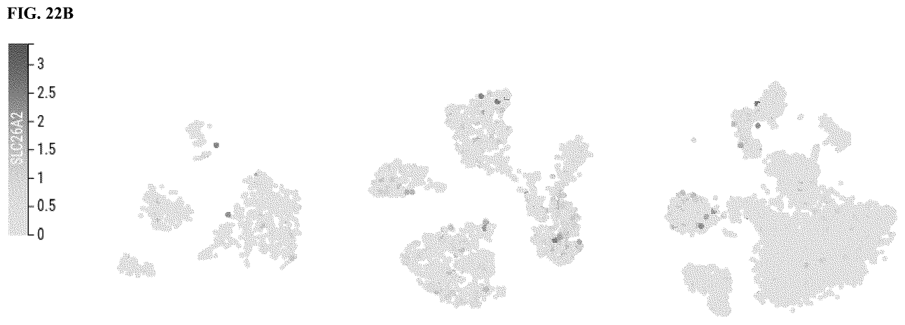

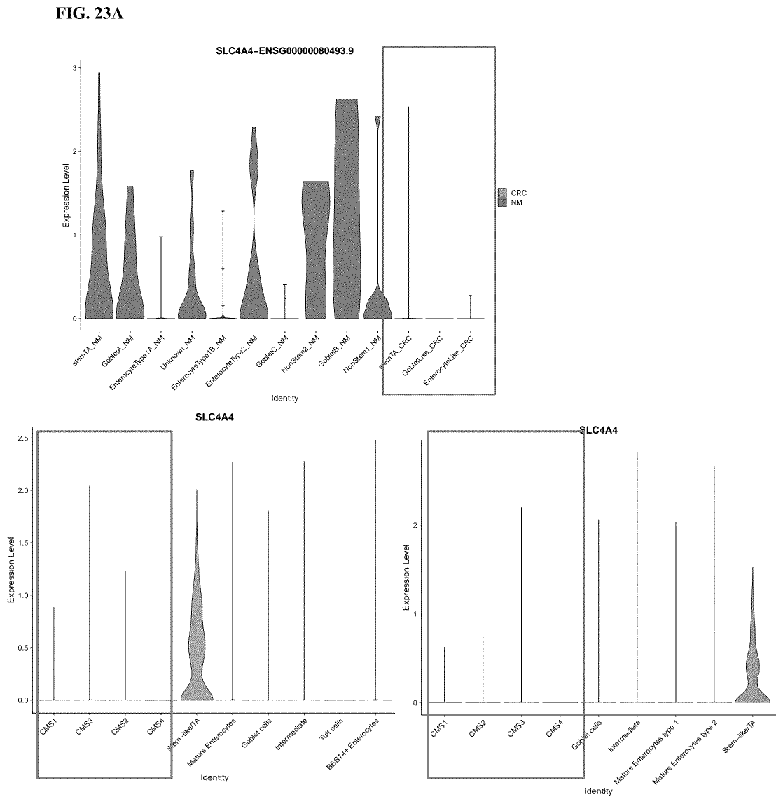

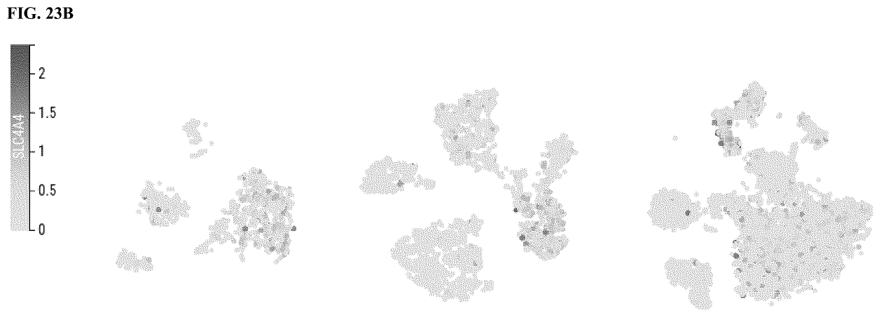

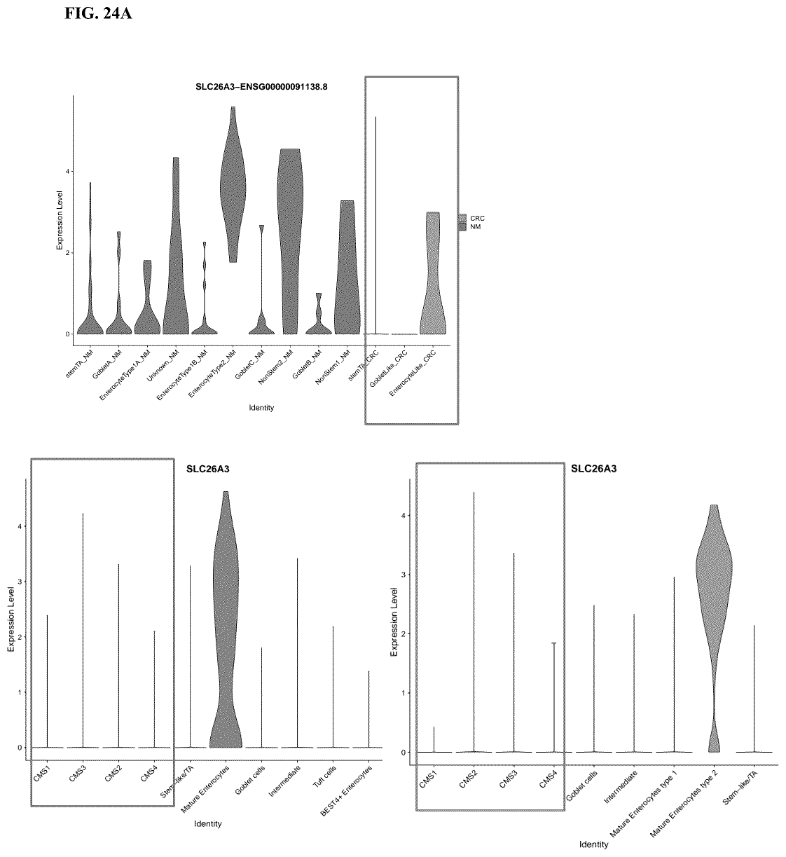

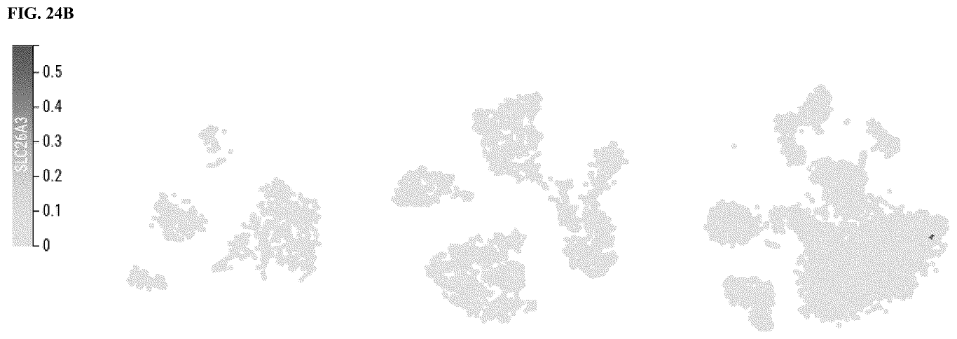

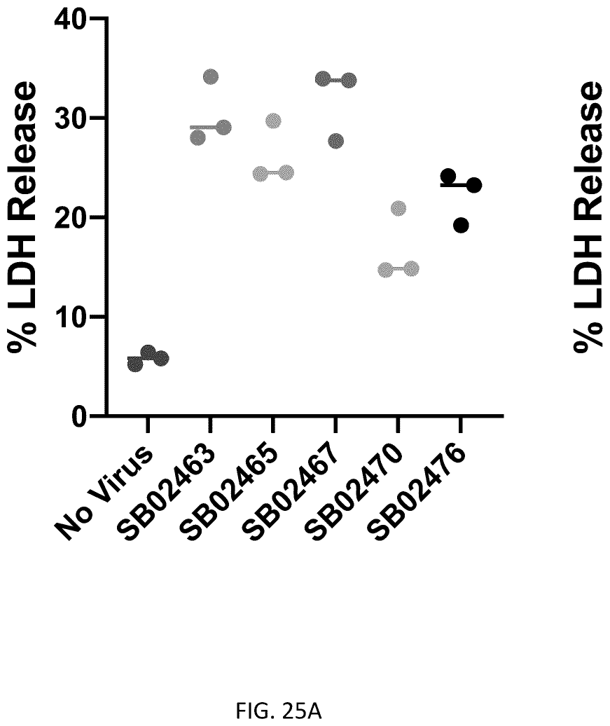

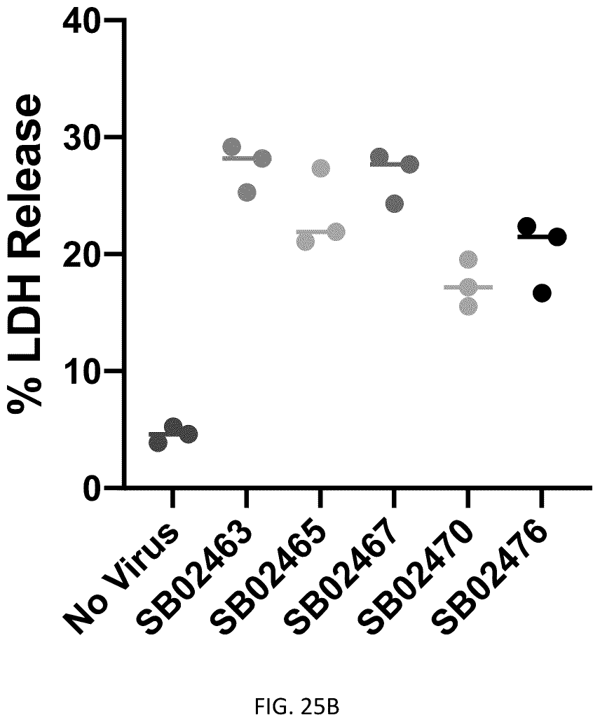

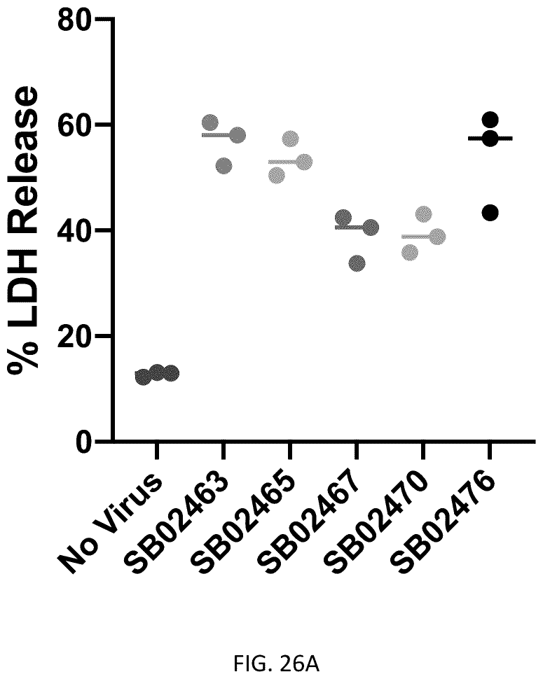

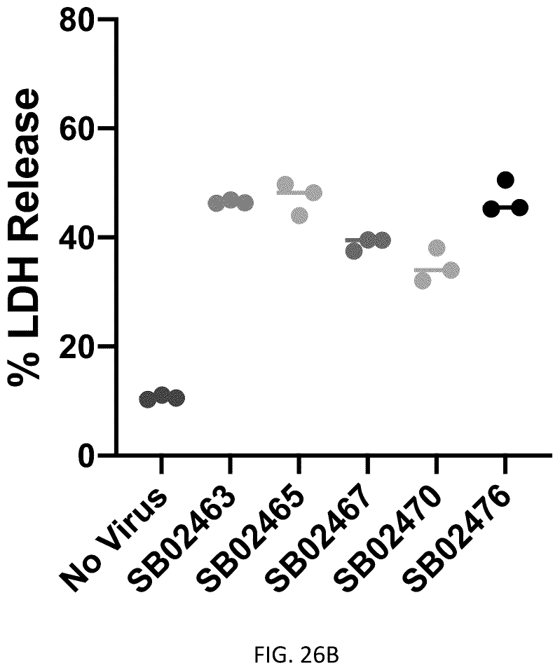

Killing activity of CEA aCAR-transduced T cells was assessed. CEA aCAR transduced T cells were incubated with target cells of a colon cancer cell line (LS174t) at an E:T ratio of 2:1 overnight (16-18 hrs). Ls174t target lines included “parental” LS174t cells (left panels) as well as “mKate” lines that had been transduced to express a fluorescent reporter, mKate (right panels). Two T Cell donors were used to assess potential CAR activity, donor 1 and donor 2. Cell culture supernatant was collected and tested for killing. To assess cell death due to killing activity, LDH levels were measured in the cell culture supernatant. Increased LDH release indicates killing of target cells. LDH activity was determined following manufacturer's instructions (CyQUANT LDH Cytotoxicity Assay, Thermo Fisher). Maximum killing activity was determined from chemically lysed target cells whereas spontaneous LDH release was determined from target cells alone. Percent LDH release (as a percentage of maximum LDH release) was calculated for parental target cells co-cultured with donor 1 T cells transduced with the constructs (FIG. 25A), mKate target cells co-cultured with donor 1 T cells transduced with the constructs (FIG. 25B), parental target cells co-cultured with donor 2 T cells transduced with the constructs (FIG. 26A), and mKate target cells co-cultured with donor 2 T transduced with the constructs (FIG. 26B). All CEA aCARs exhibited significant killing activity over untransduced control (No Virus) indicating CAR-mediated killing of cognate target cells. Killing activity was comparable across both target lines tested.

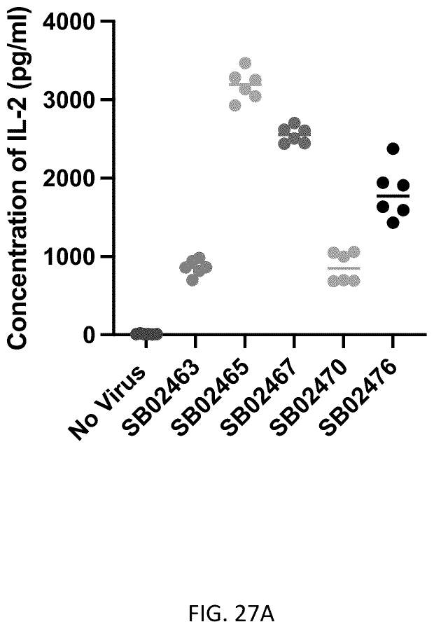

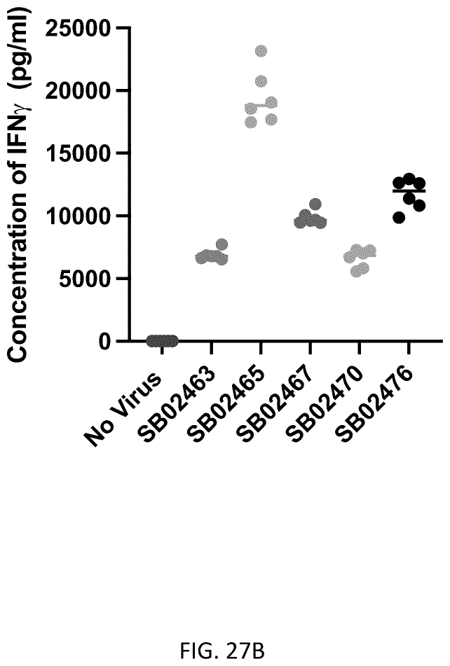

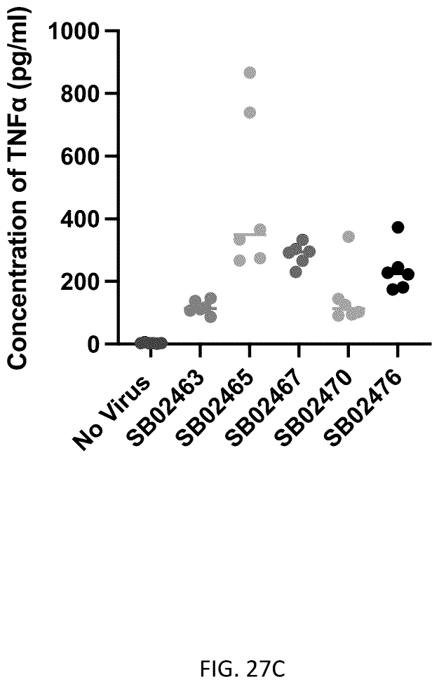

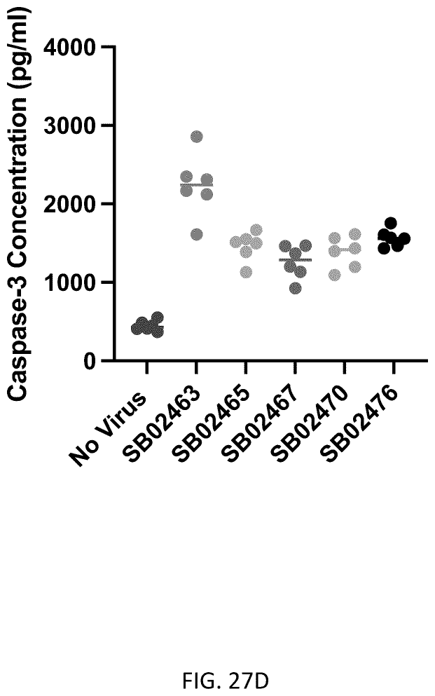

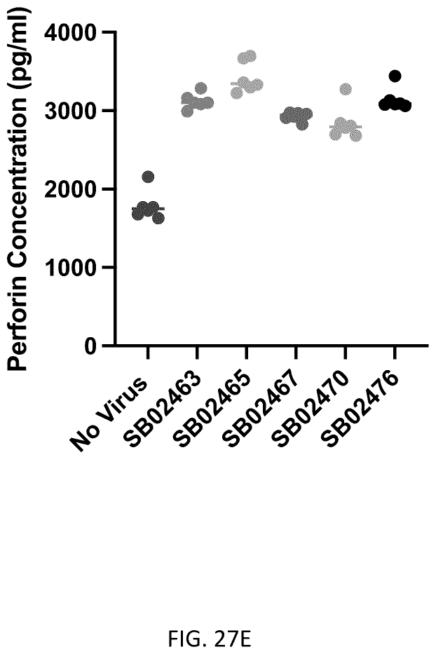

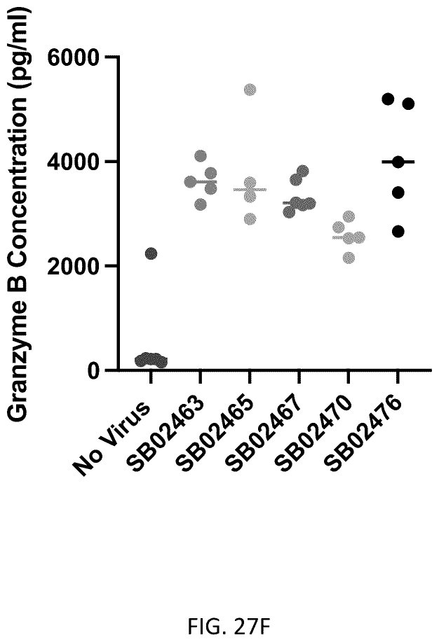

Cytokine activity of CEA aCAR-transduced T cells was assessed. Along with the killing assay as measured by LDH release and shown in FIGS. 25A, 25B, 26A, and 26B, the same cell culture supernatant was also assessed for T Cell cytokine activity. Cell culture supernatant collected from the killing assay conducted with Donor 2 was assessed using Luminex. Due to minimal differences across parental target cell versus mkate-expressing target cell lines in the LDH assay, the two target lines were assessed in the same group as technical replicates. A multiplex immunoassay (ProcartaPlex, Thermo Fisher) was used to measure levels of IL-2 (FIG. 27A), IFNγ (FIG. 27B), TNFα (FIG. 27C), Caspase-3 (FIG. 27D), Perforin (FIG. 27E), and Granzyme B (FIG. 27F) to further evaluate T cell activity. As shown in FIGS. 27A-27F, CEA aCAR-expressing T cells exhibited significantly higher cytokine and chemokine activity than untraduced control T cells (No Virus) indicating CAR-mediated activation of T cells following recognition of cognate target cells.

### Example 4: Lentivirus-Transduced CEA aCAR NK Cells Exhibit Increased Target Cell Killing

In this Example, primary donor-derived NK cells were transduced with lentiviral vectors encoding various CEA aCAR constructs and an off-target control construct expressing a CAR including an Axl binding domain, and expression of the CARs and CAR-mediated killing were assessed. Each CEA aCAR construct encoded, in the direction from N-terminus to C-terminus, a CD8 signal sequence (SEQ ID NO:76), an scFv domain (i.e., “binder”), a Myc epitope tag (SEQ ID NO: 75), a CD8 hinge (SEQ ID NO: 50), a transmembrane domain derived from either CD28 (SEQ ID NO: 46) or OX40 (SEQ ID NO: 46), a co-stimulatory domain derived either from CD28 (SEQ ID NO: 70) or OX40 (SEQ ID NO: 72), and a CD3zeta activation domain (SEQ ID NO: 74). CEA aCAR expression of transduced NK cells was assessed 5 days post-transduction. Constructs with various antigen binding domains, either a CD28 or OX40 co-stimulatory domains, and a CD3zeta intracellular domain were tested with various scFvs. The antigen binding domains and co-stimulatory domains of each construct are provided in Table 15. An off-target “control” CAR (SB02716) was also tested as a negative control. NK cells were transduced with an MOI of 30 IU per NK cell. CAR expression was assessed via flow cytometry by staining for the presence of an epitope tag (Myc). Cells were stained at 5 days post-transduction to determine stability of construct expression. Gates to determine % Myc expression were drawn using untransduced controls (No Virus) so that the <1% of Myc+ cells were in the positive gate. Median fluorescence intensity (MFI) was determined to quantify and assess levels of CAR expression. Expression levels on day 4 for each CAR are shown in Table 15.

TABLE 15

| | Binder (scFv), | TM & | | |
|---|---|---|---|---|
| Construct | Orientation, & | Co-Stim | | MFI of Myc+ |
| ID | SEQ ID NO | Domain | % Myc+ | Cells |
| SB02463 | hMN14 | CD28 | 21.2 | 2187 |
| | VL-VH | | | |
| | (SEQ ID NO: 19) | | | |
| SB02465 | hMFE23 | | 22.7 | 2414 |
| | VH-VL | | | |
| | (SEQ ID NO: 20) | | | |
| SB02467 | MG7 | | 4.56 | 1405 |
| | VL-VH | | | |
| | (SEQ ID NO: 21) | | | |
| SB02470 | MRG1 | | 20.9 | 1847 |
| | VH-VL | | | |
| | (SEQ ID NO: 22) | | | |
| SB02779 | hMN14 | OX40 | 18 | 1513 |
| | VL-VH | | | |
| | (SEQ ID NO: 19) | | | |
| SB02782 | hMFE23 | | 11.2 | 1388 |
| | VH-VL | | | |
| | (SEQ ID NO: 20) | | | |
| SB02785 | MG7 | | 1.5 | 1272 |
| | VL-VH | | | |
| | (SEQ ID NO: 21) | | | |
| SB02788 | MRG1 | | 14.2 | 1523 |
| | VH-VL | | | |
| | (SEQ ID NO: 22) | | | |
| SB02716 | aAxl (control) | 4-1BB/CD28 | 42.9 | 4311 |
| No Virus | N/A | | 0.88 | 1604 |

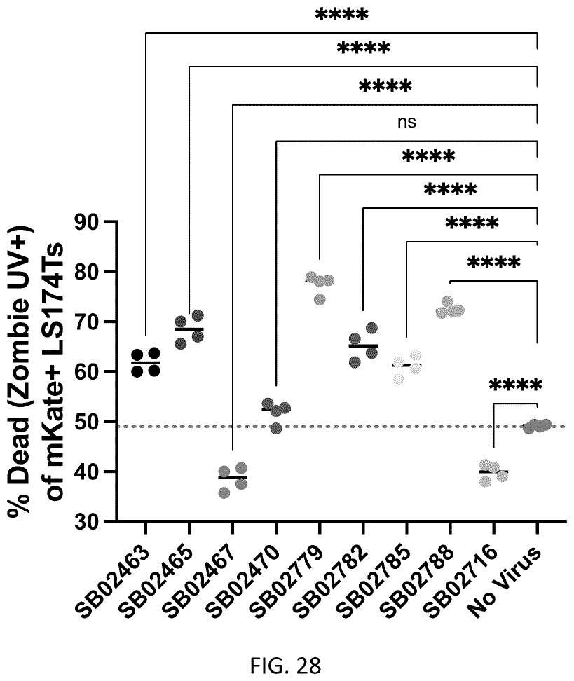

Killing activity of CEA aCAR lentivirally transduced NK cells in a flow-based Assay. Five days post-transduction, CEA aCAR transduced NK cells were incubated with colorectal cancer target line (LS174t) at an E:T ratio of 1:1 overnight (16-18 hrs). Cells were co-cultured in an ultra-low bind U-bottom plate to facilitate cell-cell interactions and mitigate plate adherence. Cells were harvested and stained for flow cytometry to determine cell phenotype and death. mKate expressing Ls174t target cells were used to distinguish target cells from CD56+NK cells. Zombie UV (Biolegend) dye was used to distinguish live from dead cells. Percentage of dead cells was calculated and shown in FIG. 28. As shown in FIG. 28, six of the CEA aCAR constructs tested exhibited significantly higher killing activity over the untransduced (No Virus) NK controls. Significant differences were determined using an ordinary one-way ANOVA with Dunnett's multiple comparison correction, **** p<0.0001. These results demonstrate significant CAR-mediated killing activity.

### Example 5: Expression and Killing Activity for Lentivirally & Gamma-Retrovirally Transduced NK Cells Expressing Various CEA aCAR Constructs

CEA aCAR expression of lentivirally and gammaretrovirally transduced NK cells was assessed. Lentivirus constructs with various hinges, transmembrane domains, and intracellular domain were tested with two CEACAM5-specific scFvs (A5B7 and Tus) either in a VH-VL orientation or a VL-VH orientation. Each CAR construct encoded, in the direction from N-terminus to C-terminus, a CD8 signal sequence (SEQ ID NO:76), an scFv domain (i.e., “binder”), a Myc epitope tag (SEQ ID NO: 75), a CD8 hinge (SEQ ID NO: 50) or a CD28 hinge (SEQ ID NO: 49), a transmembrane domain derived from either CD28 (SEQ ID NO: 46) or CD8 (SEQ ID NO: 45), a co-stimulatory domain derived either from CD28 (SEQ ID NO: 71) or 4-1BB (SEQ ID NO: 70), and a CD3zeta activation domain (SEQ ID NO: 74). In parallel, γ-retroviral constructs were tested with various scFvs (three CEACAM5-specific scFvs, hMN14, hMFE23, and Tusamitamab and one CEACAM1-specific scFv, MRG1), transmembrane domains, and costimulatory domains. Each gammaretrovirus construct also included a CD8 signal sequence (SEQ ID NO:76), a myc epitope tag (SEQ ID NO: 75), and a CD3zeta activation domain (SEQ ID NO: 74) C-terminal to the costimulatory domain. An off-target “control” CAR (SB02716) was also tested as a negative control. CAR expression was assessed via flow cytometry by staining for the presence of an epitope tag (Myc). Cells were stained at 4- and 7-days post-transduction to determine stability of construct expression. Gates to determine % Myc expression were drawn using untransduced controls (No Virus) so that the <1% of Myc+ cells were in the positive gate. Median fluorescence intensity (MFI) was determined to quantify and assess levels of CAR expression. CAR expression levels on day 4 for lentivirally transduced NK cells are shown in Table 16. CAR expression levels on day 4 for gammaretrovirally transduced NK cells are shown in Table 17. CAR expression levels on day 7 for lentivirally transduced NK cells are shown in Table 18. CAR expression levels on day 7 for gammaretrovirally transduced NK cells are shown in Table 19.

TABLE 16

| | Lentiviral Construct Components | | | | | | |
|---|---|---|---|---|---|---|---|
| | | VH-VL or VL-VH | | | | Day 4 | MFI of Myc+ |
| SB ID | scFv name | & SEQ ID NO | hinge | TM | co-stim | % Myc+ | Cells |
| SB03089 | Tus | VH-VL | CD8 | CD28 | CD28 | 67.5 | 2228 |
| | | (SEQ ID NO: 23) | | | | | |
| SB03090 | | VL-VH | | | | 51.2 | 1684 |
| | | (SEQ ID NO: 24) | | | | | |
| SB03091 | A5B7 | VH-VL | | | | 1.2 | 1064 |
| | | (SEQ ID NO: 25) | | | | | |
| SB03092 | | VL-VH | | | | 0.36 | 1184 |
| | | (SEQ ID NO: 26) | | | | | |
| SB03093 | Tus | VH-VL | CD28 | | | 34.6 | 1436 |
| | | (SEQ ID NO: 23) | | | | | |
| SB03094 | | VL-VH | | | | 12.6 | 1149 |
| | | (SEQ ID NO: 24) | | | | | |
| SB03095 | A5B7 | VH-VL | | | | 1.8 | 1593 |
| | | (SEQ ID NO: 25) | | | | | |
| SB03096 | | VL-VH | | | | 0.49 | 1140 |
| | | (SEQ ID NO: 26) | | | | | |
| SB03097 | Tus | VH-VL | CD8 | CD8 | 4-1BB | 77.5 | 3408 |
| | | (SEQ ID NO: 23) | | | | | |
| SB03098 | | VL-VH | | | | 63.2 | 2146 |
| | | (SEQ ID NO: 24) | | | | | |
| SB03099 | A5B7 | VH-VL | | | | 0.35 | 1100 |
| | | (SEQ ID NO: 25) | | | | | |
| SB03100 | | VL-VH | | | | 0.21 | 1191 |
| | | (SEQ ID NO: 26) | | | | | |

| | | | |
|---|---|---|---|
| SB02716 | Off-target (Axl) Control CAR | 18.7 | 1448 |
| No Virus | N/A | 0.44 | 998 |

TABLE 17

| | Gammaretroviral Construct Components | | | | | Day 4 | MFI of Myc+ |
|---|---|---|---|---|---|---|---|
| SB ID | scFv name | VH-VL or VL-VH | hinge | TM | co-stim | % Myc+ | Cells |
| SB03173 | hMN14 | VL-VH | CD8 | CD28 | CD28 | 15.8 | 25183 |
| | | (SEQ ID NO: 19) | | | | | |
| SB03174 | | VL-VH | CD8 | OX40 | OX40 | 21.5 | 7581 |
| | | (SEQ ID NO: 19) | | | | | |
| SB03175 | | VL-VH | CD8 | CD8 | 4-1BB | 21.1 | 11548 |
| | | (SEQ ID NO: 19) | | | | | |
| SB03176 | hMFE23 | VH-VL | CD8 | CD28 | CD28 | 34.1 | 10590 |
| | | (SEQ ID NO: 20) | | | | | |
| SB03178 | MRG1 | VH-VL | CD8 | OX40 | OX40 | 44.9 | 5859 |
| | | (SEQ ID NO: 22) | | | | | |
| SB03180 | Tus | VH-VL | CD8 | CD28 | CD28 | 34.1 | 27758 |
| | | (SEQ ID NO: 23) | | | | | |

| | | | |
|---|---|---|---|
| SB02716 | Off-target Control CAR | 1448 | 1448 |
| No Virus | N/A | 0.44 | 998 |

TABLE 18

| | Lentiviral Construct Components | | | | | Day 7 | MFI of Myc+ |
|---|---|---|---|---|---|---|---|
| SB ID | scFv name | VH-VL or VL-VH | hinge | TM | co-stim | % Myc+ | Cells |
| SB03089 | Tus | VH-VL | CD8 | CD28 | CD28 | 34.8 | 1584 |
| | | (SEQ ID NO: 23) | | | | | |
| SB03090 | | VL-VH | | | | 15.3 | 3037 |
| | | (SEQ ID NO: 24) | | | | | |
| SB03091 | A5B7 | VH-VL | | | | 1.05 | 1140 |
| | | (SEQ ID NO: 25) | | | | | |
| SB03092 | | VL-VH | | | | 0.21 | 1146 |
| | | (SEQ ID NO: 26) | | | | | |
| SB03093 | Tus | VH-VL | CD28 | | | 12.2 | 1247 |
| | | (SEQ ID NO: 23) | | | | | |
| SB03094 | | VL-VH | | | | 3.68 | 1833 |
| | | (SEQ ID NO: 24) | | | | | |
| SB03095 | A5B7 | VH-VL | | | | 0.67 | 1243 |
| | | (SEQ ID NO: 25) | | | | | |
| SB03096 | | VL-VH | | | | 0.22 | 1319 |
| | | (SEQ ID NO: 26) | | | | | |
| SB03097 | Tus | VH-VL | CD8 | CD8 | 4-1BB | 54.7 | 1996 |
| | | (SEQ ID NO: 23) | | | | | |
| SB03098 | | VL-VH | | | | 27.4 | 2023 |
| | | (SEQ ID NO: 24) | | | | | |
| SB03099 | A5B7 | VH-VL | | | | 0.17 | 1421 |
| | | (SEQ ID NO: 25) | | | | | |
| SB03100 | | VL-VH | | | | 0.21 | 1085 |
| | | (SEQ ID NO: 26) | | | | | |

| | | | |
|---|---|---|---|
| SB02716 | Off-target Control CAR | 8.34 | 3257 |
| No Virus | N/A | 0.37 | 1531 |

TABLE 19

| | Gammaretroviral Construct Components | | | | | Day 7 | MFI of Myc+ |
|---|---|---|---|---|---|---|---|
| SB ID | scFv | VH-VL or VL-VH | hinge | TM | co-stim | % Myc+ | Cells |
| SB03173 | hMN14 | VL-VH | CD8 | CD28 | CD28 | 11 | 10031 |
| | | (SEQ ID NO: 19) | | | | | |
| SB03174 | | VL-VH | CD8 | OX40 | OX40 | 17.1 | 5719 |
| | | (SEQ ID NO: 19) | | | | | |
| SB03175 | | VL-VH | CD8 | CD8 | 4-1BB | 13 | 6619 |
| | | (SEQ ID NO: 19) | | | | | |
| SB03176 | hMFE23 | VH-VL | CD8 | CD28 | CD28 | 20.5 | 5931 |
| | | (SEQ ID NO: 20) | | | | | |
| SB03178 | MRG1 | VH-VL | CD8 | OX40 | OX40 | 26.7 | 3176 |
| | | (SEQ ID NO: 22) | | | | | |
| SB03180 | Tus | VH-VL | CD8 | CD28 | CD28 | 25.9 | 14583 |
| | | (SEQ ID NO: 23) | | | | | |

| | | | |
|---|---|---|---|
| SB02716 | Off-target Control CAR | 8.34 | 3257 |
| No Virus | N/A | 0.37 | 1531 |

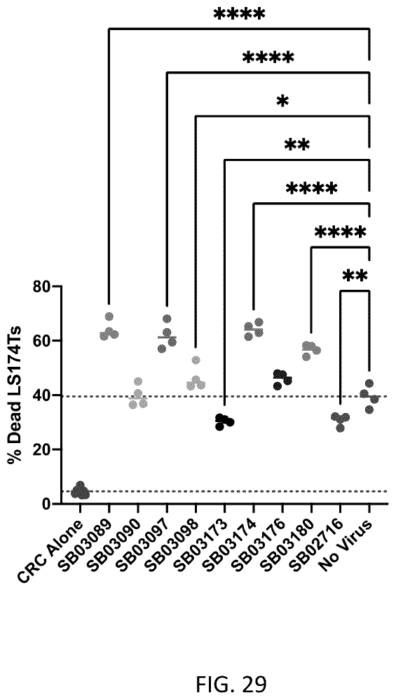

Killing activity of NK cells transduced with a subset of the constructs shown in Tables 16-19 was assessed in a flow-based assay. aCAR-transduced NK cells were incubated with colorectal cancer target line LS174t at an effector cell (i.e. NK cells, “E”) to target cell (in this case, an LS174t cell, “T”), “E:T” ratio, of 1:1 overnight (16-18 hrs). Cells were co-cultured in an ultra-low bind U-bottom plate to facilitate cell-cell interactions and mitigate plate adherence. Cells were harvested and stained for flow cytometry to determine cell phenotype and death. mKate expression of the Ls174t target cells was used to distinguish target cells from CD56+NK cells. Zombie UV (Biolegend) dye was used to distinguish live from dead cells. Percentage of dead cells was calculated and shown in FIG. 29. As shown in FIG. 29, many of the CEA aCAR constructs (SB03089, SB03097, SB03098, SB03174, SB03176, and SB03180) exhibited higher killing activity over the untransduced (No Virus) NK controls at an E to T ratio of 1:1. Significant differences were determined using an ordinary one-way ANOVA with Dunnett's multiple comparison correction, * p<0.05, ** p<0.005, **** p<0.0001. Taken together, these results demonstrate significant killing activity of CEA aCAR-expressing NK cells, produced using both lentiviral vectors and gammaretroviral vectors.

### Example 6: Killing Activity for Lentivirally Transduced NK Cells Expressing Various CEA aCAR Constructs at Various Effector Cell to Target Cell Ratios

CEA aCAR expression of NK cells lentivirally transduced with various CEA aCAR constructs was assessed 5 days post transduction. Lentivirus constructs with CD28 or 4-1BB co-stimulatory domains and a CD3zeta intracellular domain were tested with CEACAM5-specific scFvs (A5B7, Tusamitamab or “Tus” and hMN14). For A5B7 and tusamitamab-derived scFvs, both orientations, VH-VL and VL-VH, were tested. An off-target “control” CAR (SB02716) was also tested as a negative control. A description of each construct is provided in Table 20. CAR expression was assessed via flow cytometry by staining for the presence of an epitope tag (Myc). Cells were stained at 5 days post-transduction to determine stability of construct expression. Lentivirus for SB03089 to SB03100 was added to transduce cells at an MOI of 2 pg p24 per NK cell. Virus for SB02463 and SB02779 was titrated so that CAR expression would be matched for both constructs. Gates to determine % Myc expression were drawn using untransduced controls (No Virus) so that the <1% of Myc+ cells were in the positive gate. Median fluorescence intensity (MFI) was determined to quantify and assess levels of CAR expression. Expression of each construct as determined by Myc tag expression is shown in Table 20.

TABLE 20

| | Construct Components | | | | | | |
|---|---|---|---|---|---|---|---|
| Construct | | VH-VL or VL-VH | | | | Day 5 | MFI of Myc+ |
| name | scFv | & SEQ ID NO | hinge | TM | co-stim | % Myc+ | Cells |
| SB03089 | Tus | VH-VL | CD8 | CD28 | CD28 | 19.6 | 1157 |
| | | (SEQ ID NO: 23) | | | | | |
| SB03090 | | VL-VH | | | | 15.2 | 3911 |
| | | (SEQ ID NO: 24) | | | | | |
| SB03091 | A5B7 | VH-VL | | | | 2.43 | 895 |
| | | (SEQ ID NO: 25) | | | | | |
| SB03092 | | VL-VH | | | | 1.41 | 755 |
| | | (SEQ ID NO: 26) | | | | | |
| SB03093 | Tus | VH-VL | CD28 | | | 8.37 | 1021 |
| | | (SEQ ID NO: 23) | | | | | |
| SB03094 | | VL-VH | | | | 4.84 | 1232 |
| | | (SEQ ID NO: 24) | | | | | |
| SB03095 | A5B7 | VH-VL | | | | 2.17 | 925 |
| | | (SEQ ID NO: 25) | | | | | |
| SB03096 | | VL-VH | | | | 1.52 | 805 |
| | | (SEQ ID NO: 26) | | | | | |
| SB03097 | Tus | VH-VL | CD8 | CD8 | 4-1BB | 33.1 | 1427 |
| | | (SEQ ID NO: 23) | | | | | |
| SB03098 | | VL-VH | | | | 22.9 | 1853 |
| | | (SEQ ID NO: 24) | | | | | |
| SB03099 | A5B7 | VH-VL | | | | 0.74 | 757 |
| | | (SEQ ID NO: 25) | | | | | |
| SB03100 | | VL-VH | | | | 0.73 | 764 |
| | | (SEQ ID NO: 26) | | | | | |
| SB02463 | hMN14 | VL-VH | CD8 | CD28 | CD28 | 16.3 | 1013 |
| | | (SEQ ID NO: 19) | | | | | |
| SB02779 | | VL-VH | CD8 | OX40 | OX40 | 16.9 | 1173 |
| | | (SEQ ID NO: 19) | | | | | |

| | | | |
|---|---|---|---|
| SB02716 | Off-target Control CAR | 13.8 | 3226 |
| No Virus | N/A | 0.88 | 760 |

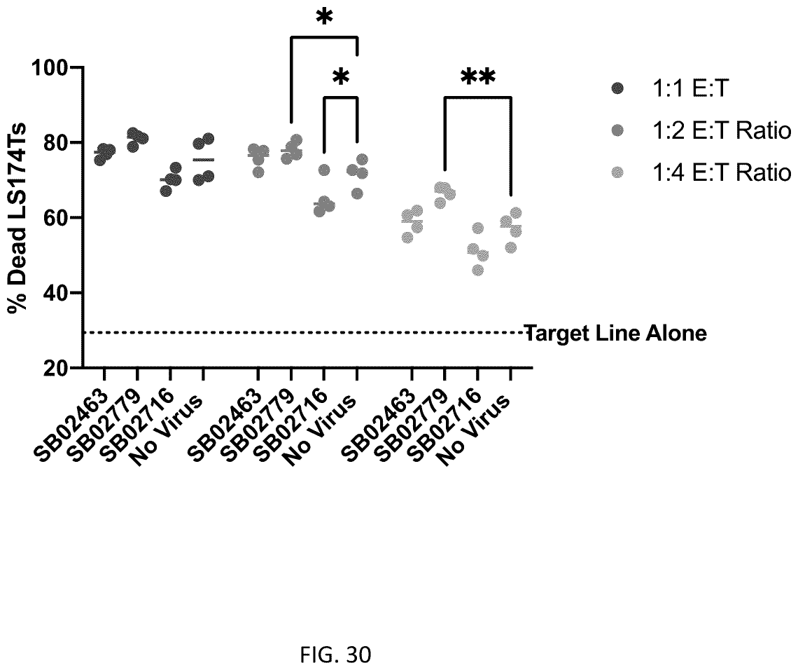

Killing activity of NK cells transduced with a subset of constructs as shown in Table 20 was assessed in a flow-based assay at different E:T ratios. NK cells transduced with two of the CEA aCAR constructs shown in Table 16 (SB02463 and SB02779) were incubated with colorectal cancer target line LS174t at E:T ratios of 1:1, 1:2 and 1:4 overnight (16-18 hrs). Cells were co-cultured in an ultra-low bind U-bottom plate to facilitate cell-cell interactions and mitigate plate adherence. Co-cultures were also performed with untransduced NK cells (No Virus) and NK cells transduced with an off-target CAR (SB02716, which targets Axl) as negative controls. Cells were harvested and stained for flow cytometry to determine cell phenotype and death. mKate expressing Ls174t target cells were used to distinguish target cells from CD56+NK cells. Zombie UV (Biolegend) dye was used to distinguish live from dead cells. Percentage of dead cells was calculated and shown in FIG. 30. As shown in FIG. 30, both CEA aCAR constructs exhibited higher killing activity over the untransduced (No Virus) NK controls at lower E:T ratios indicating CAR-mediated killing of cognate target cells. Significant differences were determined using an ordinary two-way ANOVA with Dunnett's multiple comparison correction, * p<0.05, ** p<0.005.

### Example 7: Expression of CEA aCARs with Various Binding Domains and Signalling Domains Following Gammaretrovirus Transduction of NK Cells

CEA aCAR expression on gammaretrovirus-transduced NK cells was assessed. Multiple gammaretroviral constructs were tested with four distinct scFvs (Tus, hMN14, hMFE23, and MG7); four different transmembrane domains: CD8 (SEQ ID NO: 45), CD28 (SEQ ID NO: 46), OX40 (SEQ ID NO: 47), and 2B4 (SEQ ID NO: 48); and four different co-stimulatory domains: CD28 (SEQ ID NO: _71), OX40 (SEQ ID NO: 72), 2B4 (SEQ ID NO: 73), and 4-1BB (SEQ ID NO: 70). Each construct also included a myc epitope tag, a CD8 hinge domain (SEQ ID NO: 50), and a CD3zeta activation domain (SEQ ID NO: 74). A description of each construct is provided in Table 21. CAR expression was assessed via flow cytometry by staining for the presence of the epitope tag (Myc). Cells were stained at 3- and 7-days post-transduction to determine stability of construct expression. Gates to determine % myc expression were drawn using untransduced controls (No Virus) so that the <1% of Myc+ cells were in the positive gate. Median fluorescence intensity (MFI) was determined to quantify and assess levels of CAR expression. CAR expression levels on day 3 and day 7 for gammaretrovirus-transduced NK cells are shown in Table 22.

TABLE 21

| | Construct Components | | | | | |
|---|---|---|---|---|---|---|
| | | VH-VL or VL-VH & | | | | |
| Construct Name | scFv | SEQ ID NO | Tag | Hinge | TM | Co-stim |
| SB03180 | Tus | VH-VL | Myc | CD8 | CD28 | CD28 |
| | | (SEQ ID NO: 23) | | | | |
| SB03290 | Tus | VH-VL | Myc | CD8 | OX40 | OX40 |
| | | (SEQ ID NO: 23) | | | | |
| SB03615 | Tus | VH-VL | Myc | CD8 | CD28 | 4-1BB |
| | | (SEQ ID NO: 23) | | | | |
| SB03173 | hMN14 | VL-VH | Myc | CD8 | CD28 | CD28 |
| | | (SEQ ID NO: 19) | | | | |
| SB03174 | hMN14 | VL-VH | Myc | CD8 | OX40 | OX40 |
| | | (SEQ ID NO: 19) | | | | |
| SB03604 | hMN14 | VL-VH | Myc | CD8 | 2B4 | 2B4 |
| | | (SEQ ID NO: 19) | | | | |
| SB03175 | hMN14 | VL-VH | Myc | CD8 | CD8 | 4-1BB |
| | | (SEQ ID NO: 19) | | | | |
| SB03176 | hMFE23 | VH-VL | Myc | CD8 | CD28 | CD28 |
| | | (SEQ ID NO: 20) | | | | |
| SB03605 | hMFE23 | VH-VL | Myc | CD8 | OX40 | OX40 |
| | | (SEQ ID NO: 20) | | | | |
| SB03606 | hMFE23 | VH-VL | Myc | CD8 | 2B4 | 2B4 |
| | | (SEQ ID NO: 20) | | | | |
| SB03607 | hMFE23 | VH-VL | Myc | CD8 | CD8 | 4-1BB |
| | | (SEQ ID NO: 20) | | | | |
| SB03603 | MG7 | VL-VH | Myc | CD8 | CD28 | CD28 |
| | | (SEQ ID NO: 21) | | | | |
| SB03608 | MG7 | VL-VH | Myc | CD8 | CD8 | OX40 |
| | | (SEQ ID NO: 21) | | | | |
| SB03609 | MG7 | VL-VH | Myc | CD8 | 2B4 | 2B4 |
| | | (SEQ ID NO: 21) | | | | |
| SB03610 | MG7 | VL-VH | Myc | CD8 | CD8 | 4-1BB |
| | | (SEQ ID NO: 21) | | | | |

| | |
|---|---|
| No Virus | N/A |

TABLE 22

| | Day 3 | | Day 7 | |
|---|---|---|---|---|
| | | MFI of Myc+ | | MFI of Myc+ |
| SB number | % Myc+ | Cells | % Myc+ | Cells |
| SB03180 | 46 | 17919 | 51.7 | 7794 |
| SB03290 | 4.35 | 7694 | 3.02 | 4103 |
| SB03615 | 45.8 | 16861 | 45 | 7823 |
| SB03173 | 75.4 | 19798 | 70.3 | 9827 |
| SB03174 | 46.6 | 6148 | 42.2 | 4635 |
| SB03604 | 54.3 | 8585 | 46.6 | 4554 |
| SB03175 | 28.7 | 9069 | 29.8 | 6119 |
| SB03176 | 47 | 11582 | 45.3 | 5339 |
| SB03605 | 47.4 | 4050 | 42 | 2729 |
| SB03606 | 37.5 | 4495 | 27.7 | 2754 |
| SB03607 | 21.4 | 6882 | 22.7 | 4385 |
| SB03603 | 43.7 | 4836 | 32.1 | 3754 |
| SB03608 | 19 | 2053 | 12.7 | 1904 |
| SB03609 | 18.1 | 3130 | 12.3 | 2505 |
| SB03610 | 16.3 | 3041 | 10.1 | 2297 |
| No Virus | 0.82 | 1475 | 0.27 | 1337 |

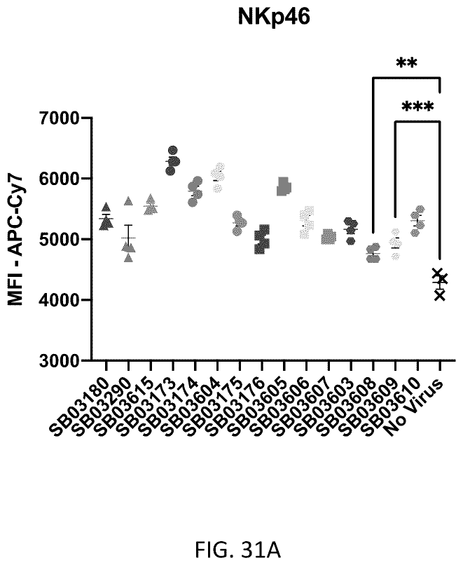

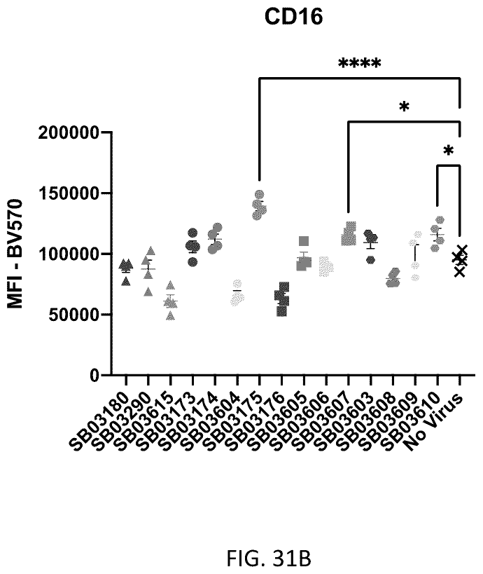

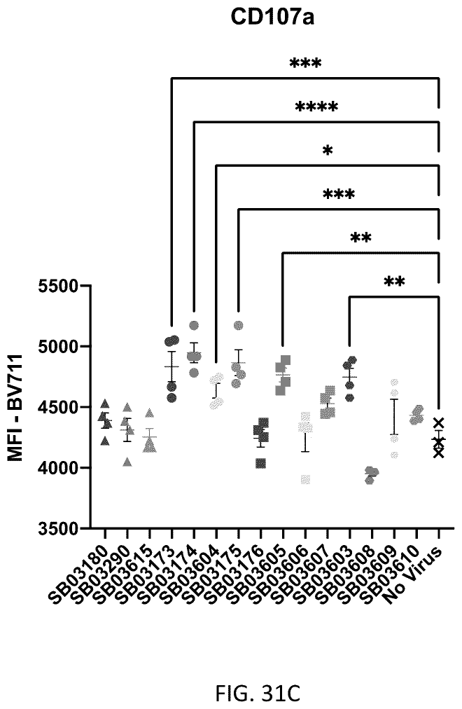

Surface marker activation of transduced CEA aCAR NK cells in culture with target cells. CEA aCAR transduced NK cells were incubated overnight (16-18 hrs) with colorectal cancer target line (LS174t) at an E:T ratio of 1:1 overnight. Cells were co-cultured in an ultra-low bind U-bottom plate to facilitate cell-cell interactions and mitigate plate adherence. Cells were harvested and stained for flow cytometry to determine cell phenotype and death. mKate expressing Ls174t target cells were used to distinguish target cells from CD56+NK cells. NK activation markers NKp46, CD16, and CD107A, were stained to assess CAR NK activity in the presence of target cells. MFI was calculated for each and is shown in FIG. 31A (NKp46), FIG. 31B (CD16), and FIG. 31C (CD107a). Significant differences were determined using an ordinary one-way ANOVA with Dunnett's multiple comparison correction, * p<0.05, ** p<0.005, *** p<0.0005, **** p<0.0001. As shown in FIG. 31A, FIG. 31B, and FIG. 31C, surface marker activity was increased for several of the tested CEA aCAR NK cell populations indicating activation of NK cells following recognition of cognate target cells.

### Example 8: Killing Activity of Transduced CEA aCAR NK Cells as Assessed by a Real-Time Fluorescence-Based Assay

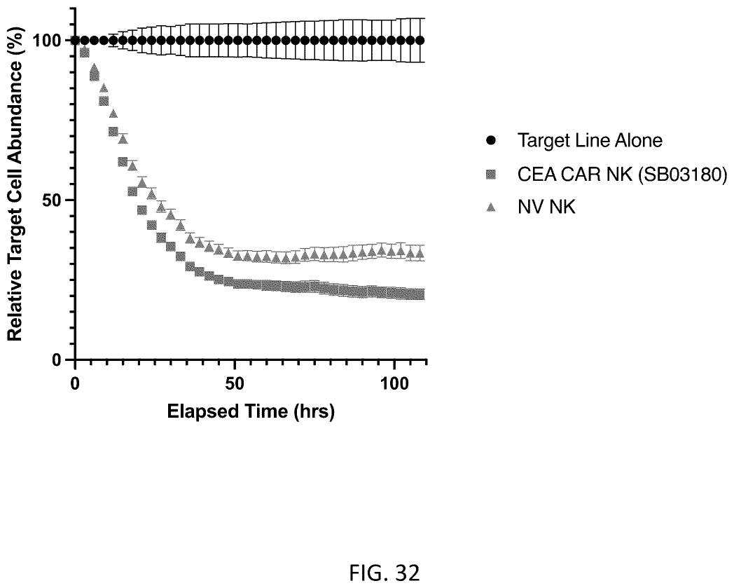

A population of CEA-expressing colorectal cancer cells (Ls174t cell line) were stably transduced with a fluorescent reporter, mKate, to track target cell growth and abundance during culture. The transduced Ls174t cells are referred to here as “target line.” NK cells transduced with a CEA aCAR (construct SB03180) and untransduced control (“no virus” or “NV”) NK cells were then incubated with LS174t at an E:T ratio of 2:1. Cells were seeded in six technical replicates for each condition: target line alone, untransduced (“NV”) NK cells with the target line, and CEA aCAR NK cells (transduced with construct SB03180, see Table 21) with the target line. During culture, imaging of the reporter signal from target cells was performed every three hours via a real-time fluorescence assay to measure mkate. Target line abundance for each condition was determined from total red integrated intensity per well. Percentage of dead cells was calculated and shown in FIG. 32. All data was normalized to values from target line alone and are plotted +/−SEM. As shown in FIG. 32, CEA CAR NK cells inhibit target line growth in vitro at a faster rate than untransduced controls (NV NK) indicating CAR-mediated killing of cognate target cells.

### Example 9: Killing Activity of Transduced CEA aCAR NK Cells In Vivo

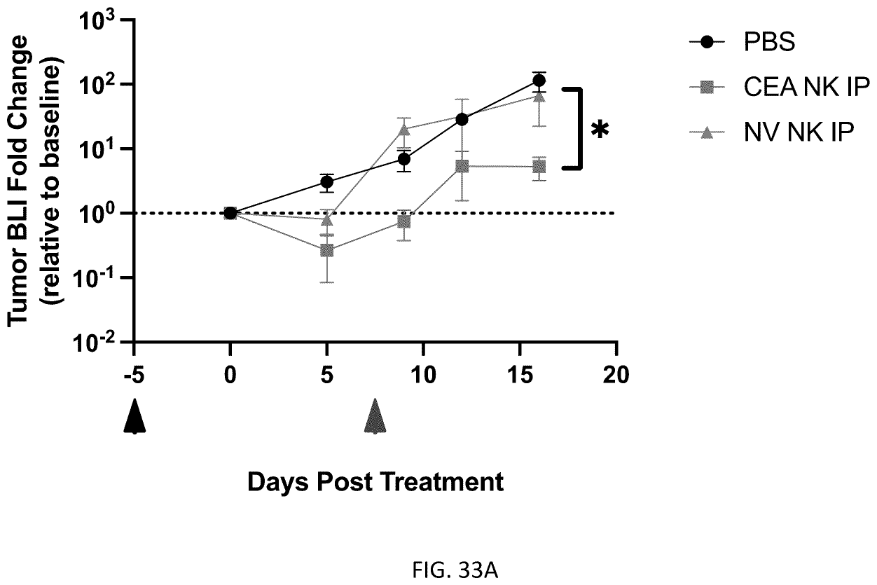

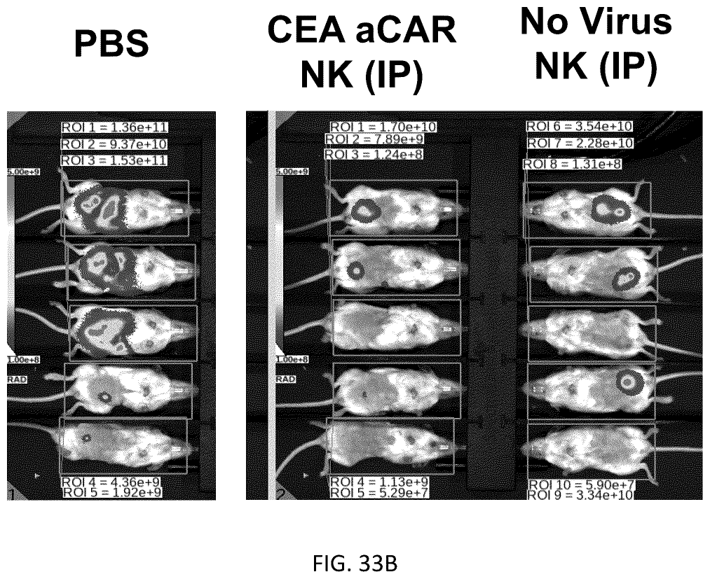

Killing activity NK cells transduced with a CEA aCAR was assessed in vivo. To track tumor progression in vivo, CEA-expressing colorectal cancer Ls174t target cells were transduced to express firefly luciferase (fLuc). Mice were inoculated intraperitoneally with 1e6 L1S174t target cells per mouse five days prior to NK cell treatment (day −5, indicated in right panel by black arrow). Each study arm was treated with 30e6 NK cells (NK cells transduced with CEA aCAR construct SB03180 or untransduced NK cells) with administration of the NK cells via intraperitoneal (IP) delivery or vehicle control (PBS). Each study arm began with eight mice per group, and three mice were sacrificed at an intermediate time point (day 6, indicated by purple arrow) for intermediate analysis (n=8 initially, n=5 post-takedown). After cancer cell inoculation, fLuc signal was assessed twice a week using total body bioluminescence imaging (BLI). Changes in BLI were normalized to initial baseline values at time of treatment (day 0) as shown in FIG. 33A. A representative image taken 16 days post-treatment is shown in FIG. 33B. As shown in FIG. 33A and FIG. 33B, after 16 days post-treatment, mice that had been treated with CEA aCAR NK cells exhibited significantly lower tumor burden than mice that had been treated with untransduced indicating CAR-mediated killing of tumor cells in vivo, no virus (NV) control NK cells (* p<0.05, 2way ANOVA with Sidak's multiple comparison test).

### Example 10: CAR Expression and CAR-NK Killing of Tumor Cells Following Baboon Envelope-Pseudotyped Gamma-Retrovirus Transduction

Primary NK cells were obtained from a first donor (“NK donor 1”) and second donor (“NK donor 2”). Gammaretroviruses encoding various CEA aCARs were produced using a self-inactivating gammaretroviral vector and a baboon endogenous virus (BaEV) envelope was used for pseudotyping. NK cells from both donors were transduced with the gammaretroviruses and CEA aCAR expression on the transduced NK cells was assessed. Retrovirus constructs, using a first gamma-retrovirus construct backbone and a second gammaretrovirus construct backbone (“retrovirus 1” and “retrovirus 2”) encoding CARs with CD28 and CD3zeta intracellular domains or OX40 and CD3zeta intracellular domains were tested for two CEACAM5-specific scFvs (hMN-14 and tusamitamab, “Tus”). CAR expression was assessed via flow cytometry by staining for the presence of an epitope tag (Myc). Cells were stained at 11-days post-transduction to determine stability of construct expression. Gates to determine % Myc expression were drawn using untransduced controls (No Virus) so that the <1% of Myc+ cells were in the positive gate. Median fluorescence intensity (MFI) was determined to quantify and assess levels of CAR expression. CAR expression for NK Donor 1 cells transduced with BaEV-pseudotyped gammaretroviruses are shown in Table 23. CAR expression for NK Donor 2 cells transduced with the same BaEV-pseudotyped gammaretroviruses are shown in Table 24. The average of percent expression (transduction) between the two donors is shown in Table 25.

TABLE 23
NK Donor 1 cells

| | Retrovirus | | | | | | |
|---|---|---|---|---|---|---|---|
| Construct | Construct | scFv | Codon | costim | signal | % transduction | |
| name | Backbone | name | Optimization | domain | sequence | (Myc+) | MFI |
| SB03173 | 1 | hMN14 | Human codon | CD28 | CD8 | 18 | 534 |
| | | | optimization | | | | |
| SB03174 | 1 | hMN14 | Human codon | OX40 | CD8 | 5.02 | 179 |
| | | | optimization | | | | |
| SB04285 | 1 | hMN14 | New codon | CD28 | CD8 | 44.2 | 1454 |
| | | | optimization | | | | |
| | | | (encodes same | | | | |
| | | | CAR a.a. sequence | | | | |
| | | | as SB03173) | | | | |
| SB04293 | 1 | hMN14 | New codon | OX40 | CD8 | 47.2 | 1695 |
| | | | optimization | | | | |
| | | | (encodes same | | | | |
| | | | CAR a.a. sequence | | | | |
| | | | as SB03174) | | | | |
| SB03180 | 1 | Tus | Human codon | CD28 | CD8 | 19.4 | 562 |
| | | | optimization | | | | |
| SB03290 | 1 | Tus | Human codon | OX40 | CD8 | 9.08 | 248 |
| | | | optimization | | | | |
| SB04288 | 1 | Tus | New codon | CD28 | CD8 | 45.7 | 1338 |
| | | | optimization | | | | |
| | | | (encodes same | | | | |
| | | | CAR a.a. sequence | | | | |
| | | | as SB03180) | | | | |
| SB04296 | 1 | Tus | New codon | OX40 | CD8 | 39.8 | 1077 |
| | | | optimization | | | | |
| | | | (encodes same | | | | |
| | | | CAR a.a. sequence | | | | |
| | | | as SB03190) | | | | |
| SB04442 | 2 | hMN14 | Human | CD28 | CD8 | 93.1 | 16783 |
| | | | optimization | | | | |
| | | | (encodes same | | | | |
| | | | CAR a.a. sequence | | | | |
| | | | as SB03173) | | | | |
| SB04443 | 2 | hMN14 | Human | OX40 | CD8 | 94.1 | 18022 |
| | | | optimization | | | | |
| | | | (encodes same | | | | |
| | | | CAR a.a. sequence | | | | |
| | | | as SB03174) | | | | |
| SB04444 | 2 | Tus | Human codon | CD28 | CD8 | 94.4 | 19568 |
| | | | optimization | | | | |
| | | | (encodes same | | | | |
| | | | CAR a.a. sequence | | | | |
| | | | as SB03180) | | | | |
| SB04445 | 2 | Tus | Human codon | OX40 | CD8 | 91.9 | 11533 |
| | | | optimization | | | | |
| | | | (encodes same | | | | |
| | | | CAR a.a. sequence | | | | |
| | | | as SB03190) | | | | |

TABLE 24
NK Donor 2 cells

| | Retrovirus | | | | | | |
|---|---|---|---|---|---|---|---|
| Construct | Construct | scFv | Codon | costim | signal | % transduction | |
| name | Backbone | name | Optimization | domain | sequence | (Myc+) | MFI |
| SB03173 | 1 | hMN14 | Human codon | CD28 | CD8 | 11.9 | 329 |
| | | | optimization | | | | |
| SB03174 | 1 | hMN14 | Human codon | OX40 | CD8 | 4.31 | 151 |
| | | | optimization | | | | |
| SB04285 | 1 | hMN14 | New codon | CD28 | CD8 | 28.4 | 768 |
| | | | optimization | | | | |
| SB04293 | 1 | hMN14 | New codon | OX40 | CD8 | 42.5 | 1422 |
| | | | optimization | | | | |
| SB03180 | 1 | Tus | Human codon | CD28 | CD8 | 19.5 | 561 |
| | | | optimization | | | | |
| SB03290 | 1 | Tus | Human codon | OX40 | CD8 | 8.93 | 233 |
| | | | optimization | | | | |
| SB04288 | 1 | Tus | New codon | CD28 | CD8 | 30.7 | 753 |
| | | | optimization | | | | |
| SB04296 | 1 | Tus | New codon | OX40 | CD8 | 38.3 | 1021 |
| | | | optimization | | | | |
| SB04442 | 2 | hMN14 | Human codon | CD28 | CD8 | 88.6 | 11862 |
| | | | optimization | | | | |
| SB04443 | 2 | hMN14 | Human codon | OX40 | CD8 | 85.1 | 6268 |
| | | | optimization | | | | |
| SB04444 | 2 | Tus | Human codon | CD28 | CD8 | | N/A |
| | | | optimization | | | | |
| SB04445 | 2 | Tus | Human codon | OX40 | CD8 | 86.4 | 7295 |
| | | | optimization | | | | |

TABLE 25
Average between donors

| | % | | |
|---|---|---|---|
| Construct | transduction | | |
| name | (Myc+) | SD | MFI |
| SB03173 | 14.95 | 4.31 | 431.5 |
| SB03174 | 4.665 | 0.50 | 165 |
| SB04285 | 36.3 | 11.17 | 1111 |
| SB04293 | 44.85 | 3.32 | 1558.5 |
| SB03180 | 19.45 | 0.07 | 561.5 |
| SB03290 | 9.005 | 0.106 | 240.5 |
| SB04288 | 38.2 | 10.606 | 1045.5 |
| SB04296 | 39.05 | 1.06 | 1049 |
| SB04442 | 90.85 | 3.18 | 14322.5 |
| SB04443 | 89.6 | 6.36 | 12145 |
| SB04444 | 94.4 | N/A | 19568 |
| SB04445 | 89.15 | 3.889 | 9414 |

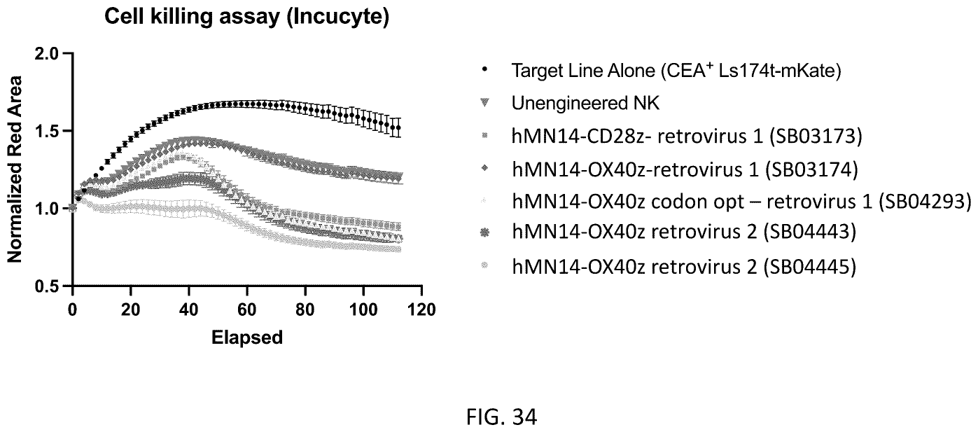

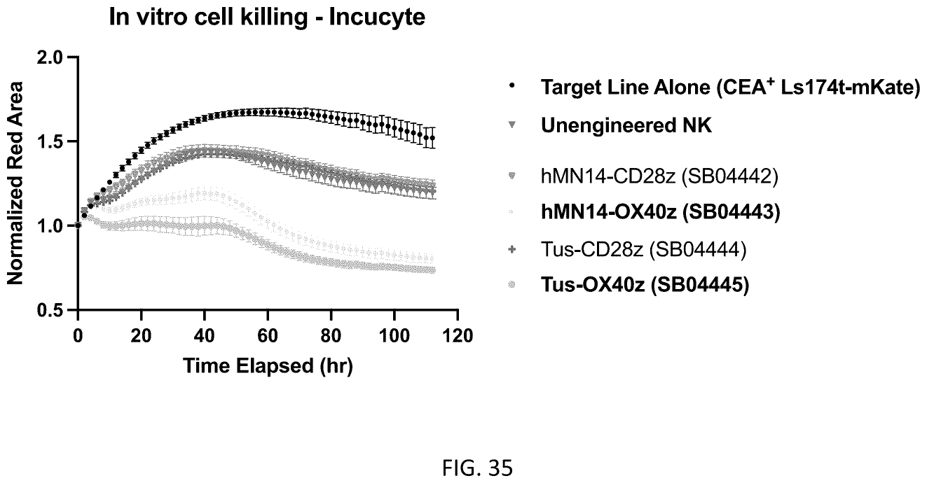

A population of CEA-expressing colorectal cancer cells (Ls174t cell line) were stably transduced with a fluorescent reporter, mKate, to track target cell growth and abundance during culture. The transduced Ls174t cells are referred to here as “target line.” NK cells transduced with a CEA aCAR (construct SB03180) and untransduced control (“no virus” or “NV”) NK cells were then incubated with LS174t at an E:T ratio of 2:1. Cells were seeded in six technical replicates for each condition: target line alone, untransduced (“NV”) NK cells with the target line, and CEA aCAR NK cells (transduced with construct SB03180) with the target line. During culture, imaging of the reporter signal from target cells was performed every three hours via a real-time fluorescence assay to measure mkate. Target line abundance for each condition was determined from total red integrated intensity per well. Percentage of dead target cells was calculated for constructs included in a first killing assay (SB03173, SB04293, SB04444, and SB04445; FIG. 34). A separate, second killing assay was conducted for SB04443, SB04444, and SB04445 and the percentage of dead target cells in this second assay are shown in FIG. 35. As shown in FIG. 34, NK cells transduced with CEA aCAR constructs SB03173, SB04293, SB04443, SB04445 demonstrated higher killing than untransduced NK cells, and as shown in FIG. 35 the killing was confirmed in a second assay for constructs SB04443 and SB04445, which both include OX40 intracellular domains. All data are normalized to values from target line alone and are plotted +/−SEM. The killing activity shown in FIG. 35 confirms the higher killing for NK cells transduced with CEA aCAR constructs SB04443, SB04445, as compared to killing by unengineered NK cells, and as also shown in FIG. 34.

### Example 11: In Vivo Anti-Tumor Activity of CAR-NK Cells Transduced with Baboon Envelope-Pseudotyped Gamma-Retrovirus

CEA aCAR NK cells were produced for assessing in vivo anti-tumor activity. Primary NK cells were transduced with various CEA aCAR constructs (constructs SB03173, SB03174, SB03180, SB03290, and SB03176) or were untransduced (“no virus” or “NV”) NK cells. CAR expression was assessed via flow cytometry by staining for the presence of an epitope tag (Myc). Cells were stained at 11 days post-transduction to determine stability of construct expression. Gates to determine % Myc expression were drawn using untransduced controls (No Virus) so that the <1% of Myc+ cells were in the positive gate. Median fluorescence intensity (MFI) was determined to quantify and assess levels of CAR expression. Percent expression for each, on day 8 and day 16 post-transduction, is shown in Table 26.

TABLE 26

| | Time point | % CAR+ NK | MFI | | % CAR+ | MFI |
|---|---|---|---|---|---|---|
| | (Days post | cells (Myc | AF647 | | NK cells | AF647 |
| Construct | transduction) | tag) | (Myc tag) | Time point | (Myc tag) | (Myc tag) |
| SB03173 | Day 8 | 32.6 | 10262 | Day 16 | 27.9 | 10316 |
| SB03174 | Day 8 | 25.6 | 5648 | Day 16 | 28.1 | 7014 |
| SB03180 | Day 8 | 32 | 13055 | Day 16 | 32.7 | 15832 |
| SB03290 | Day 8 | 18.7 | 6276 | Day 16 | 22 | 7787 |
| SB03176 | Day 8 | 29.2 | 8113 | Day 16 | 39.5 | 9819 |
| Untransduced | Day 8 | 0.58 | 1073 | Day 16 | 0.44 | 1030 |

As shown in Table 26, CAR expression was stable over the time, with at least 20% and up to about 40% expression 16-days post transduction.

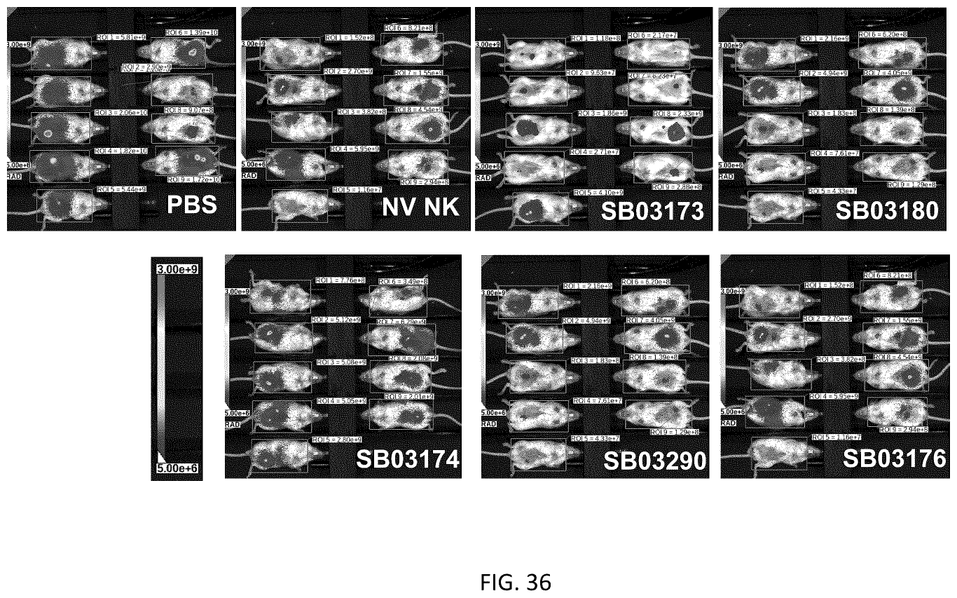

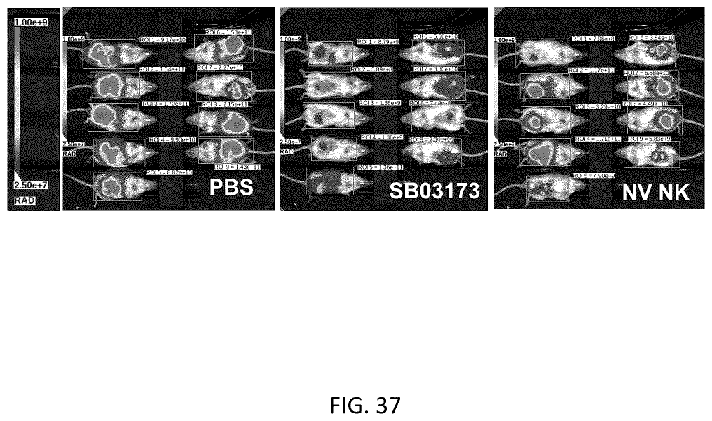

To track tumor progression in vivo, CEA-expressing colorectal cancer Ls174t target cells were transduced to express firefly luciferase (fLuc). Mice were inoculated intraperitoneally with 1e6 L1S174t target cells per mouse five days prior to NK cell treatment. Each study arm was treated with a population of 30e6 NK cells via intraperitoneal (IP) delivery or vehicle control (PBS). Each study arm began with nine mice per group. After cancer cell inoculation, fLuc signal was assessed 2 days post NK cell-treatment using total body bioluminescence imaging (BLI), and images of the nine mice of each study arm are shown in FIG. 36. fLuc signal was also assessed 13 days post NK cell-treatment using total BLI, and significantly lower tumor burden was attained for mice treated with NK cells transduced with construct SB03173 as compared to mice treated with untransduced NK cells. Representative images of the nine mice of the SB03173 NK cell study arm, the untransduced NK cell study arm, and the PBS treated (untreated) study arm are shown in FIG. 37. For these three study arms, tumor progression was assessed by calculating the BLI fold change for each mouse on day 2 of NK cell treatment or day 13 of NK cell treatment, as compared to day 0 of NK cell treatment. The disease outcome of each mouse in the SB03173 NK cells study arm, the untransduced NK cells study arm, and the PBS (untreated) study arm was categorized as progressive disease (BLI fold change of less than 1), stable disease (BLI fold change of greater than 1 or less than 10), or partial/complete remission (BLI fold change of greater than 10) on day 2 and day 13 (Table 27).

TABLE 27

| | Day 2 | | | Day 13 | | |
|---|---|---|---|---|---|---|
| Treatment arm | PD | SD | PR/CR | PD | SD | PR/CR |
| Untreated (PBS) | 7 | 2 | 0 | 9 | 0 | 0 |
| No Virus NK | 3 | 5 | 1 | 5 | 3 | 1 |
| SB03173 NK | 2 | 1 | 5 | 4 | 2 | 3 |

As shown in Table 27, treatment with NK cells transduced with a CEA aCAR (construct S1B03173) resulted in higher incidences of partial/complete remission than treatment with untransduced NK cells and no treatment, at day 2 and day 13. These results indicate the anti-tumor activity of a CEA aCAR in vivo. Sequences, both in DNA and amino acid (“AA”), are provided in Table 28.

TABLE 28

| | |
|---|---|
| SB02463 | ATGGCTCTGCCTGTGACAGCTCTGCTGCTGCCTCT |
| DNA Seq | GGCTCTGCTTCTGCATGCCGCCAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAGCAGAAGCTGATCTCCGAAG |
| | AGGACCTGACCACAACACCAGCTCCTAGACCACCT |
| | ACACCAGCACCTACAATCGCCCTGCAGCCACTGTC |
| | TCTGAGGCCAGAGGCTTGTAGACCTGCTGCTGGCG |
| | GAGCCGTGCATACAAGAGGACTGGATTTCGCCTGC |
| | GACTTCTGGGTGCTCGTGGTTGTTGGAGGCGTGCT |
| | GGCCTGTTACTCTCTGCTGGTCACCGTGGCCTTCA |
| | TCATCTTTTGGGTCCGAAGCAAGCGGAGCCGGCTG |
| | CTGCACAGCGACTACATGAACATGACCCCTAGACG |
| | GCCCGGACCTACCAGAAAGCACTACCAGCCTTACG |
| | CTCCTCCTAGAGACTTCGCCGCCTACCGGTCCAGA |
| | GTGAAGTTCTCCAGATCTGCCGACGCTCCCGCCTA |
| | TAAGCAGGGCCAGAACCAGCTGTACAACGAGCTGA |
| | ACCTGGGGAGAAGAGAAGAGTACGACGTGCTGGAC |
| | AAGCGGAGAGGCAGAGATCCTGAGATGGGCGGCAA |
| | GCCCAGACGGAAGAATCCTCAAGAGGGCCTGTATA |
| | ATGAGCTGCAGAAAGACAAGATGGCCGAGGCCTAC |
| | AGCGAGATCGGAATGAAGGGCGAGCGCAGAAGAGG |
| | CAAGGGACACGATGGACTGTACCAGGGCCTGAGCA |
| | CCGCCACCAAGGATACCTATGATGCCCTGCACATG |
| | CAGGCCCTGCCTCCAAGA |
| | (SEQ ID NO: 80) |
| SB02463 | MALPVTALLLPLALLLHAARPEVOLVESGGGVVQP |
| A.A. Seq | GRSLRLSCSASGFDFTTYWMSWVRQAPGKGLEWIG |
| | EIHPDSSTINYAPSLKDRFTISRDNAKNTLFLQMD |
| | SLRPEDTGVYFCASLYFGFPWFAYWGQGTPVTVSS |
| | EQKLISEEDLTTTPAPRPPTPAPTIALQPLSLRPE |
| | ACRPAAGGAVHTRGLDFACDFWVLVVVGGVLACYS |
| | LLVTVAFIIFWVRSKRSRLLHSDYMNMTPRRPGPT |
| | RKHYQPYAPPRDFAAYRSRVKFSRSADAPAYKQGQ |
| | NQLYNELNLGRREEYDVLDKRRGRDPEMGGKPRRK |
| | NPQEGLYNELQKDKMAEAYSEIGMKGERRRGKGHD |
| | GLYQGLSTATKDTYDALHMQALPPR |
| | (SEQ ID NO: 81) |
| SB02465 | ATGGCTCTGCCTGTGACAGCTCTGCTGCTGCCTCT |
| & | GGCTCTGCTTCTGCATGCCGCTAGACCCCAAGTGA |
| SB03176 | AGCTGGAACAGTCTGGCGCCGAGGTTGTGAAACCT |
| DNA Seq | GGCGCCTCTGTGAAGCTGAGCTGTAAAGCCAGCGG |
| | CTTCAACATCAAGGACAGCTACATGCACTGGCTGC |
| | GGCAAGGCCCTGGACAGAGACTGGAATGGATCGGC |
| | TGGATCGACCCCGAGAATGGCGATACAGAGTACGC |
| | CCCTAAGTTCCAGGGCAAAGCCACCTTCACCACCG |
| | ACACCTCTGCCAACACAGCCTACCTGGGACTGTCT |
| | AGCCTGAGGCCTGAAGATACCGCCGTGTACTACTG |
| | CAACGAGGGCACACCTACAGGCCCCTACTACTTCG |
| | ATTATTGGGGCCAGGGCACCCTGGTCACCGTTTCT |
| | AGCGGAGGCGGAGGATCTGGTGGCGGAGGAAGTGG |
| | CGGAGGCGGTTCTGAAAATGTGCTGACACAGAGCC |
| | CCAGCAGCATGTCCGTGTCTGTGGGCGACAGAGTG |
| | ACAATCGCCTGTAGCGCCTCTAGCAGCGTGCCCTA |
| | TATGCATTGGCTGCAGCAGAAGCCCGGCAAGAGCC |
| | CTAAGCTGCTGATCTACCTGACCAGCAATCTGGCC |
| | AGCGGCGTGCCAAGCAGATTTTCTGGAAGCGGCAG |
| | CGGCACCGACTACAGCCTGACAATCAGTAGCGTGC |
| | AGCCTGAGGATGCCGCCACCTACTACTGTCAGCAG |
| | AGAAGCAGCTACCCTCTGACCTTTGGAGGCGGCAC |
| | CAAGCTGGAAATCAAAGAGCAGAAGCTGATCTCCG |
| | AAGAGGACCTGACCACAACACCAGCTCCTAGACCA |
| | CCTACACCAGCACCTACAATCGCCCTGCAGCCACT |
| | GTCTCTGAGGCCAGAAGCTTGTAGACCTGCTGCTG |
| | GCGGAGCCGTGCATACAAGAGGACTGGATTTCGCC |
| | TGCGACTTCTGGGTGCTCGTGGTTGTTGGCGGAGT |
| | GCTGGCCTGTTACTCTCTGCTGGTCACAGTGGCCT |
| | TCATCATCTTTTGGGTCCGAAGCAAGCGGAGCCGG |
| | CTGCTGCACAGCGACTACATGAACATGACCCCTAG |
| | ACGGCCCGGACCTACCAGAAAGCACTACCAGCCTT |
| | ACGCTCCTCCTAGAGACTTCGCCGCCTACCGGTCC |
| | AGAGTGAAGTTCAGCAGATCCGCCGATGCTCCCGC |
| | CTATAAGCAGGGCCAGAACCAGCTGTACAACGAGC |
| | TGAACCTGGGGAGAAGAGAAGAGTACGACGTGCTG |
| | GACAAGCGGAGAGGCAGAGATCCTGAGATGGGCGG |
| | CAAGCCCAGACGGAAGAATCCTCAAGAGGGCCTGT |
| | ATAATGAGCTGCAGAAAGACAAGATGGCCGAGGCC |
| | TACAGCGAGATCGGAATGAAGGGCGAGCGCAGAAG |
| | AGGCAAGGGACACGATGGACTGTATCAGGGCCTGA |
| | GCACCGCCACCAAGGATACCTATGATGCCCTGCAC |
| | ATGCAGGCCCTGCCTCCAAGA |
| | (SEQ ID NO: 82) |
| SB02465 | MALPVTALLLPLALLLHAARPENVLTQSPSSMSVS |
| & | VGDRVTIACSASSSVPYMHWLQQKPGKSPKLLIYL |
| SB03176 | TSNLASGVPSRFSGSGSGTDYSLTISSVQPEDAAT |
| A.A. Seq | YYCQQRSSYPLTFGGGTKLEIKEQKLISEEDLTTT |
| | PAPRPPTPAPTIALQPLSLRPEACRPAAGGAVHTR |
| | GLDFACDFWVLVVVGGVLACYSLLVTVAFIIFWVR |
| | SKRSRLLHSDYMNMTPRRPGPTRKHYQPYAPPRDF |
| | AAYRSRVKFSRSADAPAYKQGQNQLYNELNLGRRE |
| | EYDVLDKRRGRDPEMGGKPRRKNPQEGLYNELQKD |
| | KMAEAYSEIGMKGERRRGKGHDGLYQGLSTATKDT |
| | YDALHMQALPPR |
| | (SEQ ID NO: 83) |
| SB02467 | ATGGCTCTGCCTGTGACAGCTCTGCTGCTGCCTCT |
| DNA Seq | GGCTCTGCTTCTGCATGCTGCCAGACCTCAGATCG |
| | TGCTGTCTCAGTCTCCCGCCATCCTGTTTGCTAGC |
| | CCTGGCGAGAAAGTGACCATGACCTGTAGAGCCAG |
| | CAGCTCCGTGTCCTACATCCACTGGTATCAGCAGA |
| | AGCCCGGCAGCAGCCCTAAGCCTTGGATCCACGGC |
| | ACAAGCAATCTGGCCAGCGGAGTGCCTGCCAGATT |
| | TTCTGGAAGCGGCAGCGGCACCAGCTACAGCCTGA |
| | CCATCTCTAGAATGGAAGCCGAGGACGCCGCCACC |
| | TACTACTGTCAGCAGTGGTCCAGCAACCTGAGCAC |
| | CTTTGGCGGCGGAACAAAGCTGGAAATCAAAGGCG |
| | GCGGAGGATCTGGCGGAGGCGGTAGCGGAGGTGGT |
| | GGATCTGAAGTGAAGCTGGTGGAATCAGGCGGAGG |
| | CCTGGTTCAACCTGGCGGATCTCTGAGACTGAGCT |
| | GCAGCATCTCCGGCTTCACCTTCACCGACTACTAC |
| | ATGAACTGGGTCCGACAGAGCCCTGGCAAGGCTCT |
| | GGAATGGCTGGGCTTCATCAGAAACAAAGTGAACG |
| | GCGACACCACCGAGTACAGCGCCTCTGTGAAGGGC |
| | AGATTCACCATCAGCCGGGACATCAGCCAGAGCAT |
| | CCTGTACCTGCAGATGAACACCCTGCGGACCGAGG |
| | ATAGCGCCACATATTACTGCGCCAGAGACAAGGGA |
| | ATCGCCTACTACTTCGACTACTGGGGCCAGGGCAC |
| | CACACTGACAGTGTCTAGCGAGCAGAAGCTGATCT |
| | CCGAAGAGGACCTGACCACAACACCAGCTCCTAGA |
| | CCACCTACACCAGCACCTACAATCGCCCTGCAGCC |
| | ACTGTCTCTGAGGCCAGAAGCTTGTAGACCTGCTG |
| | CTGGCGGAGCCGTGCATACAAGAGGACTGGATTTC |
| | GCCTGCGACTTCTGGGTGCTCGTGGTTGTTGGCGG |
| | AGTGCTGGCCTGTTACTCTCTGCTGGTCACCGTGG |
| | CCTTCATCATCTTTTGGGTCCGAAGCAAGCGGAGC |
| | CGGCTGCTGCACAGCGATTACATGAACATGACCCC |
| | TCGGAGGCCCGGACCTACCAGAAAGCACTACCAGC |
| | CTTACGCTCCTCCTAGAGACTTCGCCGCCTACCGG |
| | TCCAGAGTGAAGTTCAGCAGATCCGCTGATGCCCC |
| | TGCCTACAAGCAGGGCCAGAACCAGCTGTACAACG |
| | AGCTGAACCTGGGGAGAAGAGAAGAGTACGACGTG |
| | CTGGACAAGCGGAGAGGCAGAGATCCTGAGATGGG |
| | CGGCAAGCCCAGACGGAAGAATCCTCAAGAGGGCC |
| | TGTATAACGAACTGCAGAAAGACAAGATGGCCGAG |
| | GCCTACAGCGAGATCGGAATGAAGGGCGAGCGCAG |
| | AAGAGGCAAGGGACACGATGGACTGTACCAGGGCC |
| | TGAGCACAGCCACCAAGGATACCTATGACGCCCTG |
| | CACATGCAGGCCCTGCCTCCAAGA |
| | (SEQ ID NO: 84) |
| SB02467 | MALPVTALLLPLALLLHAARPEVKLVESGGGLVQP |
| A.A. Seq | GGSLRLSCSISGFTFTDYYMNWVRQSPGKALEWLG |
| | FIRNKVNGDTTEYSASVKGRFTISRDISQSILYLQ |
| | MNTLRTEDSATYYCARDKGIAYYFDYWGQGTTLTV |
| | SSEQKLISEEDLTTTPAPRPPTPAPTIALQPLSLR |
| | PEACRPAAGGAVHTRGLDFACDFWVLVVVGGVLAC |
| | YSLLVTVAFIIFWVRSKRSRLLHSDYMNMTPRRPG |
| | PTRKHYQPYAPPRDFAAYRSRVKFSRSADAPAYKQ |
| | GQNQLYNELNLGRREEYDVLDKRRGRDPEMGGKPR |
| | RKNPQEGLYNELQKDKMAEAYSEIGMKGERRRGKG |
| | HDGLYQGLSTATKDTYDALHMQALPPR |
| | (SEQ ID NO: 85) |
| SB02470 | MALPVTALLLPLALLLHAARPDIQMTQTTSSLSAS |
| A.A. Seq | LGDRVTISCRTSQDIGNYLNWYQQKPDGTVKLLIY |
| | YTSRLHSGVPSRFSGSGSGTDYSLTISNLEQEDIA |
| | TYFCQQGKSLPRTFGGGTKLEIKEQKLISEEDLTT |
| | TPAPRPPTPAPTIALQPLSLRPEACRPAAGGAVHT |
| | RGLDFACDFWVLVVVGGVLACYSLLVTVAFIIFWV |
| | RSKRSRLLHSDYMNMTPRRPGPTRKHYQPYAPPRD |
| | FAAYRSRVKFSRSADAPAYKQGQNQLYNELNLGRR |
| | EEYDVLDKRRGRDPEMGGKPRRKNPQEGLYNELQK |
| | DKMAEAYSEIGMKGERRRGKGHDGLYQGLSTATKD |
| | TYDALHMQALPPR |
| | (SEQ ID NO: 86) |
| SB02476 | ATGGCTCTGCCTGTGACAGCTCTGCTGCTGCCTCT |
| DNA Seq | GGCTCTGCTTCTGCATGCTGCTAGACCTCAGGTCC |
| | AGCTGCAGCAGTCTGGCGCCGAACTTGTTAGACCT |
| | GGCACCAGCGTGAAGGTGTCCTGTAAAGCCAGCGG |
| | CTACGCCTTCACCAACAACCTGATCGAGTGGGTCA |
| | AGCAGAGGCCTGGACAGGGACTCGAGTGGATCGGC |
| | GTGATCAATCCTGGCAGCGGCGACACCAACTACAA |
| | CGAGAAGTTCAAGGGCAAAGCCACACTGACCGCCG |
| | ACAAGAGCAGCAACACAGCCTACATGCAGCTGAGC |
| | AGCCTGACCTCCGATGACAGCGCCGTGTACTTTTG |
| | CGCCAGAGGCGATTACTACGGCGGCTTCGCCGTGG |
| | ATTATTGGGGCCAGGGAACAAGCGTGACCGTGTCT |
| | AGCGGAGGCGGAGGATCTGGTGGCGGAGGAAGTGG |
| | CGGAGGCGGTTCTGATATCCAGATGACCCAGACCA |
| | CCAGCAGCCTGTCTGCCTCTCTGGGCGATAGAGTG |
| | ACCATCAGCTGCAGAACCAGCCAGGACATCGGCAA |
| | CTACCTGAACTGGTATCAGCAGAAACCCGACGGCA |
| | CCGTGAAGCTGCTGATCTACTACACCAGCAGACTG |
| | CACAGCGGCGTGCCCAGCAGATTTTCTGGAAGCGG |
| | CAGCGGCACCGACTACTCCCTGACAATCAGCAACC |
| | TGGAACAAGAGGATATCGCTACCTACTTCTGCCAG |
| | CAAGGCAAGAGCCTGCCTAGAACCTTTGGAGGCGG |
| | CACCAAGCTGGAAATCAAAGAGCAGAAGCTGATCT |
| | CCGAAGAGGACCTGGAGTCTAAGTACGGCCCTCCT |
| | TGTCCTAGCTGCCCCTTTTGGGTGCTCGTGGTTGT |
| | TGGCGGAGTGCTGGCCTGTTACTCTCTGCTGGTCA |
| | CCGTGGCCTTCATCATCTTTTGGGTCCGAAGCAAG |
| | CGGAGCCGGCTGCTGCACTCCGACTACATGAACAT |
| | GACCCCTAGACGGCCCGGACCTACCAGAAAGCACT |
| | ACCAGCCTTACGCTCCTCCTAGAGACTTCGCCGCC |
| | TACCGGTCCAGAGTGAAGTTCAGCAGATCCGCCGA |
| | TGCTCCCGCCTATAAGCAGGGCCAGAATCAGCTGT |
| | ACAACGAGCTGAACCTGGGGCGCAGAGAAGAGTAC |
| | GACGTGCTGGACAAGAGAAGAGGCAGGGACCCTGA |
| | GATGGGCGGCAAGCCCAGAAGAAAGAACCCTCAAG |
| | AGGGCCTGTATAACGAACTGCAGAAAGACAAGATG |
| | GCCGAGGCCTACAGCGAGATCGGAATGAAGGGCGA |
| | ACGCAGAAGAGGAAAGGGCCACGACGGACTGTATC |
| | AGGGCCTGAGCACAGCCACCAAGGACACCTATGAT |
| | GCCCTGCACATGCAGGCCCTGCCTCCAAGA |
| | (SEQ ID NO: 87) |
| SB02476 | MALPVTALLLPLALLLHAARPDIQMTQTTSSLSAS |
| A.A. Seq | LGDRVTISCRTSQDIGNYLNWYQQKPDGTVKLLIY |
| | YTSRLHSGVPSRFSGSGSGTDYSLTISNLEQEDIA |
| | TYFCQQGKSLPRTFGGGTKLEIKEQKLISEEDLES |
| | KYGPPCPSCPFWVLVVVGGVLACYSLLVTVAFIIF |
| | WVRSKRSRLLHSDYMNMTPRRPGPTRKHYQPYAPP |
| | RDFAAYRSRVKFSRSADAPAYKQGQNQLYNELNLG |
| | RREEYDVLDKRRGRDPEMGGKPRRKNPQEGLYNEL |
| | QKDKMAEAYSEIGMKGERRRGKGHDGLYQGLSTAT |
| | KDTYDALHMQALPPR |
| | (SEQ ID NO: 88) |
| SB02779 | ATGGCTCTGCCTGTGACAGCTCTGCTGCTGCCTCT |
| & | GGCTCTGCTTCTGCATGCCGCCAGACCTGACATCC |
| SB03174 | AGCTGACACAGAGCCCTAGCAGCCTGTCTGCCTCT |
| DNA Seq | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAGCAGAAGCTGATCTCCGAAG |
| | AGGACCTGACCACAACACCAGCTCCTAGACCACCT |
| | ACACCAGCACCTACAATCGCCCTGCAGCCACTGTC |
| | TCTGAGGCCAGAGGCTTGTAGACCTGCTGCTGGCG |
| | GAGCCGTGCATACAAGAGGACTGGATTTCGCCTGC |
| | GACGTGGCCGCCATTCTCGGACTGGGACTTGTTCT |
| | GGGACTGCTGGGACCTCTGGCCATTCTGCTGGCTC |
| | TGTACCTGCTGAGAAGGGACCAGAGACTGCCTCCT |
| | GACGCTCACAAACCTCCAGGCGGAGGCAGCTTCAG |
| | AACCCCTATCCAAGAGGAACAGGCTGACGCCCACA |
| | GCACCCTGGCCAAGATCAGAGTGAAGTTCAGCAGA |
| | TCCGCCGACGCTCCCGCCTATAAGCAGGGACAGAA |
| | TCAGCTGTACAACGAGCTGAACCTGGGGCGCAGAG |
| | AAGAGTACGACGTGCTGGACAAGAGAAGAGGCAGG |
| | GATCCTGAAATGGGCGGCAAGCCCAGACGGAAGAA |
| | TCCTCAAGAGGGCCTGTATAATGAGCTGCAGAAAG |
| | ACAAGATGGCCGAGGCCTACAGCGAGATCGGAATG |
| | AAGGGCGAACGCAGAAGAGGAAAGGGCCACGACGG |
| | ACTGTATCAGGGCCTGAGCACAGCCACCAAGGACA |
| | CCTATGATGCCCTGCACATGCAGGCCCTGCCTCCA |
| | AGA |
| | (SEQ ID NO: 136) |
| SB02779 | MALPVTALLLPLALLLHAARPDIQLTQSPSSLSAS |
| & | VGDRVTITCKASQDVGTSVAWYQQKPGKAPKLLIY |
| SB03174 | WTSTRHTGVPSRFSGSGSGTDFTFTISSLQPEDIA |
| A.A. Seq | TYYCQQYSLYRSFGQGTKVEIKGGSGSGGSGSGGS |
| | GSEVOLVESGGGVVQPGRSLRLSCSASGFDFTTYW |
| | MSWVRQAPGKGLEWIGEIHPDSSTINYAPSLKDRF |
| | TISRDNAKNTLFLQMDSLRPEDTGVYFCASLYFGF |
| | PWFAYWGQGTPVTVSSEQKLISEEDLTTTPAPRPP |
| | TPAPTIALQPLSLRPEACRPAAGGAVHTRGLDFAC |
| | DVAAILGLGLVLGLLGPLAILLALYLLRRDQRLPP |
| | DAHKPPGGGSFRTPIQEEQADAHSTLAKIRVKFSR |
| | SADAPAYKQGQNQLYNELNLGRREEYDVLDKRRGR |
| | DPEMGGKPRRKNPQEGLYNELQKDKMAEAYSEIGM |
| | KGERRRGKGHDGLYQGLSTATKDTYDALHMQALPP |
| | R |
| | (SEQ ID NO: 89) |
| SB02782 | ATGGCTCTGCCTGTGACAGCTCTGCTGCTGCCTCT |
| DNA Seq | GGCTCTGCTTCTGCATGCCGCTAGACCCCAAGTGA |
| | AGCTGGAACAGTCTGGCGCCGAGGTTGTGAAACCT |
| | GGCGCCTCTGTGAAGCTGAGCTGTAAAGCCAGCGG |
| | CTTCAACATCAAGGACAGCTACATGCACTGGCTGC |
| | GGCAAGGCCCTGGACAGAGACTGGAATGGATCGGC |
| | TGGATCGACCCCGAGAATGGCGATACAGAGTACGC |
| | CCCTAAGTTCCAGGGCAAAGCCACCTTCACCACCG |
| | ACACCTCTGCCAACACAGCCTACCTGGGACTGTCT |
| | AGCCTGAGGCCTGAAGATACCGCCGTGTACTACTG |
| | CAACGAGGGCACACCTACAGGCCCCTACTACTTCG |
| | ATTATTGGGGCCAGGGCACCCTGGTCACCGTTTCT |
| | AGCGGAGGCGGAGGATCTGGTGGCGGAGGAAGTGG |
| | CGGAGGCGGTTCTGAAAATGTGCTGACACAGAGCC |
| | CCAGCAGCATGTCCGTGTCTGTGGGCGACAGAGTG |
| | ACAATCGCCTGTAGCGCCTCTAGCAGCGTGCCCTA |
| | TATGCATTGGCTGCAGCAGAAGCCCGGCAAGAGCC |
| | CTAAGCTGCTGATCTACCTGACCAGCAATCTGGCC |
| | AGCGGCGTGCCAAGCAGATTTTCTGGAAGCGGCAG |
| | CGGCACCGACTACAGCCTGACAATCAGTAGCGTGC |
| | AGCCTGAGGATGCCGCCACCTACTACTGTCAGCAG |
| | AGAAGCAGCTACCCTCTGACCTTTGGAGGCGGCAC |
| | CAAGCTGGAAATCAAAGAGCAGAAGCTGATCTCCG |
| | AAGAGGACCTGACCACAACACCAGCTCCTAGACCA |
| | CCTACACCAGCACCTACAATCGCCCTGCAGCCACT |
| | GTCTCTGAGGCCAGAAGCTTGTAGACCTGCTGCTG |
| | GCGGAGCCGTGCATACAAGAGGACTGGATTTCGCC |
| | TGCGACGTGGCCGCCATTCTCGGACTGGGACTTGT |
| | TCTGGGACTGCTGGGACCTCTGGCCATTCTGCTGG |
| | CTCTGTACCTGCTGAGAAGGGACCAGAGACTGCCT |
| | CCTGACGCTCACAAACCTCCAGGCGGAGGCAGCTT |
| | CAGAACCCCTATCCAAGAGGAACAGGCTGACGCCC |
| | ACAGCACCCTGGCCAAGATCAGAGTGAAGTTCAGC |
| | AGATCCGCCGACGCTCCCGCCTATAAGCAGGGACA |
| | GAATCAGCTGTACAACGAGCTGAACCTGGGGCGCA |
| | GAGAAGAGTACGACGTGCTGGACAAGAGAAGAGGC |
| | AGGGATCCTGAAATGGGCGGCAAGCCCAGACGGAA |
| | GAATCCTCAAGAGGGCCTGTATAATGAGCTGCAGA |
| | AAGACAAGATGGCCGAGGCCTACAGCGAGATCGGA |
| | ATGAAGGGCGAACGCAGAAGAGGAAAGGGCCACGA |
| | CGGACTGTATCAGGGCCTGAGCACAGCCACCAAGG |
| | ACACCTATGATGCCCTGCACATGCAGGCCCTGCCT |
| | CCAAGA |
| | (SEQ ID NO: 90) |
| SB02782 | MALPVTALLLPLALLLHAARPQVKLEQSGAEVVKP |
| A.A. Seq | GASVKLSCKASGFNIKDSYMHWLRQGPGQRLEWIG |
| | WIDPENGDTEYAPKFQGKATFTTDTSANTAYLGLS |
| | SLRPEDTAVYYCNEGTPTGPYYFDYWGQGTLVTVS |
| | SGGGGSGGGGSGGGGSENVLTQSPSSMSVSVGDRV |
| | TIACSASSSVPYMHWLQQKPGKSPKLLIYLTSNLA |
| | SGVPSRFSGSGSGTDYSLTISSVQPEDAATYYCQQ |
| | RSSYPLTFGGGTKLEIKEQKLISEEDLTTTPAPRP |
| | PTPAPTIALQPLSLRPEACRPAAGGAVHTRGLDFA |
| | CDVAAILGLGLVLGLLGPLAILLALYLLRRDQRLP |
| | PDAHKPPGGGSFRTPIQEEQADAHSTLAKIRVKFS |
| | RSADAPAYKQGQNQLYNELNLGRREEYDVLDKRRG |
| | RDPEMGGKPRRKNPQEGLYNELQKDKMAEAYSEIG |
| | MKGERRRGKGHDGLYQGLSTATKDTYDALHMQALP |
| | PR |
| | (SEQ ID NO: 91) |
| SB02785 | ATGGCTCTGCCTGTGACAGCTCTGCTGCTGCCTCT |
| DNA Seq | GGCTCTGCTTCTGCATGCTGCCAGACCTCAGATCG |
| | TGCTGTCTCAGTCTCCCGCCATCCTGTTTGCTAGC |
| | CCTGGCGAGAAAGTGACCATGACCTGTAGAGCCAG |
| | CAGCTCCGTGTCCTACATCCACTGGTATCAGCAGA |
| | AGCCCGGCAGCAGCCCTAAGCCTTGGATCCACGGC |
| | ACAAGCAATCTGGCCAGCGGAGTGCCTGCCAGATT |
| | TTCTGGAAGCGGCAGCGGCACCAGCTACAGCCTGA |
| | CCATCTCTAGAATGGAAGCCGAGGACGCCGCCACC |
| | TACTACTGTCAGCAGTGGTCCAGCAACCTGAGCAC |
| | CTTTGGCGGCGGAACAAAGCTGGAAATCAAAGGCG |
| | GCGGAGGATCTGGCGGAGGCGGTAGCGGAGGTGGT |
| | GGATCTGAAGTGAAGCTGGTGGAATCAGGCGGAGG |
| | CCTGGTTCAACCTGGCGGATCTCTGAGACTGAGCT |
| | GCAGCATCTCCGGCTTCACCTTCACCGACTACTAC |
| | ATGAACTGGGTCCGACAGAGCCCTGGCAAGGCTCT |
| | GGAATGGCTGGGCTTCATCAGAAACAAAGTGAACG |
| | GCGACACCACCGAGTACAGCGCCTCTGTGAAGGGC |
| | AGATTCACCATCAGCCGGGACATCAGCCAGAGCAT |
| | CCTGTACCTGCAGATGAACACCCTGCGGACCGAGG |
| | ATAGCGCCACATATTACTGCGCCAGAGACAAGGGA |
| | ATCGCCTACTACTTCGACTACTGGGGCCAGGGCAC |
| | CACACTGACAGTGTCTAGCGAGCAGAAGCTGATCT |
| | CCGAAGAGGACCTGACCACAACACCAGCTCCTAGA |
| | CCACCTACACCAGCACCTACAATCGCCCTGCAGCC |
| | ACTGTCTCTGAGGCCAGAAGCTTGTAGACCTGCTG |
| | CTGGCGGAGCCGTGCATACAAGAGGACTGGATTTC |
| | GCCTGCGACGTGGCCGCCATTCTCGGACTGGGACT |
| | TGTTCTGGGACTGCTGGGACCTCTGGCCATTCTGC |
| | TGGCTCTGTACCTGCTGAGAAGGGACCAGAGACTG |
| | CCTCCTGACGCTCACAAACCTCCAGGCGGAGGCAG |
| | CTTCAGAACCCCTATCCAAGAGGAACAGGCTGACG |
| | CCCACAGCACCCTGGCCAAGATCAGAGTGAAGTTC |
| | AGCAGATCCGCCGACGCTCCCGCCTATAAGCAGGG |
| | ACAGAATCAGCTGTACAACGAGCTGAACCTGGGGC |
| | GCAGAGAAGAGTACGACGTGCTGGACAAGAGAAGA |
| | GGCAGGGATCCTGAAATGGGCGGCAAGCCCAGACG |
| | GAAGAATCCTCAAGAGGGCCTGTATAATGAGCTGC |
| | AGAAAGACAAGATGGCCGAGGCCTACAGCGAGATC |
| | GGAATGAAGGGCGAACGCAGAAGAGGAAAGGGCCA |
| | CGACGGACTGTATCAGGGCCTGAGCACAGCCACCA |
| | AGGACACCTATGATGCCCTGCACATGCAGGCCCTG |
| | CCTCCAAGA |
| | (SEQ ID NO: 92) |
| SB02785 | MALPVTALLLPLALLLHAARPQIVLSQSPAILFAS |
| A.A. Seq | PGEKVTMTCRASSSVSYIHWYQQKPGSSPKPWIHG |
| | TSNLASGVPARFSGSGSGTSYSLTISRMEAEDAAT |
| | YYCQQWSSNLSTFGGGTKLEIKGGGGSGGGGSGGG |
| | GSEVKLVESGGGLVQPGGSLRLSCSISGFTFTDYY |
| | MNWVRQSPGKALEWLGFIRNKVNGDTTEYSASVKG |
| | RFTISRDISQSILYLQMNTLRTEDSATYYCARDKG |
| | IAYYFDYWGQGTTLTVSSEQKLISEEDLTTTPAPR |
| | PPTPAPTIALQPLSLRPEACRPAAGGAVHTRGLDF |
| | ACDVAAILGLGLVLGLLGPLAILLALYLLRRDQRL |
| | PPDAHKPPGGGSFRTPIQEEQADAHSTLAKIRVKF |
| | SRSADAPAYKQGQNQLYNELNLGRREEYDVLDKRR |
| | GRDPEMGGKPRRKNPQEGLYNELQKDKMAEAYSEI |
| | GMKGERRRGKGHDGLYQGLSTATKDTYDALHMQAL |
| | PPR |
| | (SEQ ID NO: 93) |
| SB02788 | ATGGCTCTGCCTGTGACAGCTCTGCTGCTGCCTCT |
| & | GGCTCTGCTTCTGCATGCTGCTAGACCTCAGGTCC |
| SB03178 | AGCTGCAGCAGTCTGGCGCCGAACTTGTTAGACCT |
| DNA Seq | GGCACCAGCGTGAAGGTGTCCTGTAAAGCCAGCGG |
| | CTACGCCTTCACCAACAACCTGATCGAGTGGGTCA |
| | AGCAGAGGCCTGGACAGGGACTCGAGTGGATCGGC |
| | GTGATCAATCCTGGCAGCGGCGACACCAACTACAA |
| | CGAGAAGTTCAAGGGCAAAGCCACACTGACCGCCG |
| | ACAAGAGCAGCAACACAGCCTACATGCAGCTGAGC |
| | AGCCTGACCTCCGATGACAGCGCCGTGTACTTTTG |
| | CGCCAGAGGCGATTACTACGGCGGCTTCGCCGTGG |
| | ATTATTGGGGCCAGGGAACAAGCGTGACCGTGTCT |
| | AGCGGAGGCGGAGGATCTGGTGGCGGAGGAAGTGG |
| | CGGAGGCGGTTCTGATATCCAGATGACCCAGACCA |
| | CCAGCAGCCTGTCTGCCTCTCTGGGCGATAGAGTG |
| | ACCATCAGCTGCAGAACCAGCCAGGACATCGGCAA |
| | CTACCTGAACTGGTATCAGCAGAAACCCGACGGCA |
| | CCGTGAAGCTGCTGATCTACTACACCAGCAGACTG |
| | CACAGCGGCGTGCCCAGCAGATTTTCTGGAAGCGG |
| | CAGCGGCACCGACTACTCCCTGACAATCAGCAACC |
| | TGGAACAAGAGGATATCGCTACCTACTTCTGCCAG |
| | CAAGGCAAGAGCCTGCCTAGAACCTTTGGAGGCGG |
| | CACCAAGCTGGAAATCAAAGAGCAGAAGCTGATCT |
| | CCGAAGAGGACCTGACCACAACACCAGCTCCTAGA |
| | CCTCCAACTCCTGCTCCTACAATCGCCCTGCAGCC |
| | ACTGTCTCTGAGGCCTGAAGCTTGTAGACCTGCTG |
| | CTGGCGGAGCCGTGCATACCAGAGGACTGGATTTC |
| | GCCTGCGACGTGGCCGCCATTCTCGGACTGGGACT |
| | TGTTCTGGGACTGCTGGGACCTCTGGCCATTCTGC |
| | TGGCTCTGTACCTGCTGAGAAGGGACCAGAGACTG |
| | CCTCCTGACGCTCACAAACCTCCAGGCGGAGGCAG |
| | CTTCAGAACCCCTATCCAAGAGGAACAGGCTGACG |
| | CCCACAGCACCCTGGCCAAGATCAGAGTGAAGTTC |
| | AGCAGATCCGCCGACGCTCCCGCCTATAAGCAGGG |
| | ACAGAATCAGCTGTACAACGAGCTGAACCTGGGGC |
| | GCAGAGAAGAGTACGACGTGCTGGACAAGAGAAGA |
| | GGCAGGGATCCTGAAATGGGCGGCAAGCCCAGACG |
| | GAAGAATCCTCAAGAGGGCCTGTATAATGAGCTGC |
| | AGAAAGACAAGATGGCCGAGGCCTACAGCGAGATC |
| | GGAATGAAGGGCGAACGCAGAAGAGGAAAGGGCCA |
| | CGACGGACTGTATCAGGGCCTGAGCACAGCCACCA |
| | AGGACACCTATGATGCCCTGCACATGCAGGCCCTG |
| | CCTCCAAGA |
| | (SEQ ID NO: 94) |
| SB02788 | MALPVTALLLPLALLLHAARPQVQLQQSGAELVRP |
| & | GTSVKVSCKASGYAFINNLIEWVKQRPGQGLEWIG |
| SB03178 | VINPGSGDTNYNEKFKGKATLTADKSSNTAYMQLS |
| A.A. Seq | SLTSDDSAVYFCARGDYYGGFAVDYWGQGTSVTVS |
| | SGGGGSGGGGSGGGGSDIQMTQTTSSLSASLGDRV |
| | TISCRTSQDIGNYLNWYQQKPDGTVKLLIYYTSRL |
| | HSGVPSRFSGSGSGTDYSLTISNLEQEDIATYFCQ |
| | QGKSLPRTFGGGTKLEIKEQKLISEEDLTTTPAPR |
| | PPTPAPTIALQPLSLRPEACRPAAGGAVHTRGLDF |
| | ACDVAAILGLGLVLGLLGPLAILLALYLLRRDQRL |
| | PPDAHKPPGGGSFRTPIQEEQADAHSTLAKIRVKF |
| | SRSADAPAYKQGQNQLYNELNLGRREEYDVLDKRR |
| | GRDPEMGGKPRRKNPQEGLYNELQKDKMAEAYSEI |
| | GMKGERRRGKGHDGLYQGLSTATKDTYDALHMQAL |
| | PPR |
| | (SEQ ID NO: 95) |
| SB02779 | ATGGCTCTGCCTGTGACAGCTCTGCTGCTGCCTCT |
| DNA Seq | GGCTCTGCTTCTGCATGCCGCCAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAGCAGAAGCTGATCTCCGAAG |
| | AGGACCTGACCACAACACCAGCTCCTAGACCACCT |
| | ACACCAGCACCTACAATCGCCCTGCAGCCACTGTC |
| | TCTGAGGCCAGAGGCTTGTAGACCTGCTGCTGGCG |
| | GAGCCGTGCATACAAGAGGACTGGATTTCGCCTGC |
| | GACGTGGCCGCCATTCTCGGACTGGGACTTGTTCT |
| | GGGACTGCTGGGACCTCTGGCCATTCTGCTGGCTC |
| | TGTACCTGCTGAGAAGGGACCAGAGACTGCCTCCT |
| | GACGCTCACAAACCTCCAGGCGGAGGCAGCTTCAG |
| | AACCCCTATCCAAGAGGAACAGGCTGACGCCCACA |
| | GCACCCTGGCCAAGATCAGAGTGAAGTTCAGCAGA |
| | TCCGCCGACGCTCCCGCCTATAAGCAGGGACAGAA |
| | TCAGCTGTACAACGAGCTGAACCTGGGGCGCAGAG |
| | AAGAGTACGACGTGCTGGACAAGAGAAGAGGCAGG |
| | GATCCTGAAATGGGCGGCAAGCCCAGACGGAAGAA |
| | TCCTCAAGAGGGCCTGTATAATGAGCTGCAGAAAG |
| | ACAAGATGGCCGAGGCCTACAGCGAGATCGGAATG |
| | AAGGGCGAACGCAGAAGAGGAAAGGGCCACGACGG |
| | ACTGTATCAGGGCCTGAGCACAGCCACCAAGGACA |
| | CCTATGATGCCCTGCACATGCAGGCCCTGCCTCCA |
| | AGA |
| | (SEQ ID NO: 96) |
| SB02779 | MALPVTALLLPLALLLHAARPDIQLTQSPSSLSAS |
| A.A. Seq | VGDRVTITCKASQDVGTSVAWYQQKPGKAPKLLIY |
| | WTSTRHTGVPSRFSGSGSGTDFTFTISSLQPEDIA |
| | TYYCQQYSLYRSFGQGTKVEIKGGSGSGGSGSGGS |
| | GSEVQLVESGGGVVQPGRSLRLSCSASGFDFTTYW |
| | MSWVRQAPGKGLEWIGEIHPDSSTINYAPSLKDRF |
| | TISRDNAKNTLFLQMDSLRPEDTGVYFCASLYFGF |
| | PWFAYWGQGTPVTVSSEQKLISEEDLTTTPAPRPP |
| | TPAPTIALQPLSLRPEACRPAAGGAVHTRGLDFAC |
| | DVAAILGLGLVLGLLGPLAILLALYLLRRDQRLPP |
| | DAHKPPGGGSFRTPIQEEQADAHSTLAKIRVKFSR |
| | SADAPAYKQGQNQLYNELNLGRREEYDVLDKRRGR |
| | DPEMGGKPRRKNPQEGLYNELQKDKMAEAYSEIGM |
| | KGERRRGKGHDGLYQGLSTATKDTYDALHMQALPP |
| | R |
| | (SEQ ID NO: 97) |
| SB03096 | ATGGCTCTGCCTGTGACAGCTCTGCTGCTGCCTCT |
| DNA Seq | GGCTCTGCTTCTGCATGCTGCCAGACCTCAGACCG |
| | TGCTGACACAGAGCCCAAGCAGCCTGTCTGTGTCC |
| | GTGGGCGACAGAGTGACCATCACCTGTAGAGCCAG |
| | CAGCTCCGTGACCTACATCCACTGGTATCAGCAGA |
| | AGCCCGGACTGGCCCCTAAGAGCCTGATCTACGCC |
| | ACAAGCAATCTGGCCAGCGGCGTGCCAAGCAGATT |
| | TTCTGGAAGCGGCAGCGGCACCGACTACACCTTCA |
| | CCATATCTAGCCTGCAGCCTGAGGATATCGCCACC |
| | TACTACTGCCAGCACTGGTCCAGCAAGCCTCCAAC |
| | ATTTGGCCAGGGCACCAAGGTGGAAGTGAAGAGAA |
| | CAGTTGGCGGCGGAGGATCTGGCGGAGGCGGTAGC |
| | GGAGGTGGTGGATCTGAAGTTCAGCTGCTGGAATC |
| | AGGCGGCGGACTGGTTCAACCTGGCGGATCTCTGA |
| | GACTGAGCTGTGCCACCAGCGGCTTCACCTTTACC |
| | GACTACTACATGAACTGGGTCCGACAGGCCCCTGG |
| | CAAAGGACTGGAATGGCTGGGCTTCATCGGCAACA |
| | AGGCCAACGGCTACACCACCGAGTACAGCGCCTCT |
| | GTGAAGGGCAGATTCACCATCAGCCGGGACAAGAG |
| | CAAGAGCACCCTGTACCTGCAGATGAACACCCTGC |
| | AGGCCGAGGACTCCGCCATCTACTATTGCACCAGA |
| | GACAGAGGCCTGCGGTTCTACTTCGACTATTGGGG |
| | CCAGGGAACCCTGGTCACAGTGTCTAGCGCCTCTA |
| | CAAAGGGCCCCAGCGTTTTCCCACTGGCTCCCTGT |
| | AGCAGAAGCACCAGCGAATCTACAGCCGCTCTGGG |
| | CTGCCTGGTCAAGGACTACTTTCCTGAGCCAGTGA |
| | CAGTGCTGTATCTCCAGATGAATACTCTCCAAGCT |
| | GAGGACAGCGCCATCTATTACTGTACGCGCGATCG |
| | GGGCCTGAGATTCTACTTTGATTACTGGGGACAAG |
| | GCACCCTCGTGACCGTGTCCTCTGAGCAGAAGCTG |
| | ATCTCCGAAGAGGACCTGAATGGCGCCGCTGCCGC |
| | TGCTATCGAAGTGATGTACCCTCCTCCTTACCTGG |
| | ACAACGAGAAGTCCAACGGCACCATCATCCACGTG |
| | AAGGGCAAGCACCTGTGTCCTTCTCCACTGTTCCC |
| | CGGACCTAGCAAGCCTTTCTGGGTGCTCGTTGTTG |
| | TTGGCGGCGTGCTGGCCTGTTATTCCCTGCTGGTT |
| | ACCGTGGCCTTCATCATCTTTTGGGTCCGAAGCAA |
| | GCGGAGCAGACTGCTGCACTCCGACTACATGAACA |
| | TGACCCCTAGACGGCCCGGACCAACCAGAAAGCAC |
| | TACCAGCCTTACGCTCCTCCTAGAGACTTCGCCGC |
| | CTACCGGTCCAGAGTGAAGTTCTCCAGATCCGCCG |
| | ATGCTCCCGCCTATAAGCAGGGACAGAACCAGCTG |
| | TACAACGAGCTGAACCTGGGGAGAAGAGAAGAGTA |
| | CGACGTGCTGGACAAGCGGAGAGGCAGAGATCCTG |
| | AGATGGGCGGCAAGCCCAGACGGAAGAATCCTCAA |
| | GAGGGCCTGTATAATGAGCTGCAAAAGGACAAGAT |
| | GGCCGAGGCCTACAGCGAGATCGGAATGAAGGGCG |
| | AGCGCAGAAGAGGCAAGGGACACGATGGACTGTAC |
| | CAGGGCCTGAGCACCGCCACCAAGGATACCTATGA |
| | TGCCCTGCACATGCAGGCCCTGCCTCCAAGA |
| | (SEQ ID NO: 98) |
| SB03096 | MALPVTALLLPLALLLHAARPQTVLTQSPSSLSVS |
| A.A. Seq | VGDRVTITCRASSSVTYIHWYQQKPGLAPKSLIYA |
| | TSNLASGVPSRFSGSGSGTDYTFTISSLQPEDIAT |
| | YYCQHWSSKPPTFGQGTKVEVKRTVGGGGSGGGGS |
| | GGGGSEVQLLESGGGLVQPGGSLRLSCATSGFTFT |
| | DYYMNWVRQAPGKGLEWLGFIGNKANGYTTEYSAS |
| | VKGRFTISRDKSKSTLYLQMNTLQAEDSAIYYCTR |
| | DRGLRFYFDYWGQGTLVTVSSASTKGPSVFPLAPC |
| | SRSTSESTAALGCLVKDYFPEPVTVLYLQMNTLQA |
| | EDSAIYYCTRDRGLRFYFDYWGQGTLVTVSSEQKL |
| | ISEEDLNGAAAAAIEVMYPPPYLDNEKSNGTIIHV |
| | KGKHLCPSPLFPGPSKPFWVLVVVGGVLACYSLLV |
| | TVAFIIFWVRSKRSRLLHSDYMNMTPRRPGPTRKH |
| | YQPYAPPRDFAAYRSRVKFSRSADAPAYKQGQNQL |
| | YNELNLGRREEYDVLDKRRGRDPEMGGKPRRKNPQ |
| | EGLYNELQKDKMAEAYSEIGMKGERRRGKGHDGLY |
| | QGLSTATKDTYDALHMQALPPR |
| | (SEQ ID NO: 99) |
| SB03097 | ATGGCTCTGCCTGTGACAGCTCTGCTGCTGCCTCT |
| DNA Seq | GGCTCTGCTTCTGCATGCTGCCAGACCTGAGGTCC |
| | AGCTGCAAGAGTCTGGCCCTGGACTTGTGAAGCCT |
| | GGCGGAAGCCTGTCTCTGTCTTGTGCCGCCAGCGG |
| | CTTCGTGTTCAGCAGCTACGATATGAGCTGGGTCC |
| | GACAGACCCCTGAGCGAGGACTTGAGTGGGTCGCC |
| | TACATCTCTTCTGGCGGCGGAATCACATACGCCCC |
| | TAGCACAGTGAAGGGCAGATTCACCGTGTCCAGAG |
| | ACAACGCCAAGAACACCCTGTACCTGCAGATGAAC |
| | AGCCTGACCAGCGAGGACACCGCCGTGTACTATTG |
| | TGCCGCTCACTACTTTGGCAGCAGCGGCCCTTTTG |
| | CCTATTGGGGCCAGGGAACACTCGTGACCGTTTCT |
| | AGTGCTGGCGGAGGCGGATCAGGTGGCGGAGGAAG |
| | TGGCGGCGGAGGATCTGATATCCAGATGACACAGA |
| | GCCCCGCCAGCCTGTCTGCCTCTGTGGGAGATAGA |
| | GTGACCATCACCTGTCGGGCCAGCGAGAACATCTT |
| | CAGCTACCTGGCCTGGTATCAGCAGAAGCCCGGCA |
| | AGTCTCCTAAGCTGCTGGTGTACAACACCCGGACA |
| | CTGGCTGAAGGCGTGCCCAGCAGATTTTCTGGCAG |
| | CGGCTCTGGCACCGACTTCAGCCTGACAATCAGCA |
| | GCCTGCAGCCTGAGGACTTCGCCACCTACTACTGC |
| | CAGCACCACTACGGCACCCCTTTCACCTTTGGAAG |
| | CGGCACCAAGCTGGAAATCAAGAGAGAGCAGAAGC |
| | TGATCTCCGAAGAGGACCTGAATGGCGCCGCTACC |
| | ACAACACCAGCTCCTAGACCTCCAACTCCTGCTCC |
| | TACAATCGCCCTGCAGCCACTGTCTCTGAGGCCTG |
| | AAGCTTGTAGACCTGCTGCTGGCGGAGCCGTGCAT |
| | ACCAGAGGACTGGATTTCGCCTGCGACATCTACAT |
| | CTGGGCCCCTCTGGCTGGAACATGCGGAGTGTTGC |
| | TGCTGAGCCTGGTCATCACCAAGCGGGGCAGAAAG |
| | AAGCTGCTGTACATCTTCAAGCAGCCCTTCATGCG |
| | GCCCGTGCAGACCACACAAGAGGAAGATGGCTGCT |
| | CCTGCAGATTCCCCGAGGAAGAAGAAGGCGGCTGC |
| | GAGCTGAGAGTGAAGTTCAGCAGATCCGCCGACGC |
| | TCCTGCCTATCAGCAGGGACAGAACCAGCTGTACA |
| | ACGAGCTGAACCTGGGGAGAAGAGAAGAGTACGAC |
| | GTGCTGGACAAGCGGAGAGGCAGAGATCCTGAAAT |
| | GGGCGGCAAGCCCAGACGGAAGAATCCTCAAGAGG |
| | GCCTGTATAATGAGCTGCAGAAAGACAAGATGGCC |
| | GAGGCCTACAGCGAGATCGGAATGAAGGGCGAGCG |
| | CAGAAGAGGCAAGGGACACGATGGACTGTACCAGG |
| | GCCTGAGCACCGCCACCAAGGATACCTATGATGCC |
| | CTGCACATGCAGGCCCTGCCTCCAAGA |
| | (SEQ ID NO: 100) |
| SB03097 | MALPVTALLLPLALLLHAARPEVQLQESGPGLVKP |
| A.A. Seq | GGSLSLSCAASGFVFSSYDMSWVRQTPERGLEWVA |
| | YISSGGGITYAPSTVKGRFTVSRDNAKNTLYLQMN |
| | SLTSEDTAVYYCAAHYFGSSGPFAYWGQGTLVTVS |
| | SAGGGGSGGGGSGGGGSDIQMTQSPASLSASVGDR |
| | VTITCRASENIFSYLAWYQQKPGKSPKLLVYNTRT |
| | LAEGVPSRFSGSGSGTDFSLTISSLQPEDFATYYC |
| | QHHYGTPFTFGSGTKLEIKREQKLISEEDLNGAAT |
| | TTPAPRPPTPAPTIALQPLSLRPEACRPAAGGAVH |
| | TRGLDFACDIYIWAPLAGTCGVLLLSLVITKRGRK |
| | KLLYIFKQPFMRPVQTTQEEDGCSCRFPEEEEGGC |
| | ELRVKFSRSADAPAYQQGQNQLYNELNLGRREEYD |
| | VLDKRRGRDPEMGGKPRRKNPQEGLYNELQKDKMA |
| | EAYSEIGMKGERRRGKGHDGLYQGLSTATKDTYDA |
| | LHMQALPPR |
| | (SEQ ID NO: 101) |
| SB03098 | ACCTGACATCCAGATGACACAGAGCCCTGCCAGCC |
| DNA Seq | TGTCTGCCTCTGTGGGAGACAGAATGGCTCTGCCT |
| | GTGACAGCTCTGCTGCTGCCTCTGGCTCTGCTTCT |
| | GCATGCTGCCAGGTGACCATCACCTGTCGGGCCAG |
| | CGAGAACATCTTCAGCTACCTGGCCTGGTATCAGC |
| | AGAAGCCCGGCAAGTCTCCTAAGCTGCTGGTGTAC |
| | AACACCCGGACACTGGCTGAAGGCGTGCCCAGCAG |
| | ATTTTCTGGCAGCGGCAGCGGAACCGACTTCAGCC |
| | TGACAATCAGCAGCCTGCAGCCTGAGGACTTCGCC |
| | ACCTACTACTGCCAGCACCACTACGGCACCCCTTT |
| | CACCTTTGGCTCTGGCACCAAGCTGGAAATCAAGA |
| | GAGGCGGCGGAGGATCTGGCGGAGGTGGAAGTGGC |
| | GGAGGCGGATCTGAAGTTCAGCTGCAAGAGTCTGG |
| | CCCTGGCCTCGTGAAACCTGGCGGATCTCTGTCTC |
| | TGAGCTGTGCCGCCAGCGGCTTCGTGTTCAGCAGC |
| | TACGATATGAGCTGGGTCCGACAGACCCCTGAGCG |
| | AGGACTTGAGTGGGTCGCCTACATCTCTTCTGGCG |
| | GCGGAATCACATACGCCCCTAGCACAGTGAAGGGC |
| | AGATTCACCGTGTCCAGAGACAACGCCAAGAACAC |
| | CCTGTACCTGCAGATGAACAGCCTGACCAGCGAGG |
| | ACACCGCCGTGTACTATTGTGCCGCTCACTACTTT |
| | GGCAGCAGCGGCCCTTTTGCCTATTGGGGCCAGGG |
| | AACACTCGTGACCGTGTCTAGCGCCGAGCAGAAGC |
| | TGATCTCCGAAGAGGACCTGAATGGCGCCGCTACC |
| | ACAACACCAGCTCCTAGACCTCCAACTCCTGCTCC |
| | TACAATCGCCCTGCAGCCACTGTCTCTGAGGCCTG |
| | AAGCTTGTAGACCTGCTGCTGGCGGAGCCGTGCAT |
| | ACCAGAGGACTGGATTTCGCCTGCGACATCTACAT |
| | CTGGGCCCCTCTGGCTGGAACATGCGGAGTGTTGC |
| | TGCTGAGCCTGGTCATCACCAAGCGGGGCAGAAAG |
| | AAGCTGCTGTACATCTTCAAGCAGCCCTTCATGCG |
| | GCCCGTGCAGACCACACAAGAGGAAGATGGCTGCT |
| | CCTGCAGATTCCCCGAGGAAGAAGAAGGCGGCTGC |
| | GAGCTGAGAGTGAAGTTCAGCAGATCCGCCGACGC |
| | TCCTGCCTATCAGCAGGGACAGAACCAGCTGTACA |
| | ACGAGCTGAACCTGGGGAGAAGAGAAGAGTACGAC |
| | GTGCTGGACAAGCGGAGAGGCAGAGATCCTGAAAT |
| | GGGCGGCAAGCCCAGACGGAAGAATCCTCAAGAGG |
| | GCCTGTATAATGAGCTGCAGAAAGACAAGATGGCC |
| | GAGGCCTACAGCGAGATCGGAATGAAGGGCGAGCG |
| | CAGAAGAGGCAAGGGACACGATGGACTGTACCAGG |
| | GCCTGAGCACCGCCACCAAGGATACCTATGATGCC |
| | CTGCACATGCAGGCCCTGCCTCCAAGA |
| | (SEQ ID NO: 102) |
| SB03098 | MALPVTALLLPLALLLHAARPDIQMTQSPASLSAS |
| A.A. Seq | VGDRVTITCRASENIFSYLAWYQQKPGKSPKLLVY |
| | NTRTLAEGVPSRFSGSGSGTDFSLTISSLQPEDFA |
| | TYYCQHHYGTPFTFGSGTKLEIKRGGGGSGGGGSG |
| | GGGSEVQLQESGPGLVKPGGSLSLSCAASGFVFSS |
| | YDMSWVRQTPERGLEWVAYISSGGGITYAPSTVKG |
| | RFTVSRDNAKNTLYLQMNSLTSEDTAVYYCAAHYF |
| | GSSGPFAYWGQGTLVTVSSAEQKLISEEDLNGAAT |
| | TTPAPRPPTPAPTIALQPLSLRPEACRPAAGGAVH |
| | TRGLDFACDIYIWAPLAGTCGVLLLSLVITKRGRK |
| | KLLYIFKQPFMRPVQTTQEEDGCSCRFPEEEEGGC |
| | ELRVKFSRSADAPAYQQGQNQLYNELNLGRREEYD |
| | VLDKRRGRDPEMGGKPRRKNPQEGLYNELQKDKMA |
| | EAYSEIGMKGERRRGKGHDGLYQGLSTATKDTYDA |
| | LHMQALPPR |
| | (SEQ ID NO: 103) |
| SB03099 | ATGGCTCTGCCTGTGACAGCTCTGCTGCTGCCTCT |
| DNA Seq | GGCTCTGCTTCTGCATGCTGCCAGACCTGAAGTGC |
| | AGCTGCTGGAATCTGGCGGAGGACTGGTTCAACCT |
| | GGCGGCTCTCTGAGACTGAGCTGTGCCACAAGCGG |
| | CTTCACCTTCACCGACTACTACATGAACTGGGTCC |
| | GACAGGCCCCTGGCAAAGGACTGGAATGGCTGGGC |
| | TTCATCGGCAACAAGGCCAACGGCTACACCACCGA |
| | GTACAGCGCCTCTGTGAAGGGCAGATTCACCATCA |
| | GCCGGGACAAGAGCAAGAGCACCCTGTACCTGCAG |
| | ATGAACACCCTGCAGGCCGAGGACTCCGCCATCTA |
| | CTACTGCACCAGAGACAGAGGCCTGCGGTTCTACT |
| | TCGACTATTGGGGCCAGGGCACCCTGGTCACAGTG |
| | TCTAGCGCCTCTACAAAGGGCCCCAGCGTTTTCCC |
| | ACTGGCTCCCTGTAGCAGAAGCACCAGCGAATCTA |
| | CAGCCGCTCTGGGCTGCCTGGTCAAGGACTACTTT |
| | CCTGAGCCTGTGACCGTGCTGTATCTCCAGATGAA |
| | TACTCTCCAGGCTGAAGATAGCGCCATATATTACT |
| | GTACGCGCGACCGGGGCCTGAGATTCTACTTTGAT |
| | TACTGGGGACAAGGGACCCTCGTGACCGTTTCTTC |
| | TGGCGGCGGAGGAAGCGGAGGCGGAGGATCCGGTG |
| | GTGGTGGATCTCAGACAGTGCTGACACAGAGCCCT |
| | AGCAGCCTGTCTGTGTCCGTGGGCGACAGAGTGAC |
| | CATCACCTGTAGAGCCAGCAGCTCCGTGACCTACA |
| | TCCACTGGTATCAGCAGAAGCCCGGACTGGCCCCT |
| | AAGAGCCTGATCTACGCCACAAGCAATCTGGCCAG |
| | CGGCGTGCCAAGCAGATTTTCTGGAAGCGGCAGCG |
| | GCACCGACTATACCTTTACAATCAGCAGCCTGCAG |
| | CCTGAGGATATCGCCACCTACTATTGCCAGCACTG |
| | GTCCAGCAAGCCTCCAACCTTTGGACAGGGCACCA |
| | AGGTGGAAGTGAAGCGGACCGTGGAGCAGAAGCTG |
| | ATCTCCGAAGAGGACCTGAATGGCGCCGCTACCAC |
| | AACACCAGCTCCTAGACCTCCAACTCCTGCTCCTA |
| | CAATCGCCCTGCAGCCACTGTCTCTGAGGCCTGAA |
| | GCTTGTAGACCTGCTGCTGGCGGAGCCGTGCATAC |
| | CAGAGGACTGGATTTCGCCTGCGACATCTACATCT |
| | GGGCCCCTCTGGCTGGAACATGCGGAGTGTTGCTG |
| | CTGAGCCTGGTCATCACCAAGCGGGGCAGAAAGAA |
| | GCTGCTGTACATCTTCAAGCAGCCCTTCATGCGGC |
| | CCGTGCAGACCACACAAGAGGAAGATGGCTGCTCC |
| | TGCAGATTCCCCGAGGAAGAAGAAGGCGGCTGCGA |
| | GCTGAGAGTGAAGTTCAGCAGATCCGCCGACGCTC |
| | CTGCCTATCAGCAGGGACAGAACCAGCTGTACAAC |
| | GAGCTGAACCTGGGGAGAAGAGAAGAGTACGACGT |
| | GCTGGACAAGCGGAGAGGCAGAGATCCTGAAATGG |
| | GCGGCAAGCCCAGACGGAAGAATCCTCAAGAGGGC |
| | CTGTATAATGAGCTGCAGAAAGACAAGATGGCCGA |
| | GGCCTACAGCGAGATCGGAATGAAGGGCGAGCGCA |
| | GAAGAGGCAAGGGACACGATGGACTGTACCAGGGC |
| | CTGAGCACCGCCACCAAGGATACCTATGATGCCCT |
| | GCACATGCAGGCCCTGCCTCCAAGA |
| | (SEQ ID NO: 104) |
| SB03099 | MALPVTALLLPLALLLHAARPEVQLLESGGGLVQP |
| A.A. Seq | GGSLRLSCATSGFTFTDYYMNWVRQAPGKGLEWLG |
| | FIGNKANGYTTEYSASVKGRFTISRDKSKSTLYLQ |
| | MNTLQAEDSAIYYCTRDRGLRFYFDYWGQGTLVTV |
| | SSASTKGPSVFPLAPCSRSTSESTAALGCLVKDYF |
| | PEPVTVLYLQMNTLQAEDSAIYYCTRDRGLRFYFD |
| | YWGQGTLVTVSSGGGGSGGGGSGGGGSQTVLTQSP |
| | SSLSVSVGDRVTITCRASSSVTYIHWYQQKPGLAP |
| | KSLIYATSNLASGVPSRFSGSGSGTDYTFTISSLQ |
| | PEDIATYYCQHWSSKPPTFGQGTKVEVKRTVEQKL |
| | ISEEDLNGAATTTPAPRPPTPAPTIALQPLSLRPE |
| | ACRPAAGGAVHTRGLDFACDIYIWAPLAGTCGVLL |
| | LSLVITKRGRKKLLYIFKQPFMRPVQTTQEEDGCS |
| | CRFPEEEEGGCELRVKFSRSADAPAYQQGQNQLYN |
| | ELNLGRREEYDVLDKRRGRDPEMGGKPRRKNPQEG |
| | LYNELQKDKMAEAYSEIGMKGERRRGKGHDGLYQG |
| | LSTATKDTYDALHMQALPPR |
| | (SEQ ID NO: 105) |
| SB03100 | ATGGCTCTGCCTGTGACAGCTCTGCTGCTGCCTCT |
| DNA Seq | GGCTCTGCTTCTGCATGCTGCCAGACCTCAGACCG |
| | TGCTGACACAGAGCCCAAGCAGCCTGTCTGTGTCC |
| | GTGGGCGACAGAGTGACCATCACCTGTAGAGCCAG |
| | CAGCTCCGTGACCTACATCCACTGGTATCAGCAGA |
| | AGCCCGGACTGGCCCCTAAGAGCCTGATCTACGCC |
| | ACAAGCAATCTGGCCAGCGGCGTGCCAAGCAGATT |
| | TTCTGGAAGCGGCAGCGGCACCGACTACACCTTCA |
| | CCATATCTAGCCTGCAGCCTGAGGATATCGCCACC |
| | TACTACTGCCAGCACTGGTCCAGCAAGCCTCCAAC |
| | ATTTGGCCAGGGCACCAAGGTGGAAGTGAAGAGAA |
| | CAGTTGGCGGCGGAGGATCTGGCGGAGGCGGTAGC |
| | GGAGGTGGTGGATCTGAAGTTCAGCTGCTGGAATC |
| | AGGCGGCGGACTGGTTCAACCTGGCGGATCTCTGA |
| | GACTGAGCTGTGCCACCAGCGGCTTCACCTTTACC |
| | GACTACTACATGAACTGGGTCCGACAGGCCCCTGG |
| | CAAAGGACTGGAATGGCTGGGCTTCATCGGCAACA |
| | AGGCCAACGGCTACACCACCGAGTACAGCGCCTCT |
| | GTGAAGGGCAGATTCACCATCAGCCGGGACAAGAG |
| | CAAGAGCACCCTGTACCTGCAGATGAACACCCTGC |
| | AGGCCGAGGACTCCGCCATCTACTATTGCACCAGA |
| | GACAGAGGCCTGCGGTTCTACTTCGACTATTGGGG |
| | CCAGGGAACCCTGGTCACAGTGTCTAGCGCCTCTA |
| | CAAAGGGCCCCAGCGTTTTCCCACTGGCTCCCTGT |
| | AGCAGAAGCACCAGCGAATCTACAGCCGCTCTGGG |
| | CTGCCTGGTCAAGGACTACTTTCCTGAGCCAGTGA |
| | CAGTGCTGTATCTCCAGATGAATACTCTCCAAGCT |
| | GAGGACAGCGCCATCTATTACTGTACGCGCGATCG |
| | GGGCCTGAGATTCTACTTTGATTACTGGGGACAAG |
| | GCACCCTCGTGACCGTGTCCTCTGAGCAGAAGCTG |
| | ATCTCCGAAGAGGACCTGAATGGCGCCGCTACCAC |
| | AACACCAGCTCCTAGACCTCCAACTCCTGCTCCTA |
| | CAATCGCCCTGCAGCCACTGTCTCTGAGGCCTGAA |
| | GCTTGTAGACCTGCTGCTGGCGGAGCCGTGCATAC |
| | CAGAGGACTGGATTTCGCCTGCGACATCTACATCT |
| | GGGCCCCTCTGGCTGGAACATGCGGAGTGTTGCTG |
| | CTGAGCCTGGTCATCACCAAGCGGGGCAGAAAGAA |
| | GCTGCTGTACATCTTCAAGCAGCCCTTCATGCGGC |
| | CCGTGCAGACCACACAAGAGGAAGATGGCTGCTCC |
| | TGCAGATTCCCCGAGGAAGAAGAAGGCGGCTGCGA |
| | GCTGAGAGTGAAGTTCAGCAGATCCGCCGACGCTC |
| | CTGCCTATCAGCAGGGACAGAACCAGCTGTACAAC |
| | GAGCTGAACCTGGGGAGAAGAGAAGAGTACGACGT |
| | GCTGGACAAGCGGAGAGGCAGAGATCCTGAAATGG |
| | GCGGCAAGCCCAGACGGAAGAATCCTCAAGAGGGC |
| | CTGTATAATGAGCTGCAGAAAGACAAGATGGCCGA |
| | GGCCTACAGCGAGATCGGAATGAAGGGCGAGCGCA |
| | GAAGAGGCAAGGGACACGATGGACTGTACCAGGGC |
| | CTGAGCACCGCCACCAAGGATACCTATGATGCCCT |
| | GCACATGCAGGCCCTGCCTCCAAGA |
| | (SEQ ID NO: 106) |
| SB03100 | MALPVTALLLPLALLLHAARPQTVLTQSPSSLSVS |
| A.A. Seq | VGDRVTITCRASSSVTYIHWYQQKPGLAPKSLIYA |
| | TSNLASGVPSRFSGSGSGTDYTFTISSLQPEDIAT |
| | YYCQHWSSKPPTFGQGTKVEVKRTVGGGGSGGGGS |
| | GGGGSEVQLLESGGGLVQPGGSLRLSCATSGFTFT |
| | DYYMNWVRQAPGKGLEWLGFIGNKANGYTTEYSAS |
| | VKGRFTISRDKSKSTLYLQMNTLQAEDSAIYYCTR |
| | DRGLRFYFDYWGQGTLVTVSSASTKGPSVFPLAPC |
| | SRSTSESTAALGCLVKDYFPEPVTVLYLQMNTLQA |
| | EDSAIYYCTRDRGLRFYFDYWGQGTLVTVSSEQKL |
| | ISEEDLNGAATTTPAPRPPTPAPTIALQPLSLRPE |
| | ACRPAAGGAVHTRGLDFACDIYIWAPLAGTCGVLL |
| | LSLVITKRGRKKLLYIFKQPFMRPVQTTQEEDGCS |
| | CRFPEEEEGGCELRVKFSRSADAPAYQQGQNQLYN |
| | ELNLGRREEYDVLDKRRGRDPEMGGKPRRKNPQEG |
| | LYNELQKDKMAEAYSEIGMKGERRRGKGHDGLYQG |
| | LSTATKDTYDALHMQALPPR |
| | (SEQ ID NO: 107) |
| SB03173 | ATGGCTCTGCCTGTGACAGCTCTGCTGCTGCCTCT |
| DNA Seq | GGCTCTGCTTCTGCATGCCGCCAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAGCAGAAGCTGATCTCCGAAG |
| | AGGACCTGACCACAACACCAGCTCCTAGACCACCT |
| | ACACCAGCACCTACAATCGCCCTGCAGCCACTGTC |
| | TCTGAGGCCTGAAGCTTGTAGACCTGCTGCTGGCG |
| | GAGCCGTGCATACAAGAGGACTGGATTTCGCCTGC |
| | GACTTCTGGGTGCTCGTTGTTGTTGGCGGCGTGCT |
| | GGCCTGTTATTCCCTGCTGGTTACCGTGGCCTTCA |
| | TCATCTTTTGGGTCCGAAGCAAGCGGAGCAGACTG |
| | CTGCACTCCGACTACATGAACATGACCCCTAGACG |
| | GCCCGGACCAACCAGAAAGCACTACCAGCCTTACG |
| | CTCCTCCTAGAGACTTCGCCGCCTACCGGTCCAGA |
| | GTGAAGTTCTCCAGATCCGCCGATGCTCCCGCCTA |
| | TAAGCAGGGACAGAACCAGCTGTACAACGAGCTGA |
| | ACCTGGGGAGAAGAGAAGAGTACGACGTGCTGGAC |
| | AAGCGGAGAGGCAGAGATCCTGAGATGGGCGGCAA |
| | GCCCAGACGGAAGAATCCTCAAGAGGGCCTGTATA |
| | ATGAGCTGCAAAAGGACAAGATGGCCGAGGCCTAC |
| | AGCGAGATCGGAATGAAGGGCGAGCGCAGAAGAGG |
| | CAAGGGACACGATGGACTGTACCAGGGCCTGAGCA |
| | CCGCCACCAAGGATACCTATGATGCCCTGCACATG |
| | CAGGCCCTGCCTCCAAGA |
| | (SEQ ID NO: 137) |
| SB03173 | MALPVTALLLPLALLLHAARPDIQLTQSPSSLSAS |
| A.A. Seq | VGDRVTITCKASQDVGTSVAWYQQKPGKAPKLLIY |
| | WTSTRHTGVPSRFSGSGSGTDFTFTISSLQPEDIA |
| | TYYCQQYSLYRSFGQGTKVEIKGGSGSGGSGSGGS |
| | GSEVOLVESGGGVVQPGRSLRLSCSASGFDFTTYW |
| | MSWVRQAPGKGLEWIGEIHPDSSTINYAPSLKDRF |
| | TISRDNAKNTLFLQMDSLRPEDTGVYFCASLYFGF |
| | PWFAYWGQGTPVTVSSEQKLISEEDLNGAATTTPA |
| | PRPPTPAPTIALQPLSLRPEACRPAAGGAVHTRGL |
| | DFACDFWVLVVVGGVLACYSLLVTVAFIIFWVRSK |
| | RSRLLHSDYMNMTPRRPGPTRKHYQPYAPPRDFAA |
| | YRSRVKFSRSADAPAYKQGQNQLYNELNLGRREEY |
| | DVLDKRRGRDPEMGGKPRRKNPQEGLYNELQKDKM |
| | AEAYSEIGMKGERRRGKGHDGLYQGLSTATKDTYD |
| | ALHMQALPPR |
| | (SEQ ID NO: 108) |
| SB03175 | ATGGCTCTGCCTGTGACAGCTCTGCTGCTGCCTCT |
| DNA Seq | GGCTCTGCTTCTGCATGCCGCCAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAGCAGAAGCTGATCTCCGAAG |
| | AGGACCTGACCACAACACCAGCTCCTAGACCTCCA |
| | ACTCCTGCTCCTACAATCGCCCTGCAGCCACTGTC |
| | TCTGAGGCCTGAAGCTTGTAGACCTGCTGCTGGCG |
| | GAGCCGTGCATACCAGAGGACTGGATTTCGCCTGC |
| | GACATCTACATCTGGGCCCCTCTGGCTGGAACATG |
| | CGGAGTGTTGCTGCTGAGCCTGGTCATCACCAAGC |
| | GGGGCAGAAAGAAGCTGCTGTACATCTTCAAGCAG |
| | CCCTTCATGCGGCCCGTGCAGACCACACAAGAGGA |
| | AGATGGCTGCTCCTGCAGATTCCCCGAGGAAGAAG |
| | AAGGCGGCTGCGAGCTGAGAGTGAAGTTCAGCAGA |
| | TCCGCCGACGCTCCTGCCTATCAGCAGGGACAGAA |
| | CCAGCTGTACAACGAGCTGAACCTGGGGAGAAGAG |
| | AAGAGTACGACGTGCTGGACAAGCGGAGAGGCAGA |
| | GATCCTGAAATGGGCGGCAAGCCCAGACGGAAGAA |
| | TCCTCAAGAGGGCCTGTATAATGAGCTGCAGAAAG |
| | ACAAGATGGCCGAGGCCTACAGCGAGATCGGAATG |
| | AAGGGCGAGCGCAGAAGAGGCAAGGGACACGATGG |
| | ACTGTACCAGGGCCTGAGCACCGCCACCAAGGATA |
| | CCTATGATGCCCTGCACATGCAGGCCCTGCCTCCA |
| | AGA |
| | (SEQ ID NO: 109) |
| SB03175 | MALPVTALLLPLALLLHAARPDIQLTQSPSSLSAS |
| A.A. Seq | VGDRVTITCKASQDVGTSVAWYQQKPGKAPKLLIY |
| | WTSTRHTGVPSRFSGSGSGTDFTFTISSLQPEDIA |
| | TYYCQQYSLYRSFGQGTKVEIKGGSGSGGSGSGGS |
| | GSEVQLVESGGGVVQPGRSLRLSCSASGFDFTTYW |
| | MSWVRQAPGKGLEWIGEIHPDSSTINYAPSLKDRF |
| | TISRDNAKNTLFLQMDSLRPEDTGVYFCASLYFGF |
| | PWFAYWGQGTPVTVSSEQKLISEEDLNGAATTTPA |
| | PRPPTPAPTIALQPLSLRPEACRPAAGGAVHTRGL |
| | DFACDIYIWAPLAGTCGVLLLSLVITKRGRKKLLY |
| | IFKQPFMRPVQTTQEEDGCSCRFPEEEEGGCELRV |
| | KFSRSADAPAYQQGQNQLYNELNLGRREEYDVLDK |
| | RRGRDPEMGGKPRRKNPQEGLYNELQKDKMAEAYS |
| | EIGMKGERRRGKGHDGLYQGLSTATKDTYDALHMQ |
| | ALPPR |
| | (SEQ ID NO: 110) |
| SB03180 | ATGGCTCTGCCTGTGACAGCTCTGCTGCTGCCTCT |
| DNA Seq | GGCTCTGCTTCTGCATGCCGCTAGACCTGAGGTCC |
| | AGCTGCAAGAGTCTGGCCCTGGACTTGTGAAGCCT |
| | GGCGGAAGCCTGTCTCTGTCTTGTGCCGCCAGCGG |
| | CTTCGTGTTCAGCAGCTACGATATGAGCTGGGTCC |
| | GACAGACCCCTGAGCGAGGACTTGAGTGGGTCGCC |
| | TACATCTCTTCTGGCGGCGGAATCACATACGCCCC |
| | TAGCACAGTGAAGGGCAGATTCACCGTGTCCAGAG |
| | ACAACGCCAAGAACACCCTGTACCTGCAGATGAAC |
| | AGCCTGACCAGCGAGGACACCGCCGTGTACTATTG |
| | TGCCGCTCACTACTTTGGCAGCAGCGGCCCTTTTG |
| | CCTATTGGGGCCAGGGAACACTCGTGACCGTTTCT |
| | AGTGCTGGCGGAGGCGGATCAGGTGGCGGAGGAAG |
| | TGGCGGCGGAGGATCTGATATCCAGATGACACAGA |
| | GCCCCGCCAGCCTGTCTGCCTCTGTGGGAGATAGA |
| | GTGACCATCACCTGTCGGGCCAGCGAGAACATCTT |
| | CAGCTACCTGGCCTGGTATCAGCAGAAGCCCGGCA |
| | AGTCTCCTAAGCTGCTGGTGTACAACACCCGGACA |
| | CTGGCTGAAGGCGTGCCCAGCAGATTTTCTGGCAG |
| | CGGCTCTGGCACCGACTTCAGCCTGACAATCAGCA |
| | GCCTGCAGCCTGAGGACTTCGCCACCTACTACTGC |
| | CAGCACCACTACGGCACCCCTTTCACCTTTGGAAG |
| | CGGCACCAAGCTGGAAATCAAGAGAGAGCAGAAGC |
| | TGATCTCCGAAGAGGACCTGACCACAACTCCTGCT |
| | CCTAGACCTCCTACACCAGCTCCTACAATCGCCCT |
| | GCAGCCACTGTCTCTGAGGCCAGAAGCTTGTAGAC |
| | CTGCTGCAGGCGGAGCCGTGCATACAAGAGGACTG |
| | GATTTCGCCTGCGACTTCTGGGTGCTCGTGGTTGT |
| | TGGCGGAGTGCTGGCCTGTTACTCTCTGCTGGTCA |
| | CCGTGGCCTTCATCATCTTTTGGGTCCGAAGCAAG |
| | CGGAGCCGGCTGCTGCACAGCGACTACATGAACAT |
| | GACCCCTAGACGGCCCGGACCTACCAGAAAGCACT |
| | ACCAGCCTTACGCTCCTCCTAGAGATTTCGCCGCC |
| | TACCGGTCCAGAGTGAAGTTCAGCAGATCCGCCGA |
| | TGCTCCCGCCTATAAGCAGGGACAGAACCAGCTGT |
| | ACAACGAGCTGAACCTGGGGAGAAGAGAAGAGTAC |
| | GACGTGCTGGACAAGCGGAGAGGCAGAGATCCTGA |
| | GATGGGCGGCAAGCCCAGACGGAAGAATCCTCAAG |
| | AGGGCCTGTATAATGAGCTGCAGAAAGACAAGATG |
| | GCCGAGGCCTACAGCGAGATCGGAATGAAGGGCGA |
| | GCGCAGAAGAGGCAAGGGACACGATGGACTGTACC |
| | AGGGCCTGAGCACCGCCACCAAGGATACCTATGAT |
| | GCCCTGCACATGCAGGCCCTGCCTCCAAGA |
| | (SEQ ID NO: 111) |
| SB03180 | MALPVTALLLPLALLLHAARPEVQLQESGPGLVKP |
| A.A. Seq | GGSLSLSCAASGFVFSSYDMSWVRQTPERGLEWVA |
| | YISSGGGITYAPSTVKGRFTVSRDNAKNTLYLQMN |
| | SLTSEDTAVYYCAAHYFGSSGPFAYWGQGTLVTVS |
| | SAGGGGSGGGGSGGGGSDIQMTQSPASLSASVGDR |
| | VTITCRASENIFSYLAWYQQKPGKSPKLLVYNTRT |
| | LAEGVPSRFSGSGSGTDFSLTISSLQPEDFATYYC |
| | QHHYGTPFTFGSGTKLEIKREQKLISEEDLNGAAT |
| | TTPAPRPPTPAPTIALQPLSLRPEACRPAAGGAVH |
| | TRGLDFACDFWVLVVVGGVLACYSLLVTVAFIIFW |
| | VRSKRSRLLHSDYMNMTPRRPGPTRKHYQPYAPPR |
| | DFAAYRSRVKFSRSADAPAYKQGQNQLYNELNLGR |
| | REEYDVLDKRRGRDPEMGGKPRRKNPQEGLYNELQ |
| | KDKMAEAYSEIGMKGERRRGKGHDGLYQGLSTATK |
| | DTYDALHMQALPPR |
| | (SEQ ID NO: 112) |
| SB03290 | ATGGCTCTGCCTGTGACAGCTCTGCTGCTGCCTCT |
| DNA Seq | GGCTCTGCTTCTGCATGCCGCTAGACCTGAGGTCC |
| | AGCTGCAAGAGTCTGGCCCTGGACTTGTGAAGCCT |
| | GGCGGAAGCCTGTCTCTGTCTTGTGCCGCCAGCGG |
| | CTTCGTGTTCAGCAGCTACGATATGAGCTGGGTCC |
| | GACAGACCCCTGAGCGAGGACTTGAGTGGGTCGCC |
| | TACATCTCTTCTGGCGGCGGAATCACATACGCCCC |
| | TAGCACAGTGAAGGGCAGATTCACCGTGTCCAGAG |
| | ACAACGCCAAGAACACCCTGTACCTGCAGATGAAC |
| | AGCCTGACCAGCGAGGACACCGCCGTGTACTATTG |
| | TGCCGCTCACTACTTTGGCAGCAGCGGCCCTTTTG |
| | CCTATTGGGGCCAGGGAACACTCGTGACCGTTTCT |
| | AGTGCTGGCGGAGGCGGATCAGGTGGCGGAGGAAG |
| | TGGCGGCGGAGGATCTGATATCCAGATGACACAGA |
| | GCCCCGCCAGCCTGTCTGCCTCTGTGGGAGATAGA |
| | GTGACCATCACCTGTCGGGCCAGCGAGAACATCTT |
| | CAGCTACCTGGCCTGGTATCAGCAGAAGCCCGGCA |
| | AGTCTCCTAAGCTGCTGGTGTACAACACCCGGACA |
| | CTGGCTGAAGGCGTGCCCAGCAGATTTTCTGGCAG |
| | CGGCTCTGGCACCGACTTCAGCCTGACAATCAGCA |
| | GCCTGCAGCCTGAGGACTTCGCCACCTACTACTGC |
| | CAGCACCACTACGGCACCCCTTTCACCTTTGGAAG |
| | CGGCACCAAGCTGGAAATCAAGAGAGAGCAGAAGC |
| | TGATCTCCGAAGAGGACCTGACCACAACACCAGCT |
| | CCTAGACCACCTACACCAGCACCTACAATCGCCCT |
| | GCAGCCACTGTCTCTGAGGCCAGAGGCTTGTAGAC |
| | CTGCTGCTGGCGGAGCCGTGCATACAAGAGGACTG |
| | GATTTCGCCTGCGACGTGGCCGCCATTCTCGGACT |
| | GGGACTTGTTCTGGGACTGCTGGGACCTCTGGCCA |
| | TTCTGCTGGCTCTGTACCTGCTGAGAAGGGACCAG |
| | AGACTGCCTCCTGACGCTCACAAACCTCCAGGCGG |
| | AGGCAGCTTCAGAACCCCTATCCAAGAGGAACAGG |
| | CTGACGCCCACAGCACCCTGGCCAAGATCAGAGTG |
| | AAGTTCAGCAGATCCGCCGACGCTCCCGCCTATAA |
| | GCAGGGACAGAATCAGCTGTACAACGAGCTGAACC |
| | TGGGGCGCAGAGAAGAGTACGACGTGCTGGACAAG |
| | AGAAGAGGCAGGGATCCTGAAATGGGCGGCAAGCC |
| | CAGACGGAAGAATCCTCAAGAGGGCCTGTATAATG |
| | AGCTGCAGAAAGACAAGATGGCCGAGGCCTACAGC |
| | GAGATCGGAATGAAGGGCGAACGCAGAAGAGGAAA |
| | GGGCCACGACGGACTGTATCAGGGCCTGAGCACAG |
| | CCACCAAGGACACCTATGATGCCCTGCACATGCAG |
| | GCCCTGCCTCCAAGA |
| | (SEQ ID NO: 113) |
| SB03290 | MALPVTALLLPLALLLHAARPEVQLQESGPGLVKP |
| A.A. Seq | GGSLSLSCAASGFVFSSYDMSWVRQTPERGLEWVA |
| | YISSGGGITYAPSTVKGRFTVSRDNAKNTLYLOMN |
| | SLTSEDTAVYYCAAHYFGSSGPFAYWGQGTLVTVS |
| | SAGGGGSGGGGSGGGGSDIQMTQSPASLSASVGDR |
| | VTITCRASENIFSYLAWYQQKPGKSPKLLVYNTRT |
| | LAEGVPSRFSGSGSGTDFSLTISSLOPEDFATYYC |
| | QHHYGTPFTFGSGTKLEIKREQKLISEEDLNGAAT |
| | TTPAPRPPTPAPTIALQPLSLRPEACRPAAGGAVH |
| | TRGLDFACDVAAILGLGLVLGLLGPLAILLALYLL |
| | RRDQRLPPDAHKPPGGGSFRTPIQEEQADAHSTLA |
| | KIRVKFSRSADAPAYKQGQNQLYNELNLGRREEYD |
| | VLDKRRGRDPEMGGKPRRKNPQEGLYNELQKDKMA |
| | EAYSEIGMKGERRRGKGHDGLYQGLSTATKDTYDA |
| | LHMQALPPR |
| | (SEQ ID NO: 114) |
| SB03604 | ATGGCTCTGCCTGTGACAGCTCTGCTGCTGCCTCT |
| DNA Seq | GGCTCTGCTTCTGCATGCCGCCAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAGCAGAAGCTGATCTCCGAAG |
| | AGGACCTGACCACAACACCAGCTCCTAGACCTCCA |
| | ACTCCTGCTCCTACAATCGCCCTGCAGCCACTGTC |
| | TCTGAGGCCTGAAGCTTGTAGACCTGCTGCTGGCG |
| | GAGCCGTGCATACCAGAGGACTGGATTTCGCCTGC |
| | GACTTCCTGGTCATCATCGTGATCCTGAGCGCCCT |
| | GTTCCTGGGCACACTGGCCTGTTTCTGCGTGTGGC |
| | GCAGAAAGCGCAAAGAGAAGCAGAGCGAGACAAGC |
| | CCCAAAGAGTTCCTGACCATCTACGAGGACGTGAA |
| | GGACCTGAAAACCCGGCGGAACCACGAGCAAGAGC |
| | AGACCTTTCCTGGCGGCGGATCCACCATCTACAGC |
| | ATGATCCAGAGCCAGAGCAGCGCCCCTACAAGCCA |
| | AGAGCCTGCCTACACACTGTACAGCCTGATTCAGC |
| | CCAGCAGAAAGAGCGGCTCCCGGAAGAGAAATCAC |
| | AGCCCCAGCTTCAACAGCACGATCTACGAAGTGAT |
| | CGGCAAGAGCCAGCCAAAGGCTCAGAACCCTGCCA |
| | GACTGAGCAGAAAAGAGCTGGAAAACTTCGACGTG |
| | TACAGCCGCGTGAAGTTCAGCAGATCCGCCGATGC |
| | TCCTGCCTATCAGCAGGGCCAGAACCAGCTGTACA |
| | ACGAGCTGAACCTGGGGAGAAGAGAAGAGTACGAC |
| | GTGCTGGACAAGCGGAGAGGCAGAGATCCTGAAAT |
| | GGGCGGCAAGCCCAGACGGAAGAATCCTCAAGAGG |
| | GCCTGTATAATGAGCTGCAGAAAGACAAGATGGCC |
| | GAGGCCTACAGCGAGATCGGAATGAAGGGCGAGCG |
| | CAGAAGAGGCAAGGGACACGATGGACTGTACCAGG |
| | GCCTGAGCACCGCCACCAAGGATACCTATGATGCC |
| | CTGCACATGCAGGCCCTGCCTCCAAGA |
| | (SEQ ID NO: 115) |
| SB03604 | MALPVTALLLPLALLLHAARPDIQLTQSPSSLSAS |
| A.A. Seq | VGDRVTITCKASQDVGTSVAWYQQKPGKAPKLLIY |
| | WTSTRHTGVPSRFSGSGSGTDFTFTISSLQPEDIA |
| | TYYCQQYSLYRSFGQGTKVEIKGGSGSGGSGSGGS |
| | GSEVQLVESGGGVVQPGRSLRLSCSASGFDFTTYW |
| | MSWVRQAPGKGLEWIGEIHPDSSTINYAPSLKDRF |
| | TISRDNAKNTLFLQMDSLRPEDTGVYFCASLYFGF |
| | PWFAYWGQGTPVTVSSEQKLISEEDLNGAATTTPA |
| | PRPPTPAPTIALQPLSLRPEACRPAAGGAVHTRGL |
| | DFACDFLVIIVILSALFLGTLACFCVWRRKRKEKQ |
| | SETSPKEFLTIYEDVKDLKTRRNHEQEQTFPGGGS |
| | TIYSMIQSQSSAPTSQEPAYTLYSLIQPSRKSGSR |
| | KRNHSPSFNSTIYEVIGKSQPKAQNPARLSRKELE |
| | NFDVYSRVKFSRSADAPAYQQGQNQLYNELNLGRR |
| | EEYDVLDKRRGRDPEMGGKPRRKNPQEGLYNELQK |
| | DKMAEAYSEIGMKGERRRGKGHDGLYQGLSTATKD |
| | TYDALHMQALPPR |
| | (SEQ ID NO: 116) |
| SB03606 | ATGGCTCTGCCTGTGACAGCTCTGCTGCTGCCTCT |
| DNA Seq | GGCTCTGCTTCTGCATGCCGCTAGACCCCAAGTGA |
| | AGCTGGAACAGTCTGGCGCCGAGGTTGTGAAACCT |
| | GGCGCCTCTGTGAAGCTGAGCTGTAAAGCCAGCGG |
| | CTTCAACATCAAGGACAGCTACATGCACTGGCTGC |
| | GGCAAGGCCCTGGACAGAGACTGGAATGGATCGGC |
| | TGGATCGACCCCGAGAATGGCGATACAGAGTACGC |
| | CCCTAAGTTCCAGGGCAAAGCCACCTTCACCACCG |
| | ACACCTCTGCCAACACAGCCTACCTGGGACTGTCT |
| | AGCCTGAGGCCTGAAGATACCGCCGTGTACTACTG |
| | CAACGAGGGCACACCTACAGGCCCCTACTACTTCG |
| | ATTATTGGGGCCAGGGCACCCTGGTCACCGTTTCT |
| | AGCGGAGGCGGAGGATCTGGTGGCGGAGGAAGTGG |
| | CGGAGGCGGTTCTGAAAATGTGCTGACACAGAGCC |
| | CCAGCAGCATGTCCGTGTCTGTGGGCGACAGAGTG |
| | ACAATCGCCTGTAGCGCCTCTAGCAGCGTGCCCTA |
| | TATGCATTGGCTGCAGCAGAAGCCCGGCAAGAGCC |
| | CTAAGCTGCTGATCTACCTGACCAGCAATCTGGCC |
| | AGCGGCGTGCCAAGCAGATTTTCTGGAAGCGGCAG |
| | CGGCACCGACTACAGCCTGACAATCAGTAGCGTGC |
| | AGCCTGAGGATGCCGCCACCTACTACTGTCAGCAG |
| | AGAAGCAGCTACCCTCTGACCTTTGGAGGCGGCAC |
| | CAAGCTGGAAATCAAAGAGCAGAAGCTGATCTCCG |
| | AAGAGGACCTGACCACAACACCAGCTCCTAGACCT |
| | CCAACTCCTGCTCCTACAATCGCCCTGCAGCCACT |
| | GTCTCTGAGGCCTGAAGCTTGTAGACCTGCTGCTG |
| | GCGGAGCCGTGCATACCAGAGGACTGGATTTCGCC |
| | TGCGACTTCCTGGTCATCATCGTGATCCTGAGCGC |
| | CCTGTTCCTGGGCACACTGGCCTGTTTCTGCGTGT |
| | GGCGCAGAAAGCGCAAAGAGAAGCAGAGCGAGACA |
| | AGCCCCAAAGAGTTCCTGACCATCTACGAGGACGT |
| | GAAGGACCTGAAAACCCGGCGGAACCACGAGCAAG |
| | AGCAGACCTTTCCTGGCGGCGGATCCACCATCTAC |
| | AGCATGATCCAGAGCCAGAGCAGCGCCCCTACAAG |
| | CCAAGAGCCTGCCTACACACTGTACAGCCTGATTC |
| | AGCCCAGCAGAAAGAGCGGCTCCCGGAAGAGAAAT |
| | CACAGCCCCAGCTTCAACAGCACGATCTACGAAGT |
| | GATCGGCAAGAGCCAGCCAAAGGCTCAGAACCCTG |
| | CCAGACTGAGCAGAAAAGAGCTGGAAAACTTCGAC |
| | GTGTACAGCCGCGTGAAGTTCAGCAGATCCGCCGA |
| | TGCTCCTGCCTATCAGCAGGGCCAGAACCAGCTGT |
| | ACAACGAGCTGAACCTGGGGAGAAGAGAAGAGTAC |
| | GACGTGCTGGACAAGCGGAGAGGCAGAGATCCTGA |
| | AATGGGCGGCAAGCCCAGACGGAAGAATCCTCAAG |
| | AGGGCCTGTATAATGAGCTGCAGAAAGACAAGATG |
| | GCCGAGGCCTACAGCGAGATCGGAATGAAGGGCGA |
| | GCGCAGAAGAGGCAAGGGACACGATGGACTGTACC |
| | AGGGCCTGAGCACCGCCACCAAGGATACCTATGAT |
| | GCCCTGCACATGCAGGCCCTGCCTCCAAGA |
| | (SEQ ID NO: 117) |
| SB03606 | MALPVTALLLPLALLLHAARPETVIKYLLPTAAAG |
| A.A. Seq | LLLLAAQPAMAQVKLQQSGAELVRSGTSVKLSCTA |
| | SGFNIKDSYMHWLRQGPEQGLEWIGWIDPENGDTE |
| | YAPKFQGKATFTTDTSSNTAYLQLSSLTSEDTAVY |
| | YCNEGTPTGPYYFDYWGQGTTVTVSSGGGGSGGGG |
| | SGGGGSENVLTQSPAIMSASPGEKVTITCSASSSV |
| | SYMHWFQQKPGTSPKLWIYSTSNLASGVPARFSGS |
| | GSGTSYSLTISRMEAEDAATYYCQQRSSYPLTFGA |
| | GTKLELKRAAEQKLISEEDLTTTPAPRPPTPAPTI |
| | ALQPLSLRPEACRPAAGGAVHTRGLDFACDFLVII |
| | VILSALFLGTLACFCVWRRKRKEKQSETSPKEFLT |
| | IYEDVKDLKTRRNHEQEQTFPGGGSTIYSMIQSQS |
| | SAPTSQEPAYTLYSLIQPSRKSGSRKRNHSPSFNS |
| | TIYEVIGKSQPKAQNPARLSRKELENFDVYSRVKF |
| | SRSADAPAYQQGQNQLYNELNLGRREEYDVLDKRR |
| | GRDPEMGGKPRRKNPQEGLYNELQKDKMAEAYSEI |
| | GMKGERRRGKGHDGLYQGLSTATKDTYDALHMQAL |
| | PPR |
| | (SEQ ID NO: 118) |
| SB03607 | ATGGCTCTGCCTGTGACAGCTCTGCTGCTGCCTCT |
| DNA Seq | GGCTCTGCTTCTGCATGCCGCTAGACCCCAAGTGA |
| | AGCTGGAACAGTCTGGCGCCGAGGTTGTGAAACCT |
| | GGCGCCTCTGTGAAGCTGAGCTGTAAAGCCAGCGG |
| | CTTCAACATCAAGGACAGCTACATGCACTGGCTGC |
| | GGCAAGGCCCTGGACAGAGACTGGAATGGATCGGC |
| | TGGATCGACCCCGAGAATGGCGATACAGAGTACGC |
| | CCCTAAGTTCCAGGGCAAAGCCACCTTCACCACCG |
| | ACACCTCTGCCAACACAGCCTACCTGGGACTGTCT |
| | AGCCTGAGGCCTGAAGATACCGCCGTGTACTACTG |
| | CAACGAGGGCACACCTACAGGCCCCTACTACTTCG |
| | ATTATTGGGGCCAGGGCACCCTGGTCACCGTTTCT |
| | AGCGGAGGCGGAGGATCTGGTGGCGGAGGAAGTGG |
| | CGGAGGCGGTTCTGAAAATGTGCTGACACAGAGCC |
| | CCAGCAGCATGTCCGTGTCTGTGGGCGACAGAGTG |
| | ACAATCGCCTGTAGCGCCTCTAGCAGCGTGCCCTA |
| | TATGCATTGGCTGCAGCAGAAGCCCGGCAAGAGCC |
| | CTAAGCTGCTGATCTACCTGACCAGCAATCTGGCC |
| | AGCGGCGTGCCAAGCAGATTTTCTGGAAGCGGCAG |
| | CGGCACCGACTACAGCCTGACAATCAGTAGCGTGC |
| | AGCCTGAGGATGCCGCCACCTACTACTGTCAGCAG |
| | AGAAGCAGCTACCCTCTGACCTTTGGAGGCGGCAC |
| | CAAGCTGGAAATCAAAGAGCAGAAGCTGATCTCCG |
| | AAGAGGACCTGACCACAACACCAGCTCCTAGACCT |
| | CCAACTCCTGCTCCTACAATCGCCCTGCAGCCACT |
| | GTCTCTGAGGCCTGAAGCTTGTAGACCTGCTGCTG |
| | GCGGAGCCGTGCATACCAGAGGACTGGATTTCGCC |
| | TGCGACATCTACATCTGGGCCCCTCTGGCTGGAAC |
| | ATGCGGAGTGTTGCTGCTGAGCCTGGTCATCACCA |
| | AGCGGGGCAGAAAGAAGCTGCTGTACATCTTCAAG |
| | CAGCCCTTCATGCGGCCCGTGCAGACCACACAAGA |
| | GGAAGATGGCTGCTCCTGCAGATTCCCCGAGGAAG |
| | AAGAAGGCGGCTGCGAGCTGAGAGTGAAGTTCAGC |
| | AGATCCGCCGACGCTCCTGCCTATCAGCAGGGACA |
| | GAACCAGCTGTACAACGAGCTGAACCTGGGGAGAA |
| | GAGAAGAGTACGACGTGCTGGACAAGCGGAGAGGC |
| | AGAGATCCTGAAATGGGCGGCAAGCCCAGACGGAA |
| | GAATCCTCAAGAGGGCCTGTATAATGAGCTGCAGA |
| | AAGACAAGATGGCCGAGGCCTACAGCGAGATCGGA |
| | ATGAAGGGCGAGCGCAGAAGAGGCAAGGGACACGA |
| | TGGACTGTACCAGGGCCTGAGCACCGCCACCAAGG |
| | ATACCTATGATGCCCTGCACATGCAGGCCCTGCCT |
| | CCAAGA |
| | (SEQ ID NO: 119) |
| SB03607 | MALPVTALLLPLALLLHAARPETVIKYLLPTAAAG |
| A.A. Seq | LLLLAAQPAMAQVKLQQSGAELVRSGTSVKLSCTA |
| | SGFNIKDSYMHWLRQGPEQGLEWIGWIDPENGDTE |
| | YAPKFQGKATFTTDTSSNTAYLQLSSLTSEDTAVY |
| | YCNEGTPTGPYYFDYWGQGTTVTVSSGGGGSGGGG |
| | SGGGGSENVLTQSPAIMSASPGEKVTITCSASSSV |
| | SYMHWFQQKPGTSPKLWIYSTSNLASGVPARFSGS |
| | GSGTSYSLTISRMEAEDAATYYCQQRSSYPLTFGA |
| | GTKLELKRAAEQKLISEEDLTTTPAPRPPTPAPTI |
| | ALQPLSLRPEACRPAAGGAVHTRGLDFACDIYIWA |
| | PLAGTCGVLLLSLVITKRGRKKLLYIFKQPFMRPV |
| | QTTQEEDGCSCRFPEEEEGGCELRVKFSRSADAPA |
| | YQQGQNQLYNELNLGRREEYDVLDKRRGRDPEMGG |
| | KPRRKNPQEGLYNELQKDKMAEAYSEIGMKGERRR |
| | GKGHDGLYQGLSTATKDTYDALHMQALPPR |
| | (SEQ ID NO: 120) |
| SB03609 | ATGGCTCTGCCTGTGACAGCTCTGCTGCTGCCTCT |
| DNA Seq | GGCTCTGCTTCTGCATGCTGCCAGACCTCAGATCG |
| | TGCTGTCTCAGTCTCCCGCCATCCTGTTTGCTAGC |
| | CCTGGCGAGAAAGTGACCATGACCTGTAGAGCCAG |
| | CAGCTCCGTGTCCTACATCCACTGGTATCAGCAGA |
| | AGCCCGGCAGCAGCCCTAAGCCTTGGATCCACGGC |
| | ACAAGCAATCTGGCCAGCGGAGTGCCTGCCAGATT |
| | TTCTGGAAGCGGCAGCGGCACCAGCTACAGCCTGA |
| | CCATCTCTAGAATGGAAGCCGAGGACGCCGCCACC |
| | TACTACTGTCAGCAGTGGTCCAGCAACCTGAGCAC |
| | CTTTGGCGGCGGAACAAAGCTGGAAATCAAAGGCG |
| | GCGGAGGATCTGGCGGAGGCGGTAGCGGAGGTGGT |
| | GGATCTGAAGTGAAGCTGGTGGAATCAGGCGGAGG |
| | CCTGGTTCAACCTGGCGGATCTCTGAGACTGAGCT |
| | GCAGCATCTCCGGCTTCACCTTCACCGACTACTAC |
| | ATGAACTGGGTCCGACAGAGCCCTGGCAAGGCTCT |
| | GGAATGGCTGGGCTTCATCAGAAACAAAGTGAACG |
| | GCGACACCACCGAGTACAGCGCCTCTGTGAAGGGC |
| | AGATTCACCATCAGCCGGGACATCAGCCAGAGCAT |
| | CCTGTACCTGCAGATGAACACCCTGCGGACCGAGG |
| | ATAGCGCCACATATTACTGCGCCAGAGACAAGGGA |
| | ATCGCCTACTACTTCGACTACTGGGGCCAGGGCAC |
| | CACACTGACAGTGTCTAGCGAGCAGAAGCTGATCT |
| | CCGAAGAGGACCTGACCACAACACCAGCTCCTAGA |
| | CCACCTACACCAGCACCTACAATCGCCCTGCAGCC |
| | ACTGTCTCTGAGGCCAGAAGCTTGTAGACCTGCTG |
| | CTGGCGGAGCCGTGCATACAAGAGGACTGGATTTC |
| | GCCTGCGACTTCCTGGTCATCATCGTGATCCTGAG |
| | CGCCCTGTTCCTGGGCACACTGGCCTGTTTCTGCG |
| | TGTGGCGCAGAAAGCGCAAAGAGAAGCAGAGCGAG |
| | ACAAGCCCCAAAGAGTTCCTGACCATCTACGAGGA |
| | CGTGAAGGACCTGAAAACCCGGCGGAACCACGAGC |
| | AAGAGCAGACCTTTCCTGGCGGCGGATCCACCATC |
| | TACAGCATGATCCAGAGCCAGAGCAGCGCCCCTAC |
| | AAGCCAAGAGCCTGCCTACACACTGTACAGCCTGA |
| | TTCAGCCCAGCAGAAAGAGCGGCTCCCGGAAGAGA |
| | AATCACAGCCCCAGCTTCAACAGCACGATCTACGA |
| | AGTGATCGGCAAGAGCCAGCCAAAGGCTCAGAACC |
| | CTGCCAGACTGAGCAGAAAAGAGCTGGAAAACTTC |
| | GACGTGTACAGCCGCGTGAAGTTCAGCAGATCCGC |
| | CGATGCTCCTGCCTATCAGCAGGGCCAGAACCAGC |
| | TGTACAACGAGCTGAACCTGGGGAGAAGAGAAGAG |
| | TACGACGTGCTGGACAAGCGGAGAGGCAGAGATCC |
| | TGAAATGGGCGGCAAGCCCAGACGGAAGAATCCTC |
| | AAGAGGGCCTGTATAATGAGCTGCAGAAAGACAAG |
| | ATGGCCGAGGCCTACAGCGAGATCGGAATGAAGGG |
| | CGAGCGCAGAAGAGGCAAGGGACACGATGGACTGT |
| | ACCAGGGCCTGAGCACCGCCACCAAGGATACCTAT |
| | GATGCCCTGCACATGCAGGCCCTGCCTCCAAGA |
| | (SEQ ID NO: 121) |
| SB03609 | MALPVTALLLPLALLLHAARPQIVLSQSPAILFAS |
| A.A. Seq | PGEKVTMTCRASSSVSYIHWYQQKPGSSPKPWIHG |
| | TSNLASGVPARFSGSGSGTSYSLTISRMEAEDAAT |
| | YYCQQWSSNLSTFGGGTKLEIKGGGGSGGGGSGGG |
| | GSEVKLVESGGGLVQPGGSLRLSCSISGFTFTDYY |
| | MNWVRQSPGKALEWLGFIRNKVNGDTTEYSASVKG |
| | RFTISRDISQSILYLQMNTLRTEDSATYYCARDKG |
| | IAYYFDYWGQGTTLTVSSEQKLISEEDLTTTPAPR |
| | PPTPAPTIALQPLSLRPEACRPAAGGAVHTRGLDF |
| | ACDFLVIIVILSALFLGTLACFCVWRRKRKEKQSE |
| | TSPKEFLTIYEDVKDLKTRRNHEQEQTFPGGGSTI |
| | YSMIQSQSSAPTSQEPAYTLYSLIQPSRKSGSRKR |
| | NHSPSFNSTIYEVIGKSQPKAQNPARLSRKELENF |
| | DVYSRVKFSRSADAPAYQQGQNQLYNELNLGRREE |
| | YDVLDKRRGRDPEMGGKPRRKNPQEGLYNELQKDK |
| | MAEAYSEIGMKGERRRGKGHDGLYQGLSTATKDTY |
| | DALHMQALPPR |
| | (SEQ ID NO: 122) |
| SB03610 | ATGGCTCTGCCTGTGACAGCTCTGCTGCTGCCTCT |
| DNA Seq | GGCTCTGCTTCTGCATGCTGCCAGACCTCAGATCG |
| | TGCTGTCTCAGTCTCCCGCCATCCTGTTTGCTAGC |
| | CCTGGCGAGAAAGTGACCATGACCTGTAGAGCCAG |
| | CAGCTCCGTGTCCTACATCCACTGGTATCAGCAGA |
| | AGCCCGGCAGCAGCCCTAAGCCTTGGATCCACGGC |
| | ACAAGCAATCTGGCCAGCGGAGTGCCTGCCAGATT |
| | TTCTGGAAGCGGCAGCGGCACCAGCTACAGCCTGA |
| | CCATCTCTAGAATGGAAGCCGAGGACGCCGCCACC |
| | TACTACTGTCAGCAGTGGTCCAGCAACCTGAGCAC |
| | CTTTGGCGGCGGAACAAAGCTGGAAATCAAAGGCG |
| | GCGGAGGATCTGGCGGAGGCGGTAGCGGAGGTGGT |
| | GGATCTGAAGTGAAGCTGGTGGAATCAGGCGGAGG |
| | CCTGGTTCAACCTGGCGGATCTCTGAGACTGAGCT |
| | GCAGCATCTCCGGCTTCACCTTCACCGACTACTAC |
| | ATGAACTGGGTCCGACAGAGCCCTGGCAAGGCTCT |
| | GGAATGGCTGGGCTTCATCAGAAACAAAGTGAACG |
| | GCGACACCACCGAGTACAGCGCCTCTGTGAAGGGC |
| | AGATTCACCATCAGCCGGGACATCAGCCAGAGCAT |
| | CCTGTACCTGCAGATGAACACCCTGCGGACCGAGG |
| | ATAGCGCCACATATTACTGCGCCAGAGACAAGGGA |
| | ATCGCCTACTACTTCGACTACTGGGGCCAGGGCAC |
| | CACACTGACAGTGTCTAGCGAGCAGAAGCTGATCT |
| | CCGAAGAGGACCTGACCACAACACCAGCTCCTAGA |
| | CCTCCAACTCCTGCTCCTACAATCGCCCTGCAGCC |
| | ACTGTCTCTGAGGCCTGAAGCTTGTAGACCTGCTG |
| | CTGGCGGAGCCGTGCATACCAGAGGACTGGATTTC |
| | GCCTGCGACATCTACATCTGGGCCCCTCTGGCTGG |
| | AACATGCGGAGTGTTGCTGCTGAGCCTGGTCATCA |
| | CCAAGCGGGGCAGAAAGAAGCTGCTGTACATCTTC |
| | AAGCAGCCCTTCATGCGGCCCGTGCAGACCACACA |
| | AGAGGAAGATGGCTGCTCCTGCAGATTCCCCGAGG |
| | AAGAAGAAGGCGGCTGCGAGCTGAGAGTGAAGTTC |
| | AGCAGATCCGCCGACGCTCCTGCCTATCAGCAGGG |
| | ACAGAACCAGCTGTACAACGAGCTGAACCTGGGGA |
| | GAAGAGAAGAGTACGACGTGCTGGACAAGCGGAGA |
| | GGCAGAGATCCTGAAATGGGCGGCAAGCCCAGACG |
| | GAAGAATCCTCAAGAGGGCCTGTATAATGAGCTGC |
| | AGAAAGACAAGATGGCCGAGGCCTACAGCGAGATC |
| | GGAATGAAGGGCGAGCGCAGAAGAGGCAAGGGACA |
| | CGATGGACTGTACCAGGGCCTGAGCACCGCCACCA |
| | AGGATACCTATGATGCCCTGCACATGCAGGCCCTG |
| | CCTCCAAGA |
| | (SEQ ID NO: 123) |
| SB03610 | MALPVTALLLPLALLLHAARPQIVLSQSPAILFAS |
| A.A. Seq | PGEKVTMTCRASSSVSYIHWYQQKPGSSPKPWIHG |
| | TSNLASGVPARFSGSGSGTSYSLTISRMEAEDAAT |
| | YYCQQWSSNLSTFGGGTKLEIKGGGGSGGGGSGGG |
| | GSEVKLVESGGGLVQPGGSLRLSCSISGFTFTDYY |
| | MNWVRQSPGKALEWLGFIRNKVNGDTTEYSASVKG |
| | RFTISRDISQSILYLQMNTLRTEDSATYYCARDKG |
| | IAYYFDYWGQGTTLTVSSEQKLISEEDLTTTPAPR |
| | PPTPAPTIALQPLSLRPEACRPAAGGAVHTRGLDF |
| | ACDIYIWAPLAGTCGVLLLSLVITKRGRKKLLYIF |
| | KQPFMRPVQTTQEEDGCSCRFPEEEEGGCELRVKF |
| | SRSADAPAYQQGQNQLYNELNLGRREEYDVLDKRR |
| | GRDPEMGGKPRRKNPQEGLYNELQKDKMAEAYSEI |
| | GMKGERRRGKGHDGLYQGLSTATKDTYDALHMQAL |
| | PPR |
| | (SEQ ID NO: 124) |
| SB04285 | ATGGCCCTGCCTGTGACAGCTCTGCTGCTGCCTCT |
| DNA Seq | GGCCCTGCTGCTGCATGCTGCTAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAACAAAAACTCATCTCAGAAG |
| | AAGATCTGAATGGGGCCGCAACCACGACGCCAGCG |
| | CCGCGACCACCAACACCGGCGCCCACCATCGCGTT |
| | GCAGCCCCTGTCCCTGCGCCCAGAGGCGTGCCGGC |
| | CAGCGGCGGGGGGCGCAGTGCACACGAGGGGGCTG |
| | GACTTCGCCTGTGATTTTTGGGTGCTGGTGGTGGT |
| | TGGTGGAGTCCTGGCTTGCTATAGCTTGCTAGTAA |
| | CAGTGGCCTTTATTATTTTCTGGGTGAGGAGTAAG |
| | AGGAGCAGGCTCCTGCACAGTGACTACATGAACAT |
| | GACTCCCCGCCGCCCCGGGCCCACCCGCAAGCATT |
| | ACCAGCCCTATGCCCCACCACGCGACTTCGCAGCC |
| | TATCGCTCCAGAGTGAAGTTCAGCAGGAGCGCAGA |
| | CGCCCCCGCGTACAAGCAGGGCCAGAACCAGCTCT |
| | ATAACGAGCTCAATCTAGGACGAAGAGAGGAGTAC |
| | GATGTTTTGGACAAGAGACGTGGCCGGGACCCTGA |
| | GATGGGGGGAAAGCCGAGAAGGAAGAACCCTCAGG |
| | AAGGCCTGTACAATGAACTGCAGAAAGATAAGATG |
| | GCGGAGGCCTACAGTGAGATTGGGATGAAAGGCGA |
| | GCGCCGGAGGGGCAAGGGGCACGATGGCCTTTACC |
| | AGGGTCTCAGTACAGCCACCAAGGACACCTACGAC |
| | GCCCTTCACATGCAGGCCCTGCCCCCTCGC |
| | (SEQ ID NO: 125) |
| SB04285 | MALPVTALLLPLALLLHAARPDIQLTQSPSSLSAS |
| A.A. Seq | VGDRVTITCKASQDVGTSVAWYQQKPGKAPKLLIY |
| | WTSTRHTGVPSRFSGSGSGTDFTFTISSLQPEDIA |
| | TYYCQQYSLYRSFGQGTKVEIKGGSGSGGSGSGGS |
| | GSEVQLVESGGGVVQPGRSLRLSCSASGFDFTTYW |
| | MSWVRQAPGKGLEWIGEIHPDSSTINYAPSLKDRF |
| | TISRDNAKNTLFLQMDSLRPEDTGVYFCASLYFGF |
| | PWFAYWGQGTPVTVSSEQKLISEEDLNGAATTTPA |
| | PRPPTPAPTIALQPLSLRPEACRPAAGGAVHTRGL |
| | DFACDFWVLVVVGGVLACYSLLVTVAFIIFWVRSK |
| | RSRLLHSDYMNMTPRRPGPTRKHYQPYAPPRDFAA |
| | YRSRVKFSRSADAPAYKQGQNQLYNELNLGRREEY |
| | DVLDKRRGRDPEMGGKPRRKNPQEGLYNELQKDKM |
| | AEAYSEIGMKGERRRGKGHDGLYQGLSTATKDTYD |
| | ALHMQALPPR |
| | (SEQ ID NO: 126) |
| SB04293 | ATGGCCCTGCCTGTGACAGCTCTGCTGCTGCCTCT |
| DNA Seq | GGCCCTGCTGCTGCATGCTGCTAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAACAAAAACTCATCTCAGAAG |
| | AAGATCTGAATGGGGCCGCAACCACGACGCCAGCG |
| | CCGCGACCACCAACACCGGCGCCCACCATCGCGTT |
| | GCAGCCCCTGTCCCTGCGCCCAGAGGCGTGCCGGC |
| | CAGCGGCGGGGGGCGCAGTGCACACGAGGGGGCTG |
| | GACTTCGCCTGTGATGTGGCCGCCATTCTTGGACT |
| | GGGACTTGTTCTGGGACTGCTGGGACCTCTGGCCA |
| | TTCTGCTGGCTCTGTACCTGCTGAGAAGGGACCAG |
| | AGACTGCCTCCTGACGCTCACAAACCTCCAGGCGG |
| | AGGCAGCTTCAGAACCCCTATCCAAGAGGAACAGG |
| | CTGACGCCCACAGCACCCTGGCCAAGATCAGAGTG |
| | AAGTTCAGCAGGAGCGCAGACGCCCCCGCGTACAA |
| | GCAGGGCCAGAACCAGCTCTATAACGAGCTCAATC |
| | TAGGACGAAGAGAGGAGTACGATGTTTTGGACAAG |
| | AGACGTGGCCGGGACCCTGAGATGGGGGGAAAGCC |
| | GAGAAGGAAGAACCCTCAGGAAGGCCTGTACAATG |
| | AACTGCAGAAAGATAAGATGGCGGAGGCCTACAGT |
| | GAGATTGGGATGAAAGGCGAGCGCCGGAGGGGCAA |
| | GGGGCACGATGGCCTTTACCAGGGTCTCAGTACAG |
| | CCACCAAGGACACCTACGACGCCCTTCACATGCAG |
| | GCCCTGCCCCCTCGC |
| | (SEQ ID NO: 127) |
| SB04293 | MALPVTALLLPLALLLHAARPDIQLTQSPSSLSAS |
| A.A. Seq | VGDRVTITCKASQDVGTSVAWYQQKPGKAPKLLIY |
| | WTSTRHTGVPSRFSGSGSGTDFTFTISSLQPEDIA |
| | TYYCQQYSLYRSFGQGTKVEIKGGSGSGGSGSGGS |
| | GSEVQLVESGGGVVQPGRSLRLSCSASGFDFTTYW |
| | MSWVRQAPGKGLEWIGEIHPDSSTINYAPSLKDRF |
| | TISRDNAKNTLFLQMDSLRPEDTGVYFCASLYFGF |
| | PWFAYWGQGTPVTVSSEQKLISEEDLNGAATTTPA |
| | PRPPTPAPTIALQPLSLRPEACRPAAGGAVHTRGL |
| | DFACDVAAILGLGLVLGLLGPLAILLALYLLRRDQ |
| | RLPPDAHKPPGGGSFRTPIQEEQADAHSTLAKIRV |
| | KFSRSADAPAYKQGQNQLYNELNLGRREEYDVLDK |
| | RRGRDPEMGGKPRRKNPQEGLYNELQKDKMAEAYS |
| | EIGMKGERRRGKGHDGLYQGLSTATKDTYDALHMQ |
| | ALPPR |
| | (SEQ ID NO: 128) |
| SB04288 | ATGGCCCTGCCTGTGACAGCTCTGCTGCTGCCTCT |
| DNA Seq | GGCCCTGCTGCTGCATGCTGCTAGACCTGAGGTCC |
| | AGCTGCAAGAGTCTGGCCCTGGACTTGTGAAGCCT |
| | GGCGGAAGCCTGTCTCTGTCTTGTGCCGCCAGCGG |
| | CTTCGTGTTCAGCAGCTACGATATGAGCTGGGTCC |
| | GACAGACCCCTGAGCGAGGACTTGAGTGGGTCGCC |
| | TACATCTCTTCTGGCGGCGGAATCACATACGCCCC |
| | TAGCACAGTGAAGGGCAGATTCACCGTGTCCAGAG |
| | ACAACGCCAAGAACACCCTGTACCTGCAGATGAAC |
| | AGCCTGACCAGCGAGGACACCGCCGTGTACTATTG |
| | TGCCGCTCACTACTTTGGCAGCAGCGGCCCTTTTG |
| | CCTATTGGGGCCAGGGAACACTCGTGACCGTTTCT |
| | AGTGCTGGCGGAGGCGGATCAGGTGGCGGAGGAAG |
| | TGGCGGCGGAGGATCTGATATCCAGATGACACAGA |
| | GCCCCGCCAGCCTGTCTGCCTCTGTGGGAGATAGA |
| | GTGACCATCACCTGTCGGGCCAGCGAGAACATCTT |
| | CAGCTACCTGGCCTGGTATCAGCAGAAGCCCGGCA |
| | AGTCTCCTAAGCTGCTGGTGTACAACACCCGGACA |
| | CTGGCTGAAGGCGTGCCCAGCAGATTTTCTGGCAG |
| | CGGCTCTGGCACCGACTTCAGCCTGACAATCAGCA |
| | GCCTGCAGCCTGAGGACTTCGCCACCTACTACTGC |
| | CAGCACCACTACGGCACCCCTTTCACCTTTGGAAG |
| | CGGCACCAAGCTGGAAATCAAGAGAGAACAAAAAC |
| | TCATCTCAGAAGAAGATCTGAATGGGGCCGCAACC |
| | ACGACGCCAGCGCCGCGACCACCAACACCGGCGCC |
| | CACCATCGCGTTGCAGCCCCTGTCCCTGCGCCCAG |
| | AGGCGTGCCGGCCAGCGGCGGGGGGCGCAGTGCAC |
| | ACGAGGGGGCTGGACTTCGCCTGTGATTTTTGGGT |
| | GCTGGTGGTGGTTGGTGGAGTCCTGGCTTGCTATA |
| | GCTTGCTAGTAACAGTGGCCTTTATTATTTTCTGG |
| | GTGAGGAGTAAGAGGAGCAGGCTCCTGCACAGTGA |
| | CTACATGAACATGACTCCCCGCCGCCCCGGGCCCA |
| | CCCGCAAGCATTACCAGCCCTATGCCCCACCACGC |
| | GACTTCGCAGCCTATCGCTCCAGAGTGAAGTTCAG |
| | CAGGAGCGCAGACGCCCCCGCGTACAAGCAGGGCC |
| | AGAACCAGCTCTATAACGAGCTCAATCTAGGACGA |
| | AGAGAGGAGTACGATGTTTTGGACAAGAGACGTGG |
| | CCGGGACCCTGAGATGGGGGGAAAGCCGAGAAGGA |
| | AGAACCCTCAGGAAGGCCTGTACAATGAACTGCAG |
| | AAAGATAAGATGGCGGAGGCCTACAGTGAGATTGG |
| | GATGAAAGGCGAGCGCCGGAGGGGCAAGGGGCACG |
| | ATGGCCTTTACCAGGGTCTCAGTACAGCCACCAAG |
| | GACACCTACGACGCCCTTCACATGCAGGCCCTGCC |
| | CCCTCGC |
| | (SEQ ID NO: 129) |
| SB04296 | ATGGCCCTGCCTGTGACAGCTCTGCTGCTGCCTCT |
| DNA Seq | GGCCCTGCTGCTGCATGCTGCTAGACCTGAGGTCC |
| | AGCTGCAAGAGTCTGGCCCTGGACTTGTGAAGCCT |
| | GGCGGAAGCCTGTCTCTGTCTTGTGCCGCCAGCGG |
| | CTTCGTGTTCAGCAGCTACGATATGAGCTGGGTCC |
| | GACAGACCCCTGAGCGAGGACTTGAGTGGGTCGCC |
| | TACATCTCTTCTGGCGGCGGAATCACATACGCCCC |
| | TAGCACAGTGAAGGGCAGATTCACCGTGTCCAGAG |
| | ACAACGCCAAGAACACCCTGTACCTGCAGATGAAC |
| | AGCCTGACCAGCGAGGACACCGCCGTGTACTATTG |
| | TGCCGCTCACTACTTTGGCAGCAGCGGCCCTTTTG |
| | CCTATTGGGGCCAGGGAACACTCGTGACCGTTTCT |
| | AGTGCTGGCGGAGGCGGATCAGGTGGCGGAGGAAG |
| | TGGCGGCGGAGGATCTGATATCCAGATGACACAGA |
| | GCCCCGCCAGCCTGTCTGCCTCTGTGGGAGATAGA |
| | GTGACCATCACCTGTCGGGCCAGCGAGAACATCTT |
| | CAGCTACCTGGCCTGGTATCAGCAGAAGCCCGGCA |
| | AGTCTCCTAAGCTGCTGGTGTACAACACCCGGACA |
| | CTGGCTGAAGGCGTGCCCAGCAGATTTTCTGGCAG |
| | CGGCTCTGGCACCGACTTCAGCCTGACAATCAGCA |
| | GCCTGCAGCCTGAGGACTTCGCCACCTACTACTGC |
| | CAGCACCACTACGGCACCCCTTTCACCTTTGGAAG |
| | CGGCACCAAGCTGGAAATCAAGAGAGAACAAAAAC |
| | TCATCTCAGAAGAAGATCTGAATGGGGCCGCAACC |
| | ACGACGCCAGCGCCGCGACCACCAACACCGGCGCC |
| | CACCATCGCGTTGCAGCCCCTGTCCCTGCGCCCAG |
| | AGGCGTGCCGGCCAGCGGCGGGGGGCGCAGTGCAC |
| | ACGAGGGGGCTGGACTTCGCCTGTGATGTGGCCGC |
| | CATTCTCGGACTGGGACTTGTTCTGGGACTGCTGG |
| | GACCTCTGGCCATTCTGCTGGCTCTGTACCTGCTG |
| | AGAAGGGACCAGAGACTGCCTCCTGACGCTCACAA |
| | ACCTCCAGGCGGAGGCAGCTTCAGAACCCCTATCC |
| | AAGAGGAACAGGCTGACGCCCACAGCACCCTGGCC |
| | AAGATCAGAGTGAAGTTCAGCAGGAGCGCAGACGC |
| | CCCCGCGTACAAGCAGGGCCAGAACCAGCTCTATA |
| | ACGAGCTCAATCTAGGACGAAGAGAGGAGTACGAT |
| | GTTTTGGACAAGAGACGTGGCCGGGACCCTGAGAT |
| | GGGGGGAAAGCCGAGAAGGAAGAACCCTCAGGAAG |
| | GCCTGTACAATGAACTGCAGAAAGATAAGATGGCG |
| | GAGGCCTACAGTGAGATTGGGATGAAAGGCGAGCG |
| | CCGGAGGGGCAAGGGGCACGATGGCCTTTACCAGG |
| | GTCTCAGTACAGCCACCAAGGACACCTACGACGCC |
| | CTTCACATGCAGGCCCTGCCCCCTCGC |
| | (SEQ ID NO: 130) |
| SB04442 | ATGGCTCTGCCTGTGACAGCTCTGCTGCTGCCTCT |
| DNA Seq | GGCTCTGCTTCTGCATGCCGCCAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAGCAGAAGCTGATCTCCGAAG |
| | AGGACCTGACCACAACACCAGCTCCTAGACCACCT |
| | ACACCAGCACCTACAATCGCCCTGCAGCCACTGTC |
| | TCTGAGGCCTGAAGCTTGTAGACCTGCTGCTGGCG |
| | GAGCCGTGCATACAAGAGGACTGGATTTCGCCTGC |
| | GACTTCTGGGTGCTCGTTGTTGTTGGCGGCGTGCT |
| | GGCCTGTTATTCCCTGCTGGTTACCGTGGCCTTCA |
| | TCATCTTTTGGGTCCGAAGCAAGCGGAGCAGACTG |
| | CTGCACTCCGACTACATGAACATGACCCCTAGACG |
| | GCCCGGACCAACCAGAAAGCACTACCAGCCTTACG |
| | CTCCTCCTAGAGACTTCGCCGCCTACCGGTCCAGA |
| | GTGAAGTTCTCCAGATCCGCCGATGCTCCCGCCTA |
| | TAAGCAGGGACAGAACCAGCTGTACAACGAGCTGA |
| | ACCTGGGGAGAAGAGAAGAGTACGACGTGCTGGAC |
| | AAGCGGAGAGGCAGAGATCCTGAGATGGGCGGCAA |
| | GCCCAGACGGAAGAATCCTCAAGAGGGCCTGTATA |
| | ATGAGCTGCAAAAGGACAAGATGGCCGAGGCCTAC |
| | AGCGAGATCGGAATGAAGGGCGAGCGCAGAAGAGG |
| | CAAGGGACACGATGGACTGTACCAGGGCCTGAGCA |
| | CCGCCACCAAGGATACCTATGATGCCCTGCACATG |
| | CAGGCCCTGCCTCCAAGA |
| | (SEQ ID NO: 131) |
| SB04443 | ATGGCTCTGCCTGTGACAGCTCTGCTGCTGCCTCT |
| DNA Seq | GGCTCTGCTTCTGCATGCCGCCAGACCTGACATCC |
| | AGCTGACACAGAGCCCTAGCAGCCTGTCTGCCTCT |
| | GTGGGCGACAGAGTGACCATCACATGCAAGGCCTC |
| | TCAGGACGTGGGCACAAGCGTGGCATGGTATCAGC |
| | AGAAGCCTGGCAAGGCCCCTAAGCTGCTGATCTAC |
| | TGGACCAGCACCAGACACACAGGCGTGCCCAGCAG |
| | ATTTTCTGGCAGCGGCTCTGGCACCGACTTCACCT |
| | TCACCATAAGCAGCCTGCAGCCTGAGGATATCGCC |
| | ACCTACTACTGCCAGCAGTACAGCCTGTACAGAAG |
| | CTTCGGCCAGGGCACCAAGGTGGAAATCAAAGGCG |
| | GATCTGGAAGCGGCGGTTCTGGATCTGGTGGAAGC |
| | GGATCTGAGGTGCAGCTGGTGGAATCTGGTGGCGG |
| | AGTTGTGCAGCCTGGCAGATCTCTGAGACTGAGCT |
| | GTAGCGCCAGCGGCTTCGATTTCACCACCTACTGG |
| | ATGAGCTGGGTCCGACAGGCCCCTGGCAAAGGACT |
| | GGAATGGATCGGCGAGATTCACCCCGACAGCAGCA |
| | CCATCAATTACGCCCCTAGCCTGAAGGACCGGTTC |
| | ACCATCTCCAGAGACAACGCCAAGAATACCCTGTT |
| | CCTGCAGATGGACAGCCTCCGGCCTGAAGATACCG |
| | GCGTGTACTTTTGCGCCAGCCTGTATTTCGGCTTC |
| | CCTTGGTTTGCCTACTGGGGCCAGGGAACACCTGT |
| | GACCGTTAGCTCTGAGCAGAAGCTGATCTCCGAAG |
| | AGGACCTGACCACAACACCAGCTCCTAGACCACCT |
| | ACACCAGCACCTACAATCGCCCTGCAGCCACTGTC |
| | TCTGAGGCCAGAGGCTTGTAGACCTGCTGCTGGCG |
| | GAGCCGTGCATACAAGAGGACTGGATTTCGCCTGC |
| | GACGTGGCCGCCATTCTCGGACTGGGACTTGTTCT |
| | GGGACTGCTGGGACCTCTGGCCATTCTGCTGGCTC |
| | TGTACCTGCTGAGAAGGGACCAGAGACTGCCTCCT |
| | GACGCTCACAAACCTCCAGGCGGAGGCAGCTTCAG |
| | AACCCCTATCCAAGAGGAACAGGCTGACGCCCACA |
| | GCACCCTGGCCAAGATCAGAGTGAAGTTCAGCAGA |
| | TCCGCCGACGCTCCCGCCTATAAGCAGGGACAGAA |
| | TCAGCTGTACAACGAGCTGAACCTGGGGCGCAGAG |
| | AAGAGTACGACGTGCTGGACAAGAGAAGAGGCAGG |
| | GATCCTGAAATGGGCGGCAAGCCCAGACGGAAGAA |
| | TCCTCAAGAGGGCCTGTATAATGAGCTGCAGAAAG |
| | ACAAGATGGCCGAGGCCTACAGCGAGATCGGAATG |
| | AAGGGCGAACGCAGAAGAGGAAAGGGCCACGACGG |
| | ACTGTATCAGGGCCTGAGCACAGCCACCAAGGACA |
| | CCTATGATGCCCTGCACATGCAGGCCCTGCCTCCA |
| | AGA |
| | (SEQ ID NO: 132) |
| SB04444 | ATGGCTCTGCCTGTGACAGCTCTGCTGCTGCCTCT |
| DNA Seq | GGCTCTGCTTCTGCATGCCGCTAGACCTGAGGTCC |
| | AGCTGCAAGAGTCTGGCCCTGGACTTGTGAAGCCT |
| | GGCGGAAGCCTGTCTCTGTCTTGTGCCGCCAGCGG |
| | CTTCGTGTTCAGCAGCTACGATATGAGCTGGGTCC |
| | GACAGACCCCTGAGCGAGGACTTGAGTGGGTCGCC |
| | TACATCTCTTCTGGCGGCGGAATCACATACGCCCC |
| | TAGCACAGTGAAGGGCAGATTCACCGTGTCCAGAG |
| | ACAACGCCAAGAACACCCTGTACCTGCAGATGAAC |
| | AGCCTGACCAGCGAGGACACCGCCGTGTACTATTG |
| | TGCCGCTCACTACTTTGGCAGCAGCGGCCCTTTTG |
| | CCTATTGGGGCCAGGGAACACTCGTGACCGTTTCT |
| | AGTGCTGGCGGAGGCGGATCAGGTGGCGGAGGAAG |
| | TGGCGGCGGAGGATCTGATATCCAGATGACACAGA |
| | GCCCCGCCAGCCTGTCTGCCTCTGTGGGAGATAGA |
| | GTGACCATCACCTGTCGGGCCAGCGAGAACATCTT |
| | CAGCTACCTGGCCTGGTATCAGCAGAAGCCCGGCA |
| | AGTCTCCTAAGCTGCTGGTGTACAACACCCGGACA |
| | CTGGCTGAAGGCGTGCCCAGCAGATTTTCTGGCAG |
| | CGGCTCTGGCACCGACTTCAGCCTGACAATCAGCA |
| | GCCTGCAGCCTGAGGACTTCGCCACCTACTACTGC |
| | CAGCACCACTACGGCACCCCTTTCACCTTTGGAAG |
| | CGGCACCAAGCTGGAAATCAAGAGAGAGCAGAAGC |
| | TGATCTCCGAAGAGGACCTGACCACAACTCCTGCT |
| | CCTAGACCTCCTACACCAGCTCCTACAATCGCCCT |
| | GCAGCCACTGTCTCTGAGGCCAGAAGCTTGTAGAC |
| | CTGCTGCAGGCGGAGCCGTGCATACAAGAGGACTG |
| | GATTTCGCCTGCGACTTCTGGGTGCTCGTGGTTGT |
| | TGGCGGAGTGCTGGCCTGTTACTCTCTGCTGGTCA |
| | CCGTGGCCTTCATCATCTTTTGGGTCCGAAGCAAG |
| | CGGAGCCGGCTGCTGCACAGCGACTACATGAACAT |
| | GACCCCTAGACGGCCCGGACCTACCAGAAAGCACT |
| | ACCAGCCTTACGCTCCTCCTAGAGATTTCGCCGCC |
| | TACCGGTCCAGAGTGAAGTTCAGCAGATCCGCCGA |
| | TGCTCCCGCCTATAAGCAGGGACAGAACCAGCTGT |
| | ACAACGAGCTGAACCTGGGGAGAAGAGAAGAGTAC |
| | GACGTGCTGGACAAGCGGAGAGGCAGAGATCCTGA |
| | GATGGGCGGCAAGCCCAGACGGAAGAATCCTCAAG |
| | AGGGCCTGTATAATGAGCTGCAGAAAGACAAGATG |
| | GCCGAGGCCTACAGCGAGATCGGAATGAAGGGCGA |
| | GCGCAGAAGAGGCAAGGGACACGATGGACTGTACC |
| | AGGGCCTGAGCACCGCCACCAAGGATACCTATGAT |
| | GCCCTGCACATGCAGGCCCTGCCTCCAAGA |
| | (SEQ ID NO: 133) |
| SB04445 | ATGGCTCTGCCTGTGACAGCTCTGCTGCTGCCTCT |
| DNA Seq | GGCTCTGCTTCTGCATGCCGCTAGACCTGAGGTCC |
| | AGCTGCAAGAGTCTGGCCCTGGACTTGTGAAGCCT |
| | GGCGGAAGCCTGTCTCTGTCTTGTGCCGCCAGCGG |
| | CTTCGTGTTCAGCAGCTACGATATGAGCTGGGTCC |
| | GACAGACCCCTGAGCGAGGACTTGAGTGGGTCGCC |
| | TACATCTCTTCTGGCGGCGGAATCACATACGCCCC |
| | TAGCACAGTGAAGGGCAGATTCACCGTGTCCAGAG |
| | ACAACGCCAAGAACACCCTGTACCTGCAGATGAAC |
| | AGCCTGACCAGCGAGGACACCGCCGTGTACTATTG |
| | TGCCGCTCACTACTTTGGCAGCAGCGGCCCTTTTG |
| | CCTATTGGGGCCAGGGAACACTCGTGACCGTTTCT |
| | AGTGCTGGCGGAGGCGGATCAGGTGGCGGAGGAAG |
| | TGGCGGCGGAGGATCTGATATCCAGATGACACAGA |
| | GCCCCGCCAGCCTGTCTGCCTCTGTGGGAGATAGA |
| | GTGACCATCACCTGTCGGGCCAGCGAGAACATCTT |
| | CAGCTACCTGGCCTGGTATCAGCAGAAGCCCGGCA |
| | AGTCTCCTAAGCTGCTGGTGTACAACACCCGGACA |
| | CTGGCTGAAGGCGTGCCCAGCAGATTTTCTGGCAG |
| | CGGCTCTGGCACCGACTTCAGCCTGACAATCAGCA |
| | GCCTGCAGCCTGAGGACTTCGCCACCTACTACTGC |
| | CAGCACCACTACGGCACCCCTTTCACCTTTGGAAG |
| | CGGCACCAAGCTGGAAATCAAGAGAGAGCAGAAGC |
| | TGATCTCCGAAGAGGACCTGACCACAACACCAGCT |
| | CCTAGACCACCTACACCAGCACCTACAATCGCCCT |
| | GCAGCCACTGTCTCTGAGGCCAGAGGCTTGTAGAC |
| | CTGCTGCTGGCGGAGCCGTGCATACAAGAGGACTG |
| | GATTTCGCCTGCGACGTGGCCGCCATTCTCGGACT |
| | GGGACTTGTTCTGGGACTGCTGGGACCTCTGGCCA |
| | TTCTGCTGGCTCTGTACCTGCTGAGAAGGGACCAG |
| | AGACTGCCTCCTGACGCTCACAAACCTCCAGGCGG |
| | AGGCAGCTTCAGAACCCCTATCCAAGAGGAACAGG |
| | CTGACGCCCACAGCACCCTGGCCAAGATCAGAGTG |
| | AAGTTCAGCAGATCCGCCGACGCTCCCGCCTATAA |
| | GCAGGGACAGAATCAGCTGTACAACGAGCTGAACC |
| | TGGGGCGCAGAGAAGAGTACGACGTGCTGGACAAG |
| | AGAAGAGGCAGGGATCCTGAAATGGGCGGCAAGCC |
| | CAGACGGAAGAATCCTCAAGAGGGCCTGTATAATG |
| | AGCTGCAGAAAGACAAGATGGCCGAGGCCTACAGC |
| | GAGATCGGAATGAAGGGCGAACGCAGAAGAGGAAA |
| | GGGCCACGACGGACTGTATCAGGGCCTGAGCACAG |
| | CCACCAAGGACACCTATGATGCCCTGCACATGCAG |
| | GCCCTGCCTCCAAGA |
| | (SEQ ID NO: 134) |

### Example 12: Lentiviral and Gamma-Retroviral Platforms for Expressing CEA CARs

In this example, lentiviral and gamma-retroviral platforms for expressing various anti-CEA activating CARs were assessed.

NK cells were transduced using lentiviral or retroviral vectors containing different CAR constructs. The various constructs are shown in Table 29.

TABLE 29
NK Donor 2 cells

| Retroviral | Lentiviral | | scFv | | | | |
|---|---|---|---|---|---|---|---|
| Construct name | Construct name | scFv name | orientation | hinge | TM | co-stim | stim |
| SB03173 | SB02463 | hMN14 | H/L | CD8 | CD28 | CD28 | CD3z |
| SB03174 | SB02779 | hMN14 | H/L | CD8 | OX40 | OX40 | CD3z |
| SB03175 | SB02781 | hMN14 | H/L | CD8 | CD8 | 4-1BB | CD3z |
| SB03176 | SB02465 | MFE-23 | H/L | CD8 | CD28 | CD28 | CD3z |
| SB03177 | SB02470 | MRG1 | H/L | CD8 | CD28 | CD28 | CD3z |
| SB03178 | SB02788 | MRG1 | H/L | CD8 | OX40 | OX40 | CD3z |
| SB03179 | SB02790 | MRG1 | H/L | CD8 | CD8 | 4-1BB | CD3z |
| SB03180 | SB03089 | Tus | H/L | CD8 | CD28 | CD28 | CD3z |

CAR expression was measured using flow cytometry and a MYC tag protein in the extracellular membrane. CAR expression was measured at different time points (expression from lentiviral and gamma-retroviral constructs were both assessed day 4, and expression from gamma-retroviral constructs was again assessed on day 7 and day 11 post transduction).

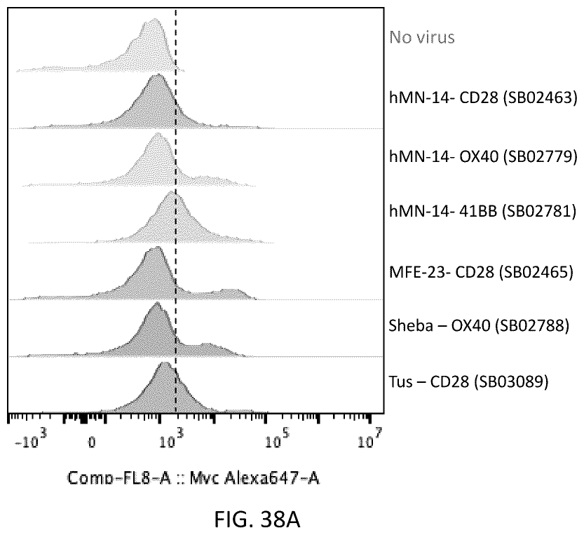

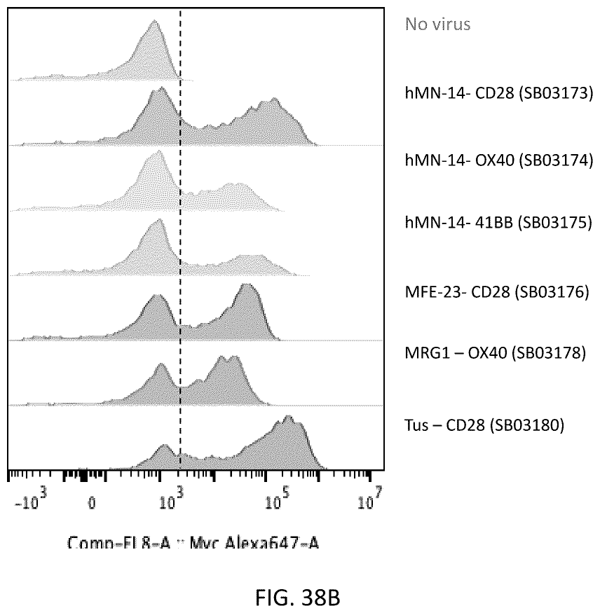

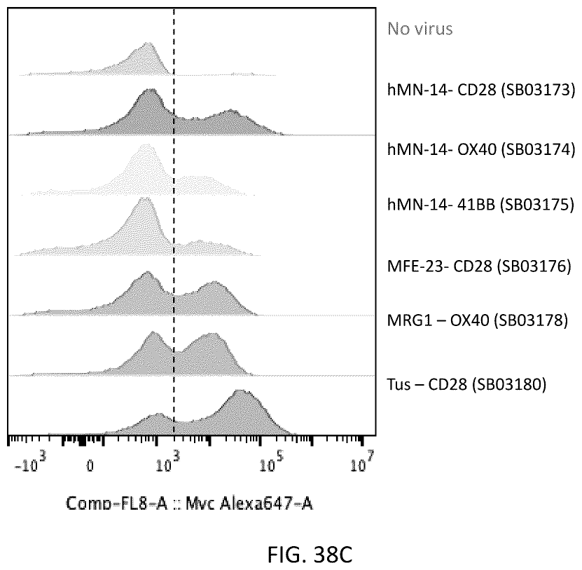

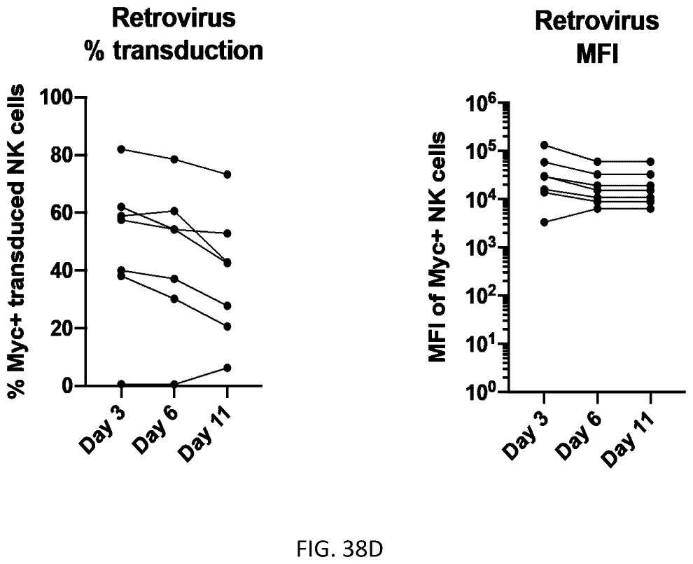

CAR expression was quantified either by measuring the % transduction (% positive cells compared to an un-transduced negative control) or by measuring the MFI (Geometric Mean of Fluorescence Intensity of the population). Expression is shown in FIG. 38A (day 4 post-transduction), FIG. 38B (day 11 post-transduction), and FIG. 38C (time-course of expression from gamma-retroviral constructs). As can be seen in FIG. 38A, retroviral constructs result in a much higher transduction efficiency as well as a clearer positive population. As can be seen in FIGS. 38B and 38C, retroviral constructs result in durable expression over at least 11 days.

### Example 13: Anti-CEA CAR Expression from Various Gamma-Retroviral Systems

In this example, various gamma-retroviral platforms for expressing s anti-CEA activating CARs were assessed. The various systems assessed are shown in Table 30.

TABLE 30
Various Gamma-Retroviral Systems

| | Retrovirus | | | | |
|---|---|---|---|---|---|
| Transduction | Construct | | scFv | Codon | costim |
| Group | Backbone | Env | name | Optimization | domain |
| A | 1 | BaEv | hMN14 | Human codon | CD28 |
| | | | | optimization | |
| | 1 | BaEv | hMN14 | Human codon | OX40 |
| | | | | optimization | |
| | 1 | BaEv | Tus | Human codon | CD28 |
| | | | | optimization | |
| | 1 | BaEv | Tus | Human codon | OX40 |
| | | | | optimization | |
| B | 1 | BaEv | hMN14 | New codon | CD28 |
| | | | | optimization | |
| | 1 | BaEv | hMN14 | New codon | OX40 |
| | | | | optimization | |
| | 1 | BaEv | Tus | New codon | CD28 |
| | | | | optimization | |
| | 1 | BaEv | Tus | New codon | OX40 |
| | | | | optimization | |
| C | 2 | BaEv | hMN14 | Human codon | CD28 |
| | | | | optimization | |
| | 2 | BaEv | hMN14 | Human codon | OX40 |
| | | | | optimization | |
| | 2 | BaEv | Tus | Human codon | CD28 |
| | | | | optimization | |
| | 2 | BaEv | Tus | Human codon | OX40 |
| | | | | optimization | |

Primary NK cells were transduced with gamma-retroviruses pseudotyped with EaEV or BaEv, transduced performed using 3e9 vector genome units per/1e6 NK cells. CAR expression was measured using flow cytometry and a MYC tag protein in the extracellular membrane. CAR expression was measured on day 11 post transduction.

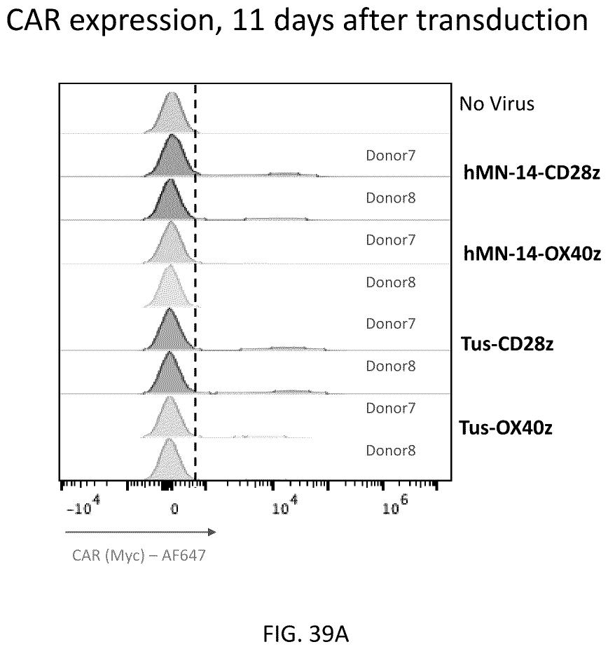

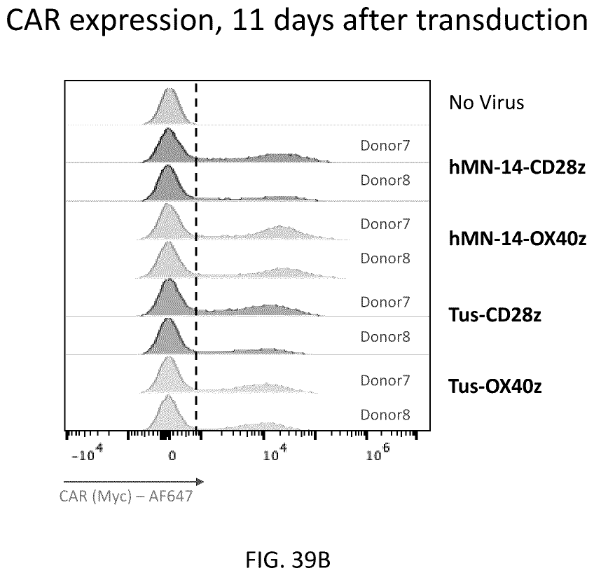

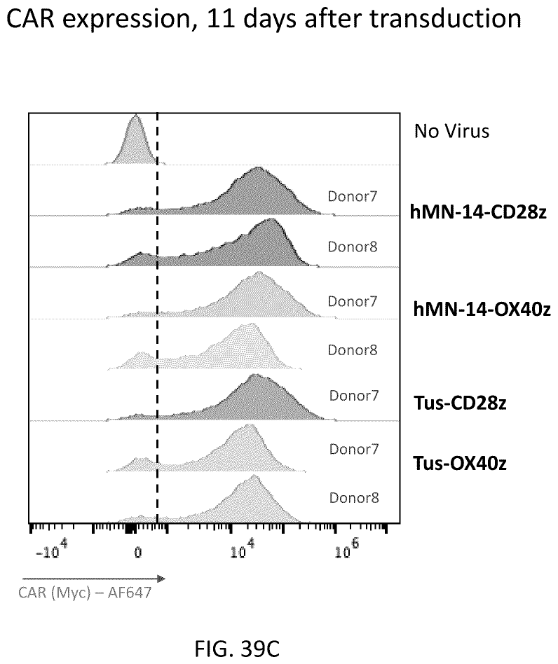

CAR expression was quantified by measuring the % transduction (% positive cells compared to an un-transduced negative control. Expression is shown in FIG. 39A (Transduction Group A), FIG. 39B (Transduction Group B), FIG. 39C (Transduction Group C. As can be seen in FIGS. 39A-39D, high expression was achieved using both retroviral backbones and both pseudotyping envelopes (see transduction groups B and D). In particular, using the second back bone pseudotyped with BaEV resulted in greater than 90% of cell positive for the CEA CAR.

### Example 14: Anti-CEA CAR Expression and Cytotoxicity Using Various CAR Architectures

In this example, anti-CEA activating CARs having an hMN-14-based scFv and various costimulatory domains were assessed using primary NK cells derived from two different donors (“donor 7” and “donor 13”). Each CAR included a Myc tag for measuring expression. Construct descriptions are provided in Table 31.

TABLE 31

| Construct Name | CAR Architecture |
|---|---|
| SB05382 | hMN14 scFv-ICD: |
| | CD28/CD3zeta |
| SB05384 | hMN14 scFV-ICD: |
| | OX40/CD3zeta |
| SB04446 | hMN14 scFv-ICD: |
| | 41bb/CD3zeta |

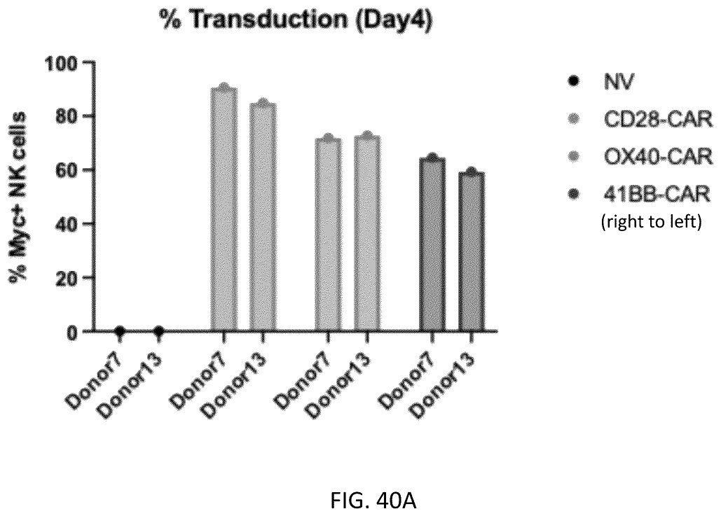

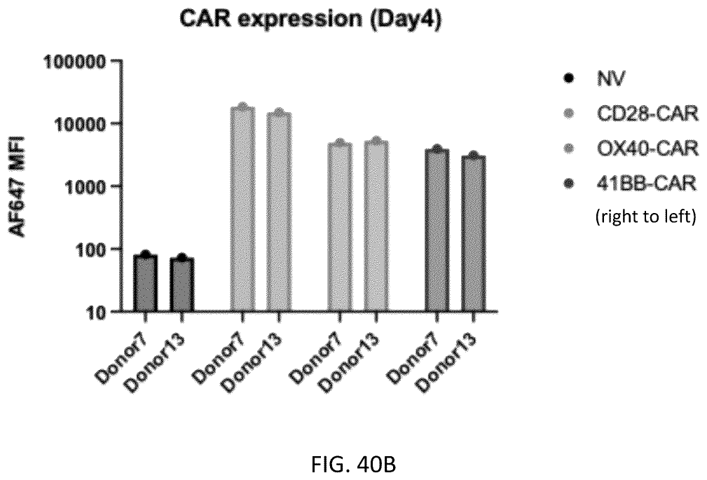

Percent transduction was measured based on the percentage of cells positive for Myc and is shown in FIG. 40A, and CAR expression was measured based on mean fluorescent intensity (MFI) and is shown in FIG. 40B.

Next, the CAR-NK cells were assessed for killing activity. CAR-NK cells were co-cultured with antigen-expressing colorectal cancer cells (Ls174t) that were transduced to stably express the fluorescent protein mKate.

25,000 target cells were seeded in 96 well plates. Engineered or control NK cells were added at various effector to target cell ratios (from 2:1 to 1:4). Target cell area (red area) was measured every 2 hours using Incucyte

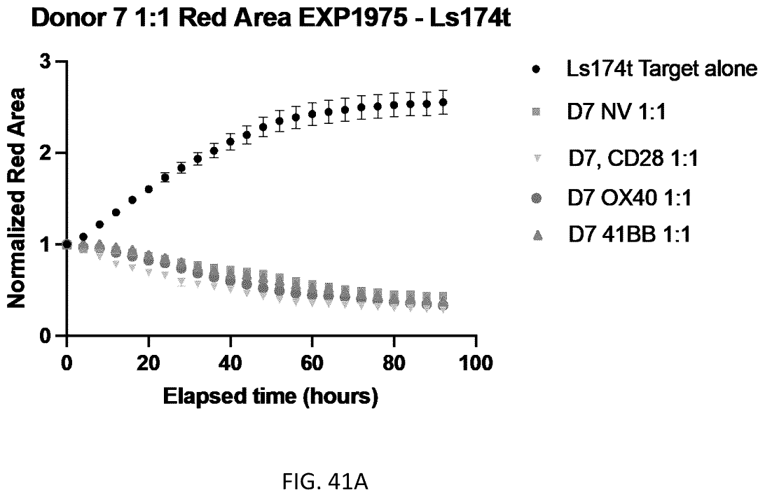

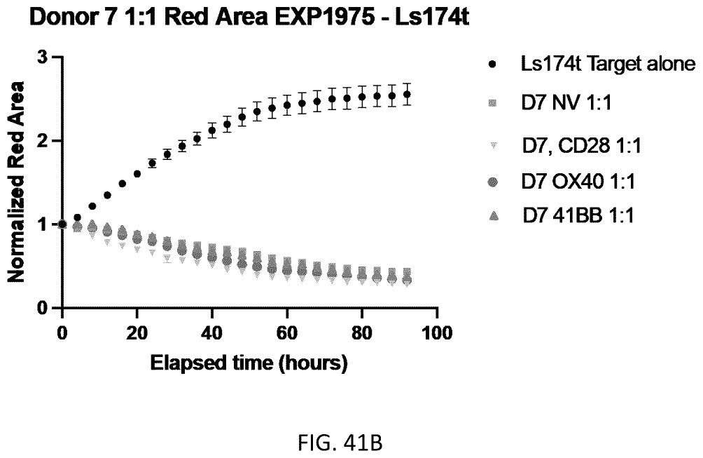

Killing assays performed with an effector to target cell ratio of 1:1 are shown in FIGS. 41A and 41B. As can be seen in FIGS. 41A and 41B, donor 7 cells had a high baseline of killing (independent of the presence of a CAR), but donor 13 cells had lower baseline killing with the presence of the CEA CAR resulting in a higher amount of killing than NK cells not expressing the CAR.

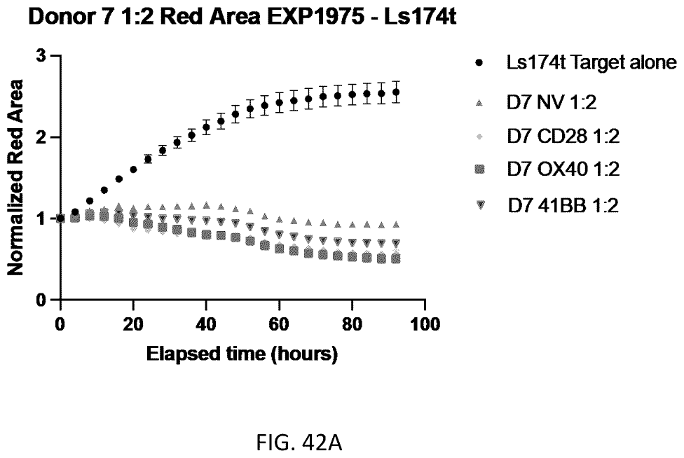

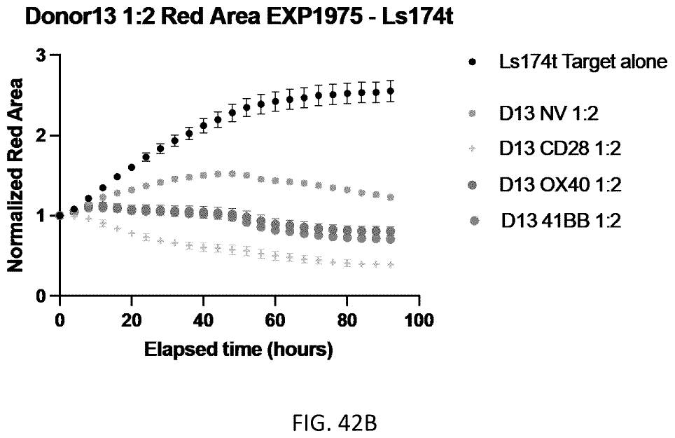

Killing assays performed with an effector to target cell ratio of 1:2 are shown in FIGS. 42A and 42B. Again, as with the effector to target ratio of 1:1, at a ratio of 1:2 donor 7 cells had a higher baseline of killing (independent of the presence of a CAR), but at this lower ratio of effector cells an increase in killing based on the presence of the CEA CAR is observed. Donor 13 cells had lower baseline killing with the presence of the CEA CAR and also an increased amount of killing based on the presence of the CEA CAR.

### Example 15: CEA CAR NK Cell Function In Vitro and In Vivo

In this example, anti-CEA activating CARs having an hMN-14-based scFv and various costimulatory domains were assessed using primary NK cells derived from two different donors.

CEA-CAR NK cells were generated by transducing expanded/activated NK cells with retroviral constructs containing various CEA-CAR constructs.

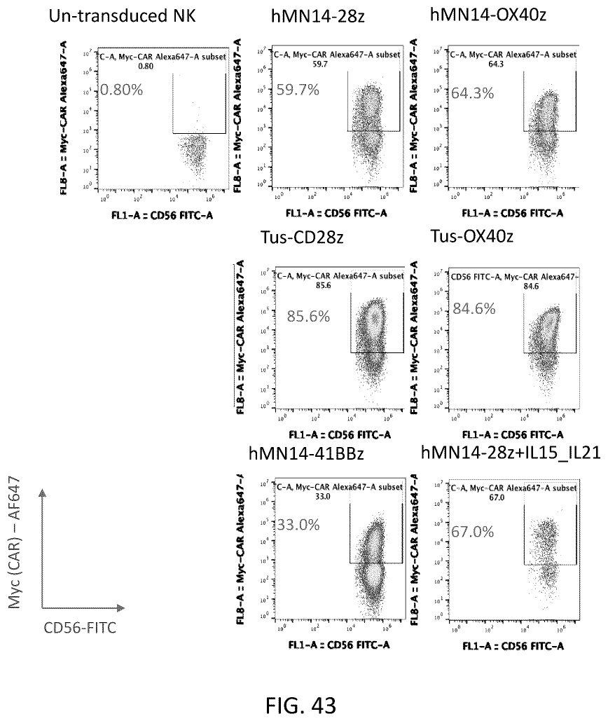

CAR expression was assessed via flow cytometry detection of a MYC tag and is shown in FIG. 43.

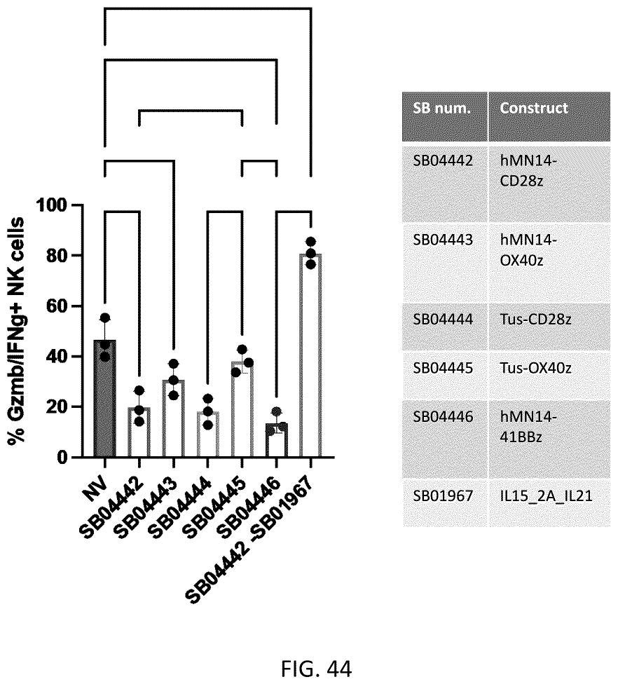

CAR-NK cells were co-cultured with CEA-expressing CRC target cells (Ls174t) at a 1:1 ratio overnight (18 hours). NK cells were fixed and stained with antibodies to detect intracellular cytokines IFNg and granzyme B (“GZMB”) which are markers of NK cell activation as shown in FIG. 44. As can be seen in FIG. 44, different constructs resulted in different degrees of activity on the NK cells.

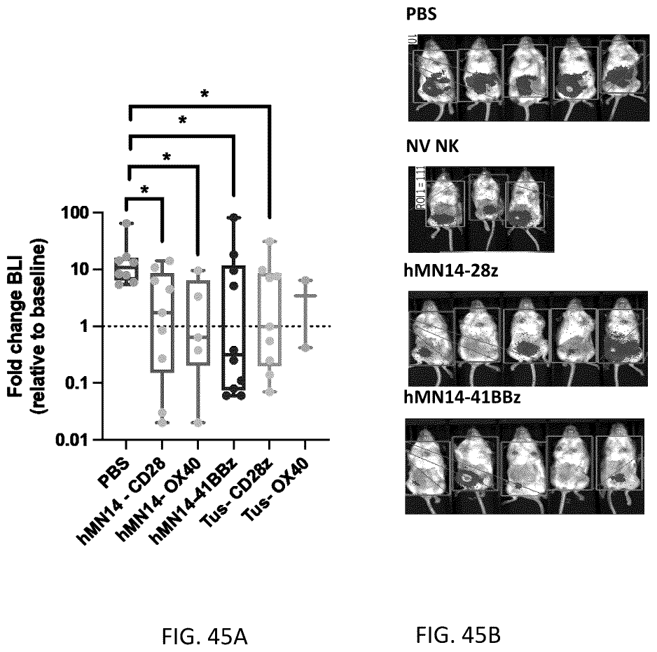

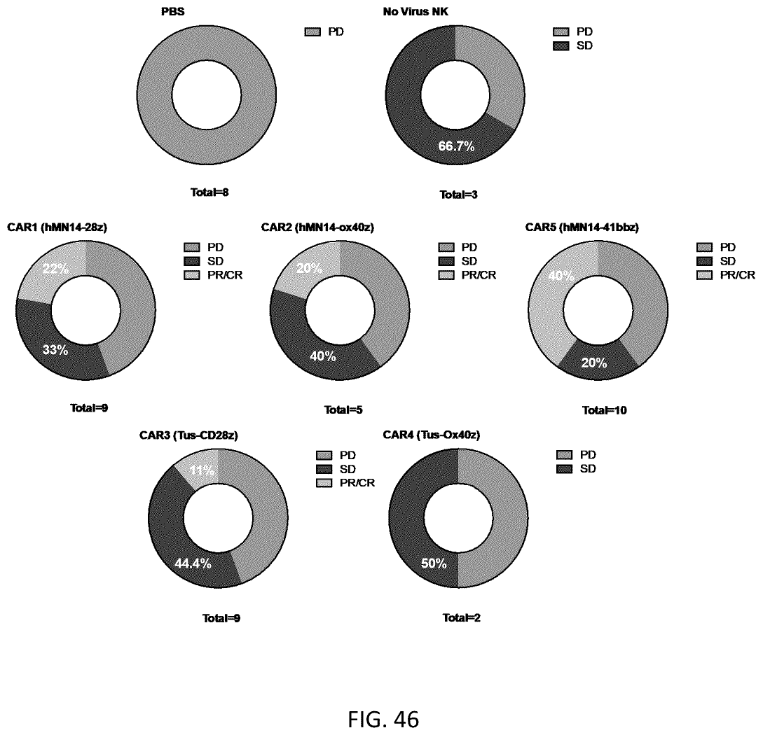

The same CEA-CAR NK cells were used to treat NSG female mice (6-8 weeks of age) with established Ls174t peritoneal tumors. No virus (NV) NK cells and PBS were used as controls. Tumor burden was measured by BLI and normalized to the pre-treatment values to calculate the fold change. Representative image of tumor burden BLI after treatment are shown in FIG. 45. Response rates at day 10 are shown in pie charts in FIG. 46. PD means progressive disease measured as a fold change of greater than 5 BLI. SD means stable disease measured as a fold change of 0.1 to 5 BLI, and PR/CR means partial or complete response measured as fold change of less than 0.1 BL1 (including negative values).

### Example 16: NOT Gate-Based Protection of Safety Antigen-Positive Cells

In this example, use of a NOT-Gate in combination with a CEA activating CAR was assessed using cell expressing a safety antigen (model safety antigen HER2, or safety antigen VSIG2)

First, cell lines expressing safety antigens (HER2 or VSIG2) were generated. CRC cells (Ls174t, DLD1, and HT29) or SEM cells were transduced using lentivirus expressing the Her2 or VSIG2 proteins and a resistance selection marker (blasticidin). Cells were selected for antibiotic resistance and expression of the desired protein was assessed via flow cytometry. The CRC cell lines Ls174t and HT29 naturally express CEACAM5, but DLD1 does not. Thus, the DLD1 CRC cells, were also transduced with lentivirus encoding for a membrane-bound form of CEACAM5-EGFP. Cells were sorted for EGFP positive and co-expression of CEACAM5 and VSIG2 was determined via flow cytometry.

Next, NK cells expressing a CEA activating CAR and either an anti-VSIG2 inhibitory CAR or an anti-HER2 inhibitory CAR were generated. An anti-HER2 inhibitory CAR having a LIR1 inhibitory domain was generated, and various anti-VSIG2 inhibitory CARs having various inhibitory domains (listed in order by proximity to the membrane: two LIR1 domains, a KIR3DL1 domain, a KIR3DL1 followed by a KIR3DL1 domain, a LIR1 domain followed by a KIR3DL1 domain, a KIR3DL1 domain followed by a LIR1 domain, a KIR2DL1 domain, a LAIR1 domain, a SIGLEC2 domain, and a SIRPa domain). Construct descriptions are provided in Table 32.

TABLE 32

| Construct Name | CAR Description |
|---|---|
| “LIR1-LIR1” | aVSIG2 (L-w-H) |
| | LIR1-LIR1 2A PuroR |
| “KIR3DL1” | aVSIG2 (L-w-H) |
| | KIR3DL1 2A PuroR |
| “KIR3DL1-KIR3DL1” | aVSIG2 (L-w-H) |
| | KIR3DL1-KIR3DL1 |
| | 2A PuroR |
| “LIR1-KIR3DL1” | aVSIG2 (L-w-H) |
| | LIR1-KIR3DL1 |
| | 2A PuroR |
| “KIR3DL1-LIR1” | aVSIG2 (L-w-H) |
| | KIR3DL1-LIR1 |
| | 2A PuroR |
| “KIR2DL1” | aVSIG2 (L-w-H) |
| | KIR2DL1 2A PuroR |
| “LAIR1” | aVSIG2 (L-w-H) |
| | LAIR1 2A PuroR |
| “SIGLEC-2” | aVSIG2 (L-w-H) |
| | SIGLEC-2 2A PuroR |
| “SIRPa” | aVSIG2 (L-w-H) |
| | SIRPa 2A PuroR |

The anti-VSIG2 scFV sequence used in the anti-VSIG inhibitory CARs is provided in Table 33.

TABLE 33

| Anti-VSIG2 scFv sequence | SEQ ID NO |
|---|---|
| DIQMTQSPASLSASVGETVTMTCRA | R1-LIR1 2A PuroR |
| SENIYSYLAWYQQKQGKSPQLLVFN | |
| AETLPEGVPSRFSGTGSGTHFSLRI | |
| NSLQPEDFGSYYCQHHYVIPWTFGG | |
| GTKLEIKGSTSGSGKPGSGEGSTKG | |
| EVQMVESGGDLVKPGGSLKLSCAA | |
| SGFTFSNSGMSWVRQTPDKRLEWVA | |
| SISDGGLYTHYPDSVKGRFTISRDN | |
| GKSTLYLQMSSLRSEDTAIYYCARQ | |
| GVRPFFDYWGQGTTLTVSS | |
| (SEQ ID NO: 135) | |

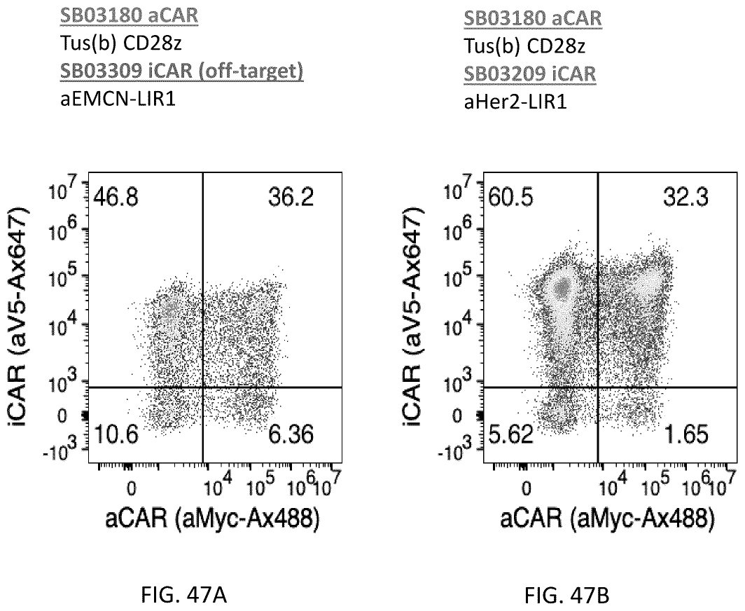

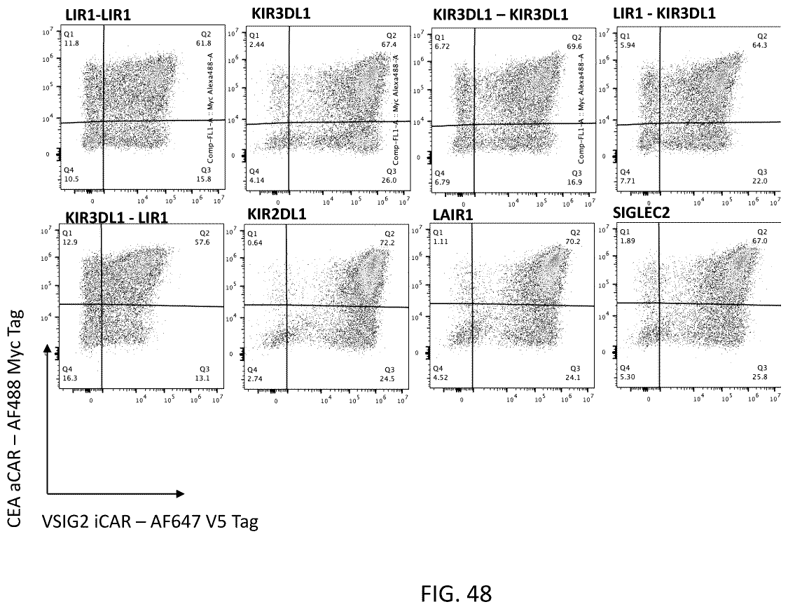

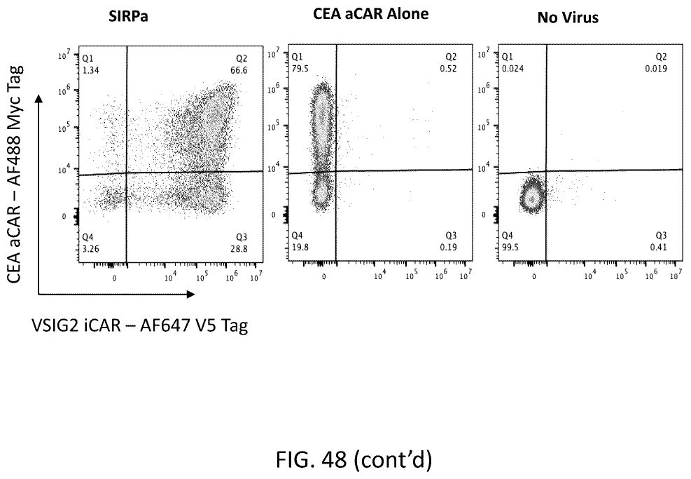

As a control, NK cells expressing an off-target inhibitory CAR (anti-EMCN) were also generated. Primary, donor-derived NK cells were first expanded and then transduced with retrovirus encoding the CARs. Expression of aCAR was determined via flow cytometry using a MYC tag. Expression of iCAR was determined via flow cytometry using a V5 tag. Expression for the model safety antigen system and the off-target control system are shown in FIGS. 47A-47B. Expression for the VSIG2 safety antigen system is shown in FIG. 48.

NK cells expressing a CEA-aCAR and various iCARs were co-cultured with target cells that expressed either CEACAM5 only or CEACAM5/Safety antigen (Her2 or VSIG2). Appropriate controls such as aCAR alone, iCAR alone and non-targeting iCAR were used.

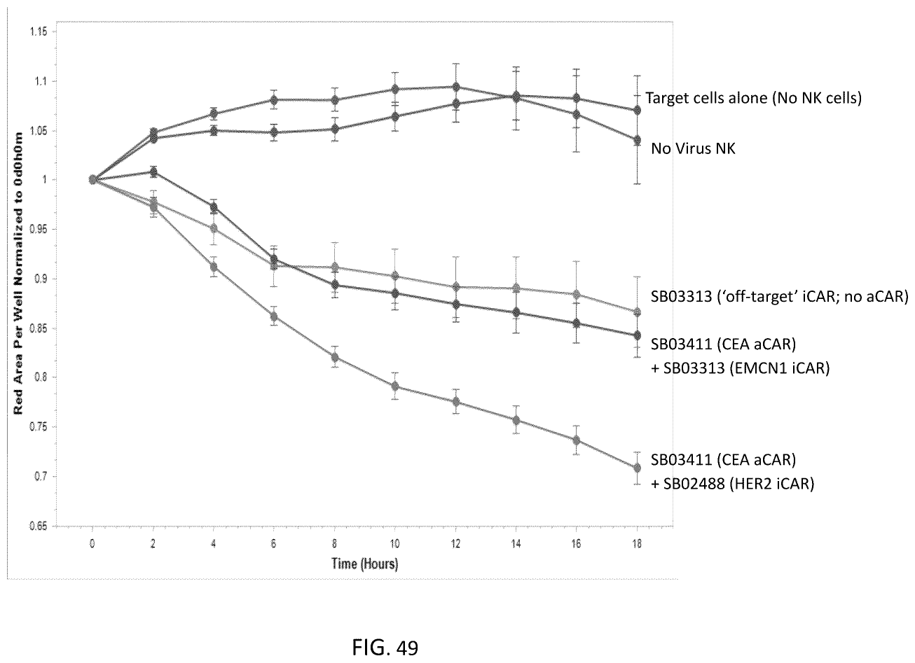

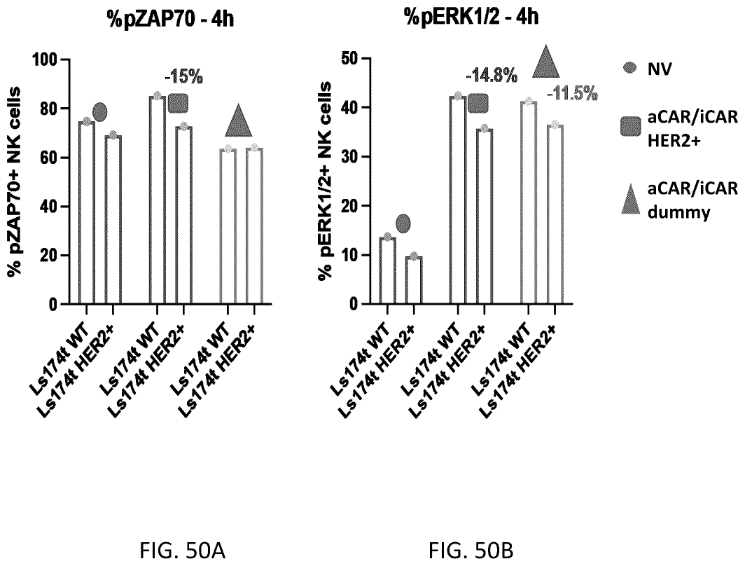

For the model antigen system (using HER2), target cell growth was measured via Incucyte and is shown in FIG. 49, and NK cell activation signaling was measured using phospho-flow of signaling downstream of the CAR (pZAP70 and pErk1/2) and is shown in FIGS. 50A and 50B. As shown in FIGS. 50A and 50B, expression of a HER2 inhibitory CAR reduces activating CAR-mediated signaling in a safety-antigen dependent manner.

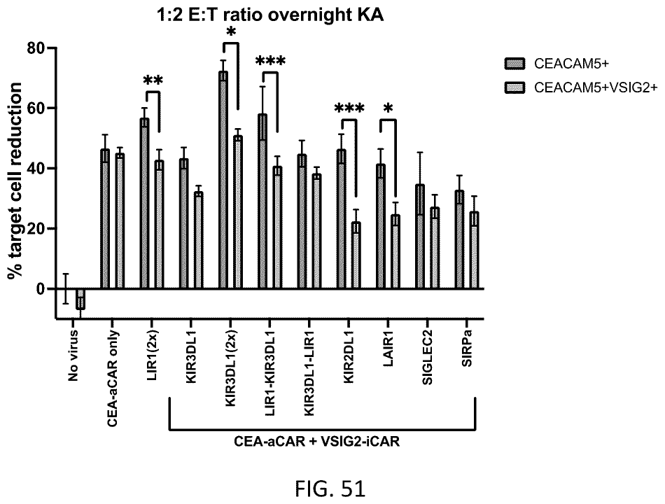

For the VSIG2 safety antigen system, percent target cell reduction was measured after an overnight co-culture with NOT-gated CAR NK cells using flow cytometry as shown in FIG. 51. Various VSIG2-inhibitory CARs containing various inhibitory domains show significant reduction of CAR-mediated cell killing in target cells that express the safety antigen (VSIG2, blue bars) and not in target cells that express only CEACAM5 (red bars). This result indicates that expression of a VSIG2 inhibitory CAR reduces activating CAR-mediated signaling in a safety-antigen dependent manner.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

1. An isolated cell comprising:
(a) an inhibitory chimeric receptor comprising an extracellular antigen-binding domain that binds to an antigen, wherein the antigen is selected from the group consisting of: VSIG2, CPM, ITM2C, SLC26A2, SLC4A4, GPA33, PLA2G2A, ABCA8, ATP1A2, CHP2, and SLC26A3; and
(b) a chimeric receptor comprising one or more extracellular antigen-binding domains, wherein the one or more extracellular antigen-binding domains bind one or more additional antigens selected from the group consisting of: CEA, CEACAM1, CEACAM5, and CEACAM6.

2. The isolated cell of claim 1, wherein:
(a) the antigen-binding domain of the inhibitory chimeric receptor comprises one or more single chain variable fragments (scFvs), optionally wherein each of the one or more scFvs comprises a heavy chain variable domain (VH) and a light chain variable domain (VL), optionally wherein the VH and VL are separated by a peptide linker, optionally the peptide linker comprises the amino acid sequence of SEQ ID NO: 39 or SEQ ID NO: 77, and/or optionally wherein each of the one or more scFvs comprises the structure VH-L-VL or VL-L-VH, wherein VH is the heavy chain variable domain, L is the peptide linker, and VL is the light chain variable domain;
(b) the inhibitory chimeric receptor comprises a transmembrane domain, and the transmembrane domain is selected from the group consisting of: a CD8 transmembrane domain, a CD28 transmembrane domain, a CD25 transmembrane domain, a CD7 transmembrane domain, a CD3zeta-chain transmembrane domain, a CD4 transmembrane domain, a 4-1BB transmembrane domain, an OX40 transmembrane domain, an ICOS transmembrane domain, a CTLA-4 transmembrane domain, a LAX transmembrane domain, a LAT transmembrane domain, a PD-1 transmembrane domain, a LAG-3 transmembrane domain, a TIM3 transmembrane domain, a KIR3DS1 transmembrane domain, a KIR3DL1 transmembrane domain, an NKG2D transmembrane domain, an NKG2A transmembrane domain, a TIGIT transmembrane domain, a 2B4 transmembrane domain, and a BTLA transmembrane domain;
(c) the inhibitory chimeric receptor comprises a spacer region between the antigen-binding domain and the transmembrane domain, optionally wherein the spacer region has an amino acid sequence selected from the group consisting of SEQ ID NOs: 49-58; and/or
(d) the inhibitory chimeric receptor comprises one or more intracellular inhibitory domains selected from the group consisting of: PD-1, CTLA4, TIGIT, LAIR1, GRB-2, Dok-1, Dok-2, SLAP, LAG3, HAVR, BTLA, LIR1, NKG2A, KIR3DL1, GITR, PD-L1, CSK, SHP-1, PTEN, CD45, CD148, PTP-MEG1, PTP-PEST, c-CBL, CBL-b, PTPN22, LAR, PTPH1, SHIP-1, RasGAP, CD94, and CD161

3. The isolated cell of claim 1, wherein the chimeric receptor is a CAR,
optionally wherein the CAR comprises one or more intracellular signaling domains, and the one or more intracellular signaling domains are selected from the group consisting of: a CD3zeta-chain intracellular signaling domain, a CD3epsilon-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, an NKp46 intracellular signaling domain, an NKp30 intracellular signaling domain, an NKp44 intracellular signaling domain, an NKG2D intracellular signaling domain, a CD226 intracellular signaling domain, and a CD160 intracellular signaling domain,
optionally wherein the CAR comprises a transmembrane domain, and the transmembrane domain is selected from the group consisting of: a CD8 transmembrane domain, a CD28 transmembrane domain, a CD25 transmembrane domain, a CD7 transmembrane domain, a CD3zeta-chain transmembrane domain, a CD4 transmembrane domain, a 4-1BB transmembrane domain, an OX40 transmembrane domain, an ICOS transmembrane domain, a CTLA-4 transmembrane domain, a LAX transmembrane domain, a LAT transmembrane domain, a PD-1 transmembrane domain, a LAG-3 transmembrane domain, a TIM3 transmembrane domain, a KIR3DS1 transmembrane domain, a KIR3DL1 transmembrane domain, an NKG2D transmembrane domain, an NKG2A transmembrane domain, a TIGIT transmembrane domain, a 2B4 transmembrane domain, and a BTLA transmembrane domain,
optionally wherein the CAR comprises a spacer region between the antigen-binding domain and the transmembrane domain, and the spacer region has an amino acid sequence selected from the group consisting of SEQ ID NOs: 49-58, and
optionally wherein the antigen-binding domain of the inhibitory chimeric receptor and/or the chimeric receptor comprises one or more single chain variable fragments (scFvs), optionally wherein each of the one or more scFvs comprises a heavy chain variable domain (VH) and a light chain variable domain (VL), optionally wherein the VH and VL are separated by a peptide linker, optionally wherein the peptide linker comprises the amino acid sequence of SEQ ID NO: 39 or SEQ ID NO: 77,
optionally wherein each of the one or more scFvs comprises the structure VH-L-VL or VL-L-VH, wherein VH is the heavy chain variable domain, L is the peptide linker, and VL is the light chain variable domain.

4. The isolated cell of claim 1, wherein:
(a) the one or more additional antigen is CECAM1, optionally wherein the antigen-binding domain that binds to CEACAM1 comprises a heavy chain variable domain (VH) comprising the amino acid sequence of SEQ ID NO: 1 and a light chain variable domain (VL) comprising the amino acid sequence of SEQ ID NO: 2;
(b) the one or more additional antigen is CECAM5, optionally wherein the antigen-binding domain that binds to CEACAM5 comprises a heavy chain variable domain (VH) and a light chain variable domain (VL) selected from the group consisting of:
(i) a VH comprising the amino acid sequence of SEQ ID NO: 3 and a VL comprising the amino acid sequence of SEQ. ID NO: 4;
(ii) a VH comprising the amino acid sequence of SEQ ID NO: 9 and a VL comprising the amino acid sequence of SEQ. ID NO: 10;
(iii) a VH comprising the amino acid sequence of SEQ ID NO: 11 and a VL comprising the amino acid sequence of SEQ. ID NO: 12;
(iv) a VH comprising the amino acid sequence of SEQ ID NO: 13 and a VL comprising the amino acid sequence of SEQ. ID NO: 14;
(v) a VH comprising the amino acid sequence of SEQ ID NO: 78 and a VL comprising the amino acid sequence of SEQ. ID NO: 79; and
(vi) a VH comprising the amino acid sequence of SEQ ID NO: 15 and a VL comprising the amino acid sequence of SEQ. ID NO: 16; or
the antigen-binding domain that binds to CEACAM5 comprises a heavy chain (HC) and a light chain (LC) selected from the group consisting of:
(i) an HC comprising the amino acid sequence of SEQ ID NO: 5 and an LC comprising the amino acid sequence of SEQ. ID NO: 6; and
(ii) an HC comprising the amino acid sequence of SEQ ID NO: 7 and an LC comprising the amino acid sequence of SEQ. ID NO: 8; or
(c) the one or more additional antigen is CECAM6, optionally wherein the antigen-binding domain that binds to CEACAM6 comprises a heavy chain variable domain (VH) comprising the amino acid sequence of SEQ ID NO: 17 and a light chain variable domain (VL) comprising the amino acid sequence of SEQ ID NO 18.

5. The isolated cell of claim 1, wherein the cell is an immunoresponsive cell,
optionally wherein binding of the inhibitory chimeric receptor to the antigen is capable of inhibiting the immunoresponsive cell and/or wherein binding of the chimeric receptor to the one or more additional antigens is capable of activating the immunoresponsive cell.

6. The isolated cell of claim 1, wherein:
(a) the chimeric receptor binds to the one or more additional antigens with a low binding affinity;
(b) the chimeric receptor binds to the one or more additional antigens with a binding affinity that is lower than the binding affinity with which the inhibitory chimeric receptor binds to the antigen; and/or
(c) the inhibitory chimeric receptor binds to the antigen with a low binding avidity.

7. The isolated cell of claim 1, wherein the chimeric receptor is recombinantly expressed, optionally wherein the chimeric receptor is expressed from a vector or a selected locus from the genome of the cell.

8. The isolated cell of claim 1, wherein the cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell, optionally wherein the cell is autologous or the cell is allogeneic.

9. An isolated nucleic acid or set of isolated nucleic acids encoding the inhibitory chimeric receptor and/or the chimeric receptor of claim 1.

10. A vector comprising the nucleic acid of claim 9.

11. A genetically modified cell comprising the nucleic acid of claim 9.

12. A pharmaceutical composition comprising an effective amount of the isolated cell of claim 10 and a pharmaceutically acceptable carrier, a pharmaceutically acceptable excipient, or a combination thereof.

13. A method of treating a subject in need thereof, the method comprising administering to the subject an effective amount of the isolated cell of claim 10.

14. A method of stimulating a cell-mediated immune response to a tumor cell in a subject, the method comprising administering to a subject having a tumor an effective amount of the isolated cell of claim 10.

15. A method of providing an anti-tumor immunity in a subject, the method comprising administering to a subject in need thereof an effective amount of the isolated cell of claim 10.

16. A method of reducing tumor burden in a subject, comprising administering to the subject an effective amount of the isolated cell of claim 10, optionally wherein:
(a) the method reduces the number of tumor cells,
(b) the method reduces tumor size,
(c) the method reduces tumor volume, and/or
(d) the method eradicates the tumor in the subject.

17. A method of treating a subject having a tumor, the method comprising administering an effective amount of the isolated cell of claim 10.

18. A method of treating or preventing a cancer in a subject, wherein the cancer is selected from the group consisting of: colorectal carcinoma, pancreatic cancer, a lung adenocarcinoma, and gastric cancer, the method comprising administering to the subject an effective amount of the cell of claim 10.

19. The method of claim 17, wherein:
(a) the method increases progression free survival in the subject; and or
(b) the method increases survival in the subject.

20. A kit for treating and/or preventing a colorectal carcinoma, a pancreatic cancer, a lung adenocarcinoma, and/or a gastric cancer, comprising the isolated cell of claim 10, optionally wherein the kit further comprises written instructions for using the isolated cell or pharmaceutical composition for treating and/or preventing a colorectal carcinoma, a pancreatic cancer, a lung adenocarcinoma, and/or a gastric cancer in a subject, or the kit further comprises instructions for using the nucleic acid or vector for producing one or more antigen-specific cells for treating and/or preventing a colorectal carcinoma, a pancreatic cancer, a lung adenocarcinoma, and/or a gastric cancer in a subject.


SEQUENCE LISTING (140 sequences)

SEQ ID NO: 1 - protein, 120 aa, synthetic construct
  REGION 1..120: note = Description of Artificial Sequence: Synthetic polypeptide
QVQLQQSGAELVRPGTSVKVSCKASGYAFTNNLIEWVKQRPGQGLEWIGVINPGSGDTNYNEKFKGKATLTADKSSNTAYMQLSSLTSDDSAVYFCARGDYYGGFAVDYWGQGTSVTVSS

SEQ ID NO: 2 - protein, 106 aa, synthetic construct
  REGION 1..106: note = Description of Artificial Sequence: Synthetic polypeptide
DIQMTQTTSSLSASLGDRVTISCRTSQDIGNYLNWYQQKPDGTVKLLIYYTSRLHSGVPSRFSGSGSGTDYSLTISNLEQEDIATYFCQQGKSLPRTFGGGTKLEI

SEQ ID NO: 3 - protein, 119 aa, synthetic construct
  REGION 1..119: note = Description of Artificial Sequence: Synthetic polypeptide
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 4 - protein, 106 aa, synthetic construct
  REGION 1..106: note = Description of Artificial Sequence: Synthetic polypeptide
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 5 - protein, 448 aa, synthetic construct
  REGION 1..448: note = Description of Artificial Sequence: Synthetic polypeptide
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLS

SEQ ID NO: 6 - protein, 214 aa, synthetic construct
  REGION 1..214: note = Description of Artificial Sequence: Synthetic polypeptide
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYAEVTHQGLSSPVTKSFNRGEC

SEQ ID NO: 7 - protein, 448 aa, synthetic construct
  REGION 1..448: note = Description of Artificial Sequence: Synthetic polypeptide
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 8 - protein, 214 aa, synthetic construct
  REGION 1..214: note = Description of Artificial Sequence: Synthetic polypeptide
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 118 aa, synthetic construct
  REGION 1..118: note = Description of Artificial Sequence: Synthetic polypeptide
QLQESGPGLVRPSQTLSLTCTVSGFTISSGYSWHWVRQPPGRGLEWIGYIQYSGITNYNPSLKSRVTMLVDTSKNQFSLRLSSVTAADTAVYYCAREDYDYHWYFDVWGQGSLVTVSS

SEQ ID NO: 10 - protein, 110 aa, synthetic construct
  REGION 1..110: note = Description of Artificial Sequence: Synthetic polypeptide
GVHSDIQMTQSPSSLSASVGDRVTITCSTSSSVSYMHWYQQKPGKAPKLLIYSTSNLASGVPSRFSGSGSGTDFTFTISSLQPEDIATYYCHQWSSYPTFGQGTKVEIKR

SEQ ID NO: 11 - protein, 129 aa, synthetic construct
  REGION 1..129: note = Description of Artificial Sequence: Synthetic polypeptide
MDFQVQIFSFLLISASVIMSRGQTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKR

SEQ ID NO: 12 - protein, 112 aa, synthetic construct
  REGION 1..112: note = Description of Artificial Sequence: Synthetic polypeptide
MDFQVQIFSFLLISASVIMSRGQTVLSQSPAILSASPGEKVTMTCRASSSVTYIHWYQQKPGSSPARFSGSGTSYSLTISRVEAEDAATYYCQHWSSKPPTFGGGTKLEIKR

SEQ ID NO: 13 - protein, 145 aa, synthetic construct
  REGION 1..145: note = Description of Artificial Sequence: Synthetic polypeptide
ETVIKYLLPTAAAGLLLLAAQPAMAQVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSS

SEQ ID NO: 14 - protein, 109 aa, synthetic construct
  REGION 1..109: note = Description of Artificial Sequence: Synthetic polypeptide
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAA

SEQ ID NO: 15 - protein, 121 aa, synthetic construct
  REGION 1..121: note = Description of Artificial Sequence: Synthetic polypeptide
EVKLVESGGGLVQPGGSLRLSCSISGFTFTDYYMNWVRQSPGKALEWLGFIRNKVNGDTTEYSASVKGRFTISRDISQSILYLQMNTLRTEDSATYYCARDKGIAYYFDYWGQGTTLTVSS

SEQ ID NO: 16 - protein, 106 aa, synthetic construct
  REGION 1..106: note = Description of Artificial Sequence: Synthetic polypeptide
QIVLSQSPAILFASPGEKVTMTCRASSSVSYIHWYQQKPGSSPKPWIHGTSNLASGVPARFSGSGSGTSYSLTISRMEAEDAATYYCQQWSSNLSTFGGGTKLEIK

SEQ ID NO: 17 - protein, 443 aa, synthetic construct
  REGION 1..443: note = Description of Artificial Sequence: Synthetic polypeptide
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 18 - protein, 214 aa, synthetic construct
  REGION 1..214: note = Description of Artificial Sequence: Synthetic polypeptide
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 240 aa, synthetic construct
  REGION 1..240: note = Description of Artificial Sequence: Synthetic polypeptide
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 20 - protein, 241 aa, synthetic construct
  REGION 1..241: note = Description of Artificial Sequence: Synthetic polypeptide
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 21 - protein, 242 aa, synthetic construct
  REGION 1..242: note = Description of Artificial Sequence: Synthetic polypeptide
EVKLVESGGGLVQPGGSLRLSCSISGFTFTDYYMNWVRQSPGKALEWLGFIRNKVNGDTTEYSASVKGRFTISRDISQSILYLQMNTLRTEDSATYYCARDKGIAYYFDYWGQGTTLTVSSGGGGSGGGGSGGGGSQIVLSQSPAILFASPGEKVTMTCRASSSVSYIHWYQQKPGSSPKPWIHGTSNLASGVPARFSGSGSGTSYSLTISRMEAEDAATYYCQQWSSNLSTFGGGTKLEIK

SEQ ID NO: 22 - protein, 241 aa, synthetic construct
  REGION 1..241: note = Description of Artificial Sequence: Synthetic polypeptide
QVQLQQSGAELVRPGTSVKVSCKASGYAFTNNLIEWVKQRPGQGLEWIGVINPGSGDTNYNEKFKGKATLTADKSSNTAYMQLSSLTSDDSAVYFCARGDYYGGFAVDYWGQGTSVTVSSGGGGSGGGGSGGGGSDIQMTQTTSSLSASLGDRVTISCRTSQDIGNYLNWYQQKPDGTVKLLIYYTSRLHSGVPSRFSGSGSGTDYSLTISNLEQEDIATYFCQQGKSLPRTFGGGTKLEI

SEQ ID NO: 23 - protein, 244 aa, synthetic construct
  REGION 1..244: note = Description of Artificial Sequence: Synthetic polypeptide
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRGGGGSGGGGSGGGGSEVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSA

SEQ ID NO: 24 - protein, 244 aa, synthetic construct
  REGION 1..244: note = Description of Artificial Sequence: Synthetic polypeptide
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSAGGGGSGGGGSGGGGSDIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKR

SEQ ID NO: 25 - protein, 325 aa, synthetic construct
  REGION 1..325: note = Description of Artificial Sequence: Synthetic polypeptide
EVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPLAPCSRSTSESTAALGCLVKDYFPEPVTVLYLQMNTLQAEDSAIYYCTRDRGLRFYFDYWGQGTLVTVSSGGGGSGGGGSGGGGSQTVLTQSPSSLSVSVGDRVTITCRASSSVTYIHWYQQKPGLAPKSLIYATSNLASGVPSRFSGSGSGTDYTFTISSLQPEDIATYYCQHWSSKPPTFGQGTKVEVKRTV

SEQ ID NO: 26 - protein, 325 aa, synthetic construct
  REGION 1..325: note = Description of Artificial Sequence: Synthetic polypeptide
QTVLTQSPSSLSVSVGDRVTITCRASSSVTYIHWYQQKPGLAPKSLIYATSNLASGVPSRFSGSGSGTDYTFTISSLQPEDIATYYCQHWSSKPPTFGQGTKVEVKRTVGGGGSGGGGSGGGGSEVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPLAPCSRSTSESTAALGCLVKDYFPEPVTVLYLQMNTLQAEDSAIYYCTRDRGLRFYFDYWGQGTLVTVSS

SEQ ID NO: 27 - skipped

SEQ ID NO: 28 - protein, 6 aa, synthetic construct
  REGION 1..6: note = Description of Artificial Sequence: Synthetic peptide
GGSGGS

SEQ ID NO: 29 - protein, 9 aa, synthetic construct
  REGION 1..9: note = Description of Artificial Sequence: Synthetic peptide
GGSGGSGGS

SEQ ID NO: 30 - protein, 12 aa, synthetic construct
  REGION 1..12: note = Description of Artificial Sequence: Synthetic peptide
GGSGGSGGSGGS

SEQ ID NO: 31 - protein, 15 aa, synthetic construct
  REGION 1..15: note = Description of Artificial Sequence: Synthetic peptide
GGSGGSGGSGGSGGS

SEQ ID NO: 32 - protein, 4 aa, synthetic construct
  REGION 1..4: note = Description of Artificial Sequence: Synthetic peptide
GGGS

SEQ ID NO: 33 - protein, 8 aa, synthetic construct
  REGION 1..8: note = Description of Artificial Sequence: Synthetic peptide
GGGSGGGS

SEQ ID NO: 34 - protein, 12 aa, synthetic construct
  REGION 1..12: note = Description of Artificial Sequence: Synthetic peptide
GGGSGGGSGGGS

SEQ ID NO: 35 - protein, 16 aa, synthetic construct
  REGION 1..16: note = Description of Artificial Sequence: Synthetic peptide
GGGSGGGSGGGSGGGS

SEQ ID NO: 36 - protein, 20 aa, synthetic construct
  REGION 1..20: note = Description of Artificial Sequence: Synthetic peptide
GGGSGGGSGGGSGGGSGGGS

SEQ ID NO: 37 - protein, 5 aa, synthetic construct
  REGION 1..5: note = Description of Artificial Sequence: Synthetic peptide
GGGGS

SEQ ID NO: 38 - protein, 10 aa, synthetic construct
  REGION 1..10: note = Description of Artificial Sequence: Synthetic peptide
GGGGSGGGGS

SEQ ID NO: 39 - protein, 15 aa, synthetic construct
  REGION 1..15: note = Description of Artificial Sequence: Synthetic peptide
GGGGSGGGGSGGGGS

SEQ ID NO: 40 - protein, 20 aa, synthetic construct
  REGION 1..20: note = Description of Artificial Sequence: Synthetic peptide
GGGGSGGGGSGGGGSGGGGS

SEQ ID NO: 41 - protein, 25 aa, synthetic construct
  REGION 1..25: note = Description of Artificial Sequence: Synthetic peptide
GGGGSGGGGSGGGGSGGGGSGGGGS

SEQ ID NO: 42 - protein, 18 aa, synthetic construct
  REGION 1..18: note = Description of Artificial Sequence: Synthetic peptide
GSTSGSGKPGSGEGSTKG

SEQ ID NO: 43 - protein, 20 aa, synthetic construct
  REGION 1..20: note = Description of Artificial Sequence: Synthetic peptide
EAAAKEAAAKEAAAKEAAAK

SEQ ID NO: 44 - DNA, 45 bp, synthetic construct
  misc_feature 1..45: note = Description of Artificial Sequence: Synthetic oligonucleotide
  source 1..45: mol_type = other DNA
ggcggaggcggatcaggtggcggaggaagtggcggcggaggatct

SEQ ID NO: 45 - protein, 21 aa, synthetic construct
  REGION 1..21: note = Description of Artificial Sequence: Synthetic peptide
IYIWAPLAGTCGVLLLSLVIT

SEQ ID NO: 46 - protein, 27 aa, synthetic construct
  REGION 1..27: note = Description of Artificial Sequence: Synthetic peptide
FWVLVVVGGVLACYSLLVTVAFIIFWV

SEQ ID NO: 47 - protein, 21 aa, synthetic construct
  REGION 1..21: note = Description of Artificial Sequence: Synthetic peptide
VAAILGLGLVLGLLGPLAILL

SEQ ID NO: 48 - protein, 21 aa, synthetic construct
  REGION 1..21: note = Description of Artificial Sequence: Synthetic peptide
FLVIIVILSALFLGTLACFCV

SEQ ID NO: 49 - protein, 42 aa, synthetic construct
  REGION 1..42: note = Description of Artificial Sequence: Synthetic polypeptide
AAAIEVMYPPPYLDNEKSNGTIIHVKGKHLCPSPLFPGPSKP

SEQ ID NO: 50 - protein, 45 aa, synthetic construct
  REGION 1..45: note = Description of Artificial Sequence: Synthetic polypeptide
TTTPAPRPPTPAPTIALQPLSLRPEACRPAAGGAVHTRGLDFACD

SEQ ID NO: 51 - protein, 12 aa, synthetic construct
  REGION 1..12: note = Description of Artificial Sequence: Synthetic peptide
ESKYGPPCPSCP

SEQ ID NO: 52 - protein, 12 aa, synthetic construct
  REGION 1..12: note = Description of Artificial Sequence: Synthetic peptide
ESKYGPPAPSAP

SEQ ID NO: 53 - protein, 12 aa, synthetic construct
  REGION 1..12: note = Description of Artificial Sequence: Synthetic peptide
ESKYGPPCPPCP

SEQ ID NO: 54 - protein, 12 aa, synthetic construct
  REGION 1..12: note = Description of Artificial Sequence: Synthetic peptide
EPKSCDKTHTCP

SEQ ID NO: 55 - protein, 86 aa, synthetic construct
  REGION 1..86: note = Description of Artificial Sequence: Synthetic polypeptide
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 56 - protein, 132 aa, synthetic construct
  REGION 1..132: note = Description of Artificial Sequence: Synthetic polypeptide
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 57 - protein, 34 aa, synthetic construct
  REGION 1..34: note = Description of Artificial Sequence: Synthetic polypeptide
ACPTGLYTHSGECCKACNLGEGVAQPCGANQTVC

SEQ ID NO: 58 - protein, 20 aa, synthetic construct
  REGION 1..20: note = Description of Artificial Sequence: Synthetic peptide
AVGQDTQEVIVVPHSLPFKV

SEQ ID NO: 59 - DNA, 126 bp, synthetic construct
  misc_feature 1..126: note = Description of Artificial Sequence: Synthetic polynucleotide
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 60 - DNA, 135 bp, synthetic construct
  misc_feature 1..135: note = Description of Artificial Sequence: Synthetic polynucleotide
  source 1..135: mol_type = other DNA
accacaacaccagctcctagacctccaactcctgctcctacaatcgccctgcagccactgtctctgaggcctgaagcttgtagacctgctgctggcggagccgtgcataccagaggactggatttcgcctgcgac

SEQ ID NO: 61 - DNA, 36 bp, synthetic construct
  misc_feature 1..36: note = Description of Artificial Sequence: Synthetic oligonucleotide
  source 1..36: mol_type = other DNA
gaaagcaagtacggtccaccttgccctagctgtccg

SEQ ID NO: 62 - DNA, 36 bp, synthetic construct
  misc_feature 1..36: note = Description of Artificial Sequence: Synthetic oligonucleotide
  source 1..36: mol_type = other DNA
gaatccaagtacggccccccagcgcctagtgcccca

SEQ ID NO: 63 - DNA, 36 bp, synthetic construct
  misc_feature 1..36: note = Description of Artificial Sequence: Synthetic oligonucleotide
  source 1..36: mol_type = other DNA
gaatctaaatatggcccgccatgcccgccttgccca

SEQ ID NO: 64 - DNA, 36 bp, synthetic construct
  misc_feature 1..36: note = Description of Artificial Sequence: Synthetic oligonucleotide
  source 1..36: mol_type = other DNA
gaaccgaagtcttgtgataaaactcatacgtgcccg

SEQ ID NO: 65 - DNA, 258 bp, synthetic construct
  misc_feature 1..258: note = Description of Artificial Sequence: Synthetic polynucleotide
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 66 - DNA, 396 bp, synthetic construct
  misc_feature 1..396: note = Description of Artificial Sequence: Synthetic polynucleotide
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 67 - DNA, 102 bp, synthetic construct
  misc_feature 1..102: note = Description of Artificial Sequence: Synthetic polynucleotide
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 68 - DNA, 60 bp, synthetic construct
  misc_feature 1..60: note = Description of Artificial Sequence: Synthetic oligonucleotide
  source 1..60: mol_type = other DNA
gctgtgggccaggacacgcaggaggtcatcgtggtgccacactccttgccctttaaggtg

SEQ ID NO: 69 - protein, 12 aa, synthetic construct
  REGION 1..12: note = Description of Artificial Sequence: Synthetic peptide
GGCKJSGGCKJS

SEQ ID NO: 70 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 71 - protein, 41 aa, synthetic construct
  REGION 1..41: note = Description of Artificial Sequence: Synthetic polypeptide
RSKRSRLLHSDYMNMTPRRPGPTRKHYQPYAPPRDFAAYRS

SEQ ID NO: 72 - protein, 42 aa, synthetic construct
  REGION 1..42: note = Description of Artificial Sequence: Synthetic polypeptide
ALYLLRRDQRLPPDAHKPPGGGSFRTPIQEEQADAHSTLAKI

SEQ ID NO: 73 - protein, 120 aa, synthetic construct
  REGION 1..120: note = Description of Artificial Sequence: Synthetic polypeptide
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 74 - protein, 112 aa, synthetic construct
  REGION 1..112: note = Description of Artificial Sequence: Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 10 aa, synthetic construct
  REGION 1..10: note = Description of Artificial Sequence: Synthetic peptide
EQKLISEEDL

SEQ ID NO: 76 - protein, 21 aa, synthetic construct
  REGION 1..21: note = Description of Artificial Sequence: Synthetic peptide
MALPVTALLLPLALLLHAARP

SEQ ID NO: 77 - protein, 15 aa, synthetic construct
  REGION 1..15: note = Description of Artificial Sequence: Synthetic peptide
GGSGSGGSGSGGSGS

SEQ ID NO: 78 - protein, 120 aa, synthetic construct
  REGION 1..120: note = Description of Artificial Sequence: Synthetic polypeptide
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 79 - protein, 106 aa, synthetic construct
  REGION 1..106: note = Description of Artificial Sequence: Synthetic polypeptide
ENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 80 - DNA, 1488 bp, synthetic construct
  misc_feature 1..1488: note = Description of Artificial Sequence: Synthetic polynucleotide
  source 1..1488: mol_type = other DNA
atggctctgcctgtgacagctctgctgctgcctctggctctgcttctgcatgccgccagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgagcagaagctgatctccgaagaggacctgaccacaacaccagctcctagaccacctacaccagcacctacaatcgccctgcagccactgtctctgaggccagaggcttgtagacctgctgctggcggagccgtgcatacaagaggactggatttcgcctgcgacttctgggtgctcgtggttgttggaggcgtgctggcctgttactctctgctggtcaccgtggccttcatcatcttttgggtccgaagcaagcggagccggctgctgcacagcgactacatgaacatgacccctagacggcccggacctaccagaaagcactaccagccttacgctcctcctagagacttcgccgcctaccggtccagagtgaagttctccagatctgccgacgctcccgcctataagcagggccagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 81 - protein, 375 aa, synthetic construct
  REGION 1..375: note = Description of Artificial Sequence: Synthetic polypeptide
MALPVTALLLPLALLLHAARPEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 1491 bp, synthetic construct
  misc_feature 1..1491: note = Description of Artificial Sequence: Synthetic polynucleotide
  source 1..1491: mol_type = other DNA
atggctctgcctgtgacagctctgctgctgcctctggctctgcttctgcatgccgctagaccccaagtgaagctggaacagtctggcgccgaggttgtgaaacctggcgcctctgtgaagctgagctgtaaagccagcggcttcaacatcaaggacagctacatgcactggctgcggcaaggccctggacagagactggaatggatcggctggatcgaccccgagaatggcgatacagagtacgcccctaagttccagggcaaagccaccttcaccaccgacacctctgccaacacagcctacctgggactgtctagcctgaggcctgaagataccgccgtgtactactgcaacgagggcacacctacaggcccctactacttcgattattggggccagggcaccctggtcaccgtttctagcggaggcggaggatctggtggcggaggaagtggcggaggcggttctgaaaatgtgctgacacagagccccagcagcatgtccgtgtctgtgggcgacagagtgacaatcgcctgtagcgcctctagcagcgtgccctatatgcattggctgcagcagaagcccggcaagagccctaagctgctgatctacctgaccagcaatctggccagcggcgtgccaagcagattttctggaagcggcagcggcaccgactacagcctgacaatcagtagcgtgcagcctgaggatgccgccacctactactgtcagcagagaagcagctaccctctgacctttggaggcggcaccaagctggaaatcaaagagcagaagctgatctccgaagaggacctgaccacaacaccagctcctagaccacctacaccagcacctacaatcgccctgcagccactgtctctgaggccagaagcttgtagacctgctgctggcggagccgtgcatacaagaggactggatttcgcctgcgacttctgggtgctcgtggttgttggcggagtgctggcctgttactctctgctggtcacagtggccttcatcatcttttgggtccgaagcaagcggagccggctgctgcacagcgactacatgaacatgacccctagacggcccggacctaccagaaagcactaccagccttacgctcctcctagagacttcgccgcctaccggtccagagtgaagttcagcagatccgccgatgctcccgcctataagcagggccagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 83 - protein, 362 aa, synthetic construct
  REGION 1..362: note = Description of Artificial Sequence: Synthetic polypeptide
MALPVTALLLPLALLLHAARPENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIKEQKLISEEDLTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - DNA, 1494 bp, synthetic construct
  misc_feature 1..1494: note = Description of Artificial Sequence: Synthetic polynucleotide
  source 1..1494: mol_type = other DNA
atggctctgcctgtgacagctctgctgctgcctctggctctgcttctgcatgctgccagacctcagatcgtgctgtctcagtctcccgccatcctgtttgctagccctggcgagaaagtgaccatgacctgtagagccagcagctccgtgtcctacatccactggtatcagcagaagcccggcagcagccctaagccttggatccacggcacaagcaatctggccagcggagtgcctgccagattttctggaagcggcagcggcaccagctacagcctgaccatctctagaatggaagccgaggacgccgccacctactactgtcagcagtggtccagcaacctgagcacctttggcggcggaacaaagctggaaatcaaaggcggcggaggatctggcggaggcggtagcggaggtggtggatctgaagtgaagctggtggaatcaggcggaggcctggttcaacctggcggatctctgagactgagctgcagcatctccggcttcaccttcaccgactactacatgaactgggtccgacagagccctggcaaggctctggaatggctgggcttcatcagaaacaaagtgaacggcgacaccaccgagtacagcgcctctgtgaagggcagattcaccatcagccgggacatcagccagagcatcctgtacctgcagatgaacaccctgcggaccgaggatagcgccacatattactgcgccagagacaagggaatcgcctactacttcgactactggggccagggcaccacactgacagtgtctagcgagcagaagctgatctccgaagaggacctgaccacaacaccagctcctagaccacctacaccagcacctacaatcgccctgcagccactgtctctgaggccagaagcttgtagacctgctgctggcggagccgtgcatacaagaggactggatttcgcctgcgacttctgggtgctcgtggttgttggcggagtgctggcctgttactctctgctggtcaccgtggccttcatcatcttttgggtccgaagcaagcggagccggctgctgcacagcgattacatgaacatgacccctcggaggcccggacctaccagaaagcactaccagccttacgctcctcctagagacttcgccgcctaccggtccagagtgaagttcagcagatccgctgatgcccctgcctacaagcagggccagaaccagctgtacaacgagctgaacctggggagaagagaagagtacgacgtgctggacaagcggagaggcagagatcctgagatgggcggcaagcccagacggaagaatcctcaagagggcctgtataacgaactgcagaaagacaagatggccgaggcctacagcgagatcggaatgaagggcgagcgcagaagaggcaagggacacgatggactgtaccagggcctgagcacagccaccaaggatacctatgacgccctgcacatgcaggccctgcctccaaga

SEQ ID NO: 85 - protein, 377 aa, synthetic construct
  REGION 1..377: note = Description of Artificial Sequence: Synthetic polypeptide
MALPVTALLLPLALLLHAARPEVKLVESGGGLVQPGGSLRLSCSISGFTFTDYYMNWVRQSPGKALEWLGFIRNKVNGDTTEYSASVKGRFTISRDISQSILYLQMNTLRTEDSATYYCARDKGIAYYFDYWGQGTTLTVSSEQKLISEEDLTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 363 aa, synthetic construct
  REGION 1..363: note = Description of Artificial Sequence: Synthetic polypeptide
MALPVTALLLPLALLLHAARPDIQMTQTTSSLSASLGDRVTISCRTSQDIGNYLNWYQQKPDGTVKLLIYYTSRLHSGVPSRFSGSGSGTDYSLTISNLEQEDIATYFCQQGKSLPRTFGGGTKLEIKEQKLISEEDLTTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 1395 bp, synthetic construct
  misc_feature 1..1395: note = Description of Artificial Sequence: Synthetic polynucleotide
  source 1..1395: mol_type = other DNA
atggctctgcctgtgacagctctgctgctgcctctggctctgcttctgcatgctgctagacctcaggtccagctgcagcagtctggcgccgaacttgttagacctggcaccagcgtgaaggtgtcctgtaaagccagcggctacgccttcaccaacaacctgatcgagtgggtcaagcagaggcctggacagggactcgagtggatcggcgtgatcaatcctggcagcggcgacaccaactacaacgagaagttcaagggcaaagccacactgaccgccgacaagagcagcaacacagcctacatgcagctgagcagcctgacctccgatgacagcgccgtgtacttttgcgccagaggcgattactacggcggcttcgccgtggattattggggccagggaacaagcgtgaccgtgtctagcggaggcggaggatctggtggcggaggaagtggcggaggcggttctgatatccagatgacccagaccaccagcagcctgtctgcctctctgggcgatagagtgaccatcagctgcagaaccagccaggacatcggcaactacctgaactggtatcagcagaaacccgacggcaccgtgaagctgctgatctactacaccagcagactgcacagcggcgtgcccagcagattttctggaagcggcagcggcaccgactactccctgacaatcagcaacctggaacaagaggatatcgctacctacttctgccagcaaggcaagagcctgcctagaacctttggaggcggcaccaagctggaaatcaaagagcagaagctgatctccgaagaggacctggagtctaagtacggccctccttgtcctagctgccccttttgggtgctcgtggttgttggcggagtgctggcctgttactctctgctggtcaccgtggccttcatcatcttttgggtccgaagcaagcggagccggctgctgcactccgactacatgaacatgacccctagacggcccggacctaccagaaagcactaccagccttacgctcctcctagagacttcgccgcctaccggtccagagtgaagttcagcagatccgccgatgctcccgcctataagcagggccagaatcagctgtacaacgagctgaacctggggcgcagagaagagtacgacgtgctggacaagagaagaggcagggaccctgagatgggcggcaagcccagaagaaagaaccctcaagagggcctgtataacgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 88 - protein, 330 aa, synthetic construct
  REGION 1..330: note = Description of Artificial Sequence: Synthetic polypeptide
MALPVTALLLPLALLLHAARPDIQMTQTTSSLSASLGDRVTISCRTSQDIGNYLNWYQQKPDGTVKLLIYYTSRLHSGVPSRFSGSGSGTDYSLTISNLEQEDIATYFCQQGKSLPRTFGGGTKLEIKEQKLISEEDLESKYGPPCPSCPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 491 aa, synthetic construct
  REGION 1..491: note = Description of Artificial Sequence: Synthetic polypeptide
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLTTTPAPRPPTPAPTIALQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - DNA, 1476 bp, synthetic construct
  misc_feature 1..1476: note = Description of Artificial Sequence: Synthetic polynucleotide
  source 1..1476: mol_type = other DNA
atggctctgcctgtgacagctctgctgctgcctctggctctgcttctgcatgccgctagaccccaagtgaagctggaacagtctggcgccgaggttgtgaaacctggcgcctctgtgaagctgagctgtaaagccagcggcttcaacatcaaggacagctacatgcactggctgcggcaaggccctggacagagactggaatggatcggctggatcgaccccgagaatggcgatacagagtacgcccctaagttccagggcaaagccaccttcaccaccgacacctctgccaacacagcctacctgggactgtctagcctgaggcctgaagataccgccgtgtactactgcaacgagggcacacctacaggcccctactacttcgattattggggccagggcaccctggtcaccgtttctagcggaggcggaggatctggtggcggaggaagtggcggaggcggttctgaaaatgtgctgacacagagccccagcagcatgtccgtgtctgtgggcgacagagtgacaatcgcctgtagcgcctctagcagcgtgccctatatgcattggctgcagcagaagcccggcaagagccctaagctgctgatctacctgaccagcaatctggccagcggcgtgccaagcagattttctggaagcggcagcggcaccgactacagcctgacaatcagtagcgtgcagcctgaggatgccgccacctactactgtcagcagagaagcagctaccctctgacctttggaggcggcaccaagctggaaatcaaagagcagaagctgatctccgaagaggacctgaccacaacaccagctcctagaccacctacaccagcacctacaatcgccctgcagccactgtctctgaggccagaagcttgtagacctgctgctggcggagccgtgcatacaagaggactggatttcgcctgcgacgtggccgccattctcggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatcagagtgaagttcagcagatccgccgacgctcccgcctataagcagggacagaatcagctgtacaacgagctgaacctggggcgcagagaagagtacgacgtgctggacaagagaagaggcagggatcctgaaatgggcggcaagcccagacggaagaatcctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 91 - protein, 492 aa, synthetic construct
  REGION 1..492: note = Description of Artificial Sequence: Synthetic polypeptide
MALPVTALLLPLALLLHAARPQVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIKEQKLISEEDLTTTPAPRPPTPAPTIALQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - DNA, 1479 bp, synthetic construct
  misc_feature 1..1479: note = Description of Artificial Sequence: Synthetic polynucleotide
  source 1..1479: mol_type = other DNA
atggctctgcctgtgacagctctgctgctgcctctggctctgcttctgcatgctgccagacctcagatcgtgctgtctcagtctcccgccatcctgtttgctagccctggcgagaaagtgaccatgacctgtagagccagcagctccgtgtcctacatccactggtatcagcagaagcccggcagcagccctaagccttggatccacggcacaagcaatctggccagcggagtgcctgccagattttctggaagcggcagcggcaccagctacagcctgaccatctctagaatggaagccgaggacgccgccacctactactgtcagcagtggtccagcaacctgagcacctttggcggcggaacaaagctggaaatcaaaggcggcggaggatctggcggaggcggtagcggaggtggtggatctgaagtgaagctggtggaatcaggcggaggcctggttcaacctggcggatctctgagactgagctgcagcatctccggcttcaccttcaccgactactacatgaactgggtccgacagagccctggcaaggctctggaatggctgggcttcatcagaaacaaagtgaacggcgacaccaccgagtacagcgcctctgtgaagggcagattcaccatcagccgggacatcagccagagcatcctgtacctgcagatgaacaccctgcggaccgaggatagcgccacatattactgcgccagagacaagggaatcgcctactacttcgactactggggccagggcaccacactgacagtgtctagcgagcagaagctgatctccgaagaggacctgaccacaacaccagctcctagaccacctacaccagcacctacaatcgccctgcagccactgtctctgaggccagaagcttgtagacctgctgctggcggagccgtgcatacaagaggactggatttcgcctgcgacgtggccgccattctcggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatcagagtgaagttcagcagatccgccgacgctcccgcctataagcagggacagaatcagctgtacaacgagctgaacctggggcgcagagaagagtacgacgtgctggacaagagaagaggcagggatcctgaaatgggcggcaagcccagacggaagaatcctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 93 - protein, 493 aa, synthetic construct
  REGION 1..493: note = Description of Artificial Sequence: Synthetic polypeptide
MALPVTALLLPLALLLHAARPQIVLSQSPAILFASPGEKVTMTCRASSSVSYIHWYQQKPGSSPKPWIHGTSNLASGVPARFSGSGSGTSYSLTISRMEAEDAATYYCQQWSSNLSTFGGGTKLEIKGGGGSGGGGSGGGGSEVKLVESGGGLVQPGGSLRLSCSISGFTFTDYYMNWVRQSPGKALEWLGFIRNKVNGDTTEYSASVKGRFTISRDISQSILYLQMNTLRTEDSATYYCARDKGIAYYFDYWGQGTTLTVSSEQKLISEEDLTTTPAPRPPTPAPTIALQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - DNA, 1479 bp, synthetic construct
  misc_feature 1..1479: note = Description of Artificial Sequence: Synthetic polynucleotide
  source 1..1479: mol_type = other DNA
atggctctgcctgtgacagctctgctgctgcctctggctctgcttctgcatgctgctagacctcaggtccagctgcagcagtctggcgccgaacttgttagacctggcaccagcgtgaaggtgtcctgtaaagccagcggctacgccttcaccaacaacctgatcgagtgggtcaagcagaggcctggacagggactcgagtggatcggcgtgatcaatcctggcagcggcgacaccaactacaacgagaagttcaagggcaaagccacactgaccgccgacaagagcagcaacacagcctacatgcagctgagcagcctgacctccgatgacagcgccgtgtacttttgcgccagaggcgattactacggcggcttcgccgtggattattggggccagggaacaagcgtgaccgtgtctagcggaggcggaggatctggtggcggaggaagtggcggaggcggttctgatatccagatgacccagaccaccagcagcctgtctgcctctctgggcgatagagtgaccatcagctgcagaaccagccaggacatcggcaactacctgaactggtatcagcagaaacccgacggcaccgtgaagctgctgatctactacaccagcagactgcacagcggcgtgcccagcagattttctggaagcggcagcggcaccgactactccctgacaatcagcaacctggaacaagaggatatcgctacctacttctgccagcaaggcaagagcctgcctagaacctttggaggcggcaccaagctggaaatcaaagagcagaagctgatctccgaagaggacctgaccacaacaccagctcctagacctccaactcctgctcctacaatcgccctgcagccactgtctctgaggcctgaagcttgtagacctgctgctggcggagccgtgcataccagaggactggatttcgcctgcgacgtggccgccattctcggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatcagagtgaagttcagcagatccgccgacgctcccgcctataagcagggacagaatcagctgtacaacgagctgaacctggggcgcagagaagagtacgacgtgctggacaagagaagaggcagggatcctgaaatgggcggcaagcccagacggaagaatcctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 95 - protein, 493 aa, synthetic construct
  REGION 1..493: note = Description of Artificial Sequence: Synthetic polypeptide
MALPVTALLLPLALLLHAARPQVQLQQSGAELVRPGTSVKVSCKASGYAFTNNLIEWVKQRPGQGLEWIGVINPGSGDTNYNEKFKGKATLTADKSSNTAYMQLSSLTSDDSAVYFCARGDYYGGFAVDYWGQGTSVTVSSGGGGSGGGGSGGGGSDIQMTQTTSSLSASLGDRVTISCRTSQDIGNYLNWYQQKPDGTVKLLIYYTSRLHSGVPSRFSGSGSGTDYSLTISNLEQEDIATYFCQQGKSLPRTFGGGTKLEIKEQKLISEEDLTTTPAPRPPTPAPTIALQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - DNA, 1473 bp, synthetic construct
  misc_feature 1..1473: note = Description of Artificial Sequence: Synthetic polynucleotide
  source 1..1473: mol_type = other DNA
atggctctgcctgtgacagctctgctgctgcctctggctctgcttctgcatgccgccagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgagcagaagctgatctccgaagaggacctgaccacaacaccagctcctagaccacctacaccagcacctacaatcgccctgcagccactgtctctgaggccagaggcttgtagacctgctgctggcggagccgtgcatacaagaggactggatttcgcctgcgacgtggccgccattctcggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatcagagtgaagttcagcagatccgccgacgctcccgcctataagcagggacagaatcagctgtacaacgagctgaacctggggcgcagagaagagtacgacgtgctggacaagagaagaggcagggatcctgaaatgggcggcaagcccagacggaagaatcctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 97 - protein, 491 aa, synthetic construct; identical to SEQ ID NO: 89
  REGION 1..491: note = Description of Artificial Sequence: Synthetic polypeptide

SEQ ID NO: 98 - DNA, 1746 bp, synthetic construct
  misc_feature 1..1746: note = Description of Artificial Sequence: Synthetic polynucleotide
  source 1..1746: mol_type = other DNA
atggctctgcctgtgacagctctgctgctgcctctggctctgcttctgcatgctgccagacctcagaccgtgctgacacagagcccaagcagcctgtctgtgtccgtgggcgacagagtgaccatcacctgtagagccagcagctccgtgacctacatccactggtatcagcagaagcccggactggcccctaagagcctgatctacgccacaagcaatctggccagcggcgtgccaagcagattttctggaagcggcagcggcaccgactacaccttcaccatatctagcctgcagcctgaggatatcgccacctactactgccagcactggtccagcaagcctccaacatttggccagggcaccaaggtggaagtgaagagaacagttggcggcggaggatctggcggaggcggtagcggaggtggtggatctgaagttcagctgctggaatcaggcggcggactggttcaacctggcggatctctgagactgagctgtgccaccagcggcttcacctttaccgactactacatgaactgggtccgacaggcccctggcaaaggactggaatggctgggcttcatcggcaacaaggccaacggctacaccaccgagtacagcgcctctgtgaagggcagattcaccatcagccgggacaagagcaagagcaccctgtacctgcagatgaacaccctgcaggccgaggactccgccatctactattgcaccagagacagaggcctgcggttctacttcgactattggggccagggaaccctggtcacagtgtctagcgcctctacaaagggccccagcgttttcccactggctccctgtagcagaagcaccagcgaatctacagccgctctgggctgcctggtcaaggactactttcctgagccagtgacagtgctgtatctccagatgaatactctccaagctgaggacagcgccatctattactgtacgcgcgatcggggcctgagattctactttgattactggggacaaggcaccctcgtgaccgtgtcctctgagcagaagctgatctccgaagaggacctgaatggcgccgctgccgctgctatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttattccctgctggttaccgtggccttcatcatcttttgggtccgaagcaagcggagcagactgctgcactccgactacatgaacatgacccctagacggcccggaccaaccagaaagcactaccagccttacgctcctcctagagacttcgccgcctaccggtccagagtgaagttctccagatccgccgatgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 99 - protein, 582 aa, synthetic construct
  REGION 1..582: note = Description of Artificial Sequence: Synthetic polypeptide
MALPVTALLLPLALLLHAARPQTVLTQSPSSLSVSVGDRVTITCRASSSVTYIHWYQQKPGLAPKSLIYATSNLASGVPSRFSGSGSGTDYTFTISSLQPEDIATYYCQHWSSKPPTFGQGTKVEVKRTVGGGGSGGGGSGGGGSEVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPLAPCSRSTSESTAALGCLVKDYFPEPVTVLYLQMNTLQAEDSAIYYCTRDRGLRFYFDYWGQGTLVTVSSEQKLISEEDLNGAAAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 1497 bp, synthetic construct
  misc_feature 1..1497: note = Description of Artificial Sequence: Synthetic polynucleotide
  source 1..1497: mol_type = other DNA
atggctctgcctgtgacagctctgctgctgcctctggctctgcttctgcatgctgccagacctgaggtccagctgcaagagtctggccctggacttgtgaagcctggcggaagcctgtctctgtcttgtgccgccagcggcttcgtgttcagcagctacgatatgagctgggtccgacagacccctgagcgaggacttgagtgggtcgcctacatctcttctggcggcggaatcacatacgcccctagcacagtgaagggcagattcaccgtgtccagagacaacgccaagaacaccctgtacctgcagatgaacagcctgaccagcgaggacaccgccgtgtactattgtgccgctcactactttggcagcagcggcccttttgcctattggggccagggaacactcgtgaccgtttctagtgctggcggaggcggatcaggtggcggaggaagtggcggcggaggatctgatatccagatgacacagagccccgccagcctgtctgcctctgtgggagatagagtgaccatcacctgtcgggccagcgagaacatcttcagctacctggcctggtatcagcagaagcccggcaagtctcctaagctgctggtgtacaacacccggacactggctgaaggcgtgcccagcagattttctggcagcggctctggcaccgacttcagcctgacaatcagcagcctgcagcctgaggacttcgccacctactactgccagcaccactacggcacccctttcacctttggaagcggcaccaagctggaaatcaagagagagcagaagctgatctccgaagaggacctgaatggcgccgctaccacaacaccagctcctagacctccaactcctgctcctacaatcgccctgcagccactgtctctgaggcctgaagcttgtagacctgctgctggcggagccgtgcataccagaggactggatttcgcctgcgacatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 101 - protein, 499 aa, synthetic construct
  REGION 1..499: note = Description of Artificial Sequence: Synthetic polypeptide
MALPVTALLLPLALLLHAARPEVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSAGGGGSGGGGSGGGGSDIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKREQKLISEEDLNGAATTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 1497 bp, synthetic construct
  misc_feature 1..1497: note = Description of Artificial Sequence: Synthetic polynucleotide
  source 1..1497: mol_type = other DNA
atggctctgcctgtgacagctctgctgctgcctctggctctgcttctgcatgctgccagacctgacatccagatgacacagagccctgccagcctgtctgcctctgtgggagacagagtgaccatcacctgtcgggccagcgagaacatcttcagctacctggcctggtatcagcagaagcccggcaagtctcctaagctgctggtgtacaacacccggacactggctgaaggcgtgcccagcagattttctggcagcggcagcggaaccgacttcagcctgacaatcagcagcctgcagcctgaggacttcgccacctactactgccagcaccactacggcacccctttcacctttggctctggcaccaagctggaaatcaagagaggcggcggaggatctggcggaggtggaagtggcggaggcggatctgaagttcagctgcaagagtctggccctggcctcgtgaaacctggcggatctctgtctctgagctgtgccgccagcggcttcgtgttcagcagctacgatatgagctgggtccgacagacccctgagcgaggacttgagtgggtcgcctacatctcttctggcggcggaatcacatacgcccctagcacagtgaagggcagattcaccgtgtccagagacaacgccaagaacaccctgtacctgcagatgaacagcctgaccagcgaggacaccgccgtgtactattgtgccgctcactactttggcagcagcggcccttttgcctattggggccagggaacactcgtgaccgtgtctagcgccgagcagaagctgatctccgaagaggacctgaatggcgccgctaccacaacaccagctcctagacctccaactcctgctcctacaatcgccctgcagccactgtctctgaggcctgaagcttgtagacctgctgctggcggagccgtgcataccagaggactggatttcgcctgcgacatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 103 - protein, 499 aa, synthetic construct
  REGION 1..499: note = Description of Artificial Sequence: Synthetic polypeptide
MALPVTALLLPLALLLHAARPDIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRGGGGSGGGGSGGGGSEVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSAEQKLISEEDLNGAATTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - DNA, 1740 bp, synthetic construct
  misc_feature 1..1740: note = Description of Artificial Sequence: Synthetic polynucleotide
  source 1..1740: mol_type = other DNA
atggctctgcctgtgacagctctgctgctgcctctggctctgcttctgcatgctgccagacctgaagtgcagctgctggaatctggcggaggactggttcaacctggcggctctctgagactgagctgtgccacaagcggcttcaccttcaccgactactacatgaactgggtccgacaggcccctggcaaaggactggaatggctgggcttcatcggcaacaaggccaacggctacaccaccgagtacagcgcctctgtgaagggcagattcaccatcagccgggacaagagcaagagcaccctgtacctgcagatgaacaccctgcaggccgaggactccgccatctactactgcaccagagacagaggcctgcggttctacttcgactattggggccagggcaccctggtcacagtgtctagcgcctctacaaagggccccagcgttttcccactggctccctgtagcagaagcaccagcgaatctacagccgctctgggctgcctggtcaaggactactttcctgagcctgtgaccgtgctgtatctccagatgaatactctccaggctgaagatagcgccatatattactgtacgcgcgaccggggcctgagattctactttgattactggggacaagggaccctcgtgaccgtttcttctggcggcggaggaagcggaggcggaggatccggtggtggtggatctcagacagtgctgacacagagccctagcagcctgtctgtgtccgtgggcgacagagtgaccatcacctgtagagccagcagctccgtgacctacatccactggtatcagcagaagcccggactggcccctaagagcctgatctacgccacaagcaatctggccagcggcgtgccaagcagattttctggaagcggcagcggcaccgactatacctttacaatcagcagcctgcagcctgaggatatcgccacctactattgccagcactggtccagcaagcctccaacctttggacagggcaccaaggtggaagtgaagcggaccgtggagcagaagctgatctccgaagaggacctgaatggcgccgctaccacaacaccagctcctagacctccaactcctgctcctacaatcgccctgcagccactgtctctgaggcctgaagcttgtagacctgctgctggcggagccgtgcataccagaggactggatttcgcctgcgacatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 105 - protein, 580 aa, synthetic construct
  REGION 1..580: note = Description of Artificial Sequence: Synthetic polypeptide
MALPVTALLLPLALLLHAARPEVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPLAPCSRSTSESTAALGCLVKDYFPEPVTVLYLQMNTLQAEDSAIYYCTRDRGLRFYFDYWGQGTLVTVSSGGGGSGGGGSGGGGSQTVLTQSPSSLSVSVGDRVTITCRASSSVTYIHWYQQKPGLAPKSLIYATSNLASGVPSRFSGSGSGTDYTFTISSLQPEDIATYYCQHWSSKPPTFGQGTKVEVKRTVEQKLISEEDLNGAATTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - DNA, 1740 bp, synthetic construct
  misc_feature 1..1740: note = Description of Artificial Sequence: Synthetic polynucleotide
  source 1..1740: mol_type = other DNA
atggctctgcctgtgacagctctgctgctgcctctggctctgcttctgcatgctgccagacctcagaccgtgctgacacagagcccaagcagcctgtctgtgtccgtgggcgacagagtgaccatcacctgtagagccagcagctccgtgacctacatccactggtatcagcagaagcccggactggcccctaagagcctgatctacgccacaagcaatctggccagcggcgtgccaagcagattttctggaagcggcagcggcaccgactacaccttcaccatatctagcctgcagcctgaggatatcgccacctactactgccagcactggtccagcaagcctccaacatttggccagggcaccaaggtggaagtgaagagaacagttggcggcggaggatctggcggaggcggtagcggaggtggtggatctgaagttcagctgctggaatcaggcggcggactggttcaacctggcggatctctgagactgagctgtgccaccagcggcttcacctttaccgactactacatgaactgggtccgacaggcccctggcaaaggactggaatggctgggcttcatcggcaacaaggccaacggctacaccaccgagtacagcgcctctgtgaagggcagattcaccatcagccgggacaagagcaagagcaccctgtacctgcagatgaacaccctgcaggccgaggactccgccatctactattgcaccagagacagaggcctgcggttctacttcgactattggggccagggaaccctggtcacagtgtctagcgcctctacaaagggccccagcgttttcccactggctccctgtagcagaagcaccagcgaatctacagccgctctgggctgcctggtcaaggactactttcctgagccagtgacagtgctgtatctccagatgaatactctccaagctgaggacagcgccatctattactgtacgcgcgatcggggcctgagattctactttgattactggggacaaggcaccctcgtgaccgtgtcctctgagcagaagctgatctccgaagaggacctgaatggcgccgctaccacaacaccagctcctagacctccaactcctgctcctacaatcgccctgcagccactgtctctgaggcctgaagcttgtagacctgctgctggcggagccgtgcataccagaggactggatttcgcctgcgacatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 107 - protein, 580 aa, synthetic construct
  REGION 1..580: note = Description of Artificial Sequence: Synthetic polypeptide
MALPVTALLLPLALLLHAARPQTVLTQSPSSLSVSVGDRVTITCRASSSVTYIHWYQQKPGLAPKSLIYATSNLASGVPSRFSGSGSGTDYTFTISSLQPEDIATYYCQHWSSKPPTFGQGTKVEVKRTVGGGGSGGGGSGGGGSEVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPLAPCSRSTSESTAALGCLVKDYFPEPVTVLYLQMNTLQAEDSAIYYCTRDRGLRFYFDYWGQGTLVTVSSEQKLISEEDLNGAATTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 500 aa, synthetic construct
  REGION 1..500: note = Description of Artificial Sequence: Synthetic polypeptide
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - DNA, 1473 bp, synthetic construct
  misc_feature 1..1473: note = Description of Artificial Sequence: Synthetic polynucleotide
  source 1..1473: mol_type = other DNA
atggctctgcctgtgacagctctgctgctgcctctggctctgcttctgcatgccgccagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgagcagaagctgatctccgaagaggacctgaccacaacaccagctcctagacctccaactcctgctcctacaatcgccctgcagccactgtctctgaggcctgaagcttgtagacctgctgctggcggagccgtgcataccagaggactggatttcgcctgcgacatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 110 - protein, 495 aa, synthetic construct
  REGION 1..495: note = Description of Artificial Sequence: Synthetic polypeptide
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - DNA, 1500 bp, synthetic construct
  misc_feature 1..1500: note = Description of Artificial Sequence: Synthetic polynucleotide
  source 1..1500: mol_type = other DNA
atggctctgcctgtgacagctctgctgctgcctctggctctgcttctgcatgccgctagacctgaggtccagctgcaagagtctggccctggacttgtgaagcctggcggaagcctgtctctgtcttgtgccgccagcggcttcgtgttcagcagctacgatatgagctgggtccgacagacccctgagcgaggacttgagtgggtcgcctacatctcttctggcggcggaatcacatacgcccctagcacagtgaagggcagattcaccgtgtccagagacaacgccaagaacaccctgtacctgcagatgaacagcctgaccagcgaggacaccgccgtgtactattgtgccgctcactactttggcagcagcggcccttttgcctattggggccagggaacactcgtgaccgtttctagtgctggcggaggcggatcaggtggcggaggaagtggcggcggaggatctgatatccagatgacacagagccccgccagcctgtctgcctctgtgggagatagagtgaccatcacctgtcgggccagcgagaacatcttcagctacctggcctggtatcagcagaagcccggcaagtctcctaagctgctggtgtacaacacccggacactggctgaaggcgtgcccagcagattttctggcagcggctctggcaccgacttcagcctgacaatcagcagcctgcagcctgaggacttcgccacctactactgccagcaccactacggcacccctttcacctttggaagcggcaccaagctggaaatcaagagagagcagaagctgatctccgaagaggacctgaccacaactcctgctcctagacctcctacaccagctcctacaatcgccctgcagccactgtctctgaggccagaagcttgtagacctgctgcaggcggagccgtgcatacaagaggactggatttcgcctgcgacttctgggtgctcgtggttgttggcggagtgctggcctgttactctctgctggtcaccgtggccttcatcatcttttgggtccgaagcaagcggagccggctgctgcacagcgactacatgaacatgacccctagacggcccggacctaccagaaagcactaccagccttacgctcctcctagagatttcgccgcctaccggtccagagtgaagttcagcagatccgccgatgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 112 - protein, 504 aa, synthetic construct
  REGION 1..504: note = Description of Artificial Sequence: Synthetic polypeptide
MALPVTALLLPLALLLHAARPEVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSAGGGGSGGGGSGGGGSDIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKREQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - DNA, 1485 bp, synthetic construct
  misc_feature 1..1485: note = Description of Artificial Sequence: Synthetic polynucleotide
  source 1..1485: mol_type = other DNA
atggctctgcctgtgacagctctgctgctgcctctggctctgcttctgcatgccgctagacctgaggtccagctgcaagagtctggccctggacttgtgaagcctggcggaagcctgtctctgtcttgtgccgccagcggcttcgtgttcagcagctacgatatgagctgggtccgacagacccctgagcgaggacttgagtgggtcgcctacatctcttctggcggcggaatcacatacgcccctagcacagtgaagggcagattcaccgtgtccagagacaacgccaagaacaccctgtacctgcagatgaacagcctgaccagcgaggacaccgccgtgtactattgtgccgctcactactttggcagcagcggcccttttgcctattggggccagggaacactcgtgaccgtttctagtgctggcggaggcggatcaggtggcggaggaagtggcggcggaggatctgatatccagatgacacagagccccgccagcctgtctgcctctgtgggagatagagtgaccatcacctgtcgggccagcgagaacatcttcagctacctggcctggtatcagcagaagcccggcaagtctcctaagctgctggtgtacaacacccggacactggctgaaggcgtgcccagcagattttctggcagcggctctggcaccgacttcagcctgacaatcagcagcctgcagcctgaggacttcgccacctactactgccagcaccactacggcacccctttcacctttggaagcggcaccaagctggaaatcaagagagagcagaagctgatctccgaagaggacctgaccacaacaccagctcctagaccacctacaccagcacctacaatcgccctgcagccactgtctctgaggccagaggcttgtagacctgctgctggcggagccgtgcatacaagaggactggatttcgcctgcgacgtggccgccattctcggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatcagagtgaagttcagcagatccgccgacgctcccgcctataagcagggacagaatcagctgtacaacgagctgaacctggggcgcagagaagagtacgacgtgctggacaagagaagaggcagggatcctgaaatgggcggcaagcccagacggaagaatcctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 114 - protein, 499 aa, synthetic construct
  REGION 1..499: note = Description of Artificial Sequence: Synthetic polypeptide
MALPVTALLLPLALLLHAARPEVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSAGGGGSGGGGSGGGGSDIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKREQKLISEEDLNGAATTTPAPRPPTPAPTIALQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - DNA, 1707 bp, synthetic construct
  misc_feature 1..1707: note = Description of Artificial Sequence: Synthetic polynucleotide
  source 1..1707: mol_type = other DNA
atggctctgcctgtgacagctctgctgctgcctctggctctgcttctgcatgccgccagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgagcagaagctgatctccgaagaggacctgaccacaacaccagctcctagacctccaactcctgctcctacaatcgccctgcagccactgtctctgaggcctgaagcttgtagacctgctgctggcggagccgtgcataccagaggactggatttcgcctgcgacttcctggtcatcatcgtgatcctgagcgccctgttcctgggcacactggcctgtttctgcgtgtggcgcagaaagcgcaaagagaagcagagcgagacaagccccaaagagttcctgaccatctacgaggacgtgaaggacctgaaaacccggcggaaccacgagcaagagcagacctttcctggcggcggatccaccatctacagcatgatccagagccagagcagcgcccctacaagccaagagcctgcctacacactgtacagcctgattcagcccagcagaaagagcggctcccggaagagaaatcacagccccagcttcaacagcacgatctacgaagtgatcggcaagagccagccaaaggctcagaaccctgccagactgagcagaaaagagctggaaaacttcgacgtgtacagccgcgtgaagttcagcagatccgccgatgctcctgcctatcagcagggccagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 116 - protein, 573 aa, synthetic construct
  REGION 1..573: note = Description of Artificial Sequence: Synthetic polypeptide
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - DNA, 1710 bp, synthetic construct
  misc_feature 1..1710: note = Description of Artificial Sequence: Synthetic polynucleotide
  source 1..1710: mol_type = other DNA
atggctctgcctgtgacagctctgctgctgcctctggctctgcttctgcatgccgctagaccccaagtgaagctggaacagtctggcgccgaggttgtgaaacctggcgcctctgtgaagctgagctgtaaagccagcggcttcaacatcaaggacagctacatgcactggctgcggcaaggccctggacagagactggaatggatcggctggatcgaccccgagaatggcgatacagagtacgcccctaagttccagggcaaagccaccttcaccaccgacacctctgccaacacagcctacctgggactgtctagcctgaggcctgaagataccgccgtgtactactgcaacgagggcacacctacaggcccctactacttcgattattggggccagggcaccctggtcaccgtttctagcggaggcggaggatctggtggcggaggaagtggcggaggcggttctgaaaatgtgctgacacagagccccagcagcatgtccgtgtctgtgggcgacagagtgacaatcgcctgtagcgcctctagcagcgtgccctatatgcattggctgcagcagaagcccggcaagagccctaagctgctgatctacctgaccagcaatctggccagcggcgtgccaagcagattttctggaagcggcagcggcaccgactacagcctgacaatcagtagcgtgcagcctgaggatgccgccacctactactgtcagcagagaagcagctaccctctgacctttggaggcggcaccaagctggaaatcaaagagcagaagctgatctccgaagaggacctgaccacaacaccagctcctagacctccaactcctgctcctacaatcgccctgcagccactgtctctgaggcctgaagcttgtagacctgctgctggcggagccgtgcataccagaggactggatttcgcctgcgacttcctggtcatcatcgtgatcctgagcgccctgttcctgggcacactggcctgtttctgcgtgtggcgcagaaagcgcaaagagaagcagagcgagacaagccccaaagagttcctgaccatctacgaggacgtgaaggacctgaaaacccggcggaaccacgagcaagagcagacctttcctggcggcggatccaccatctacagcatgatccagagccagagcagcgcccctacaagccaagagcctgcctacacactgtacagcctgattcagcccagcagaaagagcggctcccggaagagaaatcacagccccagcttcaacagcacgatctacgaagtgatcggcaagagccagccaaaggctcagaaccctgccagactgagcagaaaagagctggaaaacttcgacgtgtacagccgcgtgaagttcagcagatccgccgatgctcctgcctatcagcagggccagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 118 - protein, 598 aa, synthetic construct
  REGION 1..598: note = Description of Artificial Sequence: Synthetic polypeptide
MALPVTALLLPLALLLHAARPETVIKYLLPTAAAGLLLLAAQPAMAQVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAEQKLISEEDLTTTPAPRPPTPAPTIALQPLSLRPEACRPAAGGAVHTRGLDFACDFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - DNA, 1476 bp, synthetic construct
  misc_feature 1..1476: note = Description of Artificial Sequence: Synthetic polynucleotide
  source 1..1476: mol_type = other DNA
atggctctgcctgtgacagctctgctgctgcctctggctctgcttctgcatgccgctagaccccaagtgaagctggaacagtctggcgccgaggttgtgaaacctggcgcctctgtgaagctgagctgtaaagccagcggcttcaacatcaaggacagctacatgcactggctgcggcaaggccctggacagagactggaatggatcggctggatcgaccccgagaatggcgatacagagtacgcccctaagttccagggcaaagccaccttcaccaccgacacctctgccaacacagcctacctgggactgtctagcctgaggcctgaagataccgccgtgtactactgcaacgagggcacacctacaggcccctactacttcgattattggggccagggcaccctggtcaccgtttctagcggaggcggaggatctggtggcggaggaagtggcggaggcggttctgaaaatgtgctgacacagagccccagcagcatgtccgtgtctgtgggcgacagagtgacaatcgcctgtagcgcctctagcagcgtgccctatatgcattggctgcagcagaagcccggcaagagccctaagctgctgatctacctgaccagcaatctggccagcggcgtgccaagcagattttctggaagcggcagcggcaccgactacagcctgacaatcagtagcgtgcagcctgaggatgccgccacctactactgtcagcagagaagcagctaccctctgacctttggaggcggcaccaagctggaaatcaaagagcagaagctgatctccgaagaggacctgaccacaacaccagctcctagacctccaactcctgctcctacaatcgccctgcagccactgtctctgaggcctgaagcttgtagacctgctgctggcggagccgtgcataccagaggactggatttcgcctgcgacatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 120 - protein, 520 aa, synthetic construct
  REGION 1..520: note = Description of Artificial Sequence: Synthetic polypeptide
MALPVTALLLPLALLLHAARPETVIKYLLPTAAAGLLLLAAQPAMAQVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAEQKLISEEDLTTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - DNA, 1713 bp, synthetic construct
  misc_feature 1..1713: note = Description of Artificial Sequence: Synthetic polynucleotide
  source 1..1713: mol_type = other DNA
atggctctgcctgtgacagctctgctgctgcctctggctctgcttctgcatgctgccagacctcagatcgtgctgtctcagtctcccgccatcctgtttgctagccctggcgagaaagtgaccatgacctgtagagccagcagctccgtgtcctacatccactggtatcagcagaagcccggcagcagccctaagccttggatccacggcacaagcaatctggccagcggagtgcctgccagattttctggaagcggcagcggcaccagctacagcctgaccatctctagaatggaagccgaggacgccgccacctactactgtcagcagtggtccagcaacctgagcacctttggcggcggaacaaagctggaaatcaaaggcggcggaggatctggcggaggcggtagcggaggtggtggatctgaagtgaagctggtggaatcaggcggaggcctggttcaacctggcggatctctgagactgagctgcagcatctccggcttcaccttcaccgactactacatgaactgggtccgacagagccctggcaaggctctggaatggctgggcttcatcagaaacaaagtgaacggcgacaccaccgagtacagcgcctctgtgaagggcagattcaccatcagccgggacatcagccagagcatcctgtacctgcagatgaacaccctgcggaccgaggatagcgccacatattactgcgccagagacaagggaatcgcctactacttcgactactggggccagggcaccacactgacagtgtctagcgagcagaagctgatctccgaagaggacctgaccacaacaccagctcctagaccacctacaccagcacctacaatcgccctgcagccactgtctctgaggccagaagcttgtagacctgctgctggcggagccgtgcatacaagaggactggatttcgcctgcgacttcctggtcatcatcgtgatcctgagcgccctgttcctgggcacactggcctgtttctgcgtgtggcgcagaaagcgcaaagagaagcagagcgagacaagccccaaagagttcctgaccatctacgaggacgtgaaggacctgaaaacccggcggaaccacgagcaagagcagacctttcctggcggcggatccaccatctacagcatgatccagagccagagcagcgcccctacaagccaagagcctgcctacacactgtacagcctgattcagcccagcagaaagagcggctcccggaagagaaatcacagccccagcttcaacagcacgatctacgaagtgatcggcaagagccagccaaaggctcagaaccctgccagactgagcagaaaagagctggaaaacttcgacgtgtacagccgcgtgaagttcagcagatccgccgatgctcctgcctatcagcagggccagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 122 - protein, 571 aa, synthetic construct
  REGION 1..571: note = Description of Artificial Sequence: Synthetic polypeptide
MALPVTALLLPLALLLHAARPQIVLSQSPAILFASPGEKVTMTCRASSSVSYIHWYQQKPGSSPKPWIHGTSNLASGVPARFSGSGSGTSYSLTISRMEAEDAATYYCQQWSSNLSTFGGGTKLEIKGGGGSGGGGSGGGGSEVKLVESGGGLVQPGGSLRLSCSISGFTFTDYYMNWVRQSPGKALEWLGFIRNKVNGDTTEYSASVKGRFTISRDISQSILYLQMNTLRTEDSATYYCARDKGIAYYFDYWGQGTTLTVSSEQKLISEEDLTTTPAPRPPTPAPTIALQPLSLRPEACRPAAGGAVHTRGLDFACDFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1479 bp, synthetic construct
  misc_feature 1..1479: note = Description of Artificial Sequence: Synthetic polynucleotide
  source 1..1479: mol_type = other DNA
atggctctgcctgtgacagctctgctgctgcctctggctctgcttctgcatgctgccagacctcagatcgtgctgtctcagtctcccgccatcctgtttgctagccctggcgagaaagtgaccatgacctgtagagccagcagctccgtgtcctacatccactggtatcagcagaagcccggcagcagccctaagccttggatccacggcacaagcaatctggccagcggagtgcctgccagattttctggaagcggcagcggcaccagctacagcctgaccatctctagaatggaagccgaggacgccgccacctactactgtcagcagtggtccagcaacctgagcacctttggcggcggaacaaagctggaaatcaaaggcggcggaggatctggcggaggcggtagcggaggtggtggatctgaagtgaagctggtggaatcaggcggaggcctggttcaacctggcggatctctgagactgagctgcagcatctccggcttcaccttcaccgactactacatgaactgggtccgacagagccctggcaaggctctggaatggctgggcttcatcagaaacaaagtgaacggcgacaccaccgagtacagcgcctctgtgaagggcagattcaccatcagccgggacatcagccagagcatcctgtacctgcagatgaacaccctgcggaccgaggatagcgccacatattactgcgccagagacaagggaatcgcctactacttcgactactggggccagggcaccacactgacagtgtctagcgagcagaagctgatctccgaagaggacctgaccacaacaccagctcctagacctccaactcctgctcctacaatcgccctgcagccactgtctctgaggcctgaagcttgtagacctgctgctggcggagccgtgcataccagaggactggatttcgcctgcgacatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 124 - protein, 493 aa, synthetic construct
  REGION 1..493: note = Description of Artificial Sequence: Synthetic polypeptide
MALPVTALLLPLALLLHAARPQIVLSQSPAILFASPGEKVTMTCRASSSVSYIHWYQQKPGSSPKPWIHGTSNLASGVPARFSGSGSGTSYSLTISRMEAEDAATYYCQQWSSNLSTFGGGTKLEIKGGGGSGGGGSGGGGSEVKLVESGGGLVQPGGSLRLSCSISGFTFTDYYMNWVRQSPGKALEWLGFIRNKVNGDTTEYSASVKGRFTISRDISQSILYLQMNTLRTEDSATYYCARDKGIAYYFDYWGQGTTLTVSSEQKLISEEDLTTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - DNA, 1500 bp, synthetic construct
  misc_feature 1..1500: note = Description of Artificial Sequence: Synthetic polynucleotide
  source 1..1500: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 126 - protein, 500 aa, synthetic construct; identical to SEQ ID NO: 108
  REGION 1..500: note = Description of Artificial Sequence: Synthetic polypeptide

SEQ ID NO: 127 - DNA, 1485 bp, synthetic construct
  misc_feature 1..1485: note = Description of Artificial Sequence: Synthetic polynucleotide
  source 1..1485: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgtggccgccattcttggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 128 - protein, 495 aa, synthetic construct
  REGION 1..495: note = Description of Artificial Sequence: Synthetic polypeptide
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1512 bp, synthetic construct
  misc_feature 1..1512: note = Description of Artificial Sequence: Synthetic polynucleotide
  source 1..1512: mol_type = other DNA
atggccctgcctgtgacagctctgctgctgcctctggccctgctgctgcatgctgctagacctgaggtccagctgcaagagtctggccctggacttgtgaagcctggcggaagcctgtctctgtcttgtgccgccagcggcttcgtgttcagcagctacgatatgagctgggtccgacagacccctgagcgaggacttgagtgggtcgcctacatctcttctggcggcggaatcacatacgcccctagcacagtgaagggcagattcaccgtgtccagagacaacgccaagaacaccctgtacctgcagatgaacagcctgaccagcgaggacaccgccgtgtactattgtgccgctcactactttggcagcagcggcccttttgcctattggggccagggaacactcgtgaccgtttctagtgctggcggaggcggatcaggtggcggaggaagtggcggcggaggatctgatatccagatgacacagagccccgccagcctgtctgcctctgtgggagatagagtgaccatcacctgtcgggccagcgagaacatcttcagctacctggcctggtatcagcagaagcccggcaagtctcctaagctgctggtgtacaacacccggacactggctgaaggcgtgcccagcagattttctggcagcggctctggcaccgacttcagcctgacaatcagcagcctgcagcctgaggacttcgccacctactactgccagcaccactacggcacccctttcacctttggaagcggcaccaagctggaaatcaagagagaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 130 - DNA, 1497 bp, synthetic construct
  misc_feature 1..1497: note = Description of Artificial Sequence: Synthetic polynucleotide
  source 1..1497: mol_type = other DNA
atggccctgcctgtgacagctctgctgctgcctctggccctgctgctgcatgctgctagacctgaggtccagctgcaagagtctggccctggacttgtgaagcctggcggaagcctgtctctgtcttgtgccgccagcggcttcgtgttcagcagctacgatatgagctgggtccgacagacccctgagcgaggacttgagtgggtcgcctacatctcttctggcggcggaatcacatacgcccctagcacagtgaagggcagattcaccgtgtccagagacaacgccaagaacaccctgtacctgcagatgaacagcctgaccagcgaggacaccgccgtgtactattgtgccgctcactactttggcagcagcggcccttttgcctattggggccagggaacactcgtgaccgtttctagtgctggcggaggcggatcaggtggcggaggaagtggcggcggaggatctgatatccagatgacacagagccccgccagcctgtctgcctctgtgggagatagagtgaccatcacctgtcgggccagcgagaacatcttcagctacctggcctggtatcagcagaagcccggcaagtctcctaagctgctggtgtacaacacccggacactggctgaaggcgtgcccagcagattttctggcagcggctctggcaccgacttcagcctgacaatcagcagcctgcagcctgaggacttcgccacctactactgccagcaccactacggcacccctttcacctttggaagcggcaccaagctggaaatcaagagagaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgtggccgccattctcggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 131 - DNA, 1488 bp, synthetic construct
  misc_feature 1..1488: note = Description of Artificial Sequence: Synthetic polynucleotide
  source 1..1488: mol_type = other DNA
atggctctgcctgtgacagctctgctgctgcctctggctctgcttctgcatgccgccagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgagcagaagctgatctccgaagaggacctgaccacaacaccagctcctagaccacctacaccagcacctacaatcgccctgcagccactgtctctgaggcctgaagcttgtagacctgctgctggcggagccgtgcatacaagaggactggatttcgcctgcgacttctgggtgctcgttgttgttggcggcgtgctggcctgttattccctgctggttaccgtggccttcatcatcttttgggtccgaagcaagcggagcagactgctgcactccgactacatgaacatgacccctagacggcccggaccaaccagaaagcactaccagccttacgctcctcctagagacttcgccgcctaccggtccagagtgaagttctccagatccgccgatgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 132 - DNA, 1473 bp, synthetic construct; identical to SEQ ID NO: 96
  misc_feature 1..1473: note = Description of Artificial Sequence: Synthetic polynucleotide
  source 1..1473: mol_type = other DNA

SEQ ID NO: 133 - DNA, 1500 bp, synthetic construct; identical to SEQ ID NO: 111
  misc_feature 1..1500: note = Description of Artificial Sequence: Synthetic polynucleotide
  source 1..1500: mol_type = other DNA

SEQ ID NO: 134 - DNA, 1485 bp, synthetic construct; identical to SEQ ID NO: 113
  misc_feature 1..1485: note = Description of Artificial Sequence: Synthetic polynucleotide
  source 1..1485: mol_type = other DNA

SEQ ID NO: 135 - protein, 243 aa, synthetic construct
  REGION 1..243: note = Description of Artificial Sequence: Synthetic polypeptide
DIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSS

SEQ ID NO: 136 - DNA, 1473 bp, synthetic construct; identical to SEQ ID NO: 96
  misc_feature 1..1473: note = Description of Artificial Sequence: Synthetic polynucleotide
  source 1..1473: mol_type = other DNA

SEQ ID NO: 137 - DNA, 1488 bp, synthetic construct; identical to SEQ ID NO: 131
  misc_feature 1..1488: note = Description of Artificial Sequence: Synthetic polynucleotide
  source 1..1488: mol_type = other DNA

SEQ ID NO: 138 - protein, 5 aa, synthetic construct
  REGION 1..5: note = Description of Artificial Sequence: Synthetic polypeptide
  REGION >5: note = The entire sequence of amino acids 1-5 can be repeated two to six times
GGGGS

SEQ ID NO: 139 - protein, 5 aa, synthetic construct
  REGION 1..5: note = Description of Artificial Sequence: Synthetic peptide
  REGION 1..5: note = See specification as filed for detailed description of substitutions and preferred embodiments
GGGGS

SEQ ID NO: 140 - protein, 4 aa, synthetic construct
  REGION 1..4: note = Description of Artificial Sequence: Synthetic peptide
  REGION 1..4: note = See specification as filed for detailed description of substitutions and preferred embodiments
GGGS